(19) United States
(12) Patent Application Publication
(10) Pub. No.: US 2024/0110201 A1
(43) Pub. Date: Apr. 4, 2024

(54) Compositions and Methods for Treating Hereditary Angioedema

(71) Applicant: Spark Therapeutics, Inc., Philadelphia, PA (US)

(72) Inventors: Christopher Riling, Exton, PA (US); Francis Pankowicz, Philadelphia, PA (US); Sean Armour, Holland, PA (US); William John Quinn, III, Philadelphia, PA (US); Michael Preston, Downingtown, PA (US); Stephen Ioele, Marlton, NJ (US); Daniel Cohen, Philadelphia, PA (US); Zhenwei Kelvin See, Wayne, PA (US)

(21) Appl. No.: 18/262,959

(22) PCT Filed: Jan. 27, 2022
(86) PCT No.: PCT/US2022/014091

§ 371 Date: Jul. 26, 2023

Related U.S. Application Data
(60) Provisional application No. 63/142,121, filed on Jan. 27, 2021.
(60) Provisional application No. 63/201,466, filed on Apr. 30, 2021.
(60) Provisional application No. 63/261,603, filed on Sep. 24, 2021.

(51) Int. Cl. C12N 15/864; C12N 15/62

(52) U.S. Cl. CPC C12N 15/8645; C12N 15/625; C12N 2830/001; C12N 2830/50

(57) ABSTRACT

Nucleic acids encoding C1 inhibitor are described. Also described are expression cassettes, vectors, cells, and cell lines containing the nucleic acids, as well as methods of using the nucleic acids to treat complement-mediated disorders, such as hereditary angioedema.

### CROSS REFERENCE TO RELATED APPLICATIONS

This application claims priority to U.S. Provisional Patent Application No. 63/142,121 filed Jan. 27, 2021, U.S. Provisional Patent Application No. 63/201,466 filed Apr. 30, 2021, and U.S. Provisional Patent Application No. 63/261,603 filed Sep. 24, 2021. The entire contents of the foregoing applications are incorporated herein by reference, including all text, tables, sequence listings, and drawings.

### FIELD OF THE INVENTION

The instant invention relates to the field of gene therapy. In particular, the instant invention relates to optimized cassettes for expression of human C1 inhibitor and methods of using the same for treating complement-mediated disorders, in particular hereditary angioedema.

### REFERENCE TO SEQUENCE LISTING SUBMITTED ELECTRONICALLY

This application contains a sequence listing that is submitted electronically via EFS-Web as an ASCII formatted sequence listing with a file name “SequenceListing5WO1” and a creation date of Jan. 27, 2022, and having a size of 727 kb. The sequence listing submitted via EFS-Web is part of the specification and is herein incorporated by reference in its entirety.

### BACKGROUND OF THE INVENTION

Hereditary angioedema (HAE), also referred to as C1 esterase inhibitor deficiency, C1 inhibitor deficiency, HANE, Quincke edema, and secondary angioneurotic edema, is a rare and potentially life-threatening autosomal dominant genetic disease that occurs in about 1 in 50,000 people. HAE is characterized by recurrent episodes of swelling that most often affect the skin or mucosal tissues of the upper respiratory and gastrointestinal tracts (Banerji, Ann Allergy Asthma Immunol, 111: 329-336 (2013); Aygoren-Pursun et al., Orphanet J Rare Dis., 9: 99 (2014)).

HAE occurs when plasma leaks through the blood vessels into tissues as a result of the overproduction of bradykinin. It is thought that the disease mechanism involves cleavage of prekallikrein (PKK) by activated factor XII (F12), and releasing active plasma kallikrein, which activates more F12. Plasma kallikrein then cleaves kininogen, releasing bradykinin, which binds to the B2 bradykinin receptor on endothelial cells, increasing the permeability of the endothelium. Normally, the C1 esterase inhibitor (encoded by the SERPING1 gene) controls bradykinin production by inhibiting plasma kallikrein and the activation of F12. (Busse et al., N Engl J Med., 382(12): 1136-1148 (2020)).

There are two types of HAE: HAE-C1-INH and HAE-nl-C1-INH. HAE-C1-INH results from mutations in the SERPING1 gene (encoding C1 inhibitor), resulting in low levels of active C1 inhibitor in the blood. Two subtypes of HAE-C1-INH exist: Type 1 HAE accounts for 85% of cases and is caused by the inability of the mutated C1 inhibitor protein to be secreted into the blood, and Type II HAE accounts for the remaining 15% of cases and is caused by a secreted but dysfunctional mutated C1 inhibitor. HAE-nl-C1-INH was first described in 2000 and is not yet fully understood. Some patients with HAE-nlC1-INH have a mutation in coagulation factor XII, but most patients do not have a known mutation, and the C1 inhibitor gene is normal. (website: www.angioedemacenter.com/patient-resources/angioedema-types/).

Currently, therapeutic agents are indicated for long-term prophylaxis, therapy for acute attacks and short-term prophylaxis (e.g., prior to dental surgery), and include agents such as danazol, which has a high adverse effect profile, plasma-derived or recombinant C1 inhibitor replacement protein, bradykinin receptor antagonists (such as icatibant), kallikrein inhibitors (such as ecallantide), fresh frozen plasma, and purified C1 inhibitor. These therapies can alleviate symptoms and maximize quality of life; however, disease recurrence and the need for long-term continued administration remains a major obstacle to therapy (Aberer, Ann Med, 44: 523-529 (2012); Charignon et al., Expert Opin Pharmacother, 13: 2233-2247 (2012); Papadopoulou-Alataki, Curr Opin Allergy Clin Immunol, 10: 20-25 (2010); Parikh et al., Curr Allergy Asthma Rep, 11: 300-308 (2011); Tourangeau et al., Curr Allergy Asthma Rep, 11: 345-351 (2011); Bowen et al., Ann Allergy Asthma Immunol, 100: S30-S40 (2008); Frank, Immunol Allergy Clin North Am, 26: 653-668 (2006); Cicardi et al., J Allergy Clin Immunol, 99: 194-196 (1997); Kreuz et al., Transfusion 49: 1987-1995 (2009); Bork et al., Ann Allergy Asthma Immunol, 100: 153-161 (2008); and Cicardi et al., JAllergy Clin Immunol, 87: 768-773 (1991)).

There is a need for effective and long-lasting therapeutic approaches to treat angioedema associated with C1 inhibitor and F12 deficiency.

### BRIEF SUMMARY OF THE INVENTION

Disclosed herein are optimized cassettes for liver-directed expression of a secretable version of human C1 inhibitor. These optimizations to the cassettes lead to an increase in C1 inhibitor secretion from liver and enable hepatic gene transfer to achieve circulating levels of C1 inhibitor sufficient to cross-correct C1 inhibitor deficiency systemically in subjects. These cassettes are useful as a gene therapy treatment of subjects with hereditary angioedema (HAE) and other diseases and disorders treatable with C1 inhibitor.

In one general aspect, the instant invention relates to a polynucleotide comprising a nucleic acid encoding a C1 inhibitor, wherein the nucleic acid is selected from the group consisting of: (1) a polynucleotide having at least 83% sequence identity to the sequence of SEQ ID NO: 105; (2) a polynucleotide having at least 83% sequence identity to the sequence of SEQ ID NO: 106; (3) a polynucleotide having at least 80% sequence identity to the sequence of SEQ ID NO: 107; (4) a polynucleotide having at least 80% sequence identity to the sequence of SEQ ID NO: 108; (5) a polynucleotide having at least 83% sequence identity to the sequence of SEQ ID NO: 109; (6) a polynucleotide having at least 84% sequence identity to the sequence of SEQ ID NO: 110; (7) a polynucleotide having at least 80% sequence identity to the sequence of SEQ ID NO: 111; (8) a polynucleotide having at least 83% sequence identity to the sequence of SEQ ID NO: 112; (9) a polynucleotide having at least 82% sequence identity to the sequence of SEQ ID NO: 113; (10) a polynucleotide having at least 82% sequence identity to the sequence of SEQ ID NO: 114; (11) a polynucleotide having at least 82% sequence identity to the sequence of SEQ ID NO: 115; (12) a polynucleotide having at least 80% sequence identity to the sequence of SEQ ID NO: 116; (13) a polynucleotide having at least 80% sequence identity to the sequence of SEQ ID NO: 117; (14) a polynucleotide having at least 83% sequence identity to the sequence of SEQ ID NO: 118; (15) a polynucleotide having at least 80% sequence identity to the sequence of SEQ ID NO: 119; (16) a polynucleotide having at least 80% sequence identity to the sequence of SEQ ID NO: 120; (17) a polynucleotide having at least 80% sequence identity to the sequence of SEQ ID NO: 121; (18) a polynucleotide having at least 83% sequence identity to the sequence of SEQ ID NO: 122; (19) a polynucleotide having at least 93% sequence identity to the sequence of SEQ ID NO: 123; (20) a polynucleotide having at least 92% sequence identity to the sequence of SEQ ID NO: 124; (21) a polynucleotide having at least 89% sequence identity to the sequence of SEQ ID NO: 125; (22) a polynucleotide having at least 86% sequence identity to the sequence of SEQ ID NO: 126; (23) a polynucleotide having at least 92% sequence identity to the sequence of SEQ ID NO: 127; (24) a polynucleotide having at least 89% sequence identity to the sequence of SEQ ID NO: 128; (25) a polynucleotide having at least 89% sequence identity to the sequence of SEQ ID NO: 129; (26) a polynucleotide having at least 91% sequence identity to the sequence of SEQ ID NO: 130; (27) a polynucleotide having at least 92% sequence identity to the sequence of SEQ ID NO: 131; (28) a polynucleotide having at least 93% sequence identity to the sequence of SEQ ID NO: 132; (29) a polynucleotide having at least 93% sequence identity to the sequence of SEQ ID NO: 133; (30) a polynucleotide having at least 87% sequence identity to the sequence of SEQ ID NO: 134; (31) a polynucleotide having at least 89% sequence identity to the sequence of SEQ ID NO: 135; (32) a polynucleotide having at least 93% sequence identity to the sequence of SEQ ID NO: 136; (33) a polynucleotide having at least 93% sequence identity to the sequence of SEQ ID NO: 137; (34) a polynucleotide having at least 87% sequence identity to the sequence of SEQ ID NO: 138; (35) a polynucleotide having at least 86% sequence identity to the sequence of SEQ ID NO: 139; (36) a polynucleotide having at least 86% sequence identity to the sequence of SEQ ID NO: 140; and (37) a polynucleotide having at least 86% sequence identity to the sequence of SEQ ID NO: 141, optionally, the C1 inhibitor comprises the amino acid sequence of SEQ ID NO: 181 (mature C1 inhibitor; no signal peptide) or 192 (nascent C1 inhibitor; includes signal peptide).

In certain embodiments, the nucleic acid contains fewer than 24 CpG dinucleotides, optionally 0 CpG dinucleotides.

In certain embodiments, the nucleic acid encoding the C1 inhibitor has a sequence at least 85%, at least 90%, at least 95% or 100% to any one of SEQ ID NOs: 105-142, 145-147, 156, 171, 172, 230 and 231.

In certain embodiments, the instant invention relates to a nucleic acid encoding a variant C1 inhibitor, wherein the variant C1 inhibitor comprises a truncated C1 inhibitor, a fusion of two or more C1 inhibitors, or a fusion of a C1 inhibitor with an Fc region or domain.

In certain embodiments, the nucleic acid has a sequence of any one of SEQ ID NOs: 143-144, 158, and 165-170, optionally the variant C1 inhibitor comprises an amino acid sequence of any one of SEQ ID NOs: 193-201, or a variant thereof.

In certain embodiments, the polynucleotide comprises a signal peptide sequence positioned at the 5′ end of the nucleic acid encoding the C1 inhibitor.

In certain embodiments, the signal peptide sequence is a heterologous signal peptide sequence.

In certain embodiments, the signal peptide sequence is an endogenous or native C1 inhibitor signal peptide sequence.

In certain embodiments, the signal peptide is selected from the group consisting of C1 inhibitor signal peptide, human chymotrypsinogen B2 signal peptide, ALB signal peptide, ORM1 signal peptide, TF signal peptide, AMBP signal peptide, LAMP1 signal peptide, BTN2A2 signal peptide, CD300 signal peptide, NOTCH2 signal peptide, STRC signal peptide, AHSG signal peptide, SYN1 signal peptide, SYN2 signal peptide, SYN3 signal peptide, and SYN4 signal peptide, or a variant thereof.

In certain embodiments, the signal peptide has a coding sequence of one of SEQ ID NOs: 84-103, optionally the signal peptide comprises an amino acid sequence of any one of SEQ ID NOs: 203-218, or a variant thereof.

In certain embodiments, the instant invention relates to a signal peptide comprising a sequence of any one of SEQ ID NOs: 215-218.

In certain embodiments, the instant invention relates to a nucleic acid encoding a signal peptide, wherein the nucleic acid comprises a sequence of any one of SEQ ID NOs: 100-103.

In certain embodiments, a signal peptide of the instant invention follows any one, two, three, or all four of the following rules: (1) an amino-terminal N-region of 2-5 amino acids with a net positive charge, (2) a hydrophobic H-region of 6-15 amino acids, (3) a carboxyl-terminal C-region of 5-10 amino acids, with the amino acid in the −3 position from the C-terminus of the signal peptide having no charge, and the amino acid in the −1 position from the C-terminus of the signal peptide containing a short side chain, and (4) a leucine residue at the −10 position from the C-terminus of the signal peptide.

In certain embodiments, the instant invention relates to an expression cassette comprising the polynucleotide comprising the nucleic acid encoding C1 inhibitor, operably linked to an expression control element.

In certain embodiments, the expression control element is a liver-specific expression control element.

In certain embodiments, the expression cassette further comprises one or more tissue specificity elements, wherein a tissue specificity element is a promoter, and wherein the promoter is optionally an hAAT promoter sequence.

In certain embodiments, the expression cassette further comprises one or more potency elements, wherein a potency element is an enhancer or a polyA sequence, and wherein the enhancer is selected from the group consisting of ApoE, 2×ApoE, 4×ApoE, hAAT enhancer, WPRE, WPRE3, and an intron that is optionally a human hemoglobin 3 (HBB)-derived intron, and wherein the polyA sequence is optionally a bovine growth hormone (bGH) polyadenylation (polyA) sequence.

In certain embodiments, the expression control element of the expression cassette is positioned 5′ of the polynucleotide, wherein the expression control element optionally comprises an ApoE/hAAT enhancer/promoter sequence.

In certain embodiments, the expression cassette further comprises one or more response elements, wherein a response element is a miRNA binding site, a regulated Irel-dependent decay (RIDD) sequence that drives degradation of mRNA, an acute phase response element (APRE), or another 5′ or 3′ UTR sequence, and wherein the miRNA binding site is optionally miR-142-3p, the RIDD sequence is selected from the group consisting of 1×RIDD, 3×RIDD, and RIDD1×Blos, the APRE is selected from the group consisting of SERPING1 5′ UTR, APRE 5′ UTR, and SAA2 5′ UTR, and the other 5′ or 3′ UTR sequence is optionally a SAA2 3′ UTR sequence.

In certain embodiments, the tissue specificity element(s), potency element(s), and/or response element(s) are CpG-reduced compared to the wild-type tissue specificity element(s), potency element(s), and/or response element(s).

In certain embodiments, the instant invention relates to an adeno-associated virus (AAV) vector comprising the polynucleotide or expression cassette.

In certain embodiments, the AAV vector comprises: (a) one or more of an AAV capsid, and (b) one or more AAV inverted terminal repeats (ITRs), wherein the AAV ITR(s) flanks the 5′ and/or 3′ terminus of the polynucleotide or the expression cassette.

In certain embodiments, at least one or more of the ITRs of the AAV vector is modified to have reduced CpGs.

In certain embodiments, the AAV vector has a capsid serotype comprising a modified or variant AAV VP1, VP2 and/or VP3 capsid having 90% or more, 95% or more, or 100% sequence identity to AAV1, AAV2, AAV3, AAV4, AAV5, AAV6, AAV7, AAV8, AAV9, AAV10, AAV11, AAV12, Rh10, Rh74 (SEQ ID NO: 188), AAV3B, AAV-2i8, SEQ ID NO: 226, SEQ ID NO: 189, SEQ ID NO: 190, and/or LK03 (SEQ ID NO: 191)

In certain embodiments, the AAV vector comprises one or more ITRs of any of AAV1, AAV2, AAV3, AAV4, AAV5, AAV6, AAV7, AAV8, AAV9, AAV10, AAV11, AAV12, Rh10, Rh74, AAV3B, AAV serotypes, or a combination thereof.

In certain embodiments, the instant invention relates to an AAV vector comprising,
(1) a 5′ AAV ITR, optionally a 5′ ITR of AAV2, optionally a 5′ ITR comprising the polynucleotide sequence of SEQ ID NO: 70 or 72;
(2) one or more tissue specificity elements, wherein the tissue specificity element is a promoter, optionally the promoter comprises the polynucleotide sequence of SEQ ID NO: 79 or 80;
(3) one or more potency elements, wherein the one or more potency elements are enhancers or polyA sequences, optionally wherein the one or more potency elements have a polynucleotide sequence selected from the group consisting of SEQ ID NOs: 225, 74-76, 81-83, and 173-178;
(4) one or more response elements, wherein the one or more response elements are miRNA binding sites, regulated Irel-dependent decay (RIDD) sequences, acute phase response elements (APREs), or a 5′ or 3′ UTR sequence, optionally wherein the one or more response elements have a polynucleotide sequence selected from the group consisting of SEQ ID NOs: 77-78, and 179, 180, and 182-187;
(5) a nucleic acid encoding a signal peptide, optionally the signal peptide comprising an amino acid sequence selected from the group consisting of SEQ ID NOs: 203-218, optionally a polynucleotide sequence selected from the group consisting of SEQ ID NOs: 84-103;
(6) a nucleic acid encoding at least one of a C1 inhibitor, a variant C1 inhibitor, and a fusion or combination thereof,
   a. optionally, a C1 inhibitor having the amino acid sequence of SEQ ID NO: 181 or 192, optionally a polynucleotide sequence selected from the group consisting of SEQ ID NOs: 104-142, 145-147, 156, and 171-172;
   b. optionally, a variant C1 inhibitor having an amino acid sequence selected from the group consisting of SEQ ID NOs: 193-201, optionally a polynucleotide selected from the group consisting of SEQ ID NOs: 143-144, 158, and 165-170; and
(7) a 3′ AAV ITR, optionally a 3′ ITR of AAV2, optionally a 3′ ITR comprising the polynucleotide sequence of SEQ ID NO: 71 or 73.

In certain embodiments, the AAV vector comprises the polynucleotide sequence of one of SEQ ID NOs: 1-69 and 227-229.

In certain embodiments, the instant invention relates to a non-viral vector comprising the polynucleotide or expression cassette.

In certain embodiments, the instant invention relates to a pharmaceutical composition comprising a plurality of the AAV vectors or non-viral vectors in a biologically compatible carrier or excipient.

In certain embodiments, the pharmaceutical composition further comprises empty AAV capsids.

In certain embodiments, the pharmaceutical composition further comprises a surfactant.

In certain embodiments, the instant invention relates to a method of treating a subject in need of C1 inhibitor, comprising administering to the subject a therapeutically effective amount of the polynucleotide, the expression cassette, the AAV vector, the non-viral vector, or the pharmaceutical composition, wherein the C1 inhibitor is expressed in the subject.

In certain embodiments, the subject is human.

In certain embodiments, the subject has hereditary angioedema (HAE).

In certain embodiments, the polynucleotide, expression cassette, AAV vector, non-viral vector, or pharmaceutical composition is administered to the subject intravenously, intra-arterially, intra-cavity, intra-mucosally, or via catheter.

In certain embodiments, the AAV vector is administered to the subject in a range from about 1×10⁸ to about 1×10¹⁴ vector genomes per kilogram (vg/kg) of the weight of the subject.

In certain embodiments, the method reduces, decreases or inhibits one or more symptoms of the need for C1 inhibitor or of HAE; or prevents or reduces progression or worsening of one or more symptoms of the need for C1 inhibitor or of HAE; or stabilizes one or more symptoms of the need for C1 inhibitor or of HAE; or improves one or more symptoms of the need for C1 inhibitor or of HAE.

In certain embodiments, the instant invention relates to a cell comprising a polynucleotide or expression cassette of the instant invention.

In certain embodiments, the instant invention relates to a cell that produces an AAV vector of the instant invention.

In certain embodiments, the instant invention relates to a method of producing an AAV vector of the instant invention, comprising (a) introducing an AAV vector genome comprising the polynucleotide or expression cassette of the instant invention into a packaging helper cell; and (b) culturing the helper cell under conditions to produce the AAV vector.

### BRIEF DESCRIPTION OF THE DRAWINGS

The foregoing summary, as well as the following detailed description of the instant invention, will be better understood when read in conjunction with the appended drawings. It should be understood that the instant invention is not limited to the precise embodiments shown in the drawings.

FIG. 1 shows a schematic of an exemplary expression vector described herein.

FIG. 2 shows graphs depicting the mean±SD C1-INH antigen levels in mouse plasma for the low dose (1.0×10¹² vg/kg, Groups 7-11) (FIG. 2A) and high dose (4.0×10¹² vg/kg, Groups 1-5) (FIG. 2B) vector cohorts of Example 2 as measured by ELISA; the gray shaded region signifies the mean±SD of all PNP measurements (low dose: 170.30±20.36 μg/mL; high dose: 181.0±35.62 μg/mL); values considered above and below the limits of quantitation were excluded from calculations of mean±SD; SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 3 shows graphs depicting the steady-state C1-INH antigen levels in mouse plasma for the low dose (1.0×10¹² vg/kg, Groups 7-11) (FIG. 3A) and high dose (4.0×10¹² vg/kg, Groups 1-5) (FIG. 3B) vector cohorts of Example 2, presented as mean±SD; steady-state was defined as no statistically significant difference in mean antigen levels across assessed timepoints (linear regression analysis not shown); statistical comparison relative to native SERPING1 signal peptide was performed by Kruskal-Wallis test with post-hoc Dunn's multiple comparisons test (ns, not significant; *, p<0.05; **, p<0.01; ***, p<0.001; ****, p<0.0001); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 4 shows a graph depicting C1-INH activity in mouse plasma from the high dose cohort of Example 2, specifically, individual C1-INH activity values are shown for the high dose cohort, with data from Weeks 22, 25, and 27 included (circles, triangles, and squares, respectively), with values presented as mean±SD; statistical comparison relative to native SERPING1 signal peptide was performed by one-way ANOVA with post-hoc Dunnett's multiple comparisons test (****, p<0.0001); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 5 shows a graph depicting correlation of C1-INH antigen with C1-INH activity in mouse plasma from the high dose cohort of Example 2, specifically, individual values for C1-INH activity are plotted relative to each animal's respective C1-INH antigen level, with the C1-INH activity and antigen measured by a chromogenic assay and ELISA, respectively, with data from Weeks 22, 25, and 27; values were log 10 transformed, and a linear fit was generated using regression analysis (R²=0.771), a two-tailed Pearson correlation indicated significance (Pearson r=0.8778; p<0.0001); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 6 shows graphs depicting vector genome concentration from terminal liver tissue of Example 2, with the vector genome copy numbers normalized to mouse Foxp1 in the low dose (FIG. 6A) and high dose (FIG. 6B) cohorts, with values are reported as BGHpA copies per Foxp1 copies and presented as mean±SD; statistical comparison relative to native SERPING1 signal peptide was performed by Kruskal-Wallis test with post-hoc Dunn's multiple comparisons test (**, p<0.01; ***, p<0.001); terminus was defined as Week 18 for the low dose cohort (Groups 7-11) and Week 28 for the high dose cohort (Groups 1-5); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 7 shows a graph depicting correlation of C1-INH antigen with vector genome concentration at terminus of Example 2, with terminal C1-INH antigen values versus vector genome concentration (normalized to Foxp1) at terminus in individual mice; terminus was defined as Week 18 for the lower dose cohort (Groups 7-11) and Week 28 for the higher dose cohort (Groups 1-5); values were log 10 transformed and a linear fit was generated using regression analysis (R²=0.866); a two-tailed Pearson correlation indicated significance (Pearson r=0.9306; p<0.0001); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 8 shows a graph depicting steady state C1-INH antigen levels relative to vector genome concentration at terminus of Example 2, with steady state C1-INH antigen levels plotted with respect to terminal liver vector genome (VG) concentration (normalized to Foxp1) in the low dose (FIG. 8A) and high dose (FIG. 8B) cohorts; steady-state C1-INH antigen was defined as no statistically significant difference in levels across the timepoints tested (statistical analysis not shown, Weeks 3-18 and Weeks 3-28 for low and high dose cohorts, respectively); values are presented as mean±SD; statistical comparison relative to native SERPING1 signal peptide was performed by Kruskal-Wallis test with post-hoc Dunn's multiple comparisons test (*, p<0.05); terminus was defined as Week 18 for the lower dose cohort (Groups 7-11) and Week 28 for the higher dose cohort (Groups 1-5); SERPING1=SEQ ID NO: 1, Syn1=SEQ ID NO: 13, Syn4=SEQ ID NO: 16, AHSG=SEQ ID NO: 12, sp7=SEQ ID NO: 2.

FIG. 9 shows graphs depicting the C1-INH antigen levels in mouse plasma for Studies A (FIG. 9A) and B (FIG. 9B) of Example 3 as measured by ELISA; groups were followed for 7 weeks (Study A) or 6 weeks (Study B) following vector administration; the gray shaded region signifies the mean±SD of all PNP measurements (Study A, 179.2±20.88 μg/mL; Study B, 165.9±20.70 μg/mL); SERPING1=SEQ ID NO: 1, 13=SEQ ID NO: 18, 14=SEQ ID NO: 19, 17=SEQ ID NO: 20, 120=SEQ ID NO: 21, 121=SEQ ID NO: 22, 124=SEQ ID NO: 23; 12=SEQ ID NO: 17, H9=SEQ ID NO: 24, H13=SEQ ID NO: 25, BC2=SEQ ID NO: 26.

FIG. 10 shows graphs depicting the steady-state C1-INH antigen levels in mouse plasma for Studies A (FIG. 10A) and B (FIG. 10B) as measured by ELISA; groups were followed for 7 weeks (Study A) or 6 weeks (Study B) following vector administration; values are presented as mean±SD; statistical comparison relative to AAV-encapsidated SEQ ID NO: 1 was performed by one-way ANOVA with post-hoc Dunnett's multiple comparisons test (*, p<0.05; **, p<0.01; ***, p<0.001); SERPING1=SEQ ID NO: 1, 13=SEQ ID NO: 18, 14=SEQ ID NO: 19, 17=SEQ ID NO: 20, 120=SEQ ID NO: 21, 121=SEQ ID NO: 22, 124=SEQ ID NO: 23, 12=SEQ ID NO: 17, H9=SEQ ID NO: 24, H13=SEQ ID NO: 25, BC2=SEQ ID NO: 26.

FIG. 11 shows graphs depicting plasma bradykinin antigen levels in mice at Week 7 as assessed by ELISA; bradykinin was assessed in mice that received AAV-encapsidated SEQ ID NO: 20 (Group 4) and SEQ ID NO: 22 (Group 6) codon optimized variants, which were associated with higher levels of C1-INH antigen expression; FIG. 11A shows summary bradykinin values in the excipient, SEQ ID NO: 20 variant, and SEQ ID NO: 22 variant groups of Study A; the gray shaded region signifies mean±SD of all pooled normal plasma (PNP) measurements (35010.16±8093.98 μg/mL); values are presented as mean±SD; statistical comparison relative to SEQ ID NO: 1 was performed by one-way ANOVA with post-hoc Dunnett's multiple comparisons test (**, p<0.01); FIG. 11B shows individual C1-INH antigen values in AAV-encapsidated SEQ ID NO: 20- and 22-treated mice at Week 7 plotted relative to their respective plasma bradykinin antigen level (n=9 total samples), and a two-tailed Pearson correlation indicated significance for the SEQ ID NO: 22 group (Pearson r=−0.9417, p<0.0168, R²=0.8867); one animal from the excipient group was euthanized prior to study end following presentation of a necrotic tail and head tilt. One animal that received AAV-encapsidated SEQ ID NO: 20 was removed from analysis following use of the ROUT method for outlier identification; 17=SEQ ID NO: 20, 121=SEQ ID NO: 22.

FIG. 12 shows graphs depicting vector genome concentration in terminal liver tissue for Studies A (FIG. 12A) and B (FIG. 12B) of Example 3, with the vector genome copy numbers normalized to mouse Foxp1 and values presented as mean±SD; study terminus is defined as Week 7 and Week 6 for Studies A and B, respectively; statistical comparison relative to the native SERPING1 transgene was performed by Kruskal-Wallis test with post-hoc Dunn's multiple comparisons test (ns, p>0.05); SERPING1=SEQ ID NO: 1, 13=SEQ ID NO: 18, 14=SEQ ID NO: 19, 17=SEQ ID NO: 20, 120=SEQ ID NO: 21, 121=SEQ ID NO: 22,124=SEQ ID NO: 23, 12=SEQ ID NO: 17, H9=SEQ ID NO: 24, H13=SEQ ID NO: 25, BC2=SEQ ID NO: 26.

FIG. 13 shows a graph depicting correlation of C1-INH antigen with vector genome concentration at terminus in individual mice; correlation was performed on codon optimized variants from Study A with the highest C1-INH expression; values were log₁₀ transformed and a linear fit was generated using regression analysis (R²=0.7243); a two-tailed Pearson correlation indicated significance (Pearson r=0.8510, p<0.0001); SERPING1=SEQ ID NO: 1, 17=SEQ ID NO: 20, 121=SEQ ID NO: 22, 124=SEQ ID NO: 23.

FIG. 14 shows a graph depicting steady state C1-INH antigen levels relative to vector genome concentration (normalized to Foxp1) at terminus (Week 7) for Study A of Example 3; steady-state C1-INH antigen was defined as no statistically significant difference in levels across the timepoints tested (statistical analysis not shown, Weeks 3-7); values are presented as mean±SD; statistical comparison relative to native SERPING1 signal peptide was performed by Kruskal-Wallis test with post-hoc Dunn's multiple comparisons test (*, p<0.05); SERPING1=SEQ ID NO: 1, 13=SEQ ID NO: 18, 14=SEQ ID NO: 19, 17=SEQ ID NO: 20, 120=SEQ ID NO: 21, 121=SEQ ID NO: 22, 124=SEQ ID NO: 23.

FIG. 15 shows graphs depicting C1-INH antigen levels in mouse plasma for the study of Example 4; specifically, C1-INH antigen levels are plotted as a function of time (FIG. 15A) and at week 8 (FIG. 15B) for all groups as measured by ELISA; pooled normal human plasma (PNP) was included as a control to monitor inter-assay variability; gray shaded region signifies the mean±SD of all measurements of PNP (175.4±41.3 μg/mL); values are presented as mean±SD; values below the limit of detection were excluded from calculations of mean±SD; 1 male and 3 females (all low dose) exhibited no detectable C1-INH antigen over the course of the study; 17=SEQ ID NO: 20.

FIG. 16 shows a graph depicting a correlation of C1-INH antigen with C1-INH activity in mouse plasma for the study of Example 4, specifically, individual values for C1-INH activity are plotted relative to each animal's respective C1-INH antigen level, with data from Weeks 5 and 8; values were log₁₀ transformed, and a linear fit was generated using regression analysis; below quantitation level (BQL) values are excluded while above quantitation level (AQL) values are included for visualization; 17=SEQ ID NO: 20.

FIG. 17 shows a graph depicting vector genome concentrations from terminal liver tissue for the study of Example 4, specifically, vector genome copy numbers normalized to mouse Foxp1 following administration of one of three doses of AAV-encapsidated SEQ ID NO: 20; values are presented as mean±SD; 17=SEQ ID NO: 20.

FIG. 18 shows a graph depicting normalized SERPING1 mRNA expression from terminal liver tissue, specifically, mRNA expression levels of SERPING1 normalized to mouse Ppie in mice that received one of three doses of AAV-encapsidated SEQ ID NO: 20; terminus is defined as Week 8; values are presented as mean±SD; 17=SEQ ID NO: 20.

FIG. 19 shows graphs depicting comparison of vector dose, normalized vector genome concentration, normalized mRNA expression levels, and terminal C1-INH antigen levels for the study of Example 4, specifically, values are plotted for male (FIG. 19A) and female (FIG. 19B) Serping1^{-/-} mice administered varying doses of AAV-encapsidated SEQ ID NO: 20; terminus was defined as Week 8; VG copy numbers were normalized to mouse Foxp1; SERPING1 mRNA expression levels were normalized to mouse Ppie; C1-INH antigen levels were measured by ELISA; individual values were log 10 transformed and fit to a nonlinear 4PL sigmoidal curve; 17=SEQ ID NO: 20.

FIG. 20 shows data characterizing Serping1 deficiency in a B6.SJL mouse background. FIG. 20A illustrates paw volume differences. FIG. 20B illustrates plasma C1-INH differences. FIG. 20C illustrates bradykinin differences. FIG. 20D illustrates complement C4a (C4a) differences. FIG. 20E illustrates tissue plasminogen activator (tPA) differences.

FIG. 21 illustrates the ability of I21 (SEQ ID NO: 22) to drive dose-dependent, durable hC1-INH antigen levels in plasma using an HAE model mice. Plasma hC1-INH levels in B6/SJL^{Serpring1+/+} (FIG. 21A), B6/SJL^{Serpring1+/-} (FIG. 21B), and B6/SJL^{Serpring1-/-} (FIG. 21C) male mice were measured after injecting one of three doses of AAV-encapsidated 121, ranging from 1.0×10¹² to 3.16×10¹² vg/kg in quarter-log increments.

FIG. 22 illustrates C1-INH expression and pharmacodynamics of I21 (SEQ ID NO: 22). FIG. 22A provides data comparing hC1-INH expression in different Serping1 genotypes. FIG. 22B shows hC1-INH dose response. FIG. 22C shows bradykinin dose response. FIG. 22D shows tPA activity dose response.

FIG. 23 shows I21 (SEQ ID NO: 22) dose-dependent, durable hC1-INH antigen levels in plasma of an HAE model mice. B6/SJL^{Serpring1-/-} male mice were injected with one of five doses of I21, ranging from 9.5×10¹¹ to 3.0×10¹³ vg/kg. FIG. 23A shows plasma C1-INH levels as measured by ELISA as a function of time. Values are in units of IU/mL and presented as mean±SD. FIG. 23B shows steady-state C1-INH antigen levels in B6/SJL^{Serpring1-/-} male mice as a function of vector dose. All values were log₁₀ transformed and a linear fit was generated using regression analysis (R²=0.88).

FIG. 24 show I21 (SEQ ID NO: 22) mediated reduction in bradykinin using an HAE disease model mice. FIG. 24A shows individual and mean±SD plasma bradykinin levels in different dose cohorts. Statistical comparison relative to excipient was performed by one-way ANOVA with post-hoc Dunnett's multiple comparisons test (**, p<0.01; ***, p<0.001). FIG. 24B shows plasma C1-INH levels.

FIG. 25 show I21 (SEQ ID NO: 22) mediated C1-INH rescues C4 levels in an HAE mouse model. Plasma C4 was analyzed at study terminus by an automated capillary-based immunoassay system (Wesm, ProteinSimple). FIG. 25A shows individual and mean±SD relative C4 expression in each dose cohort. FIG. 25B shows individual relative plasma C4 values plotted relative to each animal's respective C1-INH antigen level at week 31. All values were log₁₀ transformed and a linear fit was generated using regression analysis (R²=0.87). FIG. 25C shows the level of C4 as a function of plasma C1-INH. BQL refers below the quantitation level. AQL refers to above to quantitation level.

FIG. 26 shows C1-INH levels produced in mice dosed with AAV-encapsidated 121 (SEQ ID NO: 22), to evaluate toxicology. Male mice were dosed with AAV-encapsidated I21 at 2.5×10¹² vg/kg, 5.0×10¹² vg/kg, or 1.0×10¹³ (FIG. 26A). Female mice were dosed with AAV-encapsidated I21 at 1.0×10¹³ vg/kg, 5.0×10¹³ vg/kg, or 9.9×10¹³ (FIG. 26B).

FIG. 27 shows a I21 (SEQ ID NO: 22) dosing study in male cynomolgus macaque (FIG. 27A) and female cynomolgus macaque (FIG. 27B), where percent normal C1-INH activity was determined at different time points after AAV-encapsidated I21 administration. Cynomolgus macaque were dosed with AAV-encapsidated I21 at 1.0×10¹³ vg/kg (Low), 3.2×10¹³ vg/kg (Mid) or 1.0×10¹⁴ vg/kg (High).

FIG. 28 illustrates hC1-INH antigen level (FIG. 28A) and peak percent normal C1-INH activity (FIG. 28B) produced with different doses of AAV-encapsidated 121. Male and female cynomolgus macaque were dosed with AAV-encapsidated I21 at 1.0×10¹³ vg/kg (Low), 3.2×10¹³ vg/kg (Mid) or 1.0×10¹⁴ vg/kg (High).

FIG. 29 illustrates hC1-INH antigen levels in supernatants of transfected Huh7 cells. Huh7 cells were transfected in vitro with different SERPING1 expression plasmids and hC1-INH antigen levels were measured. Results are the average of biological replicates n=3. Error bars represent standard deviation.

FIG. 30 illustrates the effect of adding a miR-142-3p target into an expression plasmid. Huh7 cells were transfected in vitro with plasmid containing a nucleic acid of SEQ ID NO: 28 (pSwap-SERPING1_I21) or SEQ ID NO: 55 (mir 142-3p), or pCAG_GFP; and hC1-INH antigen was assayed. Results are the average of biological replicates n=3. Error bars represent standard deviation.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the instant invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element, where such element, step or ingredient is related to the claimed invention. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the instant invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

All of the features disclosed herein can be combined in any combination. Each feature disclosed in the specification can be replaced by an alternative feature serving a same, equivalent, or similar purpose. Thus, unless expressly stated otherwise, disclosed features are an example of a genus of equivalent or similar features.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a non-viral vector and/or immune cell modulator “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

In an attempt to help the reader of the application, the description has been separated into various paragraphs or sections, or is directed to various embodiments of the instant invention. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

Provided herein are modified nucleic acids encoding C1 inhibitor, expression cassettes comprising the modified nucleic acids encoding C1 inhibitor, viral vectors comprising the modified nucleic acids encoding C1 inhibitor, and non-viral vectors comprising the modified nucleic acids encoding C1 inhibitor. The instant invention also provides recombinant AAV particles comprising the modified nucleic acids encoding C1 inhibitor, non-viral particles comprising the modified nucleic acids encoding C1 inhibitor, pharmaceutical compositions comprising the modified nucleic acids encoding C1 inhibitor, and methods of treating Hereditary angioedema (HAE).

### Nucleic Acids

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA, antisense DNA/RNA, plasmid DNA, linear DNA, (poly- and oligo-nucleotide), chromosomal DNA, spliced or unspliced mRNA, rRNA, tRNA inhibitory DNA or RNA (RNAi, e.g., small or short hairpin (sh)RNA, microRNA (miRNA), small or short interfering (si)RNA, trans-splicing RNA, or antisense RNA), locked nucleic acid analogue (LNA), oligonucleotide DNA (ODN) single and double stranded, immunostimulating sequence (ISS), riboswitches and ribozymes.

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide can be described herein according to the convention of providing the sequence in the 5′ to 3′ direction.

According to certain embodiments, the polynucleotide is a single-stranded (ssDNA) or a double-stranded DNA (dsDNA) molecule. According to certain embodiments, the polynucleotide is for therapeutic use, e.g., an ssDNA or dsDNA encoding a therapeutic transgene. According to certain embodiments, the dsDNA molecule is a minicircle, a nanoplasmid, open linear duplex DNA or a closed-ended linear duplex DNA (CELiD/ceDNA/doggybone DNA). According to certain embodiments, the ssDNA molecule is a closed circular or an open linear DNA.

A “transgene” is used herein to conveniently refer to a nucleic acid that is intended or has been introduced into a cell or organism. Transgenes include any nucleic acid, such as a heterologous polynucleotide sequence, such as a modified nucleic acid encoding C1 inhibitor, or a heterologous nucleic acid encoding a protein or peptide or a nucleic acid (e.g., miRNA, etc.). The term transgene and heterologous nucleic acid/polynucleotide sequences are used interchangeably herein.

As used herein, the term “C1-inhibitor” or “C1 esterase inhibitor” or “C1-INH” or “C1EI” or “SERPING1” are all used interchangeably and refer to any nucleic acid or protein of SERPING1 or C1 inhibitor. In certain embodiments, a nucleic acid encoding a C1 inhibitor encodes a human C1 inhibitor protein. A full DNA sequence of SERPING1, including introns and exons, is available in GenBank Accession No. NG_009625.1. A human C1 inhibitor consists of 500 amino acids and is available in GenBank Accession No. NP_000053.2. Examples of C1 inhibitor include any naturally occurring C1 inhibitor, and variants thereof. As used herein, “a nucleic acid encoding a C1 inhibitor” refers to a recombinant nucleic acid molecule that encodes a protein having at least part of a function or activity of wild-type C1 inhibitor protein. Examples of such nucleic acid include modified nucleic acid encoding C1 inhibitor.

As used herein, the term “variant protein” refers to any protein that contains one or more changes in the amino acid sequence compared to that of the wild-type. The term “variant protein” includes, e.g., amino acid insertions, additions, substitutions and deletions. The term “variant” also encompasses, e.g., fusion proteins.

According to certain embodiments, a variant C1 inhibitor exhibits the function of the native protein. In certain embodiments, the variant C1 inhibitor exhibits at least 50%, optionally at least 55%, optionally at least 60%, optionally at least 65%, optionally at least 70%, optionally at least 75%, optionally at least 80%, optionally at least 85%, optionally at least 90%, optionally at least 95% of the function of the native protein. Determination of functional activity of a variant C1 inhibitor protein can be conducted, for example, by assessing inhibition of the esterase activity of complement component C1. Any suitable method known to those skilled in the art in view of the present disclosure can be used in the instant invention. Examples of methods that can be used to determine the functional activity of a variant C1 inhibitor protein include, e.g., spectrophotometric assays, such as that described in Drouet et al., Clin Chim Acta, 1988, 174(2):121-130, enzyme immunoassays, such as that described by the Mayo Clinic (website: www.mayocliniclabs.com/test-catalog/Performance/81493), chromogenic assays, such as that described in Munkvad et al., Clin Chem., 1990, 36(5):737-41, each of the references hereby incorporated by reference in its entirety, and commercially available methods such as those described in the Examples herein.

As used herein, the term “fusion protein” or “chimeric protein” refers to a protein created through the joining of two or more originally separate proteins, or portions thereof. In certain embodiments, a linker or spacer is present between each protein.

As used herein, the terms “modify” and grammatical variations thereof, mean that a nucleic acid or protein deviates from a reference or parental sequence. A modified nucleic acid encoding C1 inhibitor has been altered compared to reference (e.g., wild-type) or parental nucleic acid. Modified nucleic acids can therefore have substantially the same, greater or less activity or function than a reference or parental nucleic acid, but at least retain partial activity, function and or sequence identity to the reference or parental nucleic acid. The modified nucleic acid can be genetically modified to encode a modified or variant C1 inhibitor.

A “modified nucleic acid encoding C1 inhibitor” means that the C1 inhibitor nucleic acid has alteration compared the parental unmodified nucleic acid encoding C1 inhibitor. A particular example of a modification is a nucleotide substitution. Nucleotide substitutions can be silent mutations that code for the same amino acid, or missense mutations that code for a different amino acid. Missense mutations can be conservative or non-conservative mutations. Other examples of modifications include, e.g., truncations and insertions. The modified nucleic acid can also include a codon optimized nucleic acid that encodes the same protein as that of the wild-type protein or of the nucleic acid that has not been codon optimized. Codon optimization can be used in a broader sense, e.g., including removing the CpGs. The terms “modification” herein need not appear in each instance of a reference made to a nucleic acid encoding C1 inhibitor.

In certain embodiments, for a modified nucleic acid encoding C1 inhibitor, the C1 inhibitor protein retains at least part of a function or activity of wild-type C1 inhibitor.

As set forth herein, modified nucleic acids encoding C1 inhibitor can exhibit different features or characteristics compared to a reference or parental nucleic acid. For example, modified nucleic acids include sequences with 100% identity to a reference nucleic acid encoding C1 inhibitor as set forth herein, as well as sequences with less than 100% identity to a reference nucleic acid encoding C1 inhibitor.

The terms “identity,” “homology,” and grammatical variations thereof, mean that two or more referenced entities are the same, when they are “aligned” sequences. Thus, by way of example, when two nucleic acids are identical, they have the same sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In certain embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In certain embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In certain embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As set-forth herein, modified nucleic acids encoding C1 inhibitor can be distinct from or exhibit 100% identity or less than 100% identity to a reference nucleic acid encoding C1 inhibitor.

According to certain embodiments, a nucleic acid encoding a C1 inhibitor is selected from the group consisting of: (1) a polynucleotide having at least 83% sequence identity to the sequence of SEQ ID NO: 105, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 105; (2) a polynucleotide having at least 83% sequence identity to the sequence of SEQ ID NO: 106, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 106; (3) a polynucleotide having at least 80% sequence identity to the sequence of SEQ ID NO: 107, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 107; (4) a polynucleotide having at least 80% sequence identity to the sequence of SEQ ID NO: 108, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 108; (5) a polynucleotide having at least 83% sequence identity to the sequence of SEQ ID NO: 109, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 109; (6) a polynucleotide having at least 84% sequence identity to the sequence of SEQ ID NO: 110, such as 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 110; (7) a polynucleotide having at least 80% sequence identity to the sequence of SEQ ID NO: 111, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 111; (8) a polynucleotide having at least 83% sequence identity to the sequence of SEQ ID NO: 112, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 112; (9) a polynucleotide having at least 82% sequence identity to the sequence of SEQ ID NO: 113, such as 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 113; (10) a polynucleotide having at least 82% sequence identity to the sequence of SEQ ID NO: 114, such as 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 114; (11) a polynucleotide having at least 82% sequence identity to the sequence of SEQ ID NO: 115, such as 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 115; (12) a polynucleotide having at least 80% sequence identity to the sequence of SEQ ID NO: 116, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 116; (13) a polynucleotide having at least 80% sequence identity to the sequence of SEQ ID NO: 117, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 117; (14) a polynucleotide having at least 83% sequence identity to the sequence of SEQ ID NO: 118, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 118; (15) a polynucleotide having at least 80% sequence identity to the sequence of SEQ ID NO: 119, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 119; (16) a polynucleotide having at least 80% sequence identity to the sequence of SEQ ID NO: 120, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 120; (17) a polynucleotide having at least 80% sequence identity to the sequence of SEQ ID NO: 121, such as 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 121; (18) a polynucleotide having at least 83% sequence identity to the sequence of SEQ ID NO: 122, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 122; (19) a polynucleotide having 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 123; (20) a polynucleotide having 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 124; (21) a polynucleotide having at least 89% sequence identity to the sequence of SEQ ID NO: 125, such as 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 125; (22) a polynucleotide having at least 86% sequence identity to the sequence of SEQ ID NO: 126, such as 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 126; (23) a polynucleotide having 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 127; (24) a polynucleotide having at least 89% sequence identity to the sequence of SEQ ID NO: 128, such as 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 128; (25) a polynucleotide having at least 89% sequence identity to the sequence of SEQ ID NO: 129, such as 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 129; (26) a polynucleotide having at least 910% sequence identity to the sequence of SEQ ID NO: 130, such as 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 130; (27) a polynucleotide having at least 92% sequence identity to the sequence of SEQ ID NO: 131, such as 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 131; (28) a polynucleotide having at least 93% sequence identity to the sequence of SEQ ID NO: 132, such as 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 132; (29) a polynucleotide having at least 93% sequence identity to the sequence of SEQ ID NO: 133, such as 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 133; (30) a polynucleotide having at least 87% sequence identity to the sequence of SEQ ID NO: 134, such as 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 134; (31) a polynucleotide having at least 89% sequence identity to the sequence of SEQ ID NO: 135, such as 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 135; (32) a polynucleotide having at least 93% sequence identity to the sequence of SEQ ID NO: 136, such as 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 136; (33) a polynucleotide having at least 93% sequence identity to the sequence of SEQ ID NO: 137, such as 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 137; (34) a polynucleotide having at least 87% sequence identity to the sequence of SEQ ID NO: 138, such as 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 138; (35) a polynucleotide having at least 86% sequence identity to the sequence of SEQ ID NO: 139, such as 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 139; (36) a polynucleotide having at least 86% sequence identity to the sequence of SEQ ID NO: 140, such as 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 140; and (37) a polynucleotide having at least 86% sequence identity to the sequence of SEQ ID NO: 141, such as 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 141.

According to certain embodiments, a nucleic acid of the instant invention encodes a C1 inhibitor having the amino acid sequence of SEQ ID NO: 181 or 192.

According to certain embodiments, the polynucleotide comprises a nucleic acid that encodes a variant C1 inhibitor. Examples of the variant C1 inhibitor include, but are not limited to, a truncated C1 inhibitor, a fusion of two or more C1 inhibitors, or a fusion of a C1 inhibitor with a stabilizing moiety, such as an Fc region or domain.

As used herein, the term “Fc region” or “Fc domain” means the carboxyl-terminal portion of an immunoglobulin heavy chain constant region, or an analog or portion thereof. That is, e.g., an immunoglobulin Fc region of Ig, preferably IgG, which can comprise at least a portion of a hinge region, a CH2 domain, and a CH3 domain. The Fc region can be a native sequence Fc region or a variant Fc region. A native sequence Fc region comprises an amino acid sequence identical to the amino acid sequence of an Fc region found in nature. A variant Fc region as appreciated by one of ordinary skill in the art comprises an amino acid sequence which differs from that of a native sequence Fc region by virtue of at least one amino acid modification. According to certain embodiments, an Fc region is one of those described in Dall'Acqua et al., J Biol Chem., 281(33): 23514-24 (2006), Ishino et al., J Biol Chem., 288(23): 16529-37 (2013), Ying et al., J Biol Chem., 287(23): 19399-19408 (2012), or Zalevsky et al., Nat Biotechnol., 28(2): 157-159 (2010), incorporated herein by reference in their entirety.

According to certain embodiments, an Fc region of the instant invention has an amino acid sequence of any one of SEQ ID NOs: 219-224. According to certain embodiments, a nucleic acid encoding an Fc region of the instant invention has a sequence of any one of SEQ ID NOs: 159-164.

In certain embodiments, a nucleic acid encoding a variant C1 inhibitor has a sequence of any one of SEQ ID NOs: 143-144, 158, and 165-170.

According to certain embodiments, a nucleic acid of the instant invention encodes a variant C1 inhibitor having the amino acid sequence of any one of SEQ ID NOs: 193-201.

According to certain embodiments, the polynucleotide comprises a nucleic acid that encodes a fusion of two or more C1 inhibitor proteins. In certain embodiments, first C1 inhibitor protein or variant thereof is fused to a second C1 inhibitor protein or variant thereof via an autoprotease peptide, such as the peptide sequence of porcine teschovirus-1 2A (P2A). In certain embodiments, the nucleic acid has a sequence of SEQ ID NO: 158. According to certain embodiments, the polynucleotide comprises a nucleic acid that encodes a fusion of a first and second C1 inhibitor protein having the amino acid sequence of SEQ ID NO: 195.

Modified nucleic acids encoding C1 inhibitor that exhibit different features or characteristics compared to a reference or parental nucleic acid include substitutions of nucleotides. For example, modified nucleic acids encoding C1 inhibitor include nucleic acids with a reduced number of CpG dinucleotides compared to a reference nucleic acid encoding C1 inhibitor, referred to as CpG-reduced nucleic acids.

As used herein, the phrase “CpG-reduced” or “CpG-depleted” refers to a nucleic acid sequence which is generated, either synthetically or by mutation of a nucleic acid sequence, such that one or more of the CpG dinucleotides are removed from the nucleic acid sequence. In certain embodiments, all CpG motifs are removed to provide what is termed herein as a modified CpG-free sequence. The CpG motifs are suitably reduced or eliminated not just in a coding sequence (e.g., a transgene), but also in the non-coding sequences, including, e.g., 5′ and 3′ untranslated regions (UTRs), promoter, enhancer, signal peptides, polyA, ITRs, introns, and any other sequences present in the polynucleotide molecule.

According to certain embodiments, a nucleic acid encoding a C1 inhibitor contains less than 24 CpG dinucleotides, such as 24, 23, 22, 21, 20, 19, 18, 17, 16, 15, 14, 13, 12, 11, 10, 9, 8, 7, 6, 5, 4, 3, 2, 1, or 0 CpG dinucleotides.

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

Nucleic acids, expression vectors, AAV vector genomes, non-viral vectors, plasmids, including modified nucleic acids encoding C1 inhibitor of the instant invention can be prepared by using recombinant DNA technology methods. The availability of nucleotide sequence information enables preparation of isolated nucleic acid molecules of the instant invention by a variety of means. Nucleic acids encoding C1 inhibitor can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids can be maintained as DNA in any convenient cloning vector. In certain embodiments, clones are maintained in a plasmid cloning/expression vector, such as pBluescript (Stratagene, La Jolla, CA), which is propagated in a suitable E. coli host cell. Alternatively, nucleic acids can be maintained in vector suitable for expression in mammalian cells, for example, an AAV vector. In cases where post-translational modification affects protein function, nucleic acid molecule can be expressed in mammalian cells.

### Expression Cassettes

The instant invention also provides expression cassettes comprising the polynucleotides comprising the nucleic acids encoding C1 inhibitor as described herein, operably linked to an expression control element.

In certain embodiments, the expression cassette comprises a polynucleotide comprising a nucleic acid encoding a C1 inhibitor, wherein the nucleic acid is selected from the group consisting of: (1) a polynucleotide having at least 83% sequence identity (e.g., 83-100% identity) to the sequence of SEQ ID NO: 105; (2) a polynucleotide having at least 83% sequence identity (e.g., 83-100% identity) to the sequence of SEQ ID NO: 106; (3) a polynucleotide having at least 80% sequence identity (e.g., 80-100% identity) to the sequence of SEQ ID NO: 107; (4) a polynucleotide having at least 80% sequence identity (e.g., 80-100% identity) to the sequence of SEQ ID NO: 108; (5) a polynucleotide having at least 83% sequence identity (e.g., 83-100% identity) to the sequence of SEQ ID NO: 109; (6) a polynucleotide having at least 84% sequence identity (e.g., 84-100% identity) to the sequence of SEQ ID NO: 110; (7) a polynucleotide having at least 80% sequence identity (e.g., 80-100% identity) to the sequence of SEQ ID NO: 111; (8) a polynucleotide having at least 83% sequence identity (e.g., 83-100% identity) to the sequence of SEQ ID NO: 112; (9) a polynucleotide having at least 82% sequence identity (e.g., 82-100% identity) to the sequence of SEQ ID NO: 113; (10) a polynucleotide having at least 82% sequence identity (e.g., 82-100% identity) to the sequence of SEQ ID NO: 114; (11) a polynucleotide having at least 82% sequence identity (e.g., 82-100% identity) to the sequence of SEQ ID NO: 115; (12) a polynucleotide having at least 80% sequence identity (e.g., 80-100% identity) to the sequence of SEQ ID NO: 116; (13) a polynucleotide having at least 80% sequence identity (e.g., 80-100% identity) to the sequence of SEQ ID NO: 117; (14) a polynucleotide having at least 83% sequence identity (e.g., 83-100% identity) to the sequence of SEQ ID NO: 118; (15) a polynucleotide having at least 80% sequence identity (e.g., 80-100% identity) to the sequence of SEQ ID NO: 119; (16) a polynucleotide having at least 80% sequence identity (e.g., 80-100% identity) to the sequence of SEQ ID NO: 120; (17) a polynucleotide having at least 80% sequence identity (e.g., 80-100% identity) to the sequence of SEQ ID NO: 121; (18) a polynucleotide having at least 83% sequence identity (e.g., 83-100% identity) to the sequence of SEQ ID NO: 122; (19) a polynucleotide having at least 93% sequence identity (e.g., 93-100% identity) to the sequence of SEQ ID NO: 123; (20) a polynucleotide having at least 92% sequence identity (e.g., 92-100% identity) to the sequence of SEQ ID NO: 124; (21) a polynucleotide having at least 89% sequence identity (e.g., 89-100% identity) to the sequence of SEQ ID NO: 125; (22) a polynucleotide having at least 86% sequence identity (e.g., 86-100% identity) to the sequence of SEQ ID NO: 126; (23) a polynucleotide having at least 92% sequence identity (e.g., 92-100% identity) to the sequence of SEQ ID NO: 127; (24) a polynucleotide having at least 89% sequence identity (e.g., 89-100% identity) to the sequence of SEQ ID NO: 128; (25) a polynucleotide having at least 89% sequence identity (e.g., 89-100% identity) to the sequence of SEQ ID NO: 129; (26) a polynucleotide having at least 91% sequence identity (e.g., 91-100% identity) to the sequence of SEQ ID NO: 130; (27) a polynucleotide having at least 92% sequence identity (e.g., 92-100% identity) to the sequence of SEQ ID NO: 131; (28) a polynucleotide having at least 93% sequence identity (e.g., 93-100% identity) to the sequence of SEQ ID NO: 132; (29) a polynucleotide having at least 93% sequence identity (e.g., 93-100% identity) to the sequence of SEQ ID NO: 133; (30) a polynucleotide having at least 87% sequence identity (e.g., 87-100% identity) to the sequence of SEQ ID NO: 134; (31) a polynucleotide having at least 89% sequence identity (e.g., 89-100% identity) to the sequence of SEQ ID NO: 135; (32) a polynucleotide having at least 93% sequence identity (e.g., 93-100% identity) to the sequence of SEQ ID NO: 136; (33) a polynucleotide having at least 93% sequence identity (e.g., 93-100% identity) to the sequence of SEQ ID NO: 137; (34) a polynucleotide having at least 87% sequence identity (e.g., 87-100% identity) to the sequence of SEQ ID NO: 138; (35) a polynucleotide having at least 86% sequence identity (e.g., 86-100% identity) to the sequence of SEQ ID NO: 139; (36) a polynucleotide having at least 86% sequence identity (e.g., 86-100% identity) to the sequence of SEQ ID NO: 140; and (37) a polynucleotide having at least 86% sequence identity (e.g., 86-100% identity) to the sequence of SEQ ID NO: 141.

In certain embodiments, the C1 inhibitor comprises the amino acid sequence of SEQ ID NO: 181 or 192.

According to certain embodiments, the expression cassette comprises a polynucleotide comprising a nucleic acid encoding a variant C1 inhibitor, wherein the variant C1 inhibitor comprises a truncated C1 inhibitor, a fusion of two or more C1 inhibitors, or a fusion of a C1 inhibitor with an Fc region or domain. According to certain embodiments, the nucleic acid has a sequence of any one of SEQ ID NOs: 143-144, 158, and 165-170.

According to certain embodiments, a nucleic acid of the instant invention encodes a variant C1 inhibitor having the amino acid sequence of any one of SEQ ID NOs: 193-201.

In certain embodiments, the expression cassette comprises a coding sequence for an appropriate secretory signal sequence or signal peptide that will allow the secretion of the polypeptide encoded by the polynucleotide molecule of the instant invention. As used herein, the term “secretory signal sequence” or “signal peptide” or variations thereof are intended to refer to amino acid sequences that can function to drive secretion of an operably linked polypeptide from the cell. In certain embodiments, a secretory signal sequence or signal peptide can function to enhance secretion of an operably linked polypeptide from the cell as compared with the level of secretion seen with the native polypeptide having its native or naturally occurring signal peptide. Signal peptides are short peptides (typically 25 to 30 amino acids in length) located in the N-terminus of proteins, carrying information for protein secretion. Signal peptides direct proteins to or through the endoplasmic reticulum secretory pathway. By “enhanced” secretion, it is meant that the relative proportion of the polypeptide synthesized by the cell that is secreted from the cell is increased; it is not necessary that the absolute amount of secreted protein is also increased. In certain embodiments, essentially all (i.e., at least 95%, 97%, 98%, 99% or more) of the polypeptide is secreted. It is not necessary, however, that essentially all or even most of the polypeptide is secreted, as long as the level of secretion is enhanced as compared with the native polypeptide having no signal peptide. Generally, secretory signal sequences are cleaved within the endoplasmic reticulum and, in certain embodiments, the secretory signal sequence is cleaved prior to secretion. It is not necessary, however, that the secretory signal sequence is cleaved as long as secretion of the polypeptide from the cell is enhanced and the polypeptide is functional. Thus, in certain embodiments, the secretory signal sequence is partially or entirely retained. The secretory signal sequence can be derived in whole or in part from the secretory signal of a secreted polypeptide (i.e., from the precursor) and/or can be in whole or in part synthetic. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked polypeptide. The secretory signal sequences of the instant invention can comprise, consist essentially of, or consist of a naturally occurring secretory signal sequence or a modification thereof. Numerous secreted proteins and sequences that direct secretion from the cell are known in the art, including those described in Owji et al., Eur. J Cell Biol. 97:422-441 (2018). The secretory signal sequence of the instant invention can further be in whole or in part synthetic or artificial. Synthetic or artificial secretory signal peptides are known in the art, see, e.g., Barash et al., Biochem. Biophys. Res. Comm. 294:835-42 (2002).

Any suitable signal peptide known to those skilled in the art in view of the present disclosure can be used in the instant invention. Examples of signal peptides include, but are not limited to, those found from the Signal Peptide Database (website: www.signalpeptide.de/). Examples of signal peptides suitable for the present invention include, but are not limited to, wild-type C1 inhibitor signal peptide, a human chymotrypsinogen B2 signal peptide (“Sp7”; 18 amino acid signal peptide of NCBI reference sequence NP_001020371), albumin (ALB) signal peptide, orosomucoid 1 (ORM1) signal peptide, transferrin (TF) signal peptide, al-microglobulin/bikunin precursor (AMBP) signal peptide, lysosome-associated membrane glycoprotein 1 (LAMP1) signal peptide, butyrophilin subfamily 2 member A2 (BTN2A2) signal peptide, CD300 signal peptide, Notch2 signal peptide, stereocilin (STRC) signal peptide, alpha 2-HS-glycoprotein (AHSG) signal peptide, SYN1 signal peptide (SEQ ID NO: 215), SYN2 signal peptide (SEQ ID NO: 216), SYN3 signal peptide (SEQ ID NO: 217), SYN4 signal peptide (SEQ ID NO: 218), secrecon (artificial signal sequence described in Barash et al., Biochem Biophys Res Commun., 294: 835-842 (2002)), mouse IgKVIII, human IgKVIII, CD33, tPA, a-1 antitrypsin signal peptide, native secreted alkaline phosphatase (SEAP). Any conventional signal sequence that directs proteins through the endoplasmic reticulum secretory pathway, including variants of the above-mentioned signal peptides, can be used in the present invention.

In certain embodiments, the instant invention relates to a signal peptide following any one, two, three, or all four of the following rules: (1) an amino-terminal N-region of 2-5 amino acids with a net positive charge, (2) a hydrophobic H-region of 6-15 amino acids, (3) a carboxyl-terminal C-region of 5-10 amino acids, with the amino acid in the −3 position from the C-terminus of the signal peptide having no charge, and the amino acid in the −1 position from the C-terminus of the signal peptide containing a short side chain, and (4) a leucine residue at the −10 position from the C-terminus of the signal peptide.

In certain embodiments, the instant invention relates to a signal peptide comprising a sequence with a sequence identity of at least 90%, at least 95%, or 100% to SEQ ID NO: 215. In certain embodiments, the instant invention relates to a signal peptide comprising a sequence with a sequence identity of at least 90%, at least 95% or 100% to SEQ ID NO: 216. In certain embodiments, the instant invention relates to a signal peptide comprising a sequence with a sequence identity of at least 90%, at least 95% or 100% to SEQ ID NO: 217. In certain embodiments, the instant invention relates to a signal peptide comprising a sequence with a sequence identity of at least 90%, at least 95% or 100% to SEQ ID NO: 218.

In certain embodiments, the instant invention relates to a nucleic acid encoding a signal peptide, wherein the nucleic acid comprises a sequence with a sequence identity of at least 90%, at least 95% or 100% to SEQ ID NO: 100. In certain embodiments, the instant invention relates to a nucleic acid encoding a signal peptide, wherein the nucleic acid comprises a sequence with a sequence identity of at least 90%, at least 95% or 100% to SEQ ID NO: 101. In certain embodiments, the instant invention relates to a nucleic acid encoding a signal peptide, wherein the nucleic acid comprises a sequence with a sequence identity of at least 90%, at least 95% or 100% to SEQ ID NO: 102. In certain embodiments, the instant invention relates to a nucleic acid encoding a signal peptide, wherein the nucleic acid comprises a sequence with a sequence identity of at least 90%, at least 95% or 100% to SEQ ID NO: 103. In further embodiments, the encoded signal peptide has a sequence identity of at least 95% or 100% to any one of SEQ ID NOs: 215-218.

In certain embodiments, signal peptides of the instant invention are useful for causing or driving secretion of any therapeutic protein expressed from any therapeutic transgene known in the art. In certain embodiments, signal peptides of the instant invention are useful for causing or driving secretion from the liver of any therapeutic protein expressed from any therapeutic transgene known in the art.

In certain embodiments, signal peptides comprising a sequence of one of SEQ ID NOs: 215-218 are useful for causing or driving secretion of any therapeutic protein expressed from any therapeutic transgene known in the art. In certain embodiments, signal peptides comprising a sequence of one of SEQ ID NOs: 215-218 are useful for causing or driving secretion of any therapeutic protein expressed from any therapeutic transgene known in the art.

Certain embodiments are directed to a polypeptide comprising a sequence at least 95%, or 100% identical to any one of SEQ ID NOs: 215, 216, 217, and 218; and a nucleic acid comprising a sequence encoding for a polypeptide comprising a sequence at least 95%, or 100% identical to any one of SEQ ID NOs: 215, 216, 217, and 218.

Examples of therapeutic transgenes include, but are not limited to, myelin oligodendrocyte glycoprotein (MOG), proteolipid protein 1 (PLP1), or myelin basic protein (MBP) for treatment of multiple sclerosis, such as, e.g., those disclosed in PCT/US2020/061356, filed Nov. 19, 2020, incorporated herein by reference in its entirety; GAA (acid alpha-glucosidase) for treatment of Pompe, such as, e.g., those disclosed in WO2019/222411, incorporated herein by reference in its entirety, disease or another glycogen storage disease; ATP7B (copper transporting ATPase2) for treatment of Wilson's disease; GLA (alpha galactosidase A) for treatment of Fabry disease; ASS1 (arginosuccinate synthase) for treatment of Citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II); glucose-6-phosphatase for treatment of glycogen storage disease type I (GSDI); insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I or II (IGF-I or IGF-II), TGFβ, activins, inhibins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase; thrombopoietin (TPO), an interleukin (IL-1 through IL-36, etc.), monocyte chemoattractant protein, leukemia inhibitory factor, granulocyte-macrophage colony stimulating factor, Fas ligand, tumor necrosis factors α or β, interferons α, β, or γ, stem cell factor, flk-2/flt3 ligand, IgG, IgM, IgA, IgD or IgE, chimeric immunoglobulins, humanized antibodies, single chain antibodies, T cell receptors, chimeric T cell receptors, single chain T cell receptors, class I or class II MHC molecules; CFTR (cystic fibrosis transmembrane regulator protein), a blood coagulation (clotting) factor (Factor XIII, Factor IX (FIX), Factor VIII (FVIII), Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDRT, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein; erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alphal-antitrypsin for treatment of emphysema or chronic obstructive pulmonary disease (COPD); alpha-L-iduronidase for treatment of mucopolysaccharidosis I (MPS I); omithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; dystrophin; a gene editing nuclease, e.g., a zinc finger nuclease (ZFN), a transcription activator-like effector nuclease (TALEN), a functional Type II CRISPR-Cas9.

In certain embodiments, the signal peptide is an endogenous or native C1 inhibitor signal peptide or a variant thereof.

In certain embodiments, the signal peptide is a heterologous signal peptide or a variant thereof.

In certain embodiments, the expression cassette comprises a nucleic acid encoding a signal peptide sequence positioned at the 5′ end of the nucleic acid encoding the C1 inhibitor.

In certain embodiments, the signal peptide has a sequence of any one of SEQ ID NOs: 84-103. In certain embodiments, the signal peptide has an amino acid sequence of any one of SEQ ID NOs: 203-224.

In certain embodiments, an expression control element is positioned 5′ of a nucleic acid encoding a C1 inhibitor.

The term “expression cassette”, as used herein, refers to a nucleic acid construct comprising nucleic acid elements sufficient for the expression of the polynucleotide molecule of the instant invention. Typically, an expression cassette comprises the polynucleotide molecule of the instant invention operably linked to a promoter sequence.

An “expression control element” refers to nucleic acid sequence(s) that influence expression of an operably linked nucleic acid. Expression control elements as set forth herein include promoters and enhancers. Vector sequences including AAV vectors and non-viral vectors can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but can also act in trans.

Expression control can be affected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100-500, or more nucleotides from the polynucleotide), even at considerable distances. Nevertheless, owing to the length limitations of AAV vectors, expression control elements in AAV vectors will typically be within 1 to 1000 nucleotides from the transcription start site of the heterologous nucleic acid.

Functionally, expression of an operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

The term “operably linked” means that the regulatory sequences necessary for expression of a nucleic acid sequence are placed in the appropriate positions relative to the sequence so as to affect expression of the nucleic acid sequence. This same definition is sometimes applied to the arrangement of nucleic acid sequences and transcription control elements (e.g., promoters, enhancers, and termination elements) in an expression vector, e.g., recombinant AAV (rAAV) vector or non-viral vector. Encoding sequences can be operably linked to regulatory sequences in sense or antisense orientation. In certain embodiments, the promoter is a heterologous promoter.

The term “heterologous promoter”, as used herein, refers to a promoter that is not found to be operably linked to a given encoding sequence in nature. In certain embodiments, an expression cassette can comprise additional elements, for example, an intron, an enhancer, a polyadenylation site, a woodchuck response element (WRE), and/or other elements known to affect expression levels of the encoding sequence.

As used herein, the term “promoter” refers to a nucleotide sequence capable of controlling the expression of a coding sequence or functional RNA. In general, nucleic acid molecules of the instant invention are located 3′ of a promoter sequence. In certain embodiments, a promoter sequence consists of proximal and more distal upstream elements and can comprise an enhancer element.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expression of an operably linked nucleic acid above expression afforded by a promoter element.

An expression construct can comprise regulatory elements which serve to drive expression in a particular cell or tissue type. Expression control elements (e.g., promoters) include those active in a particular tissue or cell type, referred to herein as a “tissue-specific expression control element/promoter.” Tissue-specific expression control elements are typically active in a specific cell or tissue type (e.g., liver). Expression control elements are typically active in particular cells, tissues, or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs provides for at least partial tissue tropism for the expression of a heterologous nucleic acid encoding a protein or inhibitory RNA. Examples of promoters that are active in liver are the transthyretin (TTR) gene promoter; human alpha 1-antitrypsin (hAAT) promoter; the lipoprotein A-I promoter; albumin, Miyatake, et al., J Virol., 71:5124-32 (1997); hepatitis B virus core promoter, Sandig, et al., Gene Ther. 3: 1002-9 (1996); alpha-fetoprotein (AFP), Arbuthnot, et al., Hum. Gene. Ther., 7:1503-14 (1996); human Factor IX promoter; thyroxin binding globulin (TBG) promoter; TTR minimal enhancer/promoter, alpha-antitrypsin promoter, LSP (845 nt) (requires intronless scAAV); LSP1 promoter, among others. An example of an enhancer active in liver is apolipoprotein E (ApoE) hepatic control region 1 (HCR-1) and 2 (HCR-2) (Allan et al, J Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic beta-actin promoter and the phosphoglycerate kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268: 1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997); and the rapamycin-inducible system (Magari, et al., J. Clin. Invest. 100:2865-2872 (1997); Rivera, et al., Nat. Medicine. 2:1028-1032 (1996)). Other regulatable control elements which can be useful in this context are those which are regulated by a specific physiological state, e.g., temperature, acute phase, development.

Other examples of promoters include, but are not limited to, the phosphoglycerate kinase (PKG) promoter, CAG (composite of the CMV enhancer the chicken beta-actin promoter (CBA) and the rabbit beta globin intron) and other constitutive promoters, neuronal specific enolase (NSE) promoter, synapsin or NeuN promoters, the SV40 early promoter, mouse mammary tumor virus LTR promoter; adenovirus major late promoter (Ad MLP), a herpes simplex virus (HSV) promoter, spleen focus-forming virus (SFFV) promoter, rous sarcoma virus (RSV) promoter, rat insulin promoter, thyroxine binding globulin (TBG) promoter and other liver-specific promoters, the desmin promoter and similar muscle-specific promoters, the EF1-alpha promoter, synthetic promoters, hybrid promoters, promoters with multi-tissue specificity, and the like, all of which are promoters well known and readily available to those of skill in the art. Other promoters can be of human origin or from other species, including from mice.

Expression control elements also include the native elements(s) for the heterologous polynucleotide. A native control element (e.g., promoter) can be used when it is desired that expression of the heterologous polynucleotide should mimic the native expression. The native element can be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences can also be used.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (poly A) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In certain embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

In certain embodiments, the expression cassette further comprises one or more tissue specificity elements. As used herein, a “tissue specificity element” refers to any polynucleotide sequence that directs tissue-specific expression of a transgene. In certain embodiments, the tissue specificity element is a promoter. In certain embodiments, the promoter sequence is CpG-reduced compared to the wild-type promoter sequence. In certain embodiments, the promoter is an hAAT promoter. In certain embodiments, the hAAT promoter sequence has a polynucleotide sequence of SEQ ID NO: 79 or 80.

In certain embodiments, the expression cassette further comprises one or more potency elements. As used herein, a “potency element” refers to any polynucleotide sequence that enhances the stability of an mRNA molecule. In certain embodiments, a potency element is an enhancer or a polyadenylation (polyA) sequence. In certain embodiments, the enhancer or polyA sequence is CpG-reduced compared to the wild-type enhancer or polyA sequence. The one or more potency elements can be positioned anywhere within the expression cassette. In certain embodiments, a potency element is an enhancer such as, for example, those described in Van Linthout et al., Hum Gene Ther. 2002 May 1; 13(7):829-40, Donello et al., J Virol. 1998; 72(6):5085-5092, Zufferey et al., J Virol. 1999; 73(4):2886-2892, or Choi et al., Mol Brain 7, 17 (2014), incorporated herein by reference in their entirety. In certain embodiments, a potency element is an enhancer selected from the group consisting of ApoE, 2×ApoE, 4×ApoE, hAAT enhancer, WPRE, WPRE3, and an intron that is optionally a human hemoglobin β (HBB)-derived intron, such as, for example, those described in Ronzitti et al., Mol Ther Methods Clin Dev. 2016; 3:160, incorporated herein by reference in its entirety. In certain embodiments, the enhancer has a polynucleotide sequence selected from the group consisting of SEQ ID NOs: 225, 74-76, 81-82, and 173-178. In certain embodiments, a potency element is a polyA sequence that is optionally a bovine growth hormone (bGH) polyadenylation (polyA) sequence. In certain embodiments, the polyA sequence has a polynucleotide of SEQ ID NO: 83.

In certain embodiments, the expression control element comprises an ApoE/hAAT enhancer/promoter sequence positioned 5′ of the nucleic acid encoding C1 inhibitor. In certain embodiments, the ApoE/hAAT enhancer/promoter sequence is CpG-reduced compared to wild-type ApoE/hAAT enhancer/promoter sequence. In certain embodiments, the ApoE enhancer sequence has a sequence of any one of SEQ ID NOs: 225 and 74-76. In certain embodiments, the hAAT promoter sequence has a sequence of SEQ ID NO: 79 or 80.

In certain embodiments, the expression cassette further comprises one or more response elements. As used herein, a “response element” refers to a nucleic acid sequence, which when positioned proximate to a promoter or within the promoter is capable of regulating the transcription activity. In certain embodiments, a response element is an miRNA binding site, a regulated Irel-dependent decay (RIDD) sequence, an acute phase response element (APRE), or a 5′ or 3′ UTR sequence. In certain embodiments, the an miRNA binding site, a regulated Irel-dependent decay (RIDD) sequence, an acute phase response element (APRE), or a 5′ or 3′ UTR sequence is CpG-reduced compared to the wild-type an miRNA binding site, a regulated Irel-dependent decay (RIDD) sequence, an acute phase response element (APRE), or a 5′ or 3′ UTR sequence. In certain embodiments, the response element is an miRNA binding site, optionally a miR-142-3p sequence, such as those described in Brown et al., Nat Med 12, 585-591 (2006). In certain embodiments, the miR-142-3p sequence has a polynucleotide sequence of SEQ ID NO: 179. In certain embodiments, the response element is a regulated Irel-dependent decay (RIDD) sequence. In certain embodiments, the response element is a RIDD sequence such as, for example, those described in Oikawa et al., Nucleic Acids Res. 2010 October; 38(18):6265-73 or Moore and Hollien, Molecular Biology of the Cell 2015 26:16, 2873-2884, incorporated herein by reference in their entirety. In certain embodiments, the response element is a RIDD sequence selected from the group consisting of 1×RIDD, 3×RIDD, and RIDD1×Blos. In certain embodiments, the RIDD sequence has a polynucleotide sequence selected from the group consisting of SEQ ID NOs: 185-187. In certain embodiments, the response element is an acute phase response element (APRE). In certain embodiments, the response element is an APRE sequence such as, for example, those described in Longley et al., J Immunol. 1999 Oct. 15; 163(8):4537-45, Podvinec et al., PNAS, 2004; 101(24):9127-9132, Prada et al., Immunobiology. 1998 August; 199(2):377-88, Rygg et al., Scand J Immunol. 2001 June; 53(6):588-95, or Serrano-Mendioroz et al., Hum Gene Ther. 2018; 29(4):480-491, incorporated herein by reference in their entirety. In certain embodiments, the response element is an APRE selected from the group consisting of SAA2 APRE, 2×ADRES, SERPING1 5′ UTR, APRE 5′ UTR, and SAA2 5′ UTR. In certain embodiments, the APRE has a polynucleotide sequence selected from the group consisting of SEQ ID NOs: 77-78, 180, and 182-183. In certain embodiments, the response element is a 5′ or 3′ UTR sequence, optionally a SAA2 3′ UTR sequence, such as, for example, those described in Longley et al., J Immunol. 1999 Oct. 15; 163(8):4537-45. In certain embodiments, the SAA2 3′ UTR sequence has a polynucleotide sequence of SEQ ID NO: 184.

In certain embodiments, the one or more tissue specificity elements, one or more potency elements, and/or one or more response elements are positioned 5′ of the nucleic acid.

In certain embodiments, the one or more tissue specificity elements, one or more potency elements, and/or one or more response elements are positioned 3′ of the nucleic acid.

In certain embodiments, the expression cassette has a sequence of any one of SEQ ID NOs: 1-69 and 227-229. In certain embodiments, the expression cassette has a sequence of at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, at least 99.5% sequence identity, or 100% sequence identity to any one of SEQ ID NOs: 1-69 and 227-229.

### Gene Transfer Systems

### Viral Vectors

The instant invention further provides viral vectors, such as adeno-associated virus (AAV) vectors comprising polynucleotides comprising the nucleic acids encoding C1 inhibitor as set forth herein.

The term “vector” or “gene transfer vector” as used herein, refers to a nucleic acid molecule comprising a gene of interest. Examples of vectors include, but are not limited to, viral vectors delivered by viral particles or virus-like particles (VLPs) that resemble viral particles but are non-infectious, such as retroviral, adenoviral, adeno-associated viral, and lentiviral particles or VLPs; and non-viral vectors delivered by non-viral gene transfer systems, such as microinjection, electroporation, liposomes, large natural polymers, large synthetic polymers, and polymers comprised of both natural and synthetic components.

A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

As used herein, the term “gene transfer system” refers to any means of delivering a composition comprising a nucleic acid sequence to a cell or tissue. For example, a gene transfer system can be a viral gene transfer system, e.g., intact viruses, modified viruses and VLPs to facilitate delivery of a viral vector to a desired cell or tissue. A gene transfer system can also be a non-viral delivery system that does not comprise viral coat protein or form a viral particle or VLP, e.g., liposome-based systems, polymer-based systems, protein-based systems, metallic particle-based systems, peptide cage systems, etc.

A viral vector is derived from or based upon one or more nucleic acid elements that comprise a viral genome. Particular viral vectors include, but are not limited to, lentiviral and adeno-associated virus (AAV) vectors.

The term “recombinant,” as a modifier of vector, such as recombinant AAV (rAAV) vector, as well as a modifier of sequences such as recombinant polynucleotides and polypeptides, means that the compositions have been manipulated (i.e., engineered) in a fashion that generally does not occur in nature. Although the term “recombinant” is not always used herein in reference to AAV vectors, as well as sequences such as polynucleotides, recombinant forms including polynucleotides, are expressly included in spite of any such omission.

A “recombinant AAV vector” or “rAAV” is derived from the wild-type genome of AAV by using molecular methods to remove the wild-type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native sequence with respect to the AAV genomic nucleic acid. Incorporation of a non-native sequence therefore defines the AAV vector as a “recombinant” vector, which can be referred to as a “rAAV vector.”

An rAAV sequence can be packaged, referred to herein as a “particle,” for subsequent infection (transduction) of a cell, ex vivo, in vitro or in vivo. Where a recombinant AAV vector sequence is encapsidated or packaged into an AAV particle, the particle can also be referred to as an “rAAV vector” or “rAAV particle.” Such rAAV particles include proteins that encapsidate or package the vector genome, and in the case of AAV, they are referred to as capsid proteins.

A vector “genome” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid can be referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the polynucleotide that is packaged or encapsidated by virus (e.g., AAV).

Host cells for producing recombinant AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments and expresses the adenoviral E1a and E1b genes is used to generate recombinant AAV particles. The modified HEK293 cell line is readily transfected and provides a particularly convenient platform in which to produce rAAV particles. Other host cell lines appropriate for recombinant AAV production are described in International Application PCT/2017/024951, the disclosure of which is herein incorporated by reference in its entirety.

In certain embodiments, AAV helper functions are introduced into the host cell by transfecting the host cell with an AAV helper construct either prior to, or concurrently with, the transfection of an AAV expression vector. A host cell having AAV helper functions can be referred to as a “helper cell” or “packaging helper cell.” AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

Methods of generating rAAV particles capable of transducing mammalian cells are known in the art. For example, rAAV particles can be produced as described in U.S. Pat. No. 9,408,904; and International Applications PCT/US2017/025396 and PCT/US2016/064414, the disclosures of which are herein incorporated by reference in their entirety.

The instant invention provides cells comprising nucleic acids encoding C1 inhibitor, cells comprising expression cassettes comprising the polynucleotides comprising the nucleic acids encoding C1 inhibitor, cells comprising viral vectors such as AAV vectors comprising nucleic acids encoding C1 inhibitor, and cells comprising non-viral vectors comprising polynucleotides comprising the nucleic acids encoding C1 inhibitor. In certain embodiments, the cell produces a viral vector. In certain embodiments, the cell produces an AAV vector as set forth herein.

Also provided are methods of producing viral vectors such as AAV vectors as set forth herein. In certain embodiments, a method of producing AAV vectors includes: introducing an AAV vector genome comprising a polynucleotide comprising a nucleic acid encoding C1 inhibitor or expression cassette comprising a polynucleotide comprising a nucleic acid encoding C1 inhibitor as set forth herein into a packaging helper cell; and culturing the helper cell under conditions to produce the AAV vectors. In certain embodiments, a method of producing AAV vector includes: introducing a polynucleotide comprising a nucleic acid encoding C1 inhibitor or expression cassette comprising a polynucleotide comprising a nucleic acid encoding C1 inhibitor as set forth herein into a packaging helper cell; and culturing the helper cells under conditions to produce the AAV vector.

In certain embodiments, the cells are mammalian cells.

In certain embodiments, cells for vector production provide helper functions, such as AAV helper functions, that package the vector into a viral particle. In certain embodiments, the helper functions are Rep and/or Cap proteins for AAV vector packaging. In certain embodiments, cells for vector production can be stably or transiently transfected with polynucleotide(s) encoding Rep and/or Cap protein sequence(s). In certain embodiments, cells for vector production provide Rep78 and/or Rep68 proteins. In such cells, the cells can be stably or transiently transfected with Rep78 and/or Rep68 proteins polynucleotide encoding sequence(s).

In certain embodiments, cells for vector production are human embryonic kidney cells. In certain embodiments, cells for vector production are HEK-293 cells.

The term “transduce” and grammatical variations thereof refer to introduction of a molecule such as an rAAV vector into a cell or host organism. The heterologous nucleic acid/transgene may or may not be integrated into genomic nucleic acid of the recipient cell.

The introduced heterologous nucleic acid can also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation, for example, of a nucleic acid (e.g., a transgene) into the cell. Thus, a “transduced” cell is a cell into which, or a progeny thereof in which an exogenous nucleic acid has been introduced. The cell(s) can be propagated, and the introduced protein expressed. For gene therapy uses and methods, a transduced cell can be in a subject.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

Recombinant AAV vector, as well as methods and uses thereof, include any viral strain or serotype. As a non-limiting example, a recombinant AAV vector can be based upon any AAV genome, such as LK03, AAV1, AAV2, AAV3, AAV4, AAV5, AAV6, AAV7, AAV8, AAV9, AAV10, AAV11, AAV12, Rh10, Rh74, AAV3B or AAV-2i8, for example. Such vectors can be based on the same strain or serotype (or subgroup or variant) or be different from each other. As a non-limiting example, a recombinant AAV vector based upon a particular serotype genome can be identical to the serotype of the capsid proteins that package the vector. In addition, a recombinant AAV vector genome can be based upon an AAV serotype genome distinct from the serotype of the AAV capsid proteins that package the vector. For example, the AAV vector genome can be based upon AAV2, whereas at least one of the three capsid proteins could be an LK03, AAV1, AAV3, AAV4, AAV5, AAV6, AAV7, AAV8, AAV9, AAV10, AAV11, AAV12, Rh10, Rh74, AAV3B or AAV-2i8, or variant thereof.

In certain embodiments, adeno-associated virus (AAV) vectors include LK03, AAV1, AAV2, AAV3, AAV4, AAV5, AAV6, AAV7, AAV8, AAV9, AAV10, AAV11, AAV12, Rh10, Rh74, AAV3B and AAV-2i8, as well as variants (e.g., capsid variants, such as amino acid insertions, additions, substitutions and deletions) thereof, for example, as set forth in WO 2013/158879 (International Application PCT/US2013/037170; disclosing RHM4-1, RHM15-1, RHM15-2, RHM15-3/RHM15-5, RHM15-4 and RHM15-6), WO 2015/013313 (International Application PCT/US2014/047670), US 2013/0059732 (U.S. Pat. No. 9,169,299, discloses LK01, LK02, LK03, etc.), and WO 2016/210170, the disclosures of which are herein incorporated by reference in their entirety.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants might not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that can be within a subgroup or a variant of a given serotype.

As set forth herein, AAV capsid proteins can exhibit less than 100% sequence identity to a reference or parental AAV serotype such as AAV1, AAV2, AAV3, AAV4, AAV5, AAV6, AAV7, AAV8, AAV9, AAV10, AAV11, AAV12, Rh10, Rh74 (SEQ ID NO: 188), AAV3B, LK03 (SEQ ID NO: 191), AAV-2i8, SEQ ID NO: 226, SEQ ID NO: 189, and/or SEQ ID NO: 190, but are distinct from and not identical to known AAV genes or proteins, such as AAV1, AAV2, AAV3, AAV4, AAV5, AAV6, AAV7, AAV8, AAV9, AAV10, AAV11, AAV12, Rh10, Rh74 (SEQ ID NO: 188), AAV3B, LK03 (SEQ ID NO: 191), AAV-2i8, SEQ ID NO: 226, SEQ ID NO: 189, and/or SEQ ID NO: 190. In certain embodiments, a modified/variant AAV capsid protein includes or consists of a sequence at least 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 99.9% identical to a reference or parental AAV capsid protein, such as AAV1, AAV2, AAV3, AAV4, AAV5, AAV6, AAV7, AAV8, AAV9, AAV10, AAV11, AAV12, Rh10, Rh74 (SEQ ID NO: 188), AAV3B, LK03 (SEQ ID NO: 191), AAV-2i8, SEQ ID NO: 226, SEQ ID NO: 189, and/or SEQ ID NO: 190.

In certain embodiments, a viral vector such as an adeno-associated virus (AAV) vector comprises any of the polynucleotides comprising the nucleic acids encoding C1 inhibitor as set forth herein operably linked to an expression control element.

In certain embodiments, a viral vector such as an adeno-associated virus (AAV) vector comprises any of the expression cassettes comprising the polynucleotides comprising the nucleic acids encoding C1 inhibitor as set forth herein.

In certain embodiments, an AAV vector comprises: one or more of an AAV capsid; and one or more AAV inverted terminal repeats (ITRs), wherein the AAV ITR(s) flanks the 5′ or 3′ terminus of the polynucleotide or the expression cassette.

In certain embodiments, an AAV vector further comprises an intron positioned 5′ or 3′ of one or more ITRs.

In certain embodiments, an AAV vector comprising at least one or more ITRs or an intron has the one or more ITRs or intron modified to have reduced CpGs.

In certain embodiments, the instant invention relates to an AAV vector comprising,
(1) a 5′ AAV ITR, optionally a 5′ ITR of AAV2, optionally a 5′ ITR comprising the polynucleotide sequence of SEQ ID NO: 70 or 72;
(2) one or more enhancers, optionally one or more enhancers having a polynucleotide sequence selected from the group consisting of SEQ ID NOs: 225, 74-78 and 173-178;
(3) one or more 5′ UTRs, optionally one or more 5′ UTRs having a polynucleotide sequence selected from the group consisting of SEQ ID NOs: 180 and 183;
(4) optionally an intron, optionally an intron having the polynucleotide sequence of SEQ ID NO: 81 or 82;
(5) a nucleic acid encoding a signal peptide, optionally the signal peptide comprising an amino acid sequence selected from the group consisting of SEQ ID NOs: 203-218, optionally a polynucleotide sequence selected from the group consisting of SEQ ID NOs: 84-103;
(6) a nucleic acid encoding at least one of a C1 inhibitor, a variant C1 inhibitor, and a fusion or combination thereof,
   a. optionally, a C1 inhibitor having the amino acid sequence of SEQ ID NO: 181 or 192, optionally a polynucleotide sequence selected from the group consisting of SEQ ID NOs: 104-142, 145-147, 156, and 171-172;
   b. optionally, a variant C1 inhibitor having an amino acid sequence selected from the group consisting of SEQ ID NOs: 193-201, optionally a polynucleotide selected from the group consisting of SEQ ID NOs: 143-144, 158, and 165-170;
(7) one or more 3′ UTRs, optionally one or more 3′ UTRs having the polynucleotide sequence of SEQ ID NO: 83 or 184;
(8) optionally a miRNA binding site, optionally an miRNA binding site having the polynucleotide sequence of SEQ ID NO: 179;
(9) optionally a regulated Irel-dependent decay (RIDD) sequence, optionally a RIDD having the polynucleotide sequence selected from the group consisting of SEQ ID NOs: 185-187; and
(10) a 3′ AAV ITR, optionally a 3′ ITR of AAV2, optionally a 3′ ITR comprising the polynucleotide sequence of SEQ ID NO: 71 or 73.

In certain embodiments an AAV vector comprises a polynucleotide that comprises a nucleic acid encoding a C1 inhibitor, wherein the nucleic acid is CpG-reduced compared to a wild-type coding sequence of C1 inhibitor, and the polynucleotide is encapsidated by a capsid comprising VP1 of SEQ ID NO: 226. In certain embodiments the capsid comprises VP1 of SEQ ID NO: 226, VP2 of SEQ ID NO: 189 and VP3 of SEQ ID NO: 190. In certain embodiments, the polynucleotide comprises a nucleic acid sequence at least 99% identical to SEQ ID NO: 22, provided that the nucleic acid sequence encodes SEQ ID NO: 192. In certain embodiments, the nucleic acid sequence comprises SEQ ID NO: 22. In certain embodiments the polynucleotide comprises a nucleic acid sequence at least 99% or 100% identical to SEQ ID NO: 22; and the capsid comprises VP1 of SEQ ID NO: 226. In certain embodiments the polynucleotide comprises a nucleic acid sequence at least 99% or 100% identical to SEQ ID NO: 22; and the capsid comprises VP1 of SEQ ID NO: 226, VP2 of SEQ ID NO: 189 and VP3 of SEQ ID NO: 190.

In certain embodiments, an AAV vector of the instant invention is delivered via a non-viral delivery system, including for example, encapsulated in a lipid nanoparticle (LNP).

### Non-Viral Methods

In certain embodiments, the polynucleotides and expression cassettes of the instant invention are delivered or administered with a non-viral delivery system. Non-viral delivery systems include for example, chemical methods, such as non-viral vectors, or extracellular vesicles and physical methods, such as gene gun, electroporation, particle bombardment, ultrasound utilization and magnetofection.

Recombinant cells capable of expressing the C1 inhibitor sequences of the instant invention can be used for delivery or administration.

Naked DNA such as minicircles and transposons can be used for administration or delivery or lentiviral vectors. Additionally, gene editing technologies such as zinc finger nucleases, meganucleases, TALENs, and CRISPR can also be used to deliver the coding sequence of the instant invention.

In certain embodiments, the polynucleotides and expression cassettes of the instant invention are delivered as naked DNA, minicircles, transposons, of closed-ended linear duplex DNA.

In certain embodiments, the polynucleotides and expression cassettes of the instant invention are delivered or administered in AAV vector particles, or other viral particles, that are further encapsulated or complexed with liposomes, nanoparticles, lipid nanoparticles, polymers, microparticles, microcapsules, micelles, or extracellular vesicles.

In certain embodiments, the polynucleotides and expression cassettes of the instant invention are delivered or administered with non-viral vectors.

As used herein, a “non-viral vector” refers to a vector that is not delivered by viral particles or by viral-like particles (VLPs). According to certain embodiments, a non-viral vector is a vector that is not delivered by a capsid. The vector can be encapsulated, admixed, or otherwise associated with a non-viral delivery particle or nanoparticle.

Any suitable non-viral delivery system known to those skilled in the art in view of the present disclosure can be used in the instant invention. A non-viral delivery particle or nanoparticle can be, for example, a lipid-based nanoparticle, a polymer-based nanoparticle, a protein-based nanoparticle, a microparticle, a microcapsule, a metallic particle-based nanoparticle, a peptide cage nanoparticle, etc.

A non-viral delivery particle or nanoparticle of the instant invention can be constructed by any method known in the art, and a non-viral vector of the instant invention comprising a nucleic acid molecule comprising a therapeutic transgene can be constructed by any method known in the art.

### Lipid-Based Delivery Systems

Lipid-based delivery systems are well known in the art, and any suitable lipid-based delivery system known to those skilled in the art in view of the present disclosure can be used in the instant invention. Examples of lipid-based delivery systems include, e.g., liposomes, lipid nanoparticles, micelles, or extracellular vesicles.

A “lipid nanoparticle” or “LNP” refers to a lipid-based vesicle useful for delivery of AAV and non-viral vectors having dimensions on the nanoscale, i.e., from about 10 nm to about 1000 nm, or from about 50 to about 500 nm, or from about 75 to about 127 nm.

Without being bound by theory, an LNP is believed to provide a polynucleotide, expression cassette, AAV vector, or non-viral vector with partial or complete shielding from the immune system. Shielding allows delivery of the polynucleotide, expression cassette, AAV vector, or non-viral vector to a tissue or cell while avoiding inducing a substantial immune response against the polynucleotide, expression cassette, AAV vector, or non-viral vector in vivo.

Shielding can also allow repeated administration without inducing a substantial immune response against the polynucleotide, expression vector, AAV vector, or non-viral vector in vivo (e.g., in a subject such as a human). Shielding can also improve or increase polynucleotide, expression cassette, AAV vector, or non-viral vector delivery efficiency in vivo.

The isoelectric point (pI) of AAV is in a pH range from about 6 to about 6.5. Thus, the AAV surface carries a slight negative charge. As such it can be beneficial for an LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, the disclosures of which are herein incorporated by reference in their entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids can also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids can include, without limitation, 1,2-dilinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylami nopropane (g-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, y-DLen-C2K-DMA, and (DLin-MP-DMA) (also known as 1-B11).

Still further cationic lipids can include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECT AMINE® (comprising DOSPA and DOPE, available from GIBCO/BRL).

In certain embodiments, cationic lipid can be present in an amount from about 10% by weight of the LNP to about 85% by weight of the lipid nanoparticle, or from about 50% by weight of the LNP to about 75% by weight of the LNP.

Sterols can confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols can include campesterol, sitosterol, and stigmasterol. Sterols also include sterol-modified lipids, such as those described in U.S. Patent Application Publication 2011/0177156, the disclosure of which is herein incorporated by reference in its entirety. In certain embodiments, a sterol can be present in an amount from about 5% by weight of the LNP to about 50% by weight of the lipid nanoparticle or from about 10% by weight of the LNP to about 25% by weight of the LNP.

LNP can comprise a neutral lipid. Neutral lipids can comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized by well-known techniques. In certain embodiments, lipids containing saturated fatty acids with carbon chain lengths in the range of C14 to C22 can be used. In certain embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids can also be composed of sphingomyelin, dihydrosphingomyelin, or phospholipids with other head groups, such as serine and inositol.

In certain embodiments, the neutral lipid can be present in an amount from about 0.10% by weight of the lipid nanoparticle to about 75% by weight of the LNP, or from about 5% by weight of the LNP to about 15% by weight of the LNP.

LNP encapsulated nucleic acids, expression cassettes, AAV vectors, and non-viral vectors can be incorporated into pharmaceutical compositions, e.g., a pharmaceutically acceptable carrier or excipient. Such pharmaceutical compositions are useful for, among other things, administration and delivery of LNP encapsulated acids, expression cassettes, AAV vectors, and non-viral vectors to a subject in vivo or ex vivo.

Preparations of LNP can be combined with additional components. Non-limiting examples include polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG can be a polyethylene glycol with an average molecular weight of about 550 to about 10,000 daltons and is optionally substituted by alkyl, alkoxy, acyl or aryl. In certain embodiments, the PEG can be substituted with methyl at the terminal hydroxyl position. In certain embodiments, the PEG can have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group can be substituted with a methoxy or methyl group.

PEG-modified lipids include the PEG-dialkyloxypropyl conjugates (PEG-DAA) described in U.S. Pat. Nos. 8,936,942 and 7,803,397, the disclosures of which are herein incorporated by reference in their entirety. PEG-modified lipids (or lipid-polyoxyethylene conjugates) that are useful can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerCl4 or PEG-CerC20) which are described in U.S. Pat. No. 5,820,873, the disclosure of which is herein incorporated by reference in its entirety, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs can have a size in a range from about 10 nm to 500 nm, or from about 50 nm to about 200 nm, or from 75 nm to about 125 nm. In certain embodiments, LNP encapsulated nucleic acid, expression vector, AAV vector, or non-viral vector can have a size in a range from about 10 nm to 500 nm.

### Polymer-Based Systems

Polymer-based delivery systems are well known in the art, and any suitable polymer-based delivery system or polymeric nanoparticle known to those skilled in the art in view of the present disclosure can be used in the instant invention. DNA can be entrapped into the polymeric matrix of polymeric nanoparticles or can be adsorbed or conjugated on the surface of the nanoparticles. Examples of commonly used polymers for gene delivery include, e.g., poly(lactic-co-glycolic acid) (PLGA), poly lactic acid (PLA), poly(ethylene imine) (PEI), chitosan, dendrimers, polyanhydride, polycaprolactone, and polymethacrylates.

The polymeric-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Protein-Based Systems

Protein-based delivery systems are well known in the art, and any suitable protein-based delivery system or cell-penetrating peptide (CPP) known to those skilled in the art in view of the present disclosure can be used in the instant invention.

CPPs are short peptides (6-30 amino acid residues) that are potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include, e.g., cationic CPPs (highly positively charged) (e.g., the Tat peptide, penetratin, protamine, poly-L-lysine, polyarginine, etc.); amphipathic CPPs (chimeric or fused peptides, constructed from different sources, contain both positively and negatively charged amino acid sequences) (e.g., transportan, VT5, bactenecin-7 (Bac7), proline-rich peptide (PPR), SAP (VRLPPP)₃, TP10, pep-1, MPG, etc.); membranotropic CPPs (exhibit both hydrophobic and amphipathic nature simultaneously, and comprise both large aromatic residues and small residues) (e.g., gH625, SPIONs-PEG-CPP NPs, etc.); and hydrophobic CPPs (contain only non-polar motifs or residues) (e.g., SG3, PFVYLI, pep-7, fibroblast growth factors (FGF), etc.).

The protein-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Peptide Cage Systems

Peptide cage-based delivery systems are well known in the art, and any suitable peptide cage-based delivery system known to those skilled in the art in view of the present disclosure can be used in the instant invention. In general, any proteinaceous material that is able to be assembled into a cage-like structure, forming a constrained internal environment, can be used. Several different types of protein “shells” can be assembled and loaded with different types of materials. For example, protein cages comprising a shell of viral coat protein(s) (e.g., from the Cowpea Chlorotic Mottle Virus (CCMV) protein coat) that encapsulate a non-viral material, as well as protein cages formed from non-viral proteins have been described (see, e.g., U.S. Pat. Nos. 6,180,389 and 6,984,386, U.S. Patent Application 20040028694, and U.S. Patent Application 20090035389, the disclosures of which are herein incorporated by reference in their entirety). Peptide cages can comprise a proteinaceous shell that self-assembles to form a protein cage (e.g., a structure with an interior cavity which is either naturally accessible to the solvent or can be made to be so by altering solvent concentration, pH, equilibria ratios).

Examples of protein cages derived from non-viral proteins include, e.g., ferritins and apoferritins, derived from both eukaryotic and prokaryotic species, e.g., 12 and 24 subunit ferritins; and protein cages formed from heat shock proteins (HSPs), e.g., the class of 24 subunit heat shock proteins that form an internal core space, the small HSP of Methanococcus jannaschii, the dodecameric Dps HSP of E. coli, the MrgA protein, etc. As will be appreciated by those in the art, the monomers of the protein cages can be naturally occurring or variant forms, including amino acid substitutions, insertions and deletions (e.g., fragments) that can be made.

The protein cages can have different core sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Pharmaceutical Compositions

The instant invention additionally provides pharmaceutical compositions comprising any of the polynucleotides comprising the nucleic acids encoding C1 inhibitor, expression cassettes comprising polynucleotides comprising the nucleic acids encoding C1 inhibitor, viral vectors such as AAV vectors comprising polynucleotides comprising the nucleic acids encoding C1 inhibitor, or non-viral vectors comprising polynucleotides comprising the nucleic acids encoding C1 inhibitor as set forth herein.

rAAV vectors and non-viral vectors can be administered to a patient via infusion in a biologically compatible carrier, for example, via intravenous injection. rAAV vectors and non-viral vectors can be administered alone or in combination with other molecules.

Accordingly, rAAV vectors and non-viral vectors and other compositions, agents, drugs, biologics (proteins) can be incorporated into pharmaceutical compositions. Such pharmaceutical compositions are useful for, among other things, administration and delivery to a subject in vivo or ex vivo.

In certain embodiments, pharmaceutical compositions also contain a pharmaceutically acceptable carrier or excipient. Such excipients include any pharmaceutical agent that does not itself induce an immune response harmful to the individual receiving the composition, and which can be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material can be administered to a subject without causing substantial undesirable biological effects. Thus, such a pharmaceutical composition can be used, for example in administering a polynucleotide, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Excipients also include proteins such as albumin.

Additionally, auxiliary substances, such as wetting or emulsifying agents, pH buffering substances, and the like, can be present in such vehicles.

The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions can include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions can contain substances which increase the viscosity of the suspension, such as sodium carboxymethyl cellulose, sorbitol, or dextran.

Additionally, suspensions of the active compounds can be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase the solubility of the compounds to allow for the preparation of highly concentrated solutions.

Cosolvents and adjuvants can be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

In certain embodiments, a pharmaceutical composition comprising any of the AAV vectors as set forth herein, further comprises empty AAV capsids. In certain embodiments, in a pharmaceutical composition comprising AAV vectors and empty AAV capsids, the ratio of the empty AAV capsids to the AAV vector is within or between about 100:1-50:1, from about 50:1-25:1, from about 25:1-10:1, from about 10: 1-1:1, from about 1:1-1:10, from about 1:10-1:25, from about 1:25-1:50, or from about 1:50-1:100. In certain embodiments, in a pharmaceutical composition comprising AAV vectors and empty AAV capsids, the ratio of the of the empty AAV capsids to the AAV vector is about 2:1, 3:1, 4:1, 5:1, 6:1, 7:1, 8:1, 9:1, or 10:1.

In certain embodiments, a pharmaceutical composition includes a surfactant.

After pharmaceutical compositions have been prepared, they can be placed in an appropriate container and labeled for treatment. Such labeling could include amount, frequency, and method of administration.

Pharmaceutical compositions and delivery systems appropriate for the compositions, methods and uses of the instant invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technomic Publishing Co., Inc., Lancaster, Pa.; Ansel and Stoklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppressive agents such as a drug), treatments, protocols, or therapeutic agents, a detectable response of any duration of time (long or short term), an expected or desired outcome in or a benefit to a subject of any measurable or detectable degree or for any duration of time (e.g., for minutes, hours, days, months, years, or cured).

Compositions such as pharmaceutical compositions can be delivered to a subject, so as to allow production of the encoded protein. In certain embodiments, pharmaceutical compositions comprise sufficient genetic material to enable a recipient to produce a therapeutically effective amount of a protein in the subject.

A “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

Compositions can be formulated and/or administered in any sterile, biocompatible pharmaceutical carrier, including, but not limited to, saline, buffered saline, dextrose, and water. The compositions can be formulated and/or administered to a patient alone, or in combination with other agents (e.g., co-factors) which influence hemostasis.

### Methods of Treatment

HAE treatment with C1-INI-H-replacement therapy increases survival, reduces attack frequency and severity, and is a current standard of care for HAE disease patients. However, current treatment modalities have several drawbacks such as the potential for breakthrough attacks, safety/tolerability, high patient burden, and potential for limited compliance. HAE gene transfer treatment preferably overcomes on or more of these drawbacks to C1-INH-replacement therapy. For example, HAE gene transfer treatment described herein is expected to require less frequent dosing, preferably a single dose will be sufficient.

The instant invention still further provides methods of treating a subject in need of C1 inhibitor, comprising administering to the subject a therapeutically effective amount of a polynucleotide, expression cassette, AAV vector, non-viral vector, or pharmaceutical composition of the instant invention, wherein the C1 inhibitor is expressed in the subject.

Methods and uses of the instant invention include delivering (transducing) polynucleotide (transgene) into host cells, including dividing and/or non-dividing cells. The polynucleotides, expression cassettes, rAAV vectors, non-viral vectors, methods, uses and pharmaceutical formulations of the instant invention are additionally useful in a method of delivering, administering or providing sequence encoded by heterologous nucleic acid to a subject in need thereof, as a method of treatment. In this manner, the polynucleotide comprising the nucleic acid is transcribed and a protein produced in vivo in a subject. The subject can benefit from or be in need of the protein because the subject has a deficiency of the protein, or because production of the protein in the subject can impart some therapeutic effect, as a method of treatment or otherwise.

The instant invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of protein/enzyme deficiencies such as HAE, and others known to those of skill in the art.

Subjects appropriate for treatment in accordance with the instant invention include those having or at risk of producing an insufficient amount of C1 inhibitor, or producing an aberrant, partially functional or non-functional C1 inhibitor. Subjects can be tested for C1 inhibitor activity to determine if such subjects are appropriate for treatment according to a method of the instant invention. Subjects appropriate for treatment in accordance with the instant invention also include those subjects that would benefit from C1 inhibitor. Such subjects that can benefit from C1 inhibitor include those having a complement-mediated disorder. Treated subjects can be monitored after treatment periodically, e.g., every 1-4 weeks, 1-6 months, 6-12 months, or 1, 2, 3, 4, 5 or more years.

Subjects can be tested for an immune response, e.g., antibodies against AAV. Candidate subjects can therefore be screened prior to treatment according to a method of the instant invention. Subjects also can be tested for antibodies against AAV after treatment, and optionally monitored for a period of time after treatment. Subjects having pre-existing or developing AAV antibodies can be treated with an immunosuppressive agent, or other regimen as set forth herein.

Subjects appropriate for treatment in accordance with the instant invention also include those having or at risk of producing antibodies against AAV. rAAV vectors can be administered or delivered to such subjects using several techniques. For example, AAV empty capsid (i.e., AAV particles lacking a modified nucleic acid encoding C1 inhibitor) can be delivered to bind to the AAV antibodies in the subject thereby allowing the rAAV vector comprising the heterologous nucleic acid to transduce cells of the subject. The terms “AAV empty capsid,” “AAV empty capsid particle(s),” “empty capsid AAV,” “empty capsid AAV particle(s)” are used interchangeably herein.

The modified nucleic acids, expression cassettes, rAAV vectors, and non-viral vectors of the instant invention can be used for treatment of a C1 inhibitor deficiency. Accordingly, in certain embodiments, modified nucleic acids encoding C1 inhibitor, expression cassettes comprising modified nucleic acids encoding C1 inhibitor, rAAV vectors, and non-viral vectors of the instant invention can be used as a therapeutic and/or prophylactic agent.

In certain embodiments, the modified nucleic acids encoding C1 inhibitor, expression cassettes comprising modified nucleic acids encoding C1 inhibitor, rAAV vectors, and non-viral vectors of the instant invention can be used for treatment of HAE. Administration of modified nucleic acids encoding C1 inhibitor, expression cassettes comprising modified nucleic acids encoding C1 inhibitor, rAAV vectors, and non-viral vectors of the instant invention to a patient with HAE leads to the expression of the C1 inhibitor protein.

In certain embodiments, a method according to the instant invention can result in expression or activity of C1 inhibitor at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the C1 inhibitor protein found in a subject not in need of C1 inhibitor.

Subjects, animals or patients administered the modified nucleic acids encoding C1 inhibitor, expression cassettes comprising modified nucleic acids encoding C1 inhibitor, rAAV vectors, and non-viral vectors of the instant invention can be evaluated by a variety of tests, assays and functional assessments to demonstrate, measure and/or assess efficacy of the modified nucleic acids encoding C1 inhibitor, expression cassettes comprising modified nucleic acids encoding C1 inhibitor, rAAV vectors, and non-viral vectors of the instant invention as therapeutic and/or prophylactic agents. Such tests and assays include, but are not limited to, measurement of C1 inhibitor activity (such as by use of standard C1 inhibitor activity assays) and/or C1 inhibitor amount (such as by western blot with anti-C1 inhibitor antibody) in a biological sample such as blood or plasma; analysis of peak and steady-state vector-derived C1 inhibitor enzyme levels assessed by total C1 inhibitor protein and activity in plasma; testing for immune responses against AAV capsid; and testing for immune responses against the C1 inhibitor transgene protein product.

Additionally, the modified nucleic acids encoding C1 inhibitor, expression cassettes comprising modified nucleic acids encoding C1 inhibitor, rAAV vectors, and non-viral vectors of the instant invention can be used for treatment of a complement-mediated disorder. According to certain embodiments, the modified nucleic acids encoding C1 inhibitor, expression cassettes comprising modified nucleic acids encoding C1 inhibitor, rAAV vectors, and non-viral vectors of the instant invention can be used for treatment of a patient in need of C1 inhibitor. According to certain embodiments, the modified nucleic acids encoding C1 inhibitor, expression cassettes comprising modified nucleic acids encoding C1 inhibitor, rAAV vectors, and non-viral vectors of the instant invention can be used for treatment of hereditary angioedema (HAE).

As used herein, the term “hereditary angioedema” or “HAE” refers to a blood disorder characterized by unpredictable and recurrent attacks of inflammation. HAE is typically associated with CT-INH deficiency, which may be the result of low levels of C1-INH or C1-INH with impaired or decreased activity. Symptoms include, but are not limited to, swelling that can occur in any part of the body, such as the face, extremities, genitals, gastrointestinal tract, and upper airways. As used herein, the HAE can be Type I, Type II, or Type III.

As set forth herein, rAAV are useful as gene therapy vectors as they can penetrate cells and introduce nucleic acid/genetic material into the cells. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity, and immune responses are typically minimal or undetectable. AAV are known to infect a wide variety of cell types in vivo by receptor-mediated endocytosis or by transcytosis. These vector systems have been tested in humans targeting many tissues, such as, retinal epithelium, liver, skeletal muscle, airways, brain, joints and hematopoietic stem cells.

It can be desirable to introduce a rAAV vector that can provide, for example, multiple copies of C1 inhibitor and hence greater amounts of C1 inhibitor protein. Improved rAAV vectors and methods for producing these vectors have been described in detail in a number of references, patents, and patent applications, including: Wright J. F. (Hum. Gene Ther., 20:698-706, 2009).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration can be proportionally increased or reduced, as indicated by any adverse side effects, complications or other risk factors of the treatment or therapy and the status of the subject. The skilled artisan will appreciate the factors that can influence the dosage and timing required to provide an amount sufficient for providing a therapeutic or prophylactic benefit.

The dose to achieve a therapeutic effect, e.g., the dose in vector genomes/per kilogram of body weight (vg/kg) of rAAV, or the dose of non-viral vector, will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome or non-viral vector dose range to treat a patient having a particular disease or disorder based on the aforementioned factors, as well as other factors.

Generally, doses of rAAVs will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect.

For example, a dose of about 2×10¹¹ recombinant AAV vg/kg or greater than about 2×10¹¹ recombinant AAV vg/kg; a dose of about 3×10¹¹ recombinant AAV vg/kg or greater than about 3×10¹¹ recombinant AAV vg/kg; a dose of about 4×10¹¹ recombinant AAV vg/kg or greater than about 4×10¹¹ recombinant AAV vg/kg; a dose of about 5×10¹¹ recombinant AAV vg/kg or greater than about 5×10¹¹ recombinant AAV vg/kg; a dose of about 1×10¹² recombinant AAV vg/kg or greater than about 1×10¹² recombinant AAV vg/kg; a dose of about 2×10¹² recombinant AAV vg/kg or greater than about 2×10¹² recombinant AAV vg/kg; a dose of about 3×10¹² recombinant AAV vg/kg or greater than about 3×10¹² recombinant AAV vg/kg; a dose of about 4×10¹² recombinant AAV vg/kg or greater than about 4×10¹² recombinant AAV vg/kg; a dose of about 5×10¹² recombinant AAV vg/kg or greater than about 5×10¹² recombinant AAV vg/kg; a dose of about 1×10¹³ recombinant AAV vg/kg or greater than about 1×10¹³ recombinant AAV vg/kg; a dose of about 2×10¹³ recombinant AAV vg/kg or greater than about 2×10¹³ recombinant AAV vg/kg; a dose of about 3×10¹³ recombinant AAV vg/kg or greater than about 3×10¹³ recombinant AAV vg/kg; a dose of about 4×10¹³ recombinant AAV vg/kg or greater than about 4×10¹³ recombinant AAV vg/kg; a dose of about 5×10¹³ recombinant AAV vg/kg or greater than about 5×10¹³ recombinant AAV vg/kg.

Exemplary dose ranges of recombinant AAV vg/kg administered are a dose range from about 1.5×10¹¹ to about 5×10¹³ recombinant AAV vg/kg; a dose range from about 1.5×10¹¹ to about 2×10¹¹ recombinant AAV vg/kg; a dose range from about 2×10¹¹ to about 2.5×10¹¹ recombinant AAV vg/kg; a dose range from about 2.5×10¹¹ to about 3×10¹¹ recombinant AAV vg/kg; a dose range from about 3×10¹¹ to about 3.5×10¹¹ recombinant AAV vg/kg; a dose range from about 3.5×10¹¹ to about 4×10¹¹ recombinant AAV vg/kg; a dose range from about 4×10¹¹ to about 4.5×10¹¹ recombinant AAV vg/kg; a dose range from about 4.5×10¹¹ to about 5×10¹¹ recombinant AAV vg/kg; a dose range from about 5×10¹¹ to about 1×10¹² recombinant AAV vg/kg; a dose range from about 1×10¹² to about 1.5×10¹² recombinant AAV vg/kg; a dose range from about 1.5×10¹² to about 2×10¹² recombinant AAV vg/kg; a dose range from about 2×10¹² to about 2.5×10¹² recombinant AAV vg/kg; a dose range from about 2.5×10¹² to about 3×10¹² recombinant AAV vg/kg; a dose range from about 3×10¹² to about 3.5×10¹² recombinant AAV vg/kg; a dose range from about 3.5×10¹² to about 4×10¹² recombinant AAV vg/kg; a dose range from about 4×10¹² to about 4.5×10¹² recombinant AAV vg/kg; a dose range from about 4.5×10¹² to about 5×10¹² recombinant AAV vg/kg; a dose range from about 5×10¹² to about 1×10¹³ recombinant AAV vg/kg; a dose range from about 1×10¹³ to about 1.5×10¹³ recombinant AAV vg/kg; a dose range from about 1.5×10¹³ to about 2×10¹³ recombinant AAV vg/kg; a dose range from about 2×10¹³ to about 2.5×10¹³ recombinant AAV vg/kg; a dose range from about 2.5×10¹³ to about 3×10¹³ recombinant AAV vg/kg; a dose range from about 3×10¹³ to about 3.5×10¹³ recombinant AAV vg/kg; a dose range from about 3.5×10¹³ to about 4×10¹³ recombinant AAV vg/kg; a dose range from about 4×10¹³ to about 4.5×10¹³ recombinant AAV vg/kg; a dose range from about 4.5×10¹³ to about 5×10¹³ recombinant AAV vg/kg; and a dose range from about 5×10¹³ to about 1×10¹⁴ recombinant AAV vg/kg.

In certain embodiments, AAV vg/kg are administered at a dose of about 1×10¹¹ vg/kg, administered at a dose of about 2×10¹¹ vg/kg, administered at a dose of about 3×10¹¹ vg/kg, administered at a dose of about 4×10¹¹ vg/kg, administered at a dose of about 5×10¹¹ vg/kg, administered at a dose of about 6×10¹¹ vg/kg, administered at a dose of about 7×10¹¹ vg/kg, administered at a dose of about 8×10¹¹ vg/kg, administered at a dose of about 9×10¹¹ vg/kg, administered at a dose of about 1×10¹² vg/kg, administered at a dose of about 2×10¹² vg/kg, administered at a dose of about 3×10¹² vg/kg, administered at a dose of about 4×10¹² vg/kg, administered at a dose of about 5×10¹² vg/kg, administered at a dose of about 6×10¹² vg/kg, administered at a dose of about 7×10¹² vg/kg, administered at a dose of about 8×10¹² vg/kg, administered at a dose of about 9×10¹² vg/kg, administered at a dose of about 1×10¹³ vg/kg, administered at a dose of about 2×10¹³ vg/kg, administered at a dose of about 3×10¹³ vg/kg, administered at a dose of about 4×10¹³ vg/kg, administered at a dose of about 5×10¹³ vg/kg, administered at a dose of about 6×10¹³ vg/kg, administered at a dose of about 7×10¹³ vg/kg, administered at a dose of about 8×10¹³ vg/kg, administered at a dose of about 9×10¹³ vg/kg.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms can be within, for example, ampules and vials, which can include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, non-viral vectors, and pharmaceutical compositions thereof can be packaged in single or multiple unit dosage form for ease of administration and uniformity of dosage.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

In certain embodiments, a method according to the instant invention reduces, decreases or inhibits one or more symptoms of the need for C1 inhibitor or of HAE; or prevents or reduces progression or worsening of one or more symptoms of the need for C1 inhibitor or of HAE; or stabilizes one or more symptoms of the need for C1 inhibitor or of HAE; or improves one or more symptoms of the need for C1 inhibitor or of HAE.

An effective amount or a sufficient amount can but need not be provided in a single administration, can require multiple administrations, and, can but need not be, administered alone or in combination with another composition (e.g., agent), treatment, protocol or therapeutic regimen. For example, the amount can be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens can be included in order to be considered effective or sufficient in a given subject. Amounts considered effective also include amounts that result in a reduction of the use of another treatment, therapeutic regimen or protocol, such as administration of modified nucleic acid encoding C1 inhibitor for treatment of a C1 inhibitor deficiency (e.g., HAE).

Accordingly, methods and uses of the instant invention also include, among other things, methods and uses that result in a reduced need or use of another compound, agent, drug, therapeutic regimen, treatment protocol, process, or remedy. For example, for C1 inhibitor deficiency, a method or use of the instant invention has a therapeutic benefit if in a given subject, a less frequent or reduced dose or elimination of administration of a recombinant C1 inhibitor to supplement for the deficient or defective C1 inhibitor in the subject is needed. Thus, in accordance with the instant invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., C1 inhibitor or a protein deficiency that leads to a HAE), or that produce an aberrant, partially functional or non-functional gene product (e.g., C1 inhibitor or a protein implicated in HAE).

Administration or in vivo delivery to a subject in accordance with the methods and uses of the instant invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the instant invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For HAE disease, an effective amount would be an amount that improves markers for HAE disease, such as subcutaneous edema, and swelling in any part of the body, such as the face, extremities, genitals, gastrointestinal tract, and upper airways, and biomarkers such as levels of C1 inhibitor antigen and activity, levels of complement C4 (C4 levels are below normal in 95% of patients with HAE, even when asymptomatic), and levels of bradykinin.

Improvement of biomarkers for HAE includes increased levels of C1 inhibitor antigen and activity, increased or stabilized levels of complement C4 (low circulating levels of complement C4 is an indicator of HAE), and decreased levels of bradykinin (indicative of restoration of C1-INH function/activity). Many assays to measure or quantitate levels of complement C4 in plasma, sera or tissue are known in the art, including multiplexed assays (Lai et al., J Pharm. Biomed. Anal., 195: 113844 (2020) doi: 10.1016/j.jpba.2020.113844) and immunoassays such as radial immunodiffusion (Koelle et al., J Clin. Microbio., 16: 271-275 (1982)) and ELISA (Kaplan et al., Front. Med., 4: 206 (2017) doi: 10.3389/fmed.2017.00206).

In certain embodiments, methods and uses of the instant invention increase levels of C1 inhibitor antigen and activity in blood, plasma, sera and/or tissue of a subject. In certain embodiments, methods and uses of the instant invention decrease bradykinin levels in blood, plasma, sera and/or tissue of a subject. In certain embodiments, methods and uses of the instant invention increase or stabilize levels of complement C4 in blood plasma, sera and/or tissue of a subject.

Therapeutic doses will depend on, among other factors, the age and general condition of the subject, the severity of the disease or disorder. Thus, a therapeutically effective amount in humans will fall in a relatively broad range that can be determined by a medical practitioner based on the response of an individual patient.

Methods and uses of the instant invention include delivery and administration systemically, regionally or locally, or by any route, for example, by injection or infusion. Delivery of the pharmaceutical compositions in vivo can generally be accomplished via injection using a conventional syringe, although other delivery methods such as convection-enhanced delivery are envisioned (See e.g., U.S. Pat. No. 5,720,720, the disclosure of which is herein incorporated by reference in its entirety). For example, compositions can be delivered subcutaneously, epidermally, intradermally, intrathecally, intraorbitally, intramucosally, intranasally, intraperitoneally, intravenously, intra-pleurally, intraarterially, intracavitary, orally, intrahepatically, via the portal vein, or intramuscularly. Other modes of administration include oral and pulmonary administration, suppositories, and transdermal applications. A clinician specializing in the treatment of patients with HAE can determine the optimal route for administration of AAV vectors and non-viral vectors based on a number of criteria, including, but not limited to: the condition of the patient and the purpose of the treatment (e.g., enhanced or reduced C1 inhibitor levels).

The compositions can be administered alone. In certain embodiments, an rAAV particle or a non-viral vector provides a therapeutic effect without an immunosuppressive agent. The therapeutic effect optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, a therapeutic effect is provided for a period of time.

rAAV vectors, non-viral vectors, methods, and uses of the instant invention can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs.

Such biologics (proteins), agents, drugs, treatments and therapies can be administered or performed prior to, substantially contemporaneously with or following any other method or use of the instant invention.

The compound, agent, drug, treatment or other therapeutic regimen or protocol can be administered as a combination composition, or administered separately, such as concurrently or in series or sequentially (prior to or following) to delivery or administration of a polynucleotide, expression cassette, rAAV particle, or non-viral vector. The instant invention therefore provides combinations in which a method or use of the instant invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a polynucleotide, expression cassette, non-viral vector, or rAAV particle of the instant invention, to a subject.

In certain embodiments, nucleic acids, expression vectors, non-viral vectors, or rAAV particles of the instant invention are administered to a patient in combination with an immunosuppressive agent or regimen where the patient has or is at risk of developing an immune response against the rAAV particle and/or the C1 inhibitor protein. Such immunosuppressive agent or regimen can be administered prior to, substantially at the same time or after administering a polynucleotide, expression cassette, non-viral vector, or rAAV vector of the instant invention.

In certain embodiments, a subject or patient, such as a human patient, with HAE has developed inhibitors to the C1 inhibitor protein (including anti-C1 inhibitor antibodies and/or anti-C1 inhibitor T-cells), which can occur following treatment with traditional enzyme replacement therapy (e.g., following administration of recombinantly produced C1 inhibitor protein). The development of such C1 inhibitor inhibitors can occur in patients that receive enzyme replacement therapy, particularly where the patient has undetectable C1 inhibitor levels, leading the patient's immune system to see the replacement C1 inhibitor protein as “foreign.” In certain embodiments, an HAE patient having C1 inhibitor inhibitors is administered one or more regimen intended to achieve immune tolerance or mitigate the immune response to the C1 inhibitor protein in the patient, prior to, substantially at the same time or after administering an rAAV vector or non-viral vector of the instant invention. Such regimens to achieve immune tolerance or mitigate the immune response to the C1 inhibitor protein can include administration of one or more immunosuppressive agent, including but not limited to methotrexate, rituximab, intravenous gamma globulin (IVIG), omalizumab, and synthetic vaccine particle (SVP™)-rapamycin (rapamycin encapsulated in a biodegradable nanoparticle) and/or administration of one or more immunosuppressive protocol or procedure, such as B-cell depletion, immunoadsorption, and plasmapheresis.

In certain embodiments, rAAV vector or non-viral vector is administered in conjunction with one or more immunosuppressive agents prior to, substantially at the same time or after administering an rAAV vector or a non-viral vector. In certain embodiments, the one or more immunosuppressive agents is administered, e.g., 1-12, 12-24 or 24-48 hours, or 2-4, 4-6, 6-8, 8-10, 10-14, 14-20, 20-25, 25-30, 30-50, or more than 50 days following administering an rAAV vector or a non-viral vector. Such administration of immunosuppressive agents after a period of time following administering rAAV vector or non-viral vector can be done if there is a decrease in the encoded protein or inhibitory nucleic acid after the initial expression levels for a period of time, e.g., 20-25, 25-30, 30-50, 50-75, 75-100, 100-150, 150-200 or more than 200 days following rAAV vector or non-viral vector.

In certain embodiments, an immunosuppressive agent is an anti-inflammatory agent.

In certain embodiments, an immunosuppressive agent is a steroid, e.g., a corticosteroid. In certain embodiments, an immunosuppressive agent is prednisone, prednisolone, calcineurin inhibitor (e.g., cyclosporine, tacrolimus), CD52 inhibitor (e.g., alemtuzumab), CTLA4-Ig (e.g., abatacept, belatacept), anti-CD3 mAb, anti-LFA-1 mAb (e.g., efalizumab), anti-CD40 mAb (e.g., ASKP1240), anti-CD22 mAb (e.g., epratuzumab), anti-CD20 mAb (e.g., rituximab, orelizumab, ofatumumab, veltuzumab), proteasome inhibitor (e.g., bortezomib), TACI-Ig (e.g., atacicept), anti-C5 mAb (e.g., eculizumab), mycophenolate, azathioprine, sirolimus, everolimus, TNFR-Ig (e.g., etanercept (Enbrel®), anti-TNF mAb (e.g., adalimumab (Humira®), infliximab (Remicade®; Avsola®)), tofacitinib, anti-IL-2R (e.g., basiliximab), anti-IL-17 mAb (e.g., secukinumab), anti-IL-6 mAb (e.g., anti-IL-6 antibody sirukumab, anti-IL-6 receptor antibody tocilizumab (Actemra®), IL-10 inhibitor, TGF-beta inhibitor, a B cell targeting antibody (e.g., rituximab), a mammalian target of rapamycin (mTOR) inhibitor (e.g., rapamycin), synthetic vaccine particle (SVP™)-rapamycin (rapamycin encapsulated in a biodegradable nanoparticle), intravenous gamma globulin (IVIG), omalizumab, methotrexate, a tyrosine kinase inhibitor (e.g., ibrutinib), cyclophosphamide, fingolimod, an inhibitor of B-cell activating factor (BAFF) (e.g, anti-BAFF mAb, e.g., belimumab), an inhibitor of a proliferation-inducing ligand (APRIL), anti-IL-1b mAb (e.g., canakinumab (Haris®)), a C3a inhibitor, a Tregitope (see, e.g., U.S. Pat. No. 10,213,496), or a combination and/or derivative thereof.

In certain embodiments, rAAV vector or non-viral vector is administered in conjunction with one or more immunosuppressive agents prior to, substantially at the same time or after administering an rAAV vector or a non-viral vector, and/or in conjunction with administration of one or more immunosuppressive protocol or procedure, such as B-cell depletion, immunoadsorption, and plasmapheresis.

Immune-suppression protocols, including the use of rapamycin, alone or in combination with IL-10, can be used to decrease, reduce, inhibit, prevent or block humoral and cellular immune responses to the C1 inhibitor protein. Hepatic gene transfer with AAV vectors of the instant invention can be used to induce immune tolerance to the C1 inhibitor protein through induction of regulatory T cells (Tregs) and other mechanisms. Strategies to overcome or avoid humoral immunity to AAV in systemic gene transfer include, administering high vector doses, use of AAV empty capsids as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies (Nab), use of plasma exchange cycles to adsorb anti-AAV immunoglobulins, thereby reducing anti-AAV antibody titer, and use of delivery techniques such as balloon catheters followed by saline flushing. Such strategies are described in Mingozzi et al., 2013, Blood, 122:23-36.

Additional strategies include use of AAV-specific plasmapheresis columns to selectively deplete anti-AAV antibodies without depleting the total immunoglobulin pool from plasma, as described in Bertin et al., 2020, Sci. Rep. 10:864. Apheresis strategies to remove, deplete, capture, and/or inactivate AAV antibodies in subjects are described in WO2019018439.

Ratio of AAV empty capsids to the rAAV vector can be within or between about 100: 1-50:1, from about 50: 1-25:1, from about 25:1-10:1, from about 10:1-1:1, from about 1:1-1:10, from about 1:10-1:25, from about 1:25-1:50, or from about 1:50-1:100. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject. AAV empty capsids can be of any serotype, for example, AAV1, AAV2, AAV3, AAV4, AAV5, AAV6, AAV7, AAV8, AAV9, AAV10, AAV11, AAV12, Rh10, Rh74 (SEQ ID NO: 188), AAV3B, AAV-2i8, SEQ ID NO: 226, SEQ ID NO: 189, SEQ ID NO: 190, and/or SEQ ID NO: 191.

Alternatively, or in addition, rAAV vector or non-viral vector can be delivered by direct intramuscular injection (e.g., one or more slow-twitch fibers of a muscle). In another alternative, a catheter introduced into the femoral artery can be used to deliver rAAV vectors or non-viral vectors to liver via the hepatic artery. Non-surgical means can also be employed, such as endoscopic retrograde cholangiopancreatography (ERCP), to deliver rAAV vectors or non-viral vectors directly to the liver, thereby bypassing the bloodstream and AAV antibodies. Other ductal systems, such as the ducts of the submandibular gland, can also be used as portals for delivering rAAV vectors or non-viral vectors into a subject that develops or has preexisting anti-AAV antibodies.

Additional strategies to reduce humoral immunity to AAV include methods to remove, deplete, capture, and/or inactivate AAV antibodies, commonly referred to as apheresis and more particularly, plasmapheresis where blood products are involved. Apheresis or plasmapheresis, is a process in which a human subject's plasma is circulated ex vivo (extracorporal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure depletes, captures, inactivates, reduces or removes immunoglobulins (antibodies) that bind AAV thereby reducing the titer of AAV antibodies in the treated subject that can contribute to AAV vector neutralization. An example is a device composed of an AAV capsid affinity matrix column. Passing blood product (e.g., plasma) through an AAV capsid affinity matrix would result in binding only of AAV antibodies, and of all isotypes (including IgG, IgM, etc.).

A sufficient amount of plasmapheresis using an AAV capsid affinity matrix is predicted to substantially remove AAV capsid antibodies, and reduce the AAV capsid antibody titer (load) in the human. In certain embodiments, titer in a treated subject is reduced substantially to low levels (to <1:5, or less, such as <1:4, or <1:3, or <1:2, or <1:1). A reduction in antibody titer will be temporary because the B lymphocytes that produce the AAV capsid antibodies would be expected to gradually cause the AAV capsid antibody titer to rebound to the steady state level prior to plasmapheresis.

In the case where a pre-existing AAV antibody titer was reduced from 1:100 to 1:1, AAV antibody titer rebounds of approximately 0.15% (corresponding to a titer of 1:1.2) 0.43% (1:1.4), 0.9% (1:1.9), 1.7% (1:2.7), and 3.4% (1:4.4), occur at 1 hour, 3 hours, 6 hours, 12 hours and 24 hours, respectively, after completion of the plasmapheresis method. Temporary removal of AAV antibodies from such a subject would correspond to a window of time (for example, of about 24 hours or less, such as 12 hours or less, or 6 hours or less, or 3 hours or less, or 2 hours or less, or 1 hour or less) during which an AAV vector could be administered to the subject and predicted to efficiently transduce target tissues without substantial neutralization of the AAV vector with the AAV antibodies.

In the case where a pre-existing AAV antibody titer was reduced from 1:1000 to 1:1, AAV antibody titer rebounds of approximately 0.15% (corresponding to a titer of 1:2.5) 0.4% (1:5.3), 0.9% (1:9.7), 1.7% (1:18), and 3.4% (1:35), occur at 1 hour, 3 hours, 6 hours, 12 hours and 24 hours, respectively, after completion of the plasmapheresis method. Thus, a window for administration of AAV vector will be comparatively shorter.

AAV antibodies can be preexisting and can be present at levels that reduce or block therapeutic C1 inhibitor gene transfer vector transduction of target cells. Alternatively, AAV antibodies can develop after exposure to AAV or administration of an AAV vector. If such antibodies develop after administration of an AAV vector, these subjects can also be treated via apheresis, more particularly, plasmapheresis.

In certain embodiments, the polynucleotides, expression cassettes, AAV vectors, and non-viral vectors of the instant invention can be used in combination with methods to reduce antibody (e.g., IgG) levels in human plasma. In certain embodiments, the polynucleotides, expression cassettes, AAV vectors, and non-viral vectors of the instant invention can be used in combination with an agent that that blocks, inhibits, or reduces the interaction of IgG with the neonatal Fc receptor (FcRn), such as an anti-FcRn antibody, to reduce IgG recycling and enhance IgG clearance in vivo, and/or an agent that decreases the circulating antibodies that bind to a viral vector, such as a recombinant viral vector, or that bind to a nucleic acid or a polypeptide, protein or peptide encoded by a therapeutic heterologous polynucleotide encapsidated by a recombinant viral vector, or that bind to the therapeutic heterologous polynucleotide.

In certain embodiments, antibody binding to a viral vector is reduced or inhibited by way of an agent that reduces interaction of IgG with FcRn, a protease or a glycosidase.

In certain embodiments, the polypeptides, expression cassettes, AAV vectors, or non-viral vectors of the instant invention can be used in combination with an endopeptidase (e.g., IdeS from Streptococcus pyogenes) or a modified variant thereof, or an endoglycosidase (e.g., S. pyogenes EndoS) or a modified variant thereof. In certain embodiments poylpeptides, expression cassettes, AAV vectors, or non-viral vectors of the instant invention are administered to a subject in combination with an endopeptidase (e.g., IdeS from Streptococcus pyogenes) or a modified variant thereof, or an endoglycosidase (e.g., EndoS from S. pyogenes) or a modified variant thereof to reduce or clear neutralizing antibodies against AAV capsid and enable treatment of patients previously viewed as not eligible for gene therapy or that develop AAV antibodies after AAV gene therapy. Such strategies are described in Leborgne et al., C., Barbon, E., Alexander, J. M. et al., 2020, Nat. Med., 26:1096-1101 (2020), doi.org/10.1038/s41591-020-0911-7.

In certain embodiments, the nucleic acids, expression cassettes, AAV vectors, and non-viral vectors of the instant invention can be used in combination with symptomatic and support therapies and medications, including, for example, Berinert®, Cinryze®, Orladeyo™ (berotralstat), Ruconest® (recombinant C1-INH), Haegarda® (plasma-derived C1-INH concentrate), lanadelumab (Takhzyro®), androgens (danazol), ecallantide, icatibant, and tranexamic acid; immunosuppressive regimens including, for example rapamycin, prednisolone, tacrolimus, and tocilizumab; or FDA approved drugs such as barbiturates, sulfonamides, and estrogen.

In certain embodiments, the polynucleotides, expression cassettes and AAV vectors of the instant invention can be used in combination with pharmacological chaperone therapy (also known as enzyme enhancement therapy), where one or more pharmacological chaperones is administered before, concomitant with, or after administration of the polynucleotide, expression cassette, AAV vector, or non-viral vectors of the instant invention, for the treatment of a complement-mediated disorder, such as HAE.

In certain embodiments, the polynucleotides, and expression cassettes of the instant invention are delivered or administered via AAV vector particles. In certain embodiments, the polynucleotides and expression cassettes of the instant invention can be delivered or administered via other types of viral particles, including retroviral, adenoviral, helper-dependent adenoviral, hybrid adenoviral, herpes simplex virus, lentiviral, poxvirus, Epstein-Barr virus, vaccinia virus, and human cytomegalovirus particles. In certain embodiments, the polynucleotides and expression cassettes of the instant invention can be delivered or administered via non-viral vectors.

### Kits

The instant invention provides kits with packaging material and one or more components therein. A kit typically includes a label or packaging insert including a description of the components or instructions for use in vitro, in vivo, or ex vivo, of the components therein. A kit can contain a collection of such components, e.g., a rAAV particle or a non-viral vector, and optionally a second active component, such as another compound, agent, drug or composition.

A kit refers to a physical structure housing one or more components of the kit.

Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacture location and date, and expiration dates. Labels or inserts can include information on a disease for which a kit component can be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component can provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that can be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

All of the features disclosed herein can be combined in any combination. Each feature disclosed in the specification can be replaced by an alternative feature serving a same, equivalent, or similar purpose.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

Certain embodiments of the instant invention have been described. Nevertheless, one skilled in the art, without departing from the spirit and scope of the instant invention, can make various changes and modifications of the instant invention to adapt it to various usages and conditions. Accordingly, the following examples are intended to illustrate but not limit the scope of the instant invention claimed in any way.

Sequence Table

| | Sequence |
|---|---|
| 1 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtagggggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgtttttttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctgaccctcctgctgctgctgctggc |
| | tggggatagagcctcctcaaatccaaatgctaccagctccagctcccaggatccagagagtttgcaagacagaggcgaagggaaggtcgcaacaacagt |
| | tatctccaagatgctattcgttgaacccatcctggaggtttccagcttgccgacaaccaactcaacaaccaattcagccaccaaaataacagctaataccact |
| | gatgaacccaccacacaacccaccacagagcccaccacccaacccaccatccaacccacccaaccaactacccagctcccaacagattctcctaccca |
| | gcccactactgggtccttctgcccaggacctgttactctctgctctgacttggagagtcattcaacagaggccgtgttgggggatgctttggtagatttctccct |
| | gaagctctaccacgccttctcagcaatgaagaaggtggagaccaacatggccttttccccattcagcatcgccagcctccttacccaggtcctgctcgggg |
| | ctggggagaacaccaaaacaaacctggagagcatcctctcttaccccaaggacttcacctgtgtccaccaggccctgaagggcttcacgaccaaaggtgt |
| | cacctcagtctctcagattttccacagcccagacctggccataagggacacctttgtgaatgcctctcggaccctgtacagcagcagccccagagtcctaag |
| | caacaacagtgacgccaacttggagctcatcaacacctgggggccaagaacaccaacaacaagatcagccggctgctagacagtctgccctccgatac |
| | ccgccttgtcctcctcaatgctatctacctgagtgccaagtggaagacaacatttgatcccaagaaaaccagaatggaaccctttcacttcaaaaactcagtta |
| | taaaagtgcccatgatgaatagcaagaagtaccctgtggcccatttcattgaccaaactttgaaagccaaggtggggcagctgcagctctcccacaatctga |
| | gtttggtgatcctggtaccccagaacctgaaacatcgtcttgaagacatggaacaggctctcagcccttctgttttcaaggccatcatggagaaactggagat |
| | gtccaagttccagcccactctcctaacactaccccgcatcaaagtgacgaccagccaggatatgctctcaatcatggagaaattggaattcttcgatttttctta |
| | tgaccttaacctgtgtgggctgacagaggaccctgatcttcaggtttctgcgatgcagcaccagacagtgctggaactgacagagactggggtggaggcg |
| | gctgcagcctccgccatctctgtggcccgcaccctgctggtctttgaagtgcagcagcccttcctcttcgtgctctgggaccagcagcacaagttccctgtct |
| | tcatggggcgagtatatgaccccagggcctgaagatctagagctgaattcctgcagccagggggatcagcctctactgtgccttctagttgccagccatctg |
| | ttgtttgcccctcccccttgccttccttgaccctggaaggtgccactcccactgtcctttcctaataaaatgaggaaattgcatcacattgtctgagtaggtgtcat |
| | tctattctggggggtggggggggcaggacagcaagggggaggattgggaagacaatagcaggcatgctggggatgcagtgggctctatggcttctga |
| | ggcagaaagaaccagctggggctcgagatccactagggccgcaggaacccctagtgatggagttggccactccctctctgcgcgctcgctcgctcactg |
| | aggccgggcgaccaaaggtcgcccgacgcccgggctttgcccgggcggcctcagtgagcgagcgagcgcgcag |
| 2 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctttctgtggctgctgtcctgctgggccctgctggggaccac |
| | ctttggcaatccaaatgctaccagctccagctcccaggatccagagagtttgcaagacagaggcgaagggaaggtcgcaacaacagttatctccaagatg |
| | ctattcgttgaacccatcctggaggtttccagcttgccgacaaccaactcaacaaccaattcagccaccaaaataacagctaataccactgatgaacccacc |
| | acacaacccaccacagagcccaccacccaacccaccatccaacccacccaaccaactacccagctcccaacagattctcctacccagcccactactggg |
| | tccttctgcccaggacctgttactctctgctctgacttggagagtcattcaacagaggccgtgttgggggatgctttggtagatttctccctgaagctctaccac |
| | gccttctcagcaatgaagaaggtggagaccaacatggccttttccccattcagcatcgccagcctccttacccaggtcctgctcggggctggggagaacac |
| | caaaacaaacctggagagcatcctctcttaccccaaggacttcacctgtgtccaccaggccctgaagggcttcacgaccaaaggtgtcacctcagtctctc |
| | agattttccacagcccagacctggccataagggacacctttgtgaatgcctctcggaccctgtacagcagcagccccagagtcctaagcaacaacagtga |
| | cgccaacttggagctcatcaacacctgggtggccaagaacaccaacaacaagatcagccggctgctagacagtctgccctccgatacccgccttgtcctc |
| | ctcaatgctatctacctgagtgccaagtggaagacaacatttgatcccaagaaaaccagaatggaaccctttcacttcaaaaactcagttataaaagtgccca |
| | tgatgaatagcaagaagtaccctgtggcccatttcattgaccaaactttgaaagccaaggtggggcagctgcagctctcccacaatctgagtttggtgatcct |
| | ggtaccccagaacctgaaacatcgtcttgaagacatggaacaggctctcagcccttctgttttcaaggccatcatggagaaactggagatgtccaagttcca |
| | gcccactctcctaacactaccccgcatcaaagtgacgaccagccaggatatgctctcaatcatggagaaattggaattcttcgatttttcttatgaccttaacct |
| | gtgtgggctgacagaggaccctgatcttcaggtttctgcgatgcagcaccagacagtgctggaactgacagagactggggtggaggcggctgcagcctc |
| | cgccatctctgtggcccgcaccctgctggtctttgaagtgcagcagcccttcctcttcgtgctctgggaccagcagcacaagttccctgtcttcatggggcga |
| | gtatatgaccccagggcctgaagatctagagctgaattcctgcagccagggggatcagcctctactgtgccttctagttgccagccatctgttgtttgcccctc |
| | ccccttgccttccttgaccctggaaggtgccactcccactgtcctttcctaataaaatgaggaaattgcatcacattgtctgagtaggtgtcattctattctgggg |
| | ggtggggggggcaggacagcaagggggaggattgggaagacaatagcaggcatgctggggatgcagtgggctctatggcttctgaggcagaaaga |
| | accagctggggctcgagatccactagggccgcaggaacccctagtgatggagttggccactccctctctgcgcgctcgctcgctcactgaggccgggcg |
| | accaaaggtcgcccgacgcccgggctttgcccgggcggcctcagtgagcgagcgagcgcgcag |
| 3 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagtgggtaacctttatttcccttctttttctctttagctcggcttattc |
| | caatccaaatgctaccagctccagctcccaggatccagagagtttgcaagacagaggcgaagggaaggtcgcaacaacagttatctccaagatgctattc |
| | gttgaacccatcctggaggtttccagcttgccgacaaccaactcaacaaccaattcagccaccaaaataacagctaataccactgatgaacccaccacaca |
| | acccaccacagagcccaccacccaacccaccatccaacccacccaaccaactacccagctcccaacagattctcctacccagcccactactgggtccttc |
| | tgcccaggacctgttactctctgctctgacttggagagtcattcaacagaggccgtgttgggggatgctttggtagatttctccctgaagctctaccacgccttc |
| | tcagcaatgaagaaggtggagaccaacatggccttttccccattcagcatcgccagcctccttacccaggtcctgctcggggctggggagaacaccaaaa |
| | caaacctggagagcatcctctcttaccccaaggacttcacctgtgtccaccaggccctgaagggcttcacgaccaaaggtgtcacctcagtctctcagatttt |
| | ccacagcccagacctggccataagggacacctttgtgaatgcctctcggaccctgtacagcagcagccccagagtcctaagcaacaacagtgacgccaa |
| | cttggagctcatcaacacctgggtggccaagaacaccaacaacaagatcagccggctgctagacagtctgccctccgatacccgccttgtcctcctcaatg |
| | ctatctacctgagtgccaagtggaagacaacatttgatcccaagaaaaccagaatggaaccctttcacttcaaaaactcagttataaaagtgcccatgatgaa |
| | tagcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggcctgaagatctagagctgaattcctgcagccagggggatcagcctctactgtgccttctagttgccagccatctgttgtttgcccctccccctt |
| | gccttccttgaccctggaaggtgccactcccactgtcctttcctaataaaatgaggaaattgcatcacattgtctgagtaggtgtcattctattctggggggtgg |
| | ggtggggcaggacagcaagggggaggattgggaagacaatagcaggcatgctggggatgcagtgggctctatggcttctgaggcagaaagaaccag |
| | ctggggctcgagatccactagggccgcaggaacccctagtgatggagttggccactccctctctgcgcgctcgctcgctcactgaggccgggcgaccaa |
| | aggtcgcccgacgcccgggctttgcccgggcggcctcagtgagcgagcgagcgcgcag |
| 4 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcgctgtcctgggttcttacagtcctgagcctcctacctctgctg |
| | gaagccaatccaaatgctaccagctccagctcccaggatccagagagtttgcaagacagaggcgaagggaaggtcgcaacaacagttatctccaagatg |
| | ctattcgttgaacccatcctggaggtttccagcttgccgacaaccaactcaacaaccaattcagccaccaaaataacagctaataccactgatgaacccacc |
| | acacaacccaccacagagcccaccacccaacccaccatccaacccacccaaccaactacccagctcccaacagattctcctacccagcccactactggg |
| | tccttctgcccaggacctgttactctctgctctgacttggagagtcattcaacagaggccgtgttgggggatgctttggtagatttctccctgaagctctaccac |
| | gccttctcagcaatgaagaaggtggagaccaacatggccttttccccattcagcatcgccagcctccttacccaggtcctgctcggggctggggagaacac |
| | caaaacaaacctggagagcatcctctcttaccccaaggacttcacctgtgtccaccaggccctgaagggcttcacgaccaaaggtgtcacctcagtctctc |
| | agattttccacagcccagacctggccataagggacacctttgtgaatgcctctcggaccctgtacagcagcagccccagagtcctaagcaacaacagtga |
| | cgccaacttggagctcatcaacacctgggggccaagaacaccaacaacaagatcagccggctgctagacagtctgccctccgatacccgccttgtcctc |
| | ctcaatgctatctacctgagtgccaagtggaagacaacatttgatcccaagaaaaccagaatggaaccctttcacttcaaaaactcagttataaaagtgccca |
| | tgatgaatagcaagaagtaccctgtggcccatttcattgaccaaactttgaaagccaaggtggggcagctgcagctctcccacaatctgagtttggtgatcct |
| | ggtaccccagaacctgaaacatcgtcttgaagacatggaacaggctctcagcccttctgttttcaaggccatcatggagaaactggagatgtccaagttcca |
| | gcccactctcctaacactaccccgcatcaaagtgacgaccagccaggatatgctctcaatcatggagaaattggaattcttcgatttttcttatgaccttaacct |
| | gtgtgggctgacagaggaccctgatcttcaggtttctgcgatgcagcaccagacagtgctggaactgacagagactggggtggaggcggctgcagcctc |
| | cgccatctctgtggcccgcaccctgctggtctttgaagtgcagcagcccttcctcttcgtgctctgggaccagcagcacaagttccctgtcttcatggggcga |
| | gtatatgaccccagggcctgaagatctagagctgaattcctgcagccagggggatcagcctctactgtgccttctagttgccagccatctgttgtttgcccctc |
| | ccccttgccttccttgaccctggaaggtgccactcccactgtcctttcctaataaaatgaggaaattgcatcacattgtctgagtaggtgtcattctattctgggg |
| | ggtggggggggcaggacagcaagggggaggattgggaagacaatagcaggcatgctggggatgcagtgggctctatggcttctgaggcagaaaga |
| | accagctggggctcgagatccactagggccgcaggaacccctagtgatggagttggccactccctctctgcgcgctcgctcgctcactgaggccgggcg |
| | accaaaggtcgcccgacgcccgggctttgcccgggcggcctcagtgagcgagcgagcgcgcag |
| 5 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaggctcgccgtgggagccctgctggtctgcgccgtcctgggg |
| | ctgtgtctggctaatccaaatgctaccagctccagctcccaggatccagagagtttgcaagacagaggcgaagggaaggtcgcaacaacagttatctcca |
| | agatgctattcgttgaacccatcctggaggtttccagcttgccgacaaccaactcaacaaccaattcagccaccaaaataacagctaataccactgatgaac |
| | ccaccacacaacccaccacagagcccaccacccaacccaccatccaacccacccaaccaactacccagctcccaacagattctcctacccagcccacta |
| | ctgggtccttctgcccaggacctgttactctctgctctgacttggagagtcattcaacagaggccgtgttgggggatgctttggtagatttctccctgaagctct |
| | accacgccttctcagcaatgaagaaggtggagaccaacatggccttttccccattcagcatcgccagcctccttacccaggtcctgctcggggctggggag |
| | aacaccaaaacaaacctggagagcatcctctcttaccccaaggacttcacctgtgtccaccaggccctgaagggcttcacgaccaaaggtgtcacctcagt |
| | ctctcagattttccacagcccagacctggccataagggacacctttgtgaatgcctctcggaccctgtacagcagcagccccagagtcctaagcaacaaca |
| | gtgacgccaacttggagctcatcaacacctgggtggccaagaacaccaacaacaagatcagccggctgctagacagtctgccctccgatacccgccttgt |
| | cctcctcaatgctatctacctgagtgccaagtggaagacaacatttgatcccaagaaaaccagaatggaaccctttcacttcaaaaactcagttataaaagtg |
| | cccatgatgaatagcaagaagtaccctgtggcccatttcattgaccaaactttgaaagccaaggtggggcagctgcagctctcccacaatctgagtttggtg |
| | atcctggtaccccagaacctgaaacatcgtcttgaagacatggaacaggctctcagcccttctgttttcaaggccatcatggagaaactggagatgtccaag |
| | ttccagcccactctcctaacactaccccgcatcaaagtgacgaccagccaggatatgctctcaatcatggagaaattggaattcttcgatttttcttatgacctta |
| | acctgtgtgggctgacagaggaccctgatcttcaggtttctgcgatgcagcaccagacagtgctggaactgacagagactggggtggaggcggctgcag |
| | cctccgccatctctgtggcccgcaccctgctggtctttgaagtgcagcagcccttcctcttcgtgctctgggaccagcagcacaagttccctgtcttcatggg |
| | gcgagtatatgaccccagggcctgaagatctagagctgaattcctgcagccagggggatcagcctctactgtgccttctagttgccagccatctgttgtttgc |
| | ccctcccccttgccttccttgaccctggaaggtgccactcccactgtcctttcctaataaaatgaggaaattgcatcacattgtctgagtaggtgtcattctattct |
| | ggggggtggggtggggcaggacagcaagggggaggattgggaagacaatagcaggcatgctggggatgcagtgggctctatggcttctgaggcaga |
| | aagaaccagctggggctcgagatccactagggccgcaggaacccctagtgatggagttggccactccctctctgcgcgctcgctcgctcactgaggccg |
| | ggcgaccaaaggtcgcccgacgcccgggctttgcccgggcggcctcagtgagcgagcgagcgcgcag |
| 6 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaggagcctcggggccctgctcttgctgctgagcgcctgcctgg |
| | cggtgagcgctaatccaaatgctaccagctccagctcccaggatccagagagtttgcaagacagaggcgaagggaaggtcgcaacaacagttatctcca |
| | agatgctattcgttgaacccatcctggaggtttccagcttgccgacaaccaactcaacaaccaattcagccaccaaaataacagctaataccactgatgaac |
| | ccaccacacaacccaccacagagcccaccacccaacccaccatccaacccacccaaccaactacccagctcccaacagattctcctacccagcccacta |
| | ctgggtccttctgcccaggacctgttactctctgctctgacttggagagtcattcaacagaggccgtgttgggggatgctttggtagatttctccctgaagctct |
| | accacgccttctcagcaatgaagaaggtggagaccaacatggccttttccccattcagcatcgccagcctccttacccaggtcctgctcggggctggggag |
| | aacaccaaaacaaacctggagagcatcctctcttaccccaaggacttcacctgtgtccaccaggccctgaagggcttcacgaccaaaggtgtcacctcagt |
| | ctctcagattttccacagcccagacctggccataagggacacctttgtgaatgcctctcggaccctgtacagcagcagccccagagtcctaagcaacaaca |
| | gtgacgccaacttggagctcatcaacacctgggtggccaagaacaccaacaacaagatcagccggctgctagacagtctgccctccgatacccgccttgt |
| | cctcctcaatgctatctacctgagtgccaagtggaagacaacatttgatcccaagaaaaccagaatggaaccctttcacttcaaaaactcagttataaaagtg |
| | cccatgatgaatagcaagaagtaccctgtggcccatttcattgaccaaactttgaaagccaaggtggggcagctgcagctctcccacaatctgagtttggtg |
| | atcctggtaccccagaacctgaaacatcgtcttgaagacatggaacaggctctcagcccttctgttttcaaggccatcatggagaaactggagatgtccaag |
| | ttccagcccactctcctaacactaccccgcatcaaagtgacgaccagccaggatatgctctcaatcatggagaaattggaattcttcgatttttcttatgacctta |
| | acctgtgtgggctgacagaggaccctgatcttcaggtttctgcgatgcagcaccagacagtgctggaactgacagagactggggtggaggcggctgcag |
| | cctccgccatctctgtggcccgcaccctgctggtctttgaagtgcagcagcccttcctcttcgtgctctgggaccagcagcacaagttccctgtcttcatggg |
| | gcgagtatatgaccccagggcctgaagatctagagctgaattcctgcagccagggggatcagcctctactgtgccttctagttgccagccatctgttgtttgc |
| | ccctcccccttgccttccttgaccctggaaggtgccactcccactgtcctttcctaataaaatgaggaaattgcatcacattgtctgagtaggtgtcattctattct |
| | ggggggtggggggggcaggacagcaagggggaggattgggaagacaatagcaggcatgctggggatgcagtgggctctatggcttctgaggcaga |
| | aagaaccagctggggctcgagatccactagggccgcaggaacccctagtgatggagttggccactccctctctgcgcgctcgctcgctcactgaggccg |
| | ggcgaccaaaggtcgcccgacgcccgggctttgcccgggcggcctcagtgagcgagcgagcgcgcag |
| 7 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcggcccccggcagcgcccggcgacccctgctgctgctactg |
| | ctgttgctgctgctcggcctcatgcattgtgcgtcagcaaatccaaatgctaccagctccagctcccaggatccagagagtttgcaagacagaggcgaagg |
| | gaaggtcgcaacaacagttatctccaagatgctattcgttgaacccatcctggaggtttccagcttgccgacaaccaactcaacaaccaattcagccaccaa |
| | aataacagctaataccactgatgaacccaccacacaacccaccacagagcccaccacccaacccaccatccaacccacccaaccaactacccagctccc |
| | aacagattctcctacccagcccactactgggtccttctgcccaggacctgttactctctgctctgacttggagagtcattcaacagaggccgtgttgggggat |
| | gctttggtagatttctccctgaagctctaccacgccttctcagcaatgaagaaggtggagaccaacatggccttttccccattcagcatcgccagcctccttac |
| | ccaggtcctgctcggggctggggagaacaccaaaacaaacctggagagcatcctctcttaccccaaggacttcacctgtgtccaccaggccctgaaggg |
| | cttcacgaccaaaggtgtcacctcagtctctcagattttccacagcccagacctggccataagggacacctttgtgaatgcctctcggaccctgtacagcag |
| | cagccccagagtcctaagcaacaacagtgacgccaacttggagctcatcaacacctgggggccaagaacaccaacaacaagatcagccggctgctag |
| | acagtctgccctccgatacccgccttgtcctcctcaatgctatctacctgagtgccaagtggaagacaacatttgatcccaagaaaaccagaatggaaccctt |
| | tcacttcaaaaactcagttataaaagtgcccatgatgaatagcaagaagtaccctgtggcccatttcattgaccaaactttgaaagccaaggtggggcagctg |
| | cagctctcccacaatctgagtttggtgatcctggtaccccagaacctgaaacatcgtcttgaagacatggaacaggctctcagcccttctgttttcaaggccat |
| | catggagaaactggagatgtccaagttccagcccactctcctaacactaccccgcatcaaagtgacgaccagccaggatatgctctcaatcatggagaaat |
| | tggaattcttcgatttttcttatgaccttaacctgtgtgggctgacagaggaccctgatcttcaggtttctgcgatgcagcaccagacagtgctggaactgacag |
| | agactggggtggaggcggctgcagcctccgccatctctgtggcccgcaccctgctggtctttgaagtgcagcagcccttcctcttcgtgctctgggaccag |
| | cagcacaagttccctgtcttcatggggcgagtatatgaccccagggcctgaagatctagagctgaattcctgcagccagggggatcagcctctactgtgcc |
| | ttctagttgccagccatctgttgtttgcccctcccccttgccttccttgaccctggaaggtgccactcccactgtcctttcctaataaaatgaggaaattgcatca |
| | cattgtctgagtaggtgtcattctattctggggggtggggggggcaggacagcaagggggaggattgggaagacaatagcaggcatgctggggatgca |
| | gtgggctctatggcttctgaggcagaaagaaccagctggggctcgagatccactagggccgcaggaacccctagtgatggagttggccactccctctctg |
| | cgcgctcgctcgctcactgaggccgggcgaccaaaggtcgcccgacgcccgggctttgcccgggcggcctcagtgagcgagcgagcgcgcag |
| 8 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtagggggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggaaccagctgctgctctgcacttctccctgccagcctccctcctc |
| | ctcctcctgctcctcctccttctcagcctgtgtgcactggtctcagccaatccaaatgctaccagctccagctcccaggatccagagagtttgcaagacagag |
| | gcgaagggaaggtcgcaacaacagttatctccaagatgctattcgttgaacccatcctggaggtttccagcttgccgacaaccaactcaacaaccaattcag |
| | ccaccaaaataacagctaataccactgatgaacccaccacacaacccaccacagagcccaccacccaacccaccatccaacccacccaaccaactacc |
| | cagctcccaacagattctcctacccagcccactactgggtccttctgcccaggacctgttactctctgctctgacttggagagtcattcaacagaggccgtgtt |
| | gggggatgctttggtagatttctccctgaagctctaccacgccttctcagcaatgaagaaggtggagaccaacatggccttttccccattcagcatcgccagc |
| | ctccttacccaggtcctgctcggggctggggagaacaccaaaacaaacctggagagcatcctctcttaccccaaggacttcacctgtgtccaccaggccct |
| | gaagggcttcacgaccaaaggtgtcacctcagtctctcagattttccacagcccagacctggccataagggacacctttgtgaatgcctctcggaccctgta |
| | cagcagcagccccagagtcctaagcaacaacagtgacgccaacttggagctcatcaacacctgggggccaagaacaccaacaacaagatcagccgg |
| | ctgctagacagtctgccctccgatacccgccttgtcctcctcaatgctatctacctgagtgccaagtggaagacaacatttgatcccaagaaaaccagaatg |
| | gaaccctttcacttcaaaaactcagttataaaagtgcccatgatgaatagcaagaagtaccctgtggcccatttcattgaccaaactttgaaagccaaggtgg |
| | ggcagctgcagctctcccacaatctgagtttggtgatcctggtaccccagaacctgaaacatcgtcttgaagacatggaacaggctctcagcccttctgttttc |
| | aaggccatcatggagaaactggagatgtccaagttccagcccactctcctaacactaccccgcatcaaagtgacgaccagccaggatatgctctcaatcat |
| | ggagaaattggaattcttcgatttttcttatgaccttaacctgtgtgggctgacagaggaccctgatcttcaggtttctgcgatgcagcaccagacagtgctgg |
| | aactgacagagactggggtggaggcggctgcagcctccgccatctctgtggcccgcaccctgctggtctttgaagtgcagcagcccttcctcttcgtgctct |
| | gggaccagcagcacaagttccctgtcttcatggggcgagtatatgaccccagggcctgaagatctagagctgaattcctgcagccagggggatcagcctc |
| | tactgtgccttctagttgccagccatctgttgtttgcccctcccccttgccttccttgaccctggaaggtgccactcccactgtcctttcctaataaaatgaggaa |
| | attgcatcacattgtctgagtaggtgtcattctattctggggggtggggggggcaggacagcaagggggaggattgggaagacaatagcaggcatgctg |
| | gggatgcagtgggctctatggcttctgaggcagaaagaaccagctggggctcgagatccactagggccgcaggaacccctagtgatggagttggccact |
| | ccctctctgcgcgctcgctcgctcactgaggccgggcgaccaaaggtcgcccgacgcccgggctttgcccgggcggcctcagtgagcgagcgagcgc |
| | gcag |
| 9 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgtttttttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgtggctgccttgggctctgttgcttctctgggtcccaggatgttttg |
| | ctaatccaaatgctaccagctccagctcccaggatccagagagtttgcaagacagaggcgaagggaaggtcgcaacaacagttatctccaagatgctattc |
| | gttgaacccatcctggaggtttccagcttgccgacaaccaactcaacaaccaattcagccaccaaaataacagctaataccactgatgaacccaccacaca |
| | acccaccacagagcccaccacccaacccaccatccaacccacccaaccaactacccagctcccaacagattctcctacccagcccactactgggtccttc |
| | tgcccaggacctgttactctctgctctgacttggagagtcattcaacagaggccgtgttgggggatgctttggtagatttctccctgaagctctaccacgccttc |
| | tcagcaatgaagaaggtggagaccaacatggccttttccccattcagcatcgccagcctccttacccaggtcctgctcggggctggggagaacaccaaaa |
| | caaacctggagagcatcctctcttaccccaaggacttcacctgtgtccaccaggccctgaagggcttcacgaccaaaggtgtcacctcagtctctcagatttt |
| | ccacagcccagacctggccataagggacacctttgtgaatgcctctcggaccctgtacagcagcagccccagagtcctaagcaacaacagtgacgccaa |
| | cttggagctcatcaacacctgggtggccaagaacaccaacaacaagatcagccggctgctagacagtctgccctccgatacccgccttgtcctcctcaatg |
| | ctatctacctgagtgccaagtggaagacaacatttgatcccaagaaaaccagaatggaaccctttcacttcaaaaactcagttataaaagtgcccatgatgaa |
| | tagcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggtggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggcctgaagatctagagctgaattcctgcagccagggggatcagcctctactgtgccttctagttgccagccatctgttgtttgcccctccccctt |
| | gccttccttgaccctggaaggtgccactcccactgtcctttcctaataaaatgaggaaattgcatcacattgtctgagtaggtgtcattctattctggggggtgg |
| | ggtggggcaggacagcaagggggaggattgggaagacaatagcaggcatgctggggatgcagtgggctctatggcttctgaggcagaaagaaccag |
| | ctggggctcgagatccactagggccgcaggaacccctagtgatggagttggccactccctctctgcgcgctcgctcgctcactgaggccgggcgaccaa |
| | aggtcgcccgacgcccgggctttgcccgggcggcctcagtgagcgagcgagcgcgcag |
| 10 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgcccgccctgcgccccgctctgctgtgggcgctgctggcgctct |
| | ggctgtgctgcgcggcccccgcgcatgcaaatccaaatgctaccagctccagctcccaggatccagagagtttgcaagacagaggcgaagggaaggtc |
| | gcaacaacagttatctccaagatgctattcgttgaacccatcctggaggtttccagcttgccgacaaccaactcaacaaccaattcagccaccaaaataaca |
| | gctaataccactgatgaacccaccacacaacccaccacagagcccaccacccaacccaccatccaacccacccaaccaactacccagctcccaacaga |
| | ttctcctacccagcccactactgggtccttctgcccaggacctgttactctctgctctgacttggagagtcattcaacagaggccgtgttgggggatgctttggt |
| | agatttctccctgaagctctaccacgccttctcagcaatgaagaaggtggagaccaacatggccttttccccattcagcatcgccagcctccttacccaggtc |
| | ctgctcggggctggggagaacaccaaaacaaacctggagagcatcctctcttaccccaaggacttcacctgtgtccaccaggccctgaagggcttcacga |
| | ccaaaggtgtcacctcagtctctcagattttccacagcccagacctggccataagggacacctttgtgaatgcctctcggaccctgtacagcagcagcccca |
| | gagtcctaagcaacaacagtgacgccaacttggagctcatcaacacctgggtggccaagaacaccaacaacaagatcagccggctgctagacagtctgc |
| | cctccgatacccgccttgtcctcctcaatgctatctacctgagtgccaagtggaagacaacatttgatcccaagaaaaccagaatggaaccctttcacttcaa |
| | aaactcagttataaaagtgcccatgatgaatagcaagaagtaccctgtggcccatttcattgaccaaactttgaaagccaaggtggggcagctgcagctctc |
| | ccacaatctgagtttggtgatcctggtaccccagaacctgaaacatcgtcttgaagacatggaacaggctctcagcccttctgttttcaaggccatcatggag |
| | aaactggagatgtccaagttccagcccactctcctaacactaccccgcatcaaagtgacgaccagccaggatatgctctcaatcatggagaaattggaattc |
| | ttcgatttttcttatgaccttaacctgtgtgggctgacagaggaccctgatcttcaggtttctgcgatgcagcaccagacagtgctggaactgacagagactgg |
| | ggtggaggcggctgcagcctccgccatctctgtggcccgcaccctgctggtctttgaagtgcagcagcccttcctcttcgtgctctgggaccagcagcaca |
| | agttccctgtcttcatggggcgagtatatgaccccagggcctgaagatctagagctgaattcctgcagccagggggatcagcctctactgtgccttctagttg |
| | ccagccatctgttgtttgcccctcccccttgccttccttgaccctggaaggtgccactcccactgtcctttcctaataaaatgaggaaattgcatcacattgtctg |
| | agtaggtgtcattctattctggggggtggggggggcaggacagcaagggggaggattgggaagacaatagcaggcatgctggggatgcagtgggctc |
| | tatggcttctgaggcagaaagaaccagctggggctcgagatccactagggccgcaggaacccctagtgatggagttggccactccctctctgcgcgctcg |
| | ctcgctcactgaggccgggcgaccaaaggtcgcccgacgcccgggctttgcccgggcggcctcagtgagcgagcgagcgcgcag |
| 11 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgtttttttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggctctcagcctctggcccctgctgctgctgctgctgctgctgctg |
| | ctgctgtcctttgcaaatccaaatgctaccagctccagctcccaggatccagagagtttgcaagacagaggcgaagggaaggtcgcaacaacagttatctc |
| | caagatgctattcgttgaacccatcctggaggtttccagcttgccgacaaccaactcaacaaccaattcagccaccaaaataacagctaataccactgatga |
| | acccaccacacaacccaccacagagcccaccacccaacccaccatccaacccacccaaccaactacccagctcccaacagattctcctacccagccca |
| | ctactgggtccttctgcccaggacctgttactctctgctctgacttggagagtcattcaacagaggccgtgttgggggatgctttggtagatttctccctgaagc |
| | tctaccacgccttctcagcaatgaagaaggtggagaccaacatggccttttccccattcagcatcgccagcctccttacccaggtcctgctcggggctggg |
| | gagaacaccaaaacaaacctggagagcatcctctcttaccccaaggacttcacctgtgtccaccaggccctgaagggcttcacgaccaaaggtgtcacct |
| | cagtctctcagattttccacagcccagacctggccataagggacacctttgtgaatgcctctcggaccctgtacagcagcagccccagagtcctaagcaac |
| | aacagtgacgccaacttggagctcatcaacacctgggtggccaagaacaccaacaacaagatcagccggctgctagacagtctgccctccgatacccgc |
| | cttgtcctcctcaatgctatctacctgagtgccaagtggaagacaacatttgatcccaagaaaaccagaatggaaccctttcacttcaaaaactcagttataaa |
| | agtgcccatgatgaatagcaagaagtaccctgtggcccatttcattgaccaaactttgaaagccaaggtggggcagctgcagctctcccacaatctgagttt |
| | ggtgatcctggtaccccagaacctgaaacatcgtcttgaagacatggaacaggctctcagcccttctgttttcaaggccatcatggagaaactggagatgtc |
| | caagttccagcccactctcctaacactaccccgcatcaaagtgacgaccagccaggatatgctctcaatcatggagaaattggaattcttcgatttttcttatga |
| | ccttaacctgtgtgggctgacagaggaccctgatcttcaggtttctgcgatgcagcaccagacagtgctggaactgacagagactggggtggaggcggct |
| | gcagcctccgccatctctgtggcccgcaccctgctggtctttgaagtgcagcagcccttcctcttcgtgctctgggaccagcagcacaagttccctgtcttca |
| | tggggcgagtatatgaccccagggcctgaagatctagagctgaattcctgcagccagggggatcagcctctactgtgccttctagttgccagccatctgttg |
| | tttgcccctcccccttgccttccttgaccctggaaggtgccactcccactgtcctttcctaataaaatgaggaaattgcatcacattgtctgagtaggtgtcattct |
| | attctggggggtggggtggggcaggacagcaagggggaggattgggaagacaatagcaggcatgctggggatgcagtgggctctatggcttctgagg |
| | cagaaagaaccagctggggctcgagatccactagggccgcaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgcag |
| 12 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagtccctcgtcctgctcctttgtcttgctcagctctggggctgcc |
| | actcaaatccaaatgctaccagctccagctcccaggatccagagagtttgcaagacagaggcgaagggaaggtcgcaacaacagttatctccaagatgct |
| | attcgttgaacccatcctggaggtttccagcttgccgacaaccaactcaacaaccaattcagccaccaaaataacagctaataccactgatgaacccaccac |
| | acaacccaccacagagcccaccacccaacccaccatccaacccacccaaccaactacccagctcccaacagattctcctacccagcccactactgggtc |
| | cttctgcccaggacctgttactctctgctctgacttggagagtcattcaacagaggccgtgttgggggatgctttggtagatttctccctgaagctctaccacgc |
| | cttctcagcaatgaagaaggtggagaccaacatggccttttccccattcagcatcgccagcctccttacccaggtcctgctcggggctggggagaacacca |
| | aaacaaacctggagagcatcctctcttaccccaaggacttcacctgtgtccaccaggccctgaagggcttcacgaccaaaggtgtcacctcagtctctcag |
| | attttccacagcccagacctggccataagggacacctttgtgaatgcctctcggaccctgtacagcagcagccccagagtcctaagcaacaacagtgacg |
| | ccaacttggagctcatcaacacctgggtggccaagaacaccaacaacaagatcagccggctgctagacagtctgccctccgatacccgccttgtcctcctc |
| | aatgctatctacctgagtgccaagtggaagacaacatttgatcccaagaaaaccagaatggaaccctttcacttcaaaaactcagttataaaagtgcccatga |
| | tgaatagcaagaagtaccctgtggcccatttcattgaccaaactttgaaagccaaggtggggcagctgcagctctcccacaatctgagtttggtgatcctggt |
| | accccagaacctgaaacatcgtcttgaagacatggaacaggctctcagcccttctgttttcaaggccatcatggagaaactggagatgtccaagttccagcc |
| | cactctcctaacactaccccgcatcaaagtgacgaccagccaggatatgctctcaatcatggagaaattggaattcttcgatttttcttatgaccttaacctgtgt |
| | gggctgacagaggaccctgatcttcaggtttctgcgatgcagcaccagacagtgctggaactgacagagactggggggaggcggctgcagcctccgc |
| | catctctgtggcccgcaccctgctggtctttgaagtgcagcagcccttcctcttcgtgctctgggaccagcagcacaagttccctgtcttcatggggcgagta |
| | tatgaccccagggcctgaagatctagagctgaattcctgcagccagggggatcagcctctactgtgccttctagttgccagccatctgttgtttgcccctccc |
| | ccttgccttccttgaccctggaaggtgccactcccactgtcctttcctaataaaatgaggaaattgcatcacattgtctgagtaggtgtcattctattctgggggg |
| | tggggtggggcaggacagcaagggggaggattgggaagacaatagcaggcatgctggggatgcagtgggctctatggcttctgaggcagaaagaacc |
| | agctggggctcgagatccactagggccgcaggaacccctagtgatggagttggccactccctctctgcgcgctcgctcgctcactgaggccgggcgacc |
| | aaaggtcgcccgacgcccgggctttgcccgggcggcctcagtgagcgagcgagcgcgcag |
| 13 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagttcgccctggtggccgccctggccctgctgctgttcctgtg |
| | gagcagctgcagggccaatccaaatgctaccagctccagctcccaggatccagagagtttgcaagacagaggcgaagggaaggtcgcaacaacagtta |
| | tctccaagatgctattcgttgaacccatcctggaggtttccagcttgccgacaaccaactcaacaaccaattcagccaccaaaataacagctaataccactga |
| | tgaacccaccacacaacccaccacagagcccaccacccaacccaccatccaacccacccaaccaactacccagctcccaacagattctcctacccagcc |
| | cactactgggtccttctgcccaggacctgttactctctgctctgacttggagagtcattcaacagaggccgtgttgggggatgctttggtagatttctccctgaa |
| | gctctaccacgccttctcagcaatgaagaaggtggagaccaacatggccttttccccattcagcatcgccagcctccttacccaggtcctgctcggggctgg |
| | ggagaacaccaaaacaaacctggagagcatcctctcttaccccaaggacttcacctgtgtccaccaggccctgaagggcttcacgaccaaaggtgtcacc |
| | tcagtctctcagattttccacagcccagacctggccataagggacacctttgtgaatgcctctcggaccctgtacagcagcagccccagagtcctaagcaac |
| | aacagtgacgccaacttggagctcatcaacacctgggggccaagaacaccaacaacaagatcagccggctgctagacagtctgccctccgatacccgc |
| | cttgtcctcctcaatgctatctacctgagtgccaagtggaagacaacatttgatcccaagaaaaccagaatggaaccctttcacttcaaaaactcagttataaa |
| | agtgcccatgatgaatagcaagaagtaccctgtggcccatttcattgaccaaactttgaaagccaaggtggggcagctgcagctctcccacaatctgagttt |
| | ggtgatcctggtaccccagaacctgaaacatcgtcttgaagacatggaacaggctctcagcccttctgttttcaaggccatcatggagaaactggagatgtc |
| | caagttccagcccactctcctaacactaccccgcatcaaagtgacgaccagccaggatatgctctcaatcatggagaaattggaattcttcgatttttcttatga |
| | ccttaacctgtgtgggctgacagaggaccctgatcttcaggtttctgcgatgcagcaccagacagtgctggaactgacagagactggggtggaggcggct |
| | gcagcctccgccatctctgtggcccgcaccctgctggtctttgaagtgcagcagcccttcctcttcgtgctctgggaccagcagcacaagttccctgtcttca |
| | tggggcgagtatatgaccccagggcctgaagatctagagctgaattcctgcagccagggggatcagcctctactgtgccttctagttgccagccatctgttg |
| | tttgcccctcccccttgccttccttgaccctggaaggtgccactcccactgtcctttcctaataaaatgaggaaattgcatcacattgtctgagtaggtgtcattct |
| | attctggggggtggggggggcaggacagcaagggggaggattgggaagacaatagcaggcatgctggggatgcagtgggctctatggcttctgagg |
| | cagaaagaaccagctggggctcgagatccactagggccgcaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgcag |
| 14 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtagggggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctcttttttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagttcgccctggtggccgccctggccctgctgctgttcctgtg |
| | gggctgccacagcaatccaaatgctaccagctccagctcccaggatccagagagtttgcaagacagaggcgaagggaaggtcgcaacaacagttatctc |
| | caagatgctattcgttgaacccatcctggaggtttccagcttgccgacaaccaactcaacaaccaattcagccaccaaaataacagctaataccactgatga |
| | acccaccacacaacccaccacagagcccaccacccaacccaccatccaacccacccaaccaactacccagctcccaacagattctcctacccagccca |
| | ctactgggtccttctgcccaggacctgttactctctgctctgacttggagagtcattcaacagaggccgtgttgggggatgctttggtagatttctccctgaagc |
| | tctaccacgccttctcagcaatgaagaaggtggagaccaacatggccttttccccattcagcatcgccagcctccttacccaggtcctgctcggggctggg |
| | gagaacaccaaaacaaacctggagagcatcctctcttaccccaaggacttcacctgtgtccaccaggccctgaagggcttcacgaccaaaggtgtcacct |
| | cagtctctcagattttccacagcccagacctggccataagggacacctttgtgaatgcctctcggaccctgtacagcagcagccccagagtcctaagcaac |
| | aacagtgacgccaacttggagctcatcaacacctgggtggccaagaacaccaacaacaagatcagccggctgctagacagtctgccctccgatacccgc |
| | cttgtcctcctcaatgctatctacctgagtgccaagtggaagacaacatttgatcccaagaaaaccagaatggaaccctttcacttcaaaaactcagttataaa |
| | agtgcccatgatgaatagcaagaagtaccctgtggcccatttcattgaccaaactttgaaagccaaggtggggcagctgcagctctcccacaatctgagttt |
| | ggtgatcctggtaccccagaacctgaaacatcgtcttgaagacatggaacaggctctcagcccttctgttttcaaggccatcatggagaaactggagatgtc |
| | caagttccagcccactctcctaacactaccccgcatcaaagtgacgaccagccaggatatgctctcaatcatggagaaattggaattcttcgatttttcttatga |
| | ccttaacctgtgtgggctgacagaggaccctgatcttcaggtttctgcgatgcagcaccagacagtgctggaactgacagagactggggtggaggcggct |
| | gcagcctccgccatctctgtggcccgcaccctgctggtctttgaagtgcagcagcccttcctcttcgtgctctgggaccagcagcacaagttccctgtcttca |
| | tggggcgagtatatgaccccagggcctgaagatctagagctgaattcctgcagccagggggatcagcctctactgtgccttctagttgccagccatctgttg |
| | tttgcccctcccccttgccttccttgaccctggaaggtgccactcccactgtcctttcctaataaaatgaggaaattgcatcacattgtctgagtaggtgtcattct |
| | attctggggggggggtggggcaggacagcaagggggaggattgggaagacaatagcaggcatgctggggatgcagtgggctctatggcttctgagg |
| | cagaaagaaccagctggggctcgagatccactagggccgcaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgcag |
| 15 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgtttttttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagtgggtgaccctgctgaccctgctgctgctgctgctgttcctg |
| | tggggctgccacagcaatccaaatgctaccagctccagctcccaggatccagagagtttgcaagacagaggcgaagggaaggtcgcaacaacagttatc |
| | tccaagatgctattcgttgaacccatcctggaggtttccagcttgccgacaaccaactcaacaaccaattcagccaccaaaataacagctaataccactgatg |
| | aacccaccacacaacccaccacagagcccaccacccaacccaccatccaacccacccaaccaactacccagctcccaacagattctcctacccagccc |
| | actactgggtccttctgcccaggacctgttactctctgctctgacttggagagtcattcaacagaggccgtgttgggggatgctttggtagatttctccctgaag |
| | ctctaccacgccttctcagcaatgaagaaggtggagaccaacatggccttttccccattcagcatcgccagcctccttacccaggtcctgctcggggctggg |
| | gagaacaccaaaacaaacctggagagcatcctctcttaccccaaggacttcacctgtgtccaccaggccctgaagggcttcacgaccaaaggtgtcacct |
| | cagtctctcagattttccacagcccagacctggccataagggacacctttgtgaatgcctctcggaccctgtacagcagcagccccagagtcctaagcaac |
| | aacagtgacgccaacttggagctcatcaacacctgggggccaagaacaccaacaacaagatcagccggctgctagacagtctgccctccgatacccgc |
| | cttgtcctcctcaatgctatctacctgagtgccaagtggaagacaacatttgatcccaagaaaaccagaatggaaccctttcacttcaaaaactcagttataaa |
| | agtgcccatgatgaatagcaagaagtaccctgtggcccatttcattgaccaaactttgaaagccaaggtggggcagctgcagctctcccacaatctgagttt |
| | ggtgatcctggtaccccagaacctgaaacatcgtcttgaagacatggaacaggctctcagcccttctgttttcaaggccatcatggagaaactggagatgtc |
| | caagttccagcccactctcctaacactaccccgcatcaaagtgacgaccagccaggatatgctctcaatcatggagaaattggaattcttcgatttttcttatga |
| | ccttaacctgtgtgggctgacagaggaccctgatcttcaggtttctgcgatgcagcaccagacagtgctggaactgacagagactggggtggaggcggct |
| | gcagcctccgccatctctgtggcccgcaccctgctggtctttgaagtgcagcagcccttcctcttcgtgctctgggaccagcagcacaagttccctgtcttca |
| | tggggcgagtatatgaccccagggcctgaagatctagagctgaattcctgcagccagggggatcagcctctactgtgccttctagttgccagccatctgttg |
| | tttgcccctcccccttgccttccttgaccctggaaggtgccactcccactgtcctttcctaataaaatgaggaaattgcatcacattgtctgagtaggtgtcattct |
| | attctggggggtggggtggggcaggacagcaagggggaggattgggaagacaatagcaggcatgctggggatgcagtgggctctatggcttctgagg |
| | cagaaagaaccagctggggctcgagatccactagggccgcaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgcag |
| 16 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatgaagtgggtgaccctgctgaccctgctgctgctgctgctgttcctg |
| | tggagcagctgcagggccaatccaaatgctaccagctccagctcccaggatccagagagtttgcaagacagaggcgaagggaaggtcgcaacaacagt |
| | tatctccaagatgctattcgttgaacccatcctggaggtttccagcttgccgacaaccaactcaacaaccaattcagccaccaaaataacagctaataccact |
| | gatgaacccaccacacaacccaccacagagcccaccacccaacccaccatccaacccacccaaccaactacccagctcccaacagattctcctaccca |
| | gcccactactgggtccttctgcccaggacctgttactctctgctctgacttggagagtcattcaacagaggccgtgttgggggatgctttggtagatttctccct |
| | gaagctctaccacgccttctcagcaatgaagaaggtggagaccaacatggccttttccccattcagcatcgccagcctccttacccaggtcctgctcgggg |
| | ctggggagaacaccaaaacaaacctggagagcatcctctcttaccccaaggacttcacctgtgtccaccaggccctgaagggcttcacgaccaaaggtgt |
| | cacctcagtctctcagattttccacagcccagacctggccataagggacacctttgtgaatgcctctcggaccctgtacagcagcagccccagagtcctaag |
| | caacaacagtgacgccaacttggagctcatcaacacctgggggccaagaacaccaacaacaagatcagccggctgctagacagtctgccctccgatac |
| | ccgccttgtcctcctcaatgctatctacctgagtgccaagtggaagacaacatttgatcccaagaaaaccagaatggaaccctttcacttcaaaaactcagtta |
| | taaaagtgcccatgatgaatagcaagaagtaccctgtggcccatttcattgaccaaactttgaaagccaaggtggggcagctgcagctctcccacaatctga |
| | gtttggtgatcctggtaccccagaacctgaaacatcgtcttgaagacatggaacaggctctcagcccttctgttttcaaggccatcatggagaaactggagat |
| | gtccaagttccagcccactctcctaacactaccccgcatcaaagtgacgaccagccaggatatgctctcaatcatggagaaattggaattcttcgatttttctta |
| | tgaccttaacctgtgtgggctgacagaggaccctgatcttcaggtttctgcgatgcagcaccagacagtgctggaactgacagagactggggtggaggcg |
| | gctgcagcctccgccatctctgtggcccgcaccctgctggtctttgaagtgcagcagcccttcctcttcgtgctctgggaccagcagcacaagttccctgtct |
| | tcatggggcgagtatatgaccccagggcctgaagatctagagctgaattcctgcagccagggggatcagcctctactgtgccttctagttgccagccatctg |
| | ttgtttgcccctcccccttgccttccttgaccctggaaggtgccactcccactgtcctttcctaataaaatgaggaaattgcatcacattgtctgagtaggtgtcat |
| | tctattctggggggtggggtggggcaggacagcaagggggaggattgggaagacaatagcaggcatgctggggatgcagtgggctctatggcttctga |
| | ggcagaaagaaccagctggggctcgagatccactagggccgcaggaacccctagtgatggagttggccactccctctctgcgcgctcgctcgctcactg |
| | aggccgggcgaccaaaggtcgcccgacgcccgggctttgcccgggcggcctcagtgagcgagcgagcgcgcag |
| 17 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctcttttttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggctagcagactgactctgctgaccctgctgctgctgctgctggc |
| | tggggacagggccagcagcaaccctaatgctacctctagctctagccaggaccctgagagcctgcaggataggggggagggcaaggtggccaccact |
| | gtgatcagcaagatgctgtttgtggagcctatcctggaggtgagcagcctgcccaccaccaactctactaccaactctgctaccaagatcactgccaatacc |
| | actgatgaacccaccactcagcccactactgagcccaccacccagcccaccattcagcccactcagcccactacccagctgcctactgactctcccaccc |
| | agcccaccactggcagcttttgtcctggccctgtgaccctgtgctctgacctggagagccattctactgaggctgtgctgggggatgccctggtggacttca |
| | gcctgaagctgtaccatgccttctctgccatgaagaaggtggagaccaacatggccttcagccccttctctattgcctctctgctgacccaggtgctgctggg |
| | ggctggggagaatactaagaccaacctggagagcattctgagctatcccaaggacttcacctgtgtgcaccaggccctgaaggggttcactactaaaggg |
| | gtgacctctgtgtctcagatcttccacagccctgacctggccatcagggacacttttgtgaatgccagcaggaccctgtacagcagcagccccagagtgct |
| | gagcaataattctgatgctaatctggagctgattaacacttgggtggccaagaacaccaacaacaagatctctaggctgctggattctctgccctctgacacc |
| | aggctggtgctgctgaatgccatctatctgtctgccaagtggaagaccacctttgatcccaagaagaccaggatggagcccttccatttcaagaactctgtga |
| | ttaaagtgcccatgatgaactctaagaagtatcctgtggcccacttcattgatcagactctgaaggccaaggtggggcagctgcagctgagccacaacctg |
| | agcctggtgatcctggtgccccagaatctgaagcacaggctggaggacatggagcaggccctgagcccctctgtgttcaaggccatcatggagaagctg |
| | gagatgagcaagttccagcccaccctgctgactctgcccagaattaaggtgaccaccagccaggacatgctgagcatcatggagaagctggagttctttg |
| | acttctcttatgacctgaacctgtgtggcctgactgaggatcctgacctgcaggtgtctgccatgcagcaccagactgtgctggagctgactgagactgggg |
| | tggaggctgctgctgcctctgccatttctgtggccaggaccctgctggtgtttgaggtgcagcagcccttcctgtttgtgctgtgggaccagcagcacaagtt |
| | tcctgtgtttatgggcagggtgtatgaccctagggcctgaagatctagagctgaattcctgcagccagggggatcagcctctactgtgccttctagttgccag |
| | ccatctgttgtttgcccctcccccttgccttccttgaccctggaaggtgccactcccactgtcctttcctaataaaatgaggaaattgcatcacattgtctgagta |
| | ggtgtcattctattctggggggtggggggggcaggacagcaagggggaggattgggaagacaatagcaggcatgctggggatgcagtgggctctatg |
| | gcttctgaggcagaaagaaccagctggggctcgagatccactagggccgcaggaacccctagtgatggagttggccactccctctctgcgcgctcgctc |
| | gctcactgaggccgggcgaccaaaggtcgcccgacgcccgggctttgcccgggcggcctcagtgagcgagcgagcgcgcag |
| 18 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggccagcaggctgaccctgctgactctgctgctgctgctgctgg |
| | ctggggacagggccagcagcaaccctaatgccacctccagcagcagccaggaccctgagagcctgcaggataggggggagggcaaggtggctacta |
| | ctgtgatcagcaagatgctgtttgtggagcctatcctggaggtgagcagcctgcccaccactaactctactactaattctgccactaagatcactgccaatac |
| | cactgatgagccaaccactcagcccaccactgagcccaccacccagcccaccattcagcccactcagcctaccacccagctgcctactgacagccccac |
| | tcagcccactactggctctttctgtcctgggcctgtgactctgtgctctgacctggaatcccacagcactgaggctgtgctgggggatgccctggtggatttc |
| | agcctgaagctgtaccatgctttctctgccatgaagaaggtggagaccaacatggccttcagccctttctctattgccagcctgctgactcaggtgctgctgg |
| | gggctggggagaataccaagaccaacctggagtctatcctgagctatcccaaggacttcacttgtgtgcaccaggccctgaaggggttcaccaccaagg |
| | gggtgacctctgtgtctcagatctttcacagccctgacctggccatcagggatacctttgtgaatgccagcaggaccctgtacagcagcagccccagggtg |
| | ctgagcaacaactctgatgccaacctggagctgatcaacacctgggtggccaagaacaccaacaacaagatcagcaggctgctggacagcctgccctct |
| | gataccaggctggtgctgctgaatgccatctacctgtctgctaagtggaagaccacttttgatcctaagaagaccaggatggagccctttcacttcaagaact |
| | ctgtgatcaaggtgcccatgatgaactctaagaaataccctgtggcccacttcattgatcagaccctgaaggccaaggtgggccagctgcagctgagccac |
| | aacctgagcttggtgatcctggtgcctcagaacctgaagcacaggctggaggacatggagcaggccctgagcccctctgtgttcaaggccattatggaga |
| | agctggagatgagcaagttccagcctaccctgctgaccctgcccaggatcaaggtgactactagccaggatatgctgagcatcatggagaagctggagttt |
| | tttgatttcagctatgacctgaacctgtgtggcctgactgaggaccctgacctgcaggtgtctgccatgcagcatcagactgtgctggagctgactgagactg |
| | gggtggaggctgctgctgcctctgccatctctgtggccaggaccctgctggtgtttgaggtgcagcagcctttcctgtttgtgctgtgggatcagcagcaca |
| | agtttcctgtgttcatgggcagggtgtatgaccccagggcttgaagatctagagctgaattcctgcagccagggggatcagcctctactgtgccttctagttg |
| | ccagccatctgttgtttgcccctcccccttgccttccttgaccctggaaggtgccactcccactgtcctttcctaataaaatgaggaaattgcatcacattgtctg |
| | agtaggtgtcattctattctggggggtggggggggcaggacagcaagggggaggattgggaagacaatagcaggcatgctggggatgcagtgggctc |
| | tatggcttctgaggcagaaagaaccagctggggctcgagatccactagggccgcaggaacccctagtgatggagttggccactccctctctgcgcgctcg |
| | ctcgctcactgaggccgggcgaccaaaggtcgcccgacgcccgggctttgcccgggcggcctcagtgagcgagcgagcgcgcag |
| 19 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggctagcagactgactctgctgaccctgctgctgctgctgctggc |
| | tggggacagggccagcagcaaccctaatgctacctctagctctagccaggaccctgagagcctgcaggataggggggagggcaaggtggccaccact |
| | gtgatcagcaagatgctgtttgtggagcctatcctggaggtgagcagcctgcccaccaccaactctactaccaactctgctaccaagatcactgccaatacc |
| | actgatgaacccaccactcagcccactactgagcccaccacccagcccaccattcagcccactcagcccactacccagctgcctactgactctcccaccc |
| | agcccaccactgggagcttctgtccaggccctgtgactctgtgttctgacctggagagccacagcactgaagctgtgctgggggatgctctggtggacttc |
| | agcctgaagctgtaccatgccttctctgccatgaagaaggtggaaaccaacatggcctttagccccttcagcattgctagcctgctgactcaggtgctgctg |
| | ggggctggggagaacactaagactaacctggagtctatcctgtcttatcccaaggacttcacctgtgtgcatcaggccctgaaggggttcaccaccaaggg |
| | ggtcacctctgtgagccagatctttcacagccctgatctggccatcagggacacctttgtgaatgcctctaggactctgtacagcagcagccccagggtgct |
| | gagcaacaactctgatgccaacctggagctgatcaacacctgggtggccaagaacactaacaacaagatcagcaggctgctggacagcctgccctctga |
| | cactaggctggtgctgctgaatgccatctatctgtctgccaagtggaagaccacctttgaccccaagaagactaggatggagccctttcattttaagaactct |
| | gtgatcaaggtgcccatgatgaacagcaagaaataccctgtggcccatttcattgaccagactctgaaggctaaggtgggccagctgcagctgagccaca |
| | acctgagcctggtgatcctggtgccacagaatctgaagcacaggctggaggacatggagcaggctctgtctccctctgtgttcaaggccatcatggagaa |
| | gctggagatgtctaagttccagcccaccctgctgaccctgcccaggatcaaggtgaccacttctcaggacatgctgtctatcatggagaagctggagtttttt |
| | gacttttcttatgacctgaacctgtgtggcctgactgaggaccctgatctgcaggtgtctgccatgcagcaccagactgtgctggagctgactgagactggg |
| | gtggaggctgctgctgcttctgccatttctgtggctaggactctgctggtgtttgaggtgcagcagcccttcctgtttgtgctgtgggaccagcagcacaagtt |
| | ccctgtgttcatgggcagggtgtatgatcccagggcttgaagatctagagctgaattcctgcagccagggggatcagcctctactgtgccttctagttgccag |
| | ccatctgttgtttgcccctcccccttgccttccttgaccctggaaggtgccactcccactgtcctttcctaataaaatgaggaaattgcatcacattgtctgagta |
| | ggtgtcattctattctggggggtggggggggcaggacagcaagggggaggattgggaagacaatagcaggcatgctggggatgcagtgggctctatg |
| | gcttctgaggcagaaagaaccagctggggctcgagatccactagggccgcaggaacccctagtgatggagttggccactccctctctgcgcgctcgctc |
| | gctcactgaggccgggcgaccaaaggtcgcccgacgcccgggctttgcccgggcggcctcagtgagcgagcgagcgcgcag |
| 20 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggctagcagactgactctgctgaccctgctgctgctgctgctggc |
| | tggggacagggccagcagcaaccctaatgctacctctagctctagccaggaccctgagagcctgcaggataggggggagggcaaggtggccaccact |
| | gtgatcagcaagatgctgtttgtggagcctatcctggaggtgagcagcctgcccaccaccaactctactaccaactctgctaccaagatcactgccaatacc |
| | actgatgaacccaccactcagcccactactgagcccaccacccagcccaccattcagcccactcagcccactacccagctgcctactgactctcccaccc |
| | agcccaccactggcagcttctgccctgggcctgtgaccctgtgctctgacctggagagccattctactgaggctgtgctgggggatgccctggtggatttca |
| | gcctgaagctgtaccatgccttctctgccatgaagaaggtggagaccaacatggccttctctcccttcagcattgccagcctgctgacccaggtgctgctgg |
| | gggctggggagaacaccaagaccaacctggagagcatcctgtcttaccccaaggacttcacctgtgtgcatcaggccctgaagggcttcactactaaggg |
| | ggtgacctctgtgtctcagattttccacagccctgacctggctattagggacacttttgtgaatgcttctaggaccctgtacagcagctctcccagggtgctga |
| | gcaacaactctgatgccaacctggagctgattaacacttgggtggccaagaacactaacaataagatcagcaggctgctggacagcctgccctctgacac |
| | caggctggtgctgctgaatgccatctacctgtctgccaagtggaagactacttttgatcctaagaagaccaggatggagcctttccacttcaagaactctgtg |
| | atcaaggtgcccatgatgaattctaagaagtaccctgtggctcacttcattgaccagaccctgaaggccaaggtgggccagctgcagctgagccacaacct |
| | gagcctggtgattctggtgccccagaacctgaagcacaggctggaggacatggagcaggccctgagcccctctgtgttcaaggccatcatggagaagct |
| | ggagatgagcaagttccagcctactctgctgactctgcccaggatcaaggtgaccactagccaggacatgctgagcattatggagaagctggagttctttg |
| | acttttcttatgatctgaacctgtgtggcctgactgaggaccctgacctgcaggtgtctgccatgcagcaccagactgtgctggagctgactgagactggggt |
| | ggaggctgctgctgcctctgccatctctgtggctaggaccctgctggtgtttgaggtgcagcagcccttcctgtttgtgctgtgggaccagcagcacaagttc |
| | cctgtcttcatgggcagggtgtatgatcccagggcttgaagatctagagctgaattcctgcagccagggggatcagcctctactgtgccttctagttgccagc |
| | catctgttgtttgcccctcccccttgccttccttgaccctggaaggtgccactcccactgtcctttcctaataaaatgaggaaattgcatcacattgtctgagtag |
| | gtgtcattctattctggggggtggggggggcaggacagcaagggggaggattgggaagacaatagcaggcatgctggggatgcagtgggctctatgg |
| | cttctgaggcagaaagaaccagctggggctcgagatccactagggccgcaggaacccctagtgatggagttggccactccctctctgcgcgctcgctcg |
| | ctcactgaggccgggcgaccaaaggtcgcccgacgcccgggctttgcccgggcggcctcagtgagcgagcgagcgcgcag |
| 21 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtagggggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggccagcaggctgaccctgctgaccctgctgctgctcctgctgg |
| | ctggggatagggccagcagcaaccccaatgctactagcagcagcagccaggatcctgagagcctgcaggacagaggggaagggaaggtggccacc |
| | actgtgatcagcaagatgctgtttgtggagcctatcctggaggtgagctctctgcccaccactaacagcaccaccaattctgctactaagatcactgccaaca |
| | ccactgatgagcccaccacccagcctaccactgagccaactacccagcccaccatccagcccacccagcccaccactcagctgcctactgattctcctac |
| | tcagcctaccactgggagcttctgccctgggcctgtgaccctgtgctctgacctggagtctcattctactgaggctgtgctgggggatgccctggtggacttc |
| | agcctgaagctgtaccatgctttctctgccatgaagaaggtggagactaacatggccttctctcctttctctattgccagcctgctgactcaggtgctgctggg |
| | ggctggggagaataccaagactaacctggagagcatcctgtcttatcctaaggacttcacttgtgtgcatcaggccctgaagggcttcaccaccaaggggg |
| | tgacttctgtgagccagattttccacagccctgacctggccatcagagacacctttgtgaatgccagcaggaccctgtattctagctctcccagggtgctgag |
| | caacaactctgatgccaatctggaactgatcaacacttgggtggccaagaacaccaacaacaagatcagcaggctgctggatagcctgccctctgacacc |
| | aggctggtgctgctgaatgccatctacctgtctgccaagtggaagaccacctttgaccccaagaagactaggatggagcccttccacttcaagaactctgtg |
| | atcaaggtgcccatgatgaatagcaagaagtaccctgtggcccacttcattgaccagaccctgaaggccaaggtggggcagctgcagctgagccacaac |
| | ctgagcctggtgatcctggtgccccagaacctgaagcacagactggaggacatggagcaggccctgagcccatctgtgttcaaggctattatggagaagc |
| | tggagatgtctaagtttcagcctaccctgctgaccctgcccaggatcaaggtgactaccagccaggacatgctgagcatcatggagaagctggaattctttg |
| | acttcagctatgatctgaatctgtgtggcctgactgaagaccctgatttgcaggtgtctgctatgcagcaccagactgtgctggaactgactgagactggggt |
| | ggaggctgctgctgcctctgccatctctgtggccaggaccctgctggtgtttgaggtgcagcagcctttcctgtttgtgctgtgggatcagcagcacaagttc |
| | cctgtgttcatgggcagggtgtatgaccctagggcttgaagatctagagctgaattcctgcagccagggggatcagcctctactgtgccttctagttgccagc |
| | catctgttgtttgcccctcccccttgccttccttgaccctggaaggtgccactcccactgtcctttcctaataaaatgaggaaattgcatcacattgtctgagtag |
| | gtgtcattctattctggggggtggggtggggcaggacagcaagggggaggattgggaagacaatagcaggcatgctggggatgcagtgggctctatgg |
| | cttctgaggcagaaagaaccagctggggctcgagatccactagggccgcaggaacccctagtgatggagttggccactccctctctgcgcgctcgctcg |
| | ctcactgaggccgggcgaccaaaggtcgcccgacgcccgggctttgcccgggcggcctcagtgagcgagcgagcgcgcag |
| 22 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgaagatctagagctgaattcctgcagccagggggatcagcctctactgtgccttctagttgccagccat |
| | ctgttgtttgcccctcccccttgccttccttgaccctggaaggtgccactcccactgtcctttcctaataaaatgaggaaattgcatcacattgtctgagtaggtg |
| | tcattctattctggggggtggggggggcaggacagcaagggggaggattgggaagacaatagcaggcatgctggggatgcagtgggctctatggcttc |
| | tgaggcagaaagaaccagctggggctcgagatccactagggccgcaggaacccctagtgatggagttggccactccctctctgcgcgctcgctcgctca |
| | ctgaggccgggcgaccaaaggtcgcccgacgcccgggctttgcccgggcggcctcagtgagcgagcgagcgcgcag |
| 23 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggctagcaggctgaccctgctgaccctgctgctcctgctgctggc |
| | tggggacagggcctctagcaatcccaatgccacctctagctctagccaggatcctgagagcctgcaggacaggggggagggcaaggggccaccact |
| | gtgatcagcaagatgctgtttgtggagcccattctggaggtgtcttctctgcccaccaccaatagcaccactaattctgccaccaagatcactgctaacacca |
| | ctgatgagcccaccactcagcctaccactgagcccaccacccagcccactatccagcccacccagcccaccacccagctgcctactgacagccctaccc |
| | agcccactactgggagcttctgccctggccctgtgaccctgtgctctgacctggagagccacagcactgaggctgtgctgggggatgccctggtggactt |
| | cagcctgaagctgtaccatgccttttctgccatgaagaaggtggagaccaacatggcctttagccctttcagcattgccagcctgctgactcaggtgctgctg |
| | ggggctggggagaacaccaagaccaacctggagagcatcctgagctaccccaaggacttcacttgtgtgcaccaggccctgaagggcttcactaccaa |
| | gggggtgacttctgtgagccagatcttccacagccctgacctggccatcagggatacttttgtgaatgccagcaggaccctgtacagcagcagccccagg |
| | gtgctgagcaacaactctgatgctaacctggagctgatcaatacttgggtggccaagaacaccaacaacaagatttctaggctgctggactctctgccctct |
| | gacactaggctggtgctgctgaatgccatctacctgtctgccaagtggaagaccacctttgatcccaagaagactaggatggagcccttccacttcaagaac |
| | tctgtgatcaaggtgcccatgatgaacagcaagaagtaccctgtggcccatttcattgaccagaccctgaaggccaaggtggggcagctgcagctgagcc |
| | acaatctgagcctggtgattctggtgccccagaatctgaagcacagactggaggacatggagcaggctctgtctccctctgtgttcaaggccatcatggag |
| | aagctggagatgagcaagttccagcccactctgctgactctgcctaggatcaaggtgaccaccagccaggacatgctgagcattatggagaagctggagt |
| | tctttgacttcagctatgacctgaacctgtgtggcctgactgaggaccctgatctgcaggtgtctgccatgcagcaccagactgtgctggagctgactgaga |
| | ctggggtggaggctgctgctgcctctgctatttctgtggccaggaccctgctggtgtttgaggtgcagcagcccttcctgtttgtgctgtgggaccagcagca |
| | caagtttcctgtgttcatggggagggtgtatgaccccagggcctgaagatctagagctgaattcctgcagccagggggatcagcctctactgtgccttctagt |
| | tgccagccatctgttgtttgcccctcccccttgccttccttgaccctggaaggtgccactcccactgtcctttcctaataaaatgaggaaattgcatcacattgtc |
| | tgagtaggtgtcattctattctggggggtggggggggcaggacagcaagggggaggattgggaagacaatagcaggcatgctggggatgcagtgggc |
| | tctatggcttctgaggcagaaagaaccagctggggctcgagatccactagggccgcaggaacccctagtgatggagttggccactccctctctgcgcgct |
| | cgctcgctcactgaggccgggcgaccaaaggtcgcccgacgcccgggctttgcccgggcggcctcagtgagcgagcgagcgcgcag |
| 24 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctgaccctcctgctgctcctgctggc |
| | tggggatagagcctctagcaatccaaatgctaccagctcctcctcccaggatcctgaatcccttcaagacaggggggagggcaaggtggcaactactgtg |
| | atttccaagatgctgtttgtggagcccatcctggaggttagcagccttcctactaccaactcaacaaccaattcagccaccaaaattacagctaataccacag |
| | atgagccaacaactcaacccaccactgagcccaccactcagccaaccatccaacccacccaaccaactacccagctcccaacagattctcctacccagc |
| | ctaccactggctccttctgcccaggacctgttactctctgctctgacttggaaagccattctactgaggctgtcctgggggatgctttggtagacttctccctga |
| | agctctaccatgcattttcagcaatgaagaaggtggagaccaacatggccttttccccattcagtatagcaagtctcctgactcaggtcctgctgggggcag |
| | gggagaatacaaagaccaacctggagagcatcctcagttatcccaaggacttcacctgtgtccaccaggccctgaagggcttcacaacaaagggggtga |
| | cctcagtctctcagattttccacagcccagacctggccataagggacacctttgtgaatgcctctaggaccctgtactcaagctcccccagagtcctaagcaa |
| | caacagtgatgcaaatttggagctcatcaacacctgggtggccaagaacaccaacaacaagatcagcaggctgctagacagtctgccaagtgatacaag |
| | gcttgtcctcctcaatgctatctacctgagtgccaagtggaagacaacatttgatcccaagaaaaccagaatggaaccctttcacttcaaaaactcagttataa |
| | aagtgcccatgatgaatagcaagaagtaccctgtggcccatttcattgaccaaactttgaaagccaaggtggggcagctgcagctctcccacaatctgagtc |
| | tggtaatcctggtaccccagaacctgaaacataggcttgaagacatggaacaggctctcagcccttctgttttcaaggccataatggagaagctggagatgt |
| | ccaagttccagcccactctcctaacactacccaggatcaaagtgacaaccagccaggatatgctctcaatcatggagaaattggaattttttgatttttcttatg |
| | accttaacctgtgtgggctgacagaggaccctgatcttcaggtttctgcaatgcagcaccagacagtgctggaactgacagagactggggtggaagctgct |
| | gcagcaagtgcaatatctgtggccagaaccctgctggtctttgaagtgcagcagcccttcctgtttgtgctgtgggaccagcagcacaagttccctgtcttca |
| | tggggagggtatatgaccccagggcctgaagatctagagctgaattcctgcagccagggggatcagcctctactgtgccttctagttgccagccatctgttg |
| | tttgcccctcccccttgccttccttgaccctggaaggtgccactcccactgtcctttcctaataaaatgaggaaattgcatcacattgtctgagtaggtgtcattct |
| | attctggggggtggggtggggcaggacagcaagggggaggattgggaagacaatagcaggcatgctggggatgcagtgggctctatggcttctgagg |
| | cagaaagaaccagctggggctcgagatccactagggccgcaggaacccctagtgatggagttggccactccctctctgcgcgctcgctcgctcactgag |
| | gccgggcgaccaaaggtcgcccgacgcccgggctttgcccgggcggcctcagtgagcgagcgagcgcgcag |
| 25 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctcaccctcctgctgctcctgctggc |
| | tggggacagggcctccagcaaccccaatgcaaccagcagttcctctcaggatccagagtccctgcaggacaggggggagggcaaggtggccaccact |
| | gtgatctccaagatgctgtttgtggagcccatcctggaggtatccagcctccctaccaccaacagcaccaccaactcagccaccaaaatcactgctaacac |
| | cactgatgagcccacaacccagccaactactgagccaaccactcagcctaccatccaacccacccagcctactacccagctccccactgactctcctacc |
| | cagcccaccactggcagcttctgcccaggacctgtgaccctctgctctgacttggagtcccattccacagaggcagtgctgggggatgccctggtagactt |
| | ctccctgaagctctaccatgcattctctgccatgaagaaggtggagaccaacatggccttctcccccttcagcattgccagcctcctgacccaggtcctgctg |
| | ggggcaggggagaacacaaagacaaacctggagtccatcctcagctaccccaaggacttcacctgtgtccaccaggccctgaagggcttcacaaccaa |
| | gggggtgaccagtgtctcacagattttccacagcccagacctggccatcagggacacctttgtgaatgccagtaggaccctgtacagctccagtcccaga |
| | gtcctcagcaacaactctgatgccaacctggagctgatcaacacctgggggccaagaacaccaacaacaagatcagcaggctgctagactcactgccct |
| | ctgacacaaggctggtcctcctcaatgcaatctacctgagtgccaagtggaagaccacctttgaccccaagaagaccaggatggagcccttccacttcaag |
| | aacagtgtcattaaggtgcccatgatgaacagcaagaagtaccctgtggcccacttcattgaccagaccttgaaggccaaggtggggcagctgcagctct |
| | cccacaacctgtcactggtcatcctggttccccagaacctgaagcacaggcttgaggacatggagcaggccctcagcccttctgtattcaaggccataatg |
| | gagaagctggagatgagcaagttccagcccactctgctgaccctacccaggatcaaggtgaccaccagccaggacatgctctcaatcatggagaagttg |
| | gagttctttgacttcagctatgacctgaacctgtgtgggctgacagaggaccctgacctgcaggtgtcagccatgcagcaccagacagtgctggaactgac |
| | agagactggggtggaagctgctgcagcaagtgctatttcagtggccaggaccctgctggtctttgaggtccagcagcccttcctgtttgtgctctgggacca |
| | gcagcacaagttccctgtcttcatggggagggtctatgaccccagggcctgaagatctagagctgaattcctgcagccagggggatcagcctctactgtgc |
| | cttctagttgccagccatctgttgtttgcccctcccccttgccttccttgaccctggaaggtgccactcccactgtcctttcctaataaaatgaggaaattgcatc |
| | acattgtctgagtaggtgtcattctattctggggggtggggggggcaggacagcaagggggaggattgggaagacaatagcaggcatgctggggatgc |
| | agtgggctctatggcttctgaggcagaaagaaccagctggggctcgagatccactagggccgcaggaacccctagtgatggagttggccactccctctct |
| | gcgcgctcgctcgctcactgaggccgggcgaccaaaggtcgcccgacgcccgggctttgcccgggcggcctcagtgagcgagcgagcgcgcag |
| 26 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctgaccctcctgctgctcctgctggc |
| | tggggacagagccagcagcaaccccaatgccaccagctccagctctcaggacccagagagcctgcaggacaggggggagggcaaggtggccacca |
| | cagtgatcagcaagatgctgtttgtggagcccatcctggaggtgagcagcctccccaccaccaacagcaccaccaattctgccaccaagatcacagccaa |
| | caccacagatgagcccacaacccagcctaccacagagccaaccacacagcccaccatccaacccacccaacctactacccagctgcccacagacagc |
| | cctacccagcctaccacaggcagcttctgccctggccctgtgaccctgtgctctgacttggagagccactccacagaggctgtgctgggggatgccctggt |
| | ggatttcagcctgaagctgtaccatgccttctctgccatgaagaaggtggagaccaacatggccttcagccccttcagcattgccagcctcctgacccaggt |
| | cctgctgggggctggggagaacacaaagaccaacctggagagcatcctgagctaccccaaggacttcacctgtgtgcaccaggccctgaagggcttca |
| | ccaccaagggggtgacctctgtgagccagattttccacagccctgacctggccatcagggacacctttgtgaatgcctccaggaccctgtacagctctagc |
| | cccagggtgctgagcaacaactctgatgccaacttggagctgatcaacacctgggtggccaagaacaccaacaacaagatcagcaggctgctggacag |
| | cctgccctctgacaccaggctggtgctcctgaatgccatctacctgtctgccaagtggaagaccacctttgaccccaagaagaccagaatggagcccttcc |
| | acttcaagaactctgtgataaaggtgcccatgatgaacagcaagaagtaccctgtggcccacttcattgaccagaccctgaaagccaaggtgggccagct |
| | gcagctcagccacaacctgagcctggtcatcctggtgccccagaacctgaagcacaggctggaggacatggaacaggccctcagcccctctgtgttcaa |
| | ggccattatggagaagctggagatgagcaagttccagcccactctgctgacactgcccaggatcaaggtgaccaccagccaggacatgctgagcatcat |
| | ggagaagttggagttctttgacttcagctatgacctgaacctgtgtggcctgacagaggaccctgacctccaggtgtctgccatgcagcaccagacagtgct |
| | ggaactgacagagacaggggtggaagctgctgctgcctctgccatctctgtggccagaaccctgctggtgtttgaggtgcagcagcccttcctgtttgtgct |
| | gtgggaccagcagcacaagttccctgtgttcatgggcagggtgtatgaccccagggcctgaagatctagagctgaattcctgcagccagggggatcagc |
| | ctctactgtgccttctagttgccagccatctgttgtttgcccctcccccttgccttccttgaccctggaaggtgccactcccactgtcctttcctaataaaatgagg |
| | aaattgcatcacattgtctgagtaggtgtcattctattctggggggtggggggggcaggacagcaagggggaggattgggaagacaatagcaggcatgc |
| | tggggatgcagtgggctctatggcttctgaggcagaaagaaccagctggggctcgagatccactagggccgcaggaacccctagtgatggagttggcca |
| | ctccctctctgcgcgctcgctcgctcactgaggccgggcgaccaaaggtcgcccgacgcccgggctttgcccgggcggcctcagtgagcgagcgagc |
| | gcgcag |
| 27 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctctaggctgactctgctgactctgctgctcctgctgctggct |
| | ggggacagggccagctctaaccccaatgctacttctagctcctctcaggaccctgagagccttcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagcttcagctgagccataacctga |
| | gcctggtgatcctggtgccccagaacctgaagcataggctggaggacatggagcaggctctgagcccctctgtgttcaaggctattatggagaagctgga |
| | gatgagcaagttccagcccactctgctgaccctgcccagaatcaaggtgaccacctctcaggacatgctgagcatcatggaaaagctggagttctttgactt |
| | ttcttatgacctgaatctgtgtggcctgactgaggatcctgatcttcaggtgtctgccatgcagcaccagactgtgctggagctgactgagactggggtggag |
| | gctgctgctgcttctgccatctctgtggccaggaccctgctggtgtttgaggtgcagcagcccttcctgtttgtgctgtgggaccagcagcacaagtttcctgt |
| | gttcatgggcagggtgtatgaccccagagcctgaagatctagagctgaattccttcagccagggggatcagcctctactgtgccttctagttgccagccatct |
| | gttgtttgcccctcccccttgccttccttgaccctggaaggtgccactcccactgtcctttcctaataaaatgaggaaattgcatcacattgtctgagtaggtgtc |
| | attctattctggggggtggggtggggcaggacagcaagggggaggattgggaagacaatagcaggcatgctggggatgcagtgggctctatggcttctg |
| | aggcagaaagaaccagctggggctcgagatccactagggccgcaggaacccctagtgatggagttggccactccctctctgcgcgctcgctcgctcact |
| | gaggccgggcgaccaaaggtcgcccgacgcccgggctttgcccgggcggcctcagtgagcgagcgagcgcgcag |
| 28 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtagggggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctctttctttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagagctgaattcctgcagccagggggatcagcctctactgtgccttctagttgccagccatct |
| | gttgtttgcccctcccccttgccttccttgaccctggaaggtgccactcccactgtcctttcctaataaaatgaggaaattgcatcacattgtctgagtaggtgtc |
| | attctattctggggggtggggggggcaggacagcaagggggaggattgggaagacaatagcaggcatgctggggatgcagtgggctctatggcttctg |
| | aggcagaaagaaccagctggggctcgagatccactagggccgcaggaacccctagtgatggagttggccactccctctctgcgcgctcgctcgctcact |
| | gaggccgggcgaccaaaggtcgcccgacgcccgggctttgcccgggcggcctcagtgagcgagcgagcgcgcag |
| 29 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctctttctttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggctagcaggctgaccctgctgaccctgctgctcctgctgctgg |
| | ctggggacagggcctctagcaatcccaatgccacctctagctctagccaggatcctgagagcctgcaggacaggggggagggcaaggtggccaccact |
| | gtgatcagcaagatgctgtttgtggagcccattctggaggtgtcttctctgcccaccaccaatagcaccactaattctgccaccaagatcactgctaacacca |
| | ctgatgagcccaccactcagcctaccactgagcccaccacccagcccactatccagcccacccagcccaccacccagctgcctactgacagccctaccc |
| | agcccactactgggagcttctgccctggccctgtgaccctgtgctctgacctggagagccacagcactgaggctgtgctgggggatgccctggtggactt |
| | cagcctgaagctgtaccatgccttttctgccatgaagaaggtggagaccaacatggcctttagccctttcagcattgccagcctgctgactcaggtgctgctg |
| | ggggctggggagaacaccaagaccaacctggagagcatcctgagctaccccaaggacttcacttgtgtgcaccaggccctgaagggcttcactaccaa |
| | gggggtgacttctgtgagccagatcttccacagccctgacctggccatcagggatacttttgtgaatgccagcaggaccctgtacagcagcagccccagg |
| | gtgctgagcaacaactctgatgctaacctggagctgatcaatacttgggtggccaagaacaccaacaacaagatttctaggctgctggactctctgccctct |
| | gacactaggctggtgctgctgaatgccatctacctgtctgccaagtggaagaccacctttgatcccaagaagactaggatggagcccttccacttcaagaac |
| | tctgtgatcaaggtgcccatgatgaacagcaagaagtaccctgtggcccatttcattgaccagaccctgaaggccaaggtggggcagctgcagctgagcc |
| | acaatctgagcctggtgattctggtgccccagaatctgaagcacagactggaggacatggagcaggctctgtctccctctgtgttcaaggccatcatggag |
| | aagctggagatgagcaagttccagcccactctgctgactctgcctaggatcaaggtgaccaccagccaggacatgctgagcattatggagaagctggagt |
| | tctttgacttcagctatgacctgaacctgtgtggcctgactgaggaccctgatctgcaggtgtctgccatgcagcaccagactgtgctggagctgactgaga |
| | ctggggtggaggctgctgctgcctctgctatttctgtggccaggaccctgctggtgtttgaggtgcagcagcccttcctgtttgtgctgtgggaccagcagca |
| | caagtttcctgtgttcatggggagggtgtatgaccccagggcctgacatctagagctgaattcctgcagccagggggatcagcctctactgtgccttctagtt |
| | gccagccatctgttgtttgcccctcccccttgccttccttgaccctggaaggtgccactcccactgtcctttcctaataaaatgaggaaattgcatcacattgtct |
| | gagtaggtgtcattctattctggggggtggggtggggcaggacagcaagggggaggattgggaagacaatagcaggcatgctggggatgcagtgggct |
| | ctatggcttctgaggcagaaagaaccagctggggctcgagatccactagggccgcaggaacccctagtgatggagttggccactccctctctgcgcgctc |
| | gctcgctcactgaggccgggcgaccaaaggtcgcccgacgcccgggctttgcccgggcggcctcagtgagcgagcgagcgcgcag |
| 30 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctgaccctcctgctgctgctgctggc |
| | tggggatagagcctcctcaaatcccaccatccaacccacccaaccaactacccagctcccaacagattctcctacccagcccactactgggtccttctgcc |
| | caggacctgttactctctgctctgacttggagagtcattcaacagaggccgtgttgggggatgctttggtagatttctccctgaagctctaccacgccttctcag |
| | caatgaagaaggtggagaccaacatggccttttccccattcagcatcgccagcctccttacccaggtcctgctcggggctggggagaacaccaaaacaaa |
| | cctggagagcatcctctcttaccccaaggacttcacctgtgtccaccaggccctgaagggcttcacgaccaaaggtgtcacctcagtctctcagattttccac |
| | agcccagacctggccataagggacacctttgtgaatgcctctcggaccctgtacagcagcagccccagagtcctaagcaacaacagtgacgccaacttg |
| | gagctcatcaacacctgggtggccaagaacaccaacaacaagatcagccggctgctagacagtctgccctccgatacccgccttgtcctcctcaatgctat |
| | ctacctgagtgccaagtggaagacaacatttgatcccaagaaaaccagaatggaaccctttcacttcaaaaactcagttataaaagtgcccatgatgaatag |
| | caagaagtaccctgtggcccatttcattgaccaaactttgaaagccaaggtggggcagctgcagctctcccacaatctgagtttggtgatcctggtacccca |
| | gaacctgaaacatcgtcttgaagacatggaacaggctctcagcccttctgttttcaaggccatcatggagaaactggagatgtccaagttccagcccactctc |
| | ctaacactaccccgcatcaaagtgacgaccagccaggatatgctctcaatcatggagaaattggaattcttcgatttttcttatgaccttaacctgtgtgggctg |
| | acagaggaccctgatcttcaggtttctgcgatgcagcaccagacagtgctggaactgacagagactggggggaggcggctgcagcctccgccatctct |
| | gtggcccgcaccctgctggtctttgaagtgcagcagcccttcctcttcgtgctctgggaccagcagcacaagttccctgtcttcatggggcgagtatatgacc |
| | ccagggcctgaagatctagagctgaattcctgcagccagggggatcagcctctactgtgccttctagttgccagccatctgttgtttgcccctcccccttgcct |
| | tccttgaccctggaaggtgccactcccactgtcctttcctaataaaatgaggaaattgcatcacattgtctgagtaggtgtcattctattctggggggggggtg |
| | gggcaggacagcaagggggaggattgggaagacaatagcaggcatgctggggatgcagtgggctctatggcttctgaggcagaaagaaccagctgg |
| | ggctcgagatccactagggccgcaggaacccctagtgatggagttggccactccctctctgcgcgctcgctcgctcactgaggccgggcgaccaaaggt |
| | cgcccgacgcccgggctttgcccgggcggcctcagtgagcgagcgagcgcgcag |
| 31 | ctgcgcgctcgctcgctcactgaggccgcccgggcaaagcccgggcggcctcagtgagcgagcgagcgcgcagagagggagtggccaactccatca |
| | ctaggggttcctgcggcctagtaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagaggggtacccggggatcttgctaccagtggaacagccactaaggattctgcagtgagagcagag |
| | ggccagctaagtggtactctcccagagactgtctgactcacgccaccccctccaccttggacacaggacgctgtggtttctgagccaggtacaatgactcct |
| | ttcggtaagtgcagtggaagctgtacactgcccaggcaaagcgtccgggcagcgtaggcgggcgactcagatcccagccagtggacttagcccctgttt |
| | gctcctccgataactggggtgaccttggttaatattcaccagcagcctcccccgttgcccctctggatccactgcttaaatacggacgaggacagggccctg |
| | tctcctcagcttcaggcaccaccactgacctgggacagtgaatagatcctgagaacttcagggtgagtctatgggacccttgatgttttctttccccttcttttct |
| | atggttaagttcatgtcataggaaggggagaagtaacagggtacacatattgaccaaatcagggtaattttgcatttgtaattttaaaaaatgctttcttcttttaat |
| | atacttttttgtttatcttatttctaatactttccctaatctctttctttcagggcaataatgatacaatgtatcatgcctctttgcaccattctaaagaataacagtgataa |
| | tttctgggttaaggcaatagcaatatttctgcatataaatatttctgcatataaattgtaactgatgtaagaggtttcatattgctaatagcagctacaatccagcta |
| | ccattctgcttttattttctggttgggataaggctggattattctgagtccaagctaggcccttttgctaatcttgttcatacctcttatcttcctcccacagctcctgg |
| | gcaacctgctggtctctctgctggcccatcactttggcaaagcacgcgtgccaccatggcctccaggctgaccctgctgaccctcctgctgctgctgctggc |
| | tggggatagagcctcctcaaatgggtccttctgcccaggacctgttactctctgctctgacttggagagtcattcaacagaggccgtgttgggggatgctttg |
| | gtagatttctccctgaagctctaccacgccttctcagcaatgaagaaggtggagaccaacatggccttttccccattcagcatcgccagcctccttacccagg |
| | tcctgctcggggctggggagaacaccaaaacaaacctggagagcatcctctcttaccccaaggacttcacctgtgtccaccaggccctgaagggcttcac |
| | gaccaaaggtgtcacctcagtctctcagattttccacagcccagacctggccataagggacacctttgtgaatgcctctcggaccctgtacagcagcagcc |
| | ccagagtcctaagcaacaacagtgacgccaacttggagctcatcaacacctgggtggccaagaacaccaacaacaagatcagccggctgctagacagt |
| | ctgccctccgatacccgccttgtcctcctcaatgctatctacctgagtgccaagtggaagacaacatttgatcccaagaaaaccagaatggaaccctttcactt |
| | caaaaactcagttataaaagtgcccatgatgaatagcaagaagtaccctgtggcccatttcattgaccaaactttgaaagccaaggtggggcagctgcagct |
| | ctcccacaatctgagtttggtgatcctggtaccccagaacctgaaacatcgtcttgaagacatggaacaggctctcagcccttctgttttcaaggccatcatgg |
| | agaaactggagatgtccaagttccagcccactctcctaacactaccccgcatcaaagtgacgaccagccaggatatgctctcaatcatggagaaattggaa |
| | ttcttcgatttttcttatgaccttaacctgtgtgggctgacagaggaccctgatcttcaggtttctgcgatgcagcaccagacagtgctggaactgacagagact |
| | ggggtggaggcggctgcagcctccgccatctctgtggcccgcaccctgctggtctttgaagtgcagcagcccttcctcttcgtgctctgggaccagcagca |
| | caagttccctgtcttcatggggcgagtatatgaccccagggcctgaagatctagagctgaattcctgcagccagggggatcagcctctactgtgccttctagt |
| | tgccagccatctgttgtttgcccctcccccttgccttccttgaccctggaaggtgccactcccactgtcctttcctaataaaatgaggaaattgcatcacattgtc |
| | tgagtaggtgtcattctattctggggggtggggtggggcaggacagcaagggggaggattgggaagacaatagcaggcatgctggggatgcagtgggc |
| | tctatggcttctgaggcagaaagaaccagctggggctcgagatccactagggccgcaggaacccctagtgatggagttggccactccctctctgcgcgct |
| | cgctcgctcactgaggccgggcgaccaaaggtcgcccgacgcccgggctttgcccgggcggcctcagtgagcgagcgagcgcgcag |
| 32 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccacttgaccccttggaattttggtggagagg |
| | agcagaggttgtcctggtgtggtttaggtagtgtgagaggggtacctggggatcttgctaccagtggaacagccactaaggattctgcagtgagagcagag |
| | ggccagctaagtggtactctcccagagactgtctgactcagccaccccctccaccttggacacaggatgctgtggtttctgagccaggtacaatgactccttt |
| | ggtaagtgcagtggaagctgtacactgcccaggcaaaggtctgggcaggtagggggtgactcagatcccagccagtggacttagcccctgtttgctcctc |
| | tgataactggggtgaccttggttaatattcaccagcagcctcccctgttgcccctctggatccactgcttaaatatggatgaggacagggccctgtctcctcag |
| | cttcaggcaccaccactgacctgggacagtgaatagatcttgagaacttcagggtgagtctatgggacccttgatgttttctttccccttcttttctatggttaagt |
| | tcatgtcataggaaggggagaagtaacagggtacacatattgaccaaatcagggtaattttgcatttgtaattttaaaaaatgctttcttcttttaatatacttttttgt |
| | ttatcttatttctaatactttccctaatctctttctttcagggcaataatgatacaatgtatcatgcctctttgcaccattctaaagaataacagtgataatttctgggtta |
| | aggcaatagcaatatttctgcatataaatatttctgcatataaattgtaactgatgtaagaggtttcatattgctaatagcagctacaatccagctaccattctgcttt |
| | tattttctggttgggataaggctggattattctgagtccaagctaggcccttttgctaatcttgttcatacctcttatcttcctcccacagctcctgggcaacctgct |
| | ggtctctctgctggcccatcactttggcaaagcactagtgccaccatggcctctaggctgactctgctgactctgctgctcctgctgctggctggggacagg |
| | gccagctctaaccccaatgctacttctagctcctctcaggaccctgagagcctgcaggacaggggggagggcaaggtggccaccactgtgatctctaaga |
| | tgctgtttgtggagcccatcctggaggtgagcagcctgcccaccaccaacagcaccaccaactctgccactaagatcactgccaacaccactgatgagcc |
| | caccacccagcccactactgagcccaccacccagcctactatccagcccacccagcccactacccagctgcccactgacagccctacccagcccacca |
| | ctggcagcttctgccctgggcctgtgaccctgtgctctgacctggagagccattctactgaggctgtgctgggggatgccctggtggacttctctctgaagct |
| | gtaccatgctttctctgccatgaagaaggtggagactaacatggcttttagccccttcagcattgctagcctgctgacccaggtgctgctgggggctgggga |
| | gaacactaagaccaacctggaatctatcctgagctaccccaaggacttcacctgtgtgcaccaggccctgaagggcttcaccaccaagggggtgacctct |
| | gtgagccagatcttccacagccctgacctggccatcagggatacttttgtgaatgccagcagaaccctgtactcttctagccccagggtgctgagcaataatt |
| | ctgatgctaacctggagctgatcaatacctgggggctaagaataccaacaacaagattagcaggctgctggactctctgccttctgataccaggctggtgc |
| | tgctgaatgccatttacctgtctgccaagtggaagaccacttttgaccctaagaagactaggatggagcctttccacttcaagaactctgtcattaaggtgccc |
| | atgatgaacagcaagaagtatcctgtggcccactttattgaccagactctgaaggccaaggtgggccagctgcagctgagccataacctgagcctggtgat |
| | cctggtgccccagaacctgaagcataggctggaggacatggagcaggctctgagcccctctgtgttcaaggctattatggagaagctggagatgagcaa |
| | gttccagcccactctgctgaccctgcccagaatcaaggtgaccacctctcaggacatgctgagcatcatggaaaagctggagttctttgacttttcttatgacc |
| | tgaatctgtgtggcctgactgaggatcctgatctgcaggtgtctgccatgcagcaccagactgtgctggagctgactgagactggggtggaggctgctgct |
| | gcttctgccatctctgtggccaggaccctgctggtgtttgaggtgcagcagcccttcctgtttgtgctgtgggaccagcagcacaagtttcctgtgttcatggg |
| | cagggtgtatgaccccagagcctgacatctagagctgaattcctgcagccagggggatcagcctctactgtgccttctagttgccagccatctgttgtttgcc |
| | cctcccccttgccttccttgaccctggaaggtgccactcccactgtcctttcctaataaaatgaggaaattgcatcacattgtctgagtaggtgtcattctattctg |
| | gggggtggggtggggcaggacagcaagggggaggattgggaagacaatagcaggcatgctggggatgcagtgggctctatggcttctgaggcagaa |
| | agaaccagctggggctcgagatccactagggccgcaggaacccctagtgatggagttggccactccctctctgcgcgctcgctcgctcactgaggccgg |
| | gcgaccaaaggtcgcccgacgcccgggctttgcccgggcggcctcagtgagcgagcgagcgcgcag |
| 33 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccacttgaccccttggaattttggtggagagg |
| | agcagaggttgtcctggtgtggtttaggtagtgtgagaggggtacctggggatcttgctaccagtggaacagccactaaggattctgcagtgagagcagag |
| | ggccagctaagtggtactctcccagagactgtctgactcagccaccccctccaccttggacacaggatgtgtggtttctgagccaggtacaatgactccttt |
| | ggtaagtgcagtggaagctgtacactgcccaggcaaaggtctgggcaggtagggggtgactcagatcccagccagtggacttagcccctgtttgctcctc |
| | tgataactggggtgaccttggttaatattcaccagcagcctcccctgttgcccctctggatccactgcttaaatatggatgaggacagggccctgtctcctcag |
| | cttcaggcaccaccactgacctgggacagtgaatagatcttgagaacttcagggtgagtctatgggacccttgatgttttctttccccttcttttctatggttaagt |
| | tcatgtcataggaaggggagaagtaacagggtacacatattgaccaaatcagggtaattttgcatttgtaattttaaaaaatgctttcttcttttaatatacttttttgt |
| | ttatcttatttctaatactttccctaatctcttttttcagggcaataatgatacaatgtatcatgcctctttgcaccattctaaagaataacagtgataatttctgggtta |
| | aggcaatagcaatatttctgcatataaatatttctgcatataaattgtaactgatgtaagaggtttcatattgctaatagcagctacaatccagctaccattctgcttt |
| | tattttctggttgggataaggctggattattctgagtccaagctaggcccttttgctaatcttgttcatacctcttatcttcctcccacagctcctgggcaacctgct |
| | ggtctctctgctggcccatcactttggcaaagcactagtgccaccatggctagcaggctgaccctgctgaccctgctgctcctgctgctggctggggacag |
| | ggcctctagcaatcccaatgccacctctagctctagccaggatcctgagagcctgcaggacaggggggagggcaaggtggccaccactgtgatcagca |
| | agatgctgtttgtggagcccattctggaggtgtcttctctgcccaccaccaatagcaccactaattctgccaccaagatcactgctaacaccactgatgagcc |
| | caccactcagcctaccactgagcccaccacccagcccactatccagcccacccagcccaccacccagctgcctactgacagccctacccagcccactac |
| | tgggagcttctgccctggccctgtgaccctgtgctctgacctggagagccacagcactgaggctgtgctgggggatgccctggtggacttcagcctgaag |
| | ctgtaccatgccttttctgccatgaagaaggtggagaccaacatggcctttagccctttcagcattgccagcctgctgactcaggtgctgctgggggctggg |
| | gagaacaccaagaccaacctggagagcatcctgagctaccccaaggacttcacttgtgtgcaccaggccctgaagggcttcactaccaagggggtgact |
| | tctgtgagccagatcttccacagccctgacctggccatcagggatacttttgtgaatgccagcaggaccctgtacagcagcagccccagggtgctgagca |
| | acaactctgatgctaacctggagctgatcaatacttgggtggccaagaacaccaacaacaagatttctaggctgctggactctctgccctctgacactaggct |
| | ggtgctgctgaatgccatctacctgtctgccaagtggaagaccacctttgatcccaagaagactaggatggagcccttccacttcaagaactctgtgatcaa |
| | ggtgcccatgatgaacagcaagaagtaccctgtggcccatttcattgaccagaccctgaaggccaaggtggggcagctgcagctgagccacaatctgag |
| | cctggtgattctggtgccccagaatctgaagcacagactggaggacatggagcaggctctgtctccctctgtgttcaaggccatcatggagaagctggaga |
| | tgagcaagttccagcccactctgctgactctgcctaggatcaaggtgaccaccagccaggacatgctgagcattatggagaagctggagttctttgacttca |
| | gctatgacctgaacctgtgtggcctgactgaggaccctgatctgcaggtgtctgccatgcagcaccagactgtgctggagctgactgagactggggtgga |
| | ggctgctgctgcctctgctatttctgtggccaggaccctgctggtgtttgaggtgcagcagcccttcctgtttgtgctgtgggaccagcagcacaagtttcctg |
| | tgttcatggggagggtgtatgaccccagggcctgacatctagagctgaattcctgcagccagggggatcagcctctactgtgccttctagttgccagccatc |
| | tgttgtttgcccctcccccttgccttccttgaccctggaaggtgccactcccactgtcctttcctaataaaatgaggaaattgcatcacattgtctgagtaggtgt |
| | cattctattctggggggtggggggggcaggacagcaagggggaggattgggaagacaatagcaggcatgctggggatgcagtgggctctatggcttct |
| | gaggcagaaagaaccagctggggctcgagatccactagggccgcaggaacccctagtgatggagttggccactccctctctgcgcgctcgctcgctcac |
| | tgaggccgggcgaccaaaggtcgcccgacgcccgggctttgcccgggcggcctcagtgagcgagcgagcgcgcag |
| 34 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgggtttaggtagtgtgagagggaggctcagaggcacacaggagtttctgggctcaccctgcccccttccaacccctcagtt |
| | cccatcctccagcagctgtttgtgtgctgcctctgaagtccacactgaacaaacttcagcctactcatgtccctaaaatgggcaaacattgcaagcagcaaac |
| | agcaaacacacagccctccctgcctgctgaccttggagctggggcagaggtcagagacctctctgggcccatgccacctccaacatccactcgacccctt |
| | ggaatttcggtggagaggagcagaggttgtcctggcgtggtttaggtagtgtgagaggggtacccggggatcttgctaccagtggaacagccactaagga |
| | ttctgcagtgagagcagagggccagctaagtggtactctcccagagactgtctgactcacgccaccccctccaccttggacacaggacgctgtggtttctg |
| | agccaggtacaatgactcctttcggtaagtgcagtggaagctgtacactgcccaggcaaagcgtccgggcagcgtaggcgggcgactcagatcccagc |
| | cagtggacttagcccctgtttgctcctccgataactggggtgaccttggttaatattcaccagcagcctcccccgttgcccctctggatccactgcttaaatac |
| | ggacgaggacagggccctgtctcctcagcttcaggcaccaccactgacctgggacagtgaatagatcttgagaacttcagggtgagtctatgggaccctt |
| | gatgttttctttccccttcttttctatggttaagttcatgtcataggaaggggagaagtaacagggtacacatattgaccaaatcagggtaattttgcatttgtaattt |
| | taaaaaatgctttcttcttttaatatacttttttgtttatcttatttctaatactttccctaatctcttttttcagggcaataatgatacaatgtatcatgcctctttgcacca |
| | ttctaaagaataacagtgataatttctgggttaaggcaatagcaatatttctgcatataaatatttctgcatataaattgtaactgatgtaagaggtttcatattgcta |
| | atagcagctacaatccagctaccattctgcttttattttctggttgggataaggctggattattctgagtccaagctaggcccttttgctaatcttgttcatacctctt |
| | atcttcctcccacagctcctgggcaacctgctggtctctctgctggcccatcactttggcaaagcactagtgccaccatggcctctaggctgactctgctgact |
| | ctgctgctcctgctgctggctggggacagggccagctctaaccccaatgctacttctagctcctctcaggaccctgagagcctgcaggacaggggggag |
| | ggcaaggtggccaccactgtgatctctaagatgctgtttgtggagcccatcctggaggtgagcagcctgcccaccaccaacagcaccaccaactctgcca |
| | ctaagatcactgccaacaccactgatgagcccaccacccagcccactactgagcccaccacccagcctactatccagcccacccagcccactacccagc |
| | tgcccactgacagccctacccagcccaccactggcagcttctgccctgggcctgtgaccctgtgctctgacctggagagccattctactgaggctgtgctg |
| | ggggatgccctggtggacttctctctgaagctgtaccatgctttctctgccatgaagaaggtggagactaacatggcttttagccccttcagcattgctagcct |
| | gctgacccaggtgctgctgggggctggggagaacactaagaccaacctggaatctatcctgagctaccccaaggacttcacctgtgtgcaccaggccct |
| | gaagggcttcaccaccaagggggtgacctctgtgagccagatcttccacagccctgacctggccatcagggatacttttgtgaatgccagcagaaccctgt |
| | actcttctagccccagggtgctgagcaataattctgatgctaacctggagctgatcaatacctgggtggctaagaataccaacaacaagattagcaggctgc |
| | tggactctctgccttctgataccaggctggtgctgctgaatgccatttacctgtctgccaagtggaagaccacttttgaccctaagaagactaggatggagcct |
| | ttccacttcaagaactctgtcattaaggtgcccatgatgaacagcaagaagtatcctgtggcccactttattgaccagactctgaaggccaaggtgggccag |
| | ctgcagctgagccataacctgagcctggtgatcctggtgccccagaacctgaagcataggctggaggacatggagcaggctctgagcccctctgtgttca |
| | aggctattatggagaagctggagatgagcaagttccagcccactctgctgaccctgcccagaatcaaggtgaccacctctcaggacatgctgagcatcatg |
| | gaaaagctggagttctttgacttttcttatgacctgaatctgtgtggcctgactgaggatcctgatctgcaggtgtctgccatgcagcaccagactgtgctgga |
| | gctgactgagactggggtggaggctgctgctgcttctgccatctctgtggccaggaccctgctggtgtttgaggtgcagcagcccttcctgtttgtgctgtgg |
| | gaccagcagcacaagtttcctgtgttcatgggcagggtgtatgaccccagagcctgacatctagagctgaattcctgcagccagggggatcagcctctact |
| | gtgccttctagttgccagccatctgttgtttgcccctcccccttgccttccttgaccctggaaggtgccactcccactgtcctttcctaataaaatgaggaaattg |
| | catcacattgtctgagtaggtgtcattctattctggggggtggggggggcaggacagcaagggggaggattgggaagacaatagcaggcatgctgggg |
| | atgcagtgggctctatggcttctgaggcagaaagaaccagctggggctcgagatccactagggccgcaggaacccctagtgatggagttggccactccc |
| | tctctgcgcgctcgctcgctcactgaggccgggcgaccaaaggtcgcccgacgcccgggctttgcccgggcggcctcagtgagcgagcgagcgcgca |
| | g |
| 35 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgggtttaggtagtgtgagagggaggctcagaggcacacaggagtttctgggctcaccctgcccccttccaacccctcagtt |
| | cccatcctccagcagctgtttgtgtgctgcctctgaagtccacactgaacaaacttcagcctactcatgtccctaaaatgggcaaacattgcaagcagcaaac |
| | agcaaacacacagccctccctgcctgctgaccttggagctggggcagaggtcagagacctctctgggcccatgccacctccaacatccactcgacccctt |
| | ggaatttcggtggagaggagcagaggttgtcctggcgtggtttaggtagtgtgagagggaggctcagaggcacacaggagtttctgggctcaccctgccc |
| | ccttccaacccctcagttcccatcctccagcagctgtttgtgtgctgcctctgaagtccacactgaacaaacttcagcctactcatgtccctaaaatgggcaaa |
| | cattgcaagcagcaaacagcaaacacacagccctccctgcctgctgaccttggagctggggcagaggtcagagacctctctgggcccatgccacctcca |
| | acatccactcgaccccttggaatttcggtggagaggagcagaggttgtcctggcgtggtttaggtagtgtgagagggaggctcagaggcacacaggagtt |
| | tctgggctcaccctgcccccttccaacccctcagttcccatcctccagcagctgtttgtgtgctgcctctgaagtccacactgaacaaacttcagcctactcat |
| | gtccctaaaatgggcaaacattgcaagcagcaaacagcaaacacacagccctccctgcctgctgaccttggagctggggcagaggtcagagacctctct |
| | gggcccatgccacctccaacatccactcgaccccttggaatttcggtggagaggagcagaggttgtcctggcgtggtttaggtagtgtgagaggggtacc |
| | cggggatcttgctaccagtggaacagccactaaggattctgcagtgagagcagagggccagctaagtggtactctcccagagactgtctgactcacgcca |
| | ccccctccaccttggacacaggacgctgtggtttctgagccaggtacaatgactcctttcggtaagtgcagtggaagctgtacactgcccaggcaaagcgt |
| | ccgggcagcgtaggcgggcgactcagatcccagccagtggacttagcccctgtttgctcctccgataactggggtgaccttggttaatattcaccagcagc |
| | ctcccccgttgcccctctggatccactgcttaaatacggacgaggacagggccctgtctcctcagcttcaggcaccaccactgacctgggacagtgaatag |
| | atcttgagaacttcagggtgagtctatgggacccttgatgttttctttccccttcttttctatggttaagttcatgtcataggaaggggagaagtaacagggtaca |
| | catattgaccaaatcagggtaattttgcatttgtaattttaaaaaatgctttcttcttttaatatacttttttgtttatcttatttctaatactttccctaatctctttctttcagg |
| | gcaataatgatacaatgtatcatgcctctttgcaccattctaaagaataacagtgataatttctgggttaaggcaatagcaatatttctgcatataaatatttctgca |
| | tataaattgtaactgatgtaagaggtttcatattgctaatagcagctacaatccagctaccattctgcttttattttctggttgggataaggctggattattctgagtc |
| | caagctaggcccttttgctaatcttgttcatacctcttatcttcctcccacagctcctgggcaacctgctggtctctctgctggcccatcactttggcaaagcact |
| | agtgccaccatggcctctaggctgactctgctgactctgctgctcctgctgctggctggggacagggccagctctaaccccaatgctacttctagctcctctc |
| | aggaccctgagagcctgcaggacaggggggagggcaaggtggccaccactgtgatctctaagatgctgtttgtggagcccatcctggaggtgagcagc |
| | ctgcccaccaccaacagcaccaccaactctgccactaagatcactgccaacaccactgatgagcccaccacccagcccactactgagcccaccacccag |
| | cctactatccagcccacccagcccactacccagctgcccactgacagccctacccagcccaccactggcagcttctgccctgggcctgtgaccctgtgct |
| | ctgacctggagagccattctactgaggctgtgctgggggatgccctggtggacttctctctgaagctgtaccatgctttctctgccatgaagaaggtggaga |
| | ctaacatggcttttagccccttcagcattgctagcctgctgacccaggtgctgctgggggctggggagaacactaagaccaacctggaatctatcctgagct |
| | accccaaggacttcacctgtgtgcaccaggccctgaagggcttcaccaccaagggggtgacctctgtgagccagatcttccacagccctgacctggccat |
| | cagggatacttttgtgaatgccagcagaaccctgtactcttctagccccagggtgctgagcaataattctgatgctaacctggagctgatcaatacctgggtg |
| | gctaagaataccaacaacaagattagcaggctgctggactctctgccttctgataccaggctggtgctgctgaatgccatttacctgtctgccaagtggaaga |
| | ccacttttgaccctaagaagactaggatggagcctttccacttcaagaactctgtcattaaggtgcccatgatgaacagcaagaagtatcctgtggcccacttt |
| | attgaccagactctgaaggccaaggtgggccagctgcagctgagccataacctgagcctggtgatcctggtgccccagaacctgaagcataggctggag |
| | gacatggagcaggctctgagcccctctgtgttcaaggctattatggagaagctggagatgagcaagttccagcccactctgctgaccctgcccagaatcaa |
| | ggtgaccacctctcaggacatgctgagcatcatggaaaagctggagttctttgacttttcttatgacctgaatctgtgtggcctgactgaggatcctgatctgca |
| | ggtgtctgccatgcagcaccagactgtgctggagctgactgagactggggtggaggctgctgctgcttctgccatctctgtggccaggaccctgctggtgtt |
| | tgaggtgcagcagcccttcctgtttgtgctgtgggaccagcagcacaagtttcctgtgttcatgggcagggtgtatgaccccagagcctgacatctagagct |
| | gaattcctgcagccagggggatcagcctctactgtgccttctagttgccagccatctgttgtttgcccctcccccttgccttccttgaccctggaaggtgccact |
| | cccactgtcctttcctaataaaatgaggaaattgcatcacattgtctgagtaggtgtcattctattctggggggtggggggggcaggacagcaagggggag |
| | gattgggaagacaatagcaggcatgctggggatgcagtgggctctatggcttctgaggcagaaagaaccagctggggctcgagatccactagggccgc |
| | aggaacccctagtgatggagttggccactccctctctgcgcgctcgctcgctcactgaggccgggcgaccaaaggtcgcccgacgcccgggctttgccc |
| | gggcggcctcagtgagcgagcgagcgcgcag |
| 36 | ctgcgcgctcgctcgctcactgaggccgcccgggcaaagcccgggcggcctcagtgagcgagcgagcgcgcagagagggagtggccaactccatca |
| | ctaggggttcctgcggcctagtccggggatcttgctaccagtggaacagccactaaggattctgcagtgagagcagagggccagctaagtggtactctcc |
| | cagagactgtctgactcacgccaccccctccaccttggacacaggacgctgtggtttctgagccaggtacaatgactcctttcggtaagtgcagtggaagct |
| | gtacactgcccaggcaaagcgtccgggcagcgtaggcgggcgactcagatcccagccagtggacttagcccctgtttgctcctccgataactggggtga |
| | ccttggttaatattcaccagcagcctcccccgttgcccctctggatccactgcttaaatacggacgaggacagggccctgtctcctcagcttcaggcaccac |
| | cactgacctgggacagtgaatagatcttgagaacttcagggtgagtctatgggacccttgatgttttctttccccttcttttctatggttaagttcatgtcatagga |
| | aggggagaagtaacagggtacacatattgaccaaatcagggtaattttgcatttgtaattttaaaaaatgctttcttcttttaatatacttttttgtttatcttatttctaa |
| | tactttccctaatctctttctttcagggcaataatgatacaatgtatcatgcctctttgcaccattctaaagaataacagtgataatttctgggttaaggcaatagca |
| | atatttctgcatataaatatttctgcatataaattgtaactgatgtaagaggtttcatattgctaatagcagctacaatccagctaccattctgcttttattttctggttg |
| | ggataaggctggattattctgagtccaagctaggcccttttgctaatcttgttcatacctcttatcttcctcccacagctcctgggcaacctgctggtctctctgct |
| | ggcccatcactttggcaaagcactagtgccaccatggcctctaggctgactctgctgactctgctgctcctgctgctggctggggacagggccagctctaa |
| | ccccaatgctacttctagctcctctcaggaccctgagagcctgcaggacaggggggagggcaaggtggccaccactgtgatctctaagatgctgtttgtgg |
| | agcccatcctggaggtgagcagcctgcccaccaccaacagcaccaccaactctgccactaagatcactgccaacaccactgatgagcccaccacccag |
| | cccactactgagcccaccacccagcctactatccagcccacccagcccactacccagctgcccactgacagccctacccagcccaccactggcagcttct |
| | gccctgggcctgtgaccctgtgctctgacctggagagccattctactgaggctgtgctgggggatgccctggtggacttctctctgaagctgtaccatgcttt |
| | ctctgccatgaagaaggtggagactaacatggcttttagccccttcagcattgctagcctgctgacccaggtgctgctgggggctggggagaacactaaga |
| | ccaacctggaatctatcctgagctaccccaaggacttcacctgtgtgcaccaggccctgaagggcttcaccaccaagggggtgacctctgtgagccagat |
| | cttccacagccctgacctggccatcagggatacttttgtgaatgccagcagaaccctgtactcttctagccccagggtgctgagcaataattctgatgctaac |
| | ctggagctgatcaatacctgggtggctaagaataccaacaacaagattagcaggctgctggactctctgccttctgataccaggctggtgctgctgaatgcc |
| | atttacctgtctgccaagtggaagaccacttttgaccctaagaagactaggatggagcctttccacttcaagaactctgtcattaaggtgcccatgatgaacag |
| | caagaagtatcctgtggcccactttattgaccagactctgaaggccaaggtgggccagctgcagctgagccataacctgagcctggtgatcctggtgcccc |
| | agaacctgaagcataggctggaggacatggagcaggctctgagcccctctgtgttcaaggctattatggagaagctggagatgagcaagttccagcccac |
| | tctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctgacatctagaaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgtggtttaggtagtgtgagagggaggctcagaggcacacaggagtttctgggctcaccctgcccccttccaacccctcagttcc |
| | catcctccagcagctgtttgtgtgctgcctctgaagtccacactgaacaaacttcagcctactcatgtccctaaaatgggcaaacattgcaagcagcaaacag |
| | caaacacacagccctccctgcctgctgaccttggagctggggcagaggtcagagacctctctgggcccatgccacctccaacatccactcgaccccttgg |
| | aatttcggtggagaggagcagaggttgtcctggcgtggtttaggtagtgtgagagggtctagagctgaattcctgcagccagggggatcagcctctactgt |
| | gccttctagttgccagccatctgttgtttgcccctcccccttgccttccttgaccctggaaggtgccactcccactgtcctttcctaataaaatgaggaaattgca |
| | tcacattgtctgagtaggtgtcattctattctggggggtggggggggcaggacagcaagggggaggattgggaagacaatagcaggcatgctggggat |
| | gcagtgggctctatggcttctgaggcagaaagaaccagctggggctcgagatccactagggccgcaggaacccctagtgatggagttggccactccctc |
| | tctgcgcgctcgctcgctcactgaggccgggcgaccaaaggtcgcccgacgcccgggctttgcccgggcggcctcagtgagcgagcgagcgcgcag |
| 37 | ctgcgcgctcgctcgctcactgaggccgcccgggcaaagcccgggggcctcagtgagcgagcgagcgcgcagagagggagtggccaactccatca |
| | ctaggggttcctgcggcctagtccggggatcttgctaccagtggaacagccactaaggattctgcagtgagagcagagggccagctaagtggtactctcc |
| | cagagactgtctgactcacgccaccccctccaccttggacacaggacgctgtggtttctgagccaggtacaatgactcctttcggtaagtgcagtggaagct |
| | gtacactgcccaggcaaagcgtccgggcagcgtaggcgggcgactcagatcccagccagtggacttagcccctgtttgctcctccgataactggggtga |
| | ccttggttaatattcaccagcagcctcccccgttgcccctctggatccactgcttaaatacggacgaggacagggccctgtctcctcagcttcaggcaccac |
| | cactgacctgggacagtgaatagatcttgagaacttcagggtgagtctatgggacccttgatgttttctttccccttcttttctatggttaagttcatgtcatagga |
| | aggggagaagtaacagggtacacatattgaccaaatcagggtaattttgcatttgtaattttaaaaaatgctttcttcttttaatatacttttttgtttatcttatttctaa |
| | tactttccctaatctctttctttcagggcaataatgatacaatgtatcatgcctctttgcaccattctaaagaataacagtgataatttctgggttaaggcaatagca |
| | atatttctgcatataaatatttctgcatataaattgtaactgatgtaagaggtttcatattgctaatagcagctacaatccagctaccattctgcttttattttctggttg |
| | ggataaggctggattattctgagtccaagctaggcccttttgctaatcttgttcatacctcttatcttcctcccacagctcctgggcaacctgctggtctctctgct |
| | ggcccatcactttggcaaagcactagtgccaccatggcctctaggctgactctgctgactctgctgctcctgctgctggctggggacagggccagctctaa |
| | ccccaatgctacttctagctcctctcaggaccctgagagcctgcaggacaggggggagggcaaggtggccaccactgtgatctctaagatgctgtttgtgg |
| | agcccatcctggaggtgagcagcctgcccaccaccaacagcaccaccaactctgccactaagatcactgccaacaccactgatgagcccaccacccag |
| | cccactactgagcccaccacccagcctactatccagcccacccagcccactacccagctgcccactgacagccctacccagcccaccactggcagcttct |
| | gccctgggcctgtgaccctgtgctctgacctggagagccattctactgaggctgtgctgggggatgccctggtggacttctctctgaagctgtaccatgcttt |
| | ctctgccatgaagaaggtggagactaacatggcttttagccccttcagcattgctagcctgctgacccaggtgctgctgggggctggggagaacactaaga |
| | ccaacctggaatctatcctgagctaccccaaggacttcacctgtgtgcaccaggccctgaagggcttcaccaccaagggggtgacctctgtgagccagat |
| | cttccacagccctgacctggccatcagggatacttttgtgaatgccagcagaaccctgtactcttctagccccagggtgctgagcaataattctgatgctaac |
| | ctggagctgatcaatacctgggtggctaagaataccaacaacaagattagcaggctgctggactctctgccttctgataccaggctggtgctgctgaatgcc |
| | atttacctgtctgccaagtggaagaccacttttgaccctaagaagactaggatggagcctttccacttcaagaactctgtcattaaggtgcccatgatgaacag |
| | caagaagtatcctgtggcccactttattgaccagactctgaaggccaaggtgggccagctgcagctgagccataacctgagcctggtgatcctggtgcccc |
| | agaacctgaagcataggctggaggacatggagcaggctctgagcccctctgtgttcaaggctattatggagaagctggagatgagcaagttccagcccac |
| | tctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggtggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctgacatctagaaggctcagaggcacacaggagtttctgggctcaccctgcccccttccaacccctcagttcccatcctccagcagctgttt |
| | gtgtgctgcctctgaagtccacactgaacaaacttcagcctactcatgtccctaaaatgggcaaacattgcaagcagcaaacagcaaacacacagccctcc |
| | ctgcctgctgaccttggagctggggcagaggtcagagacctctctgggcccatgccacctccaacatccactcgaccccttggaatttcggtggagagga |
| | gcagaggttgtcctggcgggtttaggtagtgtgagagggaggctcagaggcacacaggagtttctgggctcaccctgcccccttccaacccctcagttccc |
| | atcctccagcagctgtttgtgtgctgcctctgaagtccacactgaacaaacttcagcctactcatgtccctaaaatgggcaaacattgcaagcagcaaacagc |
| | aaacacacagccctccctgcctgctgaccttggagctggggcagaggtcagagacctctctgggcccatgccacctccaacatccactcgaccccttgga |
| | atttcggtggagaggagcagaggttgtcctggcgtggtttaggtagtgtgagagggaggctcagaggcacacaggagtttctgggctcaccctgccccctt |
| | ccaacccctcagttcccatcctccagcagctgtttgtgtgctgcctctgaagtccacactgaacaaacttcagcctactcatgtccctaaaatgggcaaacatt |
| | gcaagcagcaaacagcaaacacacagccctccctgcctgctgaccttggagctggggcagaggtcagagacctctctgggcccatgccacctccaacat |
| | ccactcgaccccttggaatttcggtggagaggagcagaggttgtcctggcgtggtttaggtagtgtgagagggaggctcagaggcacacaggagtttctg |
| | ggctcaccctgcccccttccaacccctcagttcccatcctccagcagctgtttgtgtgctgcctctgaagtccacactgaacaaacttcagcctactcatgtcc |
| | ctaaaatgggcaaacattgcaagcagcaaacagcaaacacacagccctccctgcctgctgaccttggagctggggcagaggtcagagacctctctgggc |
| | ccatgccacctccaacatccactcgaccccttggaatttcggtggagaggagcagaggttgtcctggcgtggtttaggtagtgtgagagggtctagagctg |
| | aattcctgcagccagggggatcagcctctactgtgccttctagttgccagccatctgttgtttgcccctcccccttgccttccttgaccctggaaggtgccactc |
| | ccactgtcctttcctaataaaatgaggaaattgcatcacattgtctgagtaggtgtcattctattctggggggtggggggggcaggacagcaagggggagg |
| | attgggaagacaatagcaggcatgctggggatgcagtgggctctatggcttctgaggcagaaagaaccagctggggctcgagatccactagggccgca |
| | ggaacccctagtgatggagttggccactccctctctgcgcgctcgctcgctcactgaggccgggcgaccaaaggtcgcccgacgcccgggctttgcccg |
| | ggcggcctcagtgagcgagcgagcgcgcag |
| 38 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctctttctttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagacctgcagtcttcagagggccaccgcagctccagtgccacggcaggaggctgttcctg |
| | aatagcccctgtggtaagggccaggagagtccttccatcctccaaggccctgctaaaggacacagcagccaggaagtcccctgggcccctagctgaag |
| | gacagcctgctccctccgtctctaccaggaatggccttgtcctatggaaggcactgccccatcccaaactaatctaggaatcactgtctaaccactcactgtc |
| | atgaatgtgtacttaaaggatgaggttgagtcataccaaatagtgatttcgatagttcaaaatggtgaaattagcaattctacatgattcagtctaatcaatggat |
| | accgactgtttcccacacaagtctcctgttctcttaagcttactcactgacagcctttcactctccacaaatacattaaagatatggccatcaccaagcccccta |
| | ggatgacaccagacctgagagtctgaagacctggatcctctagagctgaattcctgcagccagggggatcagcctctactgtgccttctagttgccagccat |
| | ctgttgtttgcccctcccccttgccttccttgaccctggaaggtgccactcccactgtcctttcctaataaaatgaggaaattgcatcacattgtctgagtaggtg |
| | tcattctattctggggggtggggggggcaggacagcaagggggaggattgggaagacaatagcaggcatgctggggatgcagtgggctctatggcttc |
| | tgaggcagaaagaaccagctggggctcgagatccactagggccgcaggaacccctagtgatggagttggccactccctctctgcgcgctcgctcgctca |
| | ctgaggccgggcgaccaaaggtcgcccgacgcccgggctttgcccgggcggcctcagtgagcgagcgagcgcgcag |
| 39 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgtttttttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctctttctttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagacctgcagtcttcagagggccactgcagctccagtgccatggcaggaggctgttcctga |
| | atagcccctgtggtaagggccaggagagtccttccatcctccaaggccctgctaaaggacacagcagccaggaagtcccctgggcccctagctgaagg |
| | acagcctgctccctctgtctctaccaggaatggccttgtcctatggaaggcactgccccatcccaaactaatctaggaatcactgtctaaccactcactgtcat |
| | gaatgtgtacttaaaggatgaggttgagtcataccaaatagtgattttgatagttcaaaatggtgaaattagcaattctacatgattcagtctaatcaatggatact |
| | gactgtttcccacacaagtctcctgttctcttaagcttactcactgacagcctttcactctccacaaatacattaaagatatggccatcaccaagccccctaggat |
| | gacaccagacctgagagtctgaagacctggatcctctagagctgaattcctgcagccagggggatcagcctctactgtgccttctagttgccagccatctgt |
| | tgtttgcccctcccccttgccttccttgaccctggaaggtgccactcccactgtcctttcctaataaaatgaggaaattgcatcacattgtctgagtaggtgtcatt |
| | ctattctggggggtggggggggcaggacagcaagggggaggattgggaagacaatagcaggcatgctggggatgcagtgggctctatggcttctgag |
| | gcagaaagaaccagctggggctcgagatccactagggccgcaggaacccctagtgatggagttggccactccctctctgcgcgctcgctcgctcactga |
| | ggccgggcgaccaaaggtcgcccgacgcccgggctttgcccgggcggcctcagtgagcgagcgagcgcgcag |
| 40 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtagggggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctcttttttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagaaatcaacctctggattacaaaatttgtgaaagattgactggtattcttaactatgttgctcct |
| | tttacgctatgtggatacgctgctttaatgcctttgtatcatgctattgcttcccgtatggctttcattttctcctccttgtataaatcctggttagttcttgccacggcg |
| | gaactcatcgccgcctgccttgcccgctgctggacaggggctcggctgttgggcactgacaattccgtggtgttctagagctgaattcctgcagccagggg |
| | gatcagcctctactgtgccttctagttgccagccatctgttgtttgcccctcccccttgccttccttgaccctggaaggtgccactcccactgtcctttcctaataa |
| | aatgaggaaattgcatcacattgtctgagtaggtgtcattctattctggggggtggggggggcaggacagcaagggggaggattgggaagacaatagca |
| | ggcatgctggggatgcagtgggctctatggcttctgaggcagaaagaaccagctggggctcgagatccactagggccgcaggaacccctagtgatgga |
| | gttggccactccctctctgcgcgctcgctcgctcactgaggccgggcgaccaaaggtcgcccgacgcccgggctttgcccgggcggcctcagtgagcg |
| | agcgagcgcgcag |
| 41 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtagggggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctcttttttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagaaatcaacctctggattacaaaatttgtgaaagattgactggtattcttaactatgttgctcct |
| | tttatgctatgtggatatgctgctttaatgcctttgtatcatgctattgcttcctgtatggctttcattttctcctccttgtataaatcctggttagttcttgccatggtgga |
| | actcattgctgcctgccttgcctgctgctggacaggggcttggctgttgggcactgacaattctgtggtgttctagagctgaattcctgcagccagggggatc |
| | agcctctactgtgccttctagttgccagccatctgttgtttgcccctcccccttgccttccttgaccctggaaggtgccactcccactgtcctttcctaataaaatg |
| | aggaaattgcatcacattgtctgagtaggtgtcattctattctggggggtggggggggcaggacagcaagggggaggattgggaagacaatagcaggc |
| | atgctggggatgcagtgggctctatggcttctgaggcagaaagaaccagctggggctcgagatccactagggccgcaggaacccctagtgatggagttg |
| | gccactccctctctgcgcgctcgctcgctcactgaggccgggcgaccaaaggtcgcccgacgcccgggctttgcccgggcggcctcagtgagcgagc |
| | gagcgcgcag |
| 42 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctcttttttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagaaatcaacctctggattacaaaatttgtgaaagattgactggtattcttaactatgttgctcct |
| | tttacgctatgtggatacgctgctttaatgcctttgtatcatgctattgcttcccgtatggctttcattttctcctccttgtataaatcctggttgctgtctctttatgagg |
| | agttgtggcccgttgtcaggcaacgtggcgtggtgtgcactgtgtttgctgacgcaacccccactggttggggcattgccaccacctgtcagctcctttccg |
| | ggactttcgctttccccctccctattgccacggcggaactcatcgccgcctgccttgcccgctgctggacaggggctcggctgttgggcactgacaattccg |
| | tggtgttgtcggggaagctgacgtcctttccatggctgctcgcctgtgttgccacctggattctgcgcgggacgtccttctgctacgtcccttcggccctcaat |
| | ccagcggaccttccttcccgcggcctgctgccggctctgcggcctcttccgcgtcttcgccttcgccctcagacgagtcggatctccctttgggccgcctcc |
| | ccgctctagagctgaattcctgcagccagggggatcagcctctactgtgccttctagttgccagccatctgttgtttgcccctcccccttgccttccttgaccct |
| | ggaaggtgccactcccactgtcctttcctaataaaatgaggaaattgcatcacattgtctgagtaggtgtcattctattctggggggtggggggggcaggac |
| | agcaagggggaggattgggaagacaatagcaggcatgctggggatgcagtgggctctatggcttctgaggcagaaagaaccagctggggctcgagat |
| | ccactagggccgcaggaacccctagtgatggagttggccactccctctctgcgcgctcgctcgctcactgaggccgggcgaccaaaggtcgcccgacg |
| | cccgggctttgcccgggcggcctcagtgagcgagcgagcgcgcag |
| 43 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcttgagaacttcagggtgagtctatgggacccttgatgttttctttccccttcttttc |
| | tatggttaagttcatgtcataggaaggggagaagtaacagggtacacatattgaccaaatcagggtaattttgcatttgtaattttaaaaaatgctttcttcttttaa |
| | tatacttttttgtttatcttatttctaatactttccctaatctctttctttcagggcaataatgatacaatgtatcatgcctctttgcaccattctaaagaataacagtgata |
| | atttctgggttaaggcaatagcaatatttctgcatataaatatttctgcatataaattgtaactgatgtaagaggtttcatattgctaatagcagctacaatccagct |
| | accattctgcttttattttctggttgggataaggctggattattctgagtccaagctaggcccttttgctaatcttgttcatacctcttatcttcctcccacagctcctg |
| | ggcaacctgctggtctctctgctggcccatcactttggcaaagcactagtgccaccatggcctctaggctgactctgctgactctgctgctcctgctgctggct |
| | ggggacagggccagctctaaccccaatgctacttctagctcctctcaggaccctgagagcctgcaggacaggggggagggcaaggtggccaccactgt |
| | gatctctaagatgctgtttgtggagcccatcctggaggtgagcagcctgcccaccaccaacagcaccaccaactctgccactaagatcactgccaacacca |
| | ctgatgagcccaccacccagcccactactgagcccaccacccagcctactatccagcccacccagcccactacccagctgcccactgacagccctaccc |
| | agcccaccactggcagcttctgccctgggcctgtgaccctgtgctctgacctggagagccattctactgaggctgtgctgggggatgccctggtggacttct |
| | ctctgaagctgtaccatgctttctctgccatgaagaaggtggagactaacatggcttttagccccttcagcattgctagcctgctgacccaggtgctgctggg |
| | ggctggggagaacactaagaccaacctggaatctatcctgagctaccccaaggacttcacctgtgtgcaccaggccctgaagggcttcaccaccaaggg |
| | ggtgacctctgtgagccagatcttccacagccctgacctggccatcagggatacttttgtgaatgccagcagaaccctgtactcttctagccccagggtgct |
| | gagcaataattctgatgctaacctggagctgatcaatacctgggtggctaagaataccaacaacaagattagcaggctgctggactctctgccttctgatacc |
| | aggctggtgctgctgaatgccatttacctgtctgccaagtggaagaccacttttgaccctaagaagactaggatggagcctttccacttcaagaactctgtcat |
| | taaggtgcccatgatgaacagcaagaagtatcctgtggcccactttattgaccagactctgaaggccaaggtgggccagctgcagctgagccataacctg |
| | agcctggtgatcctggtgccccagaacctgaagcataggctggaggacatggagcaggctctgagcccctctgtgttcaaggctattatggagaagctgg |
| | agatgagcaagttccagcccactctgctgaccctgcccagaatcaaggtgaccacctctcaggacatgctgagcatcatggaaaagctggagttctttgact |
| | tttcttatgacctgaatctgtgtggcctgactgaggatcctgatctgcaggtgtctgccatgcagcaccagactgtgctggagctgactgagactggggtgga |
| | ggctgctgctgcttctgccatctctgtggccaggaccctgctggtgtttgaggtgcagcagcccttcctgtttgtgctgtgggaccagcagcacaagtttcctg |
| | tgttcatgggcagggtgtatgaccccagagcctgacatctagaaatcaacctctggattacaaaatttgtgaaagattgactggtattcttaactatgttgctcct |
| | tttatgctatgtggatatgctgctttaatgcctttgtatcatgctattgcttcctgtatggctttcattttctcctccttgtataaatcctggttgctgtctctttatgagga |
| | gttgtggcctgttgtcaggcaatgtggtgtggtgtgcactgtgtttgctgatgcaacccccactggttggggcattgccaccacctgtcagctcctttctggga |
| | cttttgctttccccctccctattgccatggtggaactcattgctgcctgccttgcctgctgctggacaggggcttggctgttgggcactgacaattctgtggtgtt |
| | gttggggaagctgatgtcctttccatggctgcttgcctgtgttgccacctggattctgtgtgggatgtccttctgctatgtccctttggccctcaatccagtggac |
| | cttccttcctgtggcctgctgctggctctgtggcctcttctgtgtctttgcctttgccctcagatgagttggatctccctttgggctgcctccctgctctagagctga |
| | attcctgcagccagggggatcagcctctactgtgccttctagttgccagccatctgttgtttgcccctcccccttgccttccttgaccctggaaggtgccactcc |
| | cactgtcctttcctaataaaatgaggaaattgcatcacattgtctgagtaggtgtcattctattctggggggtggggggggcaggacagcaagggggagga |
| | ttgggaagacaatagcaggcatgctggggatgcagtgggctctatggcttctgaggcagaaagaaccagctggggctcgagatccactagggccgcag |
| | gaacccctagtgatggagttggccactccctctctgcgcgctcgctcgctcactgaggccgggcgaccaaaggtcgcccgacgcccgggctttgcccgg |
| | gcggcctcagtgagcgagcgagcgcgcag |
| 44 | ctgcgcgctcgctcgctcactgaggccgcccgggcaaagcccgggcggcctcagtgagcgagcgagcgcgcagagagggagtggccaactccatca |
| | ctaggggttcctgcggcctagtactaggctcagaggcacacaggagtttctgggctcaccctgcccccttccaacccctcagttcccatcctccagcagctg |
| | tttgtgtgctgcctctgaagtccacactgaacaaacttcagcctactcatgtccctaaaatgggcaaacattgcaagcagcaaacagcaaacacacagccct |
| | ccctgcctgctgaccttggagctggggcagaggtcagagacctctctgggcccatgccacctccaacatccactcgaccccttggaatttcggtggagag |
| | gagcagaggttgtcctggcgtggtttaggtagtgtgagaggggtacccggggatcttgctaccagtggaacagccactaaggattctgcagtgagagcag |
| | agggccagctaagtggtactctcccagagactgtctgactcacgccaccccctccaccttggacacaggacgctgtggtttctgagccaggtacaatgact |
| | cctttcggtaagtgcagtggaagctgtacactgcccaggcaaagcgtccgggcagcgtaggcgggcgactcagatcccagccagtggacttagcccctg |
| | tttgctcctccgataactggggtgaccttggttaatattcaccagcagcctcccccgttgcccctctggatccactgcttaaatacggacgaggacagggccc |
| | tgtctcctcagcttcaggcaccaccactgacctgggacagtgaatagatcctgagaacttcagggtgagtctttgggacccttgttgttttctttccccttttttc |
| | tttggttaagttcttgtcataggaaggggagaagtaacagggtacacatattgaccaaatcagggtaattttgcatttgtaattttaaaaattgctttcttcttttaat |
| | atacttttttgtttatcttatttctaatactttccctaatctctttctttcagggcaatattgatacattgtatcttgcctctttgcaccattctaaagaataacagtgataat |
| | ttctgggttaaggcaatagcaatatttctgcatataaatatttctgcatataaattgtaactgttgtaagaggtttcatattgctaatagcagctacaatccagctac |
| | cattctgcttttattttctggttgggataaggctggattattctgagtccaagctaggcccttttgctaatcttgttcatacctcttatcttcctcccacagctcctggg |
| | caacctgctggtctctctgctggcccatcactttggcaaagcactagtgccaccatggcctctaggctgactctgctgactctgctgctcctgctgctggctgg |
| | ggacagggccagctctaaccccaatgctacttctagctcctctcaggaccctgagagcctgcaggacaggggggagggcaaggtggccaccactgtga |
| | tctctaagatgctgtttgtggagcccatcctggaggtgagcagcctgcccaccaccaacagcaccaccaactctgccactaagatcactgccaacaccact |
| | gatgagcccaccacccagcccactactgagcccaccacccagcctactatccagcccacccagcccactacccagctgcccactgacagccctaccca |
| | gcccaccactggcagcttctgccctgggcctgtgaccctgtgctctgacctggagagccattctactgaggctgtgctgggggatgccctggtggacttctc |
| | tctgaagctgtaccatgctttctctgccatgaagaaggtggagactaacatggcttttagccccttcagcattgctagcctgctgacccaggtgctgctgggg |
| | gctggggagaacactaagaccaacctggaatctatcctgagctaccccaaggacttcacctgtgtgcaccaggccctgaagggcttcaccaccaagggg |
| | gtgacctctgtgagccagatcttccacagccctgacctggccatcagggatacttttgtgaatgccagcagaaccctgtactcttctagccccagggtgctga |
| | gcaataattctgatgctaacctggagctgatcaatacctgggtggctaagaataccaacaacaagattagcaggctgctggactctctgccttctgataccag |
| | gctggtgctgctgaatgccatttacctgtctgccaagtggaagaccacttttgaccctaagaagactaggatggagcctttccacttcaagaactctgtcatta |
| | aggtgcccatgatgaacagcaagaagtatcctgtggcccactttattgaccagactctgaaggccaaggtgggccagctgcagctgagccataacctgag |
| | cctggtgatcctggtgccccagaacctgaagcataggctggaggacatggagcaggctctgagcccctctgtgttcaaggctattatggagaagctggag |
| | atgagcaagttccagcccactctgctgaccctgcccagaatcaaggtgaccacctctcaggacatgctgagcatcatggaaaagctggagttctttgactttt |
| | cttatgacctgaatctgtgtggcctgactgaggatcctgatctgcaggtgtctgccatgcagcaccagactgtgctggagctgactgagactggggtggag |
| | gctgctgctgcttctgccatctctgtggccaggaccctgctggtgtttgaggtgcagcagcccttcctgtttgtgctgtgggaccagcagcacaagtttcctgt |
| | gttcatgggcagggtgtatgaccccagagcctgacatctagagctgaattcctgcagccagggggatcagcctctactgtgccttctagttgccagccatct |
| | gttgtttgcccctcccccttgccttccttgaccctggaaggtgccactcccactgtcctttcctaataaaatgaggaaattgcatcacattgtctgagtaggtgtc |
| | attctattctggggggtggggggggcaggacagcaagggggaggattgggaagacaatagcaggcatgctggggatgcagtgggctctatggcttctg |
| | aggcagaaagaaccagctggggctcgagatccactagggccgcaggaacccctagtgatggagttggccactccctctctgcgcgctcgctcgctcact |
| | gaggccgggcgaccaaaggtcgcccgacgcccgggctttgcccgggcggcctcagtgagcgagcgagcgcgcag |
| 55 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtgccaccatggcctctaggctgactctgctgactctgctgct |
| | cctgctgctggctggggacagggccagctctaaccccaatgctacttctagctcctctcaggaccctgagagcctgcaggacaggggggagggcaaggt |
| | ggccaccactgtgatctctaagatgctgtttgtggagcccatcctggaggtgagcagcctgcccaccaccaacagcaccaccaactctgccactaagatca |
| | ctgccaacaccactgatgagcccaccacccagcccactactgagcccaccacccagcctactatccagcccacccagcccactacccagctgcccactg |
| | acagccctacccagcccaccactggcagcttctgccctgggcctgtgaccctgtgctctgacctggagagccattctactgaggctgtgctgggggatgcc |
| | ctggtggacttctctctgaagctgtaccatgctttctctgccatgaagaaggtggagactaacatggcttttagccccttcagcattgctagcctgctgaccca |
| | ggtgctgctgggggctggggagaacactaagaccaacctggaatctatcctgagctaccccaaggacttcacctgtgtgcaccaggccctgaagggcttc |
| | accaccaagggggtgacctctgtgagccagatcttccacagccctgacctggccatcagggatacttttgtgaatgccagcagaaccctgtactcttctagc |
| | cccagggtgctgagcaataattctgatgctaacctggagctgatcaatacctgggtggctaagaataccaacaacaagattagcaggctgctggactctctg |
| | ccttctgataccaggctggtgctgctgaatgccatttacctgtctgccaagtggaagaccacttttgaccctaagaagactaggatggagcctttccacttcaa |
| | gaactctgtcattaaggtgcccatgatgaacagcaagaagtatcctgtggcccactttattgaccagactctgaaggccaaggtgggccagctgcagctga |
| | gccataacctgagcctggtgatcctggtgccccagaacctgaagcataggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggtggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgacatctagactccataaagtaggaaacactacaccattccataaagtaggaaaca |
| | ctacaaccagttccataaagtaggaaacactacatcactccataaagtaggaaacactacactctagagctgaattcctgcagccagggggatcagcctcta |
| | ctgtgccttctagttgccagccatctgttgtttgcccctcccccttgccttccttgaccctggaaggtgccactcccactgtcctttcctaataaaatgaggaaatt |
| | gcatcacattgtctgagtaggtgtcattctattctggggggggggtggggcaggacagcaagggggaggattgggaagacaatagcaggcatgctggg |
| | gatgcagtgggctctatggcttctgaggcagaaagaaccagctggggctcgagatccactagggccgcaggaacccctagtgatggagttggccactcc |
| | ctctctgcgcgctcgctcgctcactgaggccgggcgaccaaaggtcgcccgacgcccgggctttgcccgggcggcctcagtgagcgagcgagcgcgc |
| | ag |
| 56 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtaggctcagaggcacacaggagtttctgggctcaccctgcccccttccaacccctcagttcccatcctc |
| | cagcagctgtttgtgtgctgcctctgaagtccacactgaacaaacttcagcctactcatgtccctaaaatgggcaaacattgcaagcagcaaacagcaaaca |
| | cacagccctccctgcctgctgaccttggagctggggcagaggtcagagacctctctgggcccatgccacctccaacatccactcgaccccttggaatttcg |
| | gtggagaggagcagaggttgtcctggcgtggtttaggtagtgtgagaggggtacccggggatcttgctaccagtggaacagccactaaggattctgcagt |
| | gagagcagagggccagctaagtggtactctcccagagactgtctgactcacgccaccccctccaccttggacacaggacgctgtggtttctgagccaggt |
| | acaatgactcctttcggtaagtgcagtggaagctgtacactgcccaggcaaagcgtccgggcagcgtaggcgggcgactcagatcccagccagtggact |
| | tagcccctgtttgctcctccgataactggggtgaccttggttaatattcaccagcagcctcccccgttgcccctctggatccactgcttaaatacggacgagga |
| | cagggccctgtctcctcagcttcaggcaccaccactgacctgggacagtgaatagatcctgagaacttcagggtgagtctatgggacccttgatgttttctttc |
| | cccttcttttctatggttaagttcatgtcataggaaggggagaagtaacagggtacacatattgaccaaatcagggtaattttgcatttgtaattttaaaaaatgct |
| | ttcttcttttaatatacttttttgtttatcttatttctaatactttccctaatctctttctttcagggcaataatgatacaatgtatcatgcctctttgcaccattctaaagaat |
| | aacagtgataatttctgggttaaggcaatagcaatatttctgcatataaatatttctgcatataaattgtaactgatgtaagaggtttcatattgctaatagcagcta |
| | caatccagctaccattctgcttttattttctggttgggataaggctggattattctgagtccaagctaggcccttttgctaatcttgttcatacctcttatcttcctccc |
| | acagctcctgggcaacctgctggtctctctgctggcccatcactttggcaaagcacgcgtgccaccatggcctctaggctgactctgctgactctgctgctcc |
| | tgctgctggctggggacagggccagctctaaccccaatgctacttctagctcctctcaggaccctgagagcctgcaggacaggggggagggcaaggtg |
| | gccaccactgtgatctctaagatgctgtttgtggagcccatcctggaggtgagcagcctgcccaccaccaacagcaccaccaactctgccactaagatcac |
| | tgccaacaccactgatgagcccaccacccagcccactactgagcccaccacccagcctactatccagcccacccagcccactacccagctgcccactga |
| | cagccctacccagcccaccactggcagcttctgccctgggcctgtgaccctgtgctcagacctggagagccactctactgaggctgtgctgggggatgcc |
| | ctggtggacttctctctgaagctgtatcatgctttctctgccatgaagaaggtggagactaacatggcttttagccccttcagcattgctagcctgctgacccag |
| | gtgctgctgggggctggggagaacactaagaccaacctggaatctatcctgagctaccccaaggacttcacctgtgtgcaccaggccctgaagggcttca |
| | ccaccaagggggtgacctctgtgagccagatcttccacagccctgacctggccatcagggatacttttgtgaatgccagcagaaccctgtactcttctagcc |
| | ccagggtgctgagcaataattctgatgctaacctggagctgatcaatacctgggtggctaagaataccaacaacaagattagcaggctgctggactctctgc |
| | cttctgataccaggctggtgctgctgaatgccatttacctgtctgccaagtggaagaccacttttgaccctaagaagactaggatggagcctttccacttcaag |
| | aactctgtcattaaggtgcccatgatgaacagcaagaagtatcctgtggcccactttattgaccagactctgaaggccaaggtgggccagctgcagctgag |
| | ccacaacctgagcctggtgatcctggtgccccagaacctgaagcacaggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgaagatctagagctgaattcctgcagccagggggatcagcctctactgtgccttcta |
| | gttgccagccatctgttgtttgcccctcccccttgccttccttgaccctggaaggtgccactcccactgtcctttcctaataaaatgaggaaattgcatcacattg |
| | tctgagtaggtgtcattctattctggggggggggtggggcaggacagcaagggggaggattgggaagacaatagcaggcatgctggggatgcagtgg |
| | gctctatggcttctgaggcagaaagaaccagctggggctcgagatccactagggccgcaggaacccctagtgatggagttggccactccctctctgcgcg |
| | ctcgctcgctcactgaggccgggcgaccaaaggtcgcccgacgcccgggctttgcccgggcggcctcagtgagcgagcgagcgcgcag |
| 57 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatctagtctgcactggagctgcctggtgaccagaagtttgga |
| | gtaggtttggtgctgggcaggggggggagtagggtggaaagcatggagtgaagaggtctagggagggggtctcctcacccccgccttcctgcccgcct |
| | tgatctcgggggtctctataggcttgcttccacctgggacttctgcctcctcctaccccagcccctcccgcctcaggcctgttgtgctcagccccccaggacc |
| | tcccctcccccacgcctctggcctcattgtttggttaaagcaggaccccctccccctcccaccacctcccctccgactgaacagatggacagagacccggg |
| | cccacggggagaggaagggccagccggtgccgggaaagggaagcggtttggggaaaacaaaacagagggaggagccagggagaaggtggcccc |
| | aggagggaggaggagggaattcgctaagagggactggggcctgagacggaatggggggggccccggggggggggggcccctgggctcccag |
| | ggtgggagctggctccgaggctggctggctccgcaggtccgctgacgtcgccgcccagatggcctctaggctgactctgctgactctgctgctcctgctg |
| | ctggctggggacagggccagctctaaccccaatgctacttctagctcctctcaggaccctgagagcctgcaggacaggggggagggcaaggtggccac |
| | cactgtgatctctaagatgctgtttgtggagcccatcctggaggtgagcagcctgcccaccaccaacagcaccaccaactctgccactaagatcactgcca |
| | acaccactgatgagcccaccacccagcccactactgagcccaccacccagcctactatccagcccacccagcccactacccagctgcccactgacagc |
| | cctacccagcccaccactggcagcttctgccctgggcctgtgaccctgtgctctgacctggagagccattctactgaggctgtgctgggggatgccctggt |
| | ggacttctctctgaagctgtaccatgctttctctgccatgaagaaggtggagactaacatggcttttagccccttcagcattgctagcctgctgacccaggtgc |
| | tgctgggggctggggagaacactaagaccaacctggaatctatcctgagctaccccaaggacttcacctgtgtgcaccaggccctgaagggcttcaccac |
| | caagggggtgacctctgtgagccagatcttccacagccctgacctggccatcagggatacttttgtgaatgccagcagaaccctgtactcttctagccccag |
| | ggtgctgagcaataattctgatgctaacctggagctgatcaatacctgggtggctaagaataccaacaacaagattagcaggctgctggactctctgccttct |
| | gataccaggctggtgctgctgaatgccatttacctgtctgccaagtggaagaccacttttgaccctaagaagactaggatggagcctttccacttcaagaact |
| | ctgtcattaaggtgcccatgatgaacagcaagaagtatcctgtggcccactttattgaccagactctgaaggccaaggtgggccagctgcagctgagccat |
| | aacctgagcctggtgatcctggtgccccagaacctgaagcataggctggaggacatggagcaggctctgagcccctctgtgttcaaggctattatggaga |
| | agctggagatgagcaagttccagcccactctgctgaccctgcccagaatcaaggtgaccacctctcaggacatgctgagcatcatggaaaagctggagtt |
| | ctttgacttttcttatgacctgaatctgtgtggcctgactgaggatcctgatctgcaggtgtctgccatgcagcaccagactgtgctggagctgactgagactg |
| | gggtggaggctgctgctgcttctgccatctctgtggccaggaccctgctggtgtttgaggtgcagcagcccttcctgtttgtgctgtgggaccagcagcaca |
| | agtttcctgtgttcatgggcagggtgtatgaccccagagcctgacatctagagctgaattcctgcagccagggggatcagcctctactgtgccttctagttgc |
| | cagccatctgttgtttgcccctcccccttgccttccttgaccctggaaggtgccactcccactgtcctttcctaataaaatgaggaaattgcatcacattgtctga |
| | gtaggtgtcattctattctggggggtggggggggcaggacagcaagggggaggattgggaagacaatagcaggcatgctggggatgcagtgggctct |
| | atggcttctgaggcagaaagaaccagctggggctcgagatccactagggccgcaggaacccctagtgatggagttggccactccctctctgcgcgctcg |
| | ctcgctcactgaggccgggcgaccaaaggtcgcccgacgcccgggctttgcccgggcggcctcagtgagcgagcgagcgcgcag |
| 60 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgaggaataacggaggtgagcaatttccaaaaagttcat |
| | tcctttaaaaactgcggagcacattttgtgtacatgctgtttttattgttactcatgaagacgcgggggaagctttggttgtgtaaagctgagaactgcacccaa |
| | gcttccccgttcaccccacctaccaggggatttgggctaaattcctggcctggcccacaaagaagaccaagcggtcagtcccattccgtcccatcctgattt |
| | acaggaactcacaccagcgatcaatcttccttaatttgtaactgggcagtgtcccgggccagccaatagctaagactgccccccccgcaccccaccctccc |
| | tgaccctgggggactctctactcagtctgcactggagctgcctggtgaccagaagtttggagtaggtttggtgctgggcaggggggggagtagggtgga |
| | aagcatggagtgaagaggtctagggagggggtctcctcacccccgccttcctgcccgccttgatctcgggggtctctataggcttgcttccacctgggactt |
| | ctgcctcctcctaccccagcccctcccgcctcaggcctgttgtgctcagccccccaggacctcccctcccccacgcctctggcctcattgtttggttaaagca |
| | ggaccccctccccctcccaccacctcccctccgactgaacagatggacagagacccgggcccacggggagaggaagggccagccggtgccgggaa |
| | agggaagcggtttggggaaaacaaaacagagggaggagccagggagaaggtggccccaggagggaggaggagggaattcgctaagagggactgg |
| | ggcctgagacggaatggggggggccccgggcggggtgggggcccctgggctcccagggtgggagctggctccgaggctggctggctccgcaggt |
| | ccgctgacgtcgccgcccagatggcctctaggctgactctgctgactctgctgctcctgctgctggctggggacagggccagctctaaccccaatgctact |
| | tctagctcctctcaggaccctgagagcctgcaggacaggggggagggcaaggtggccaccactgtgatctctaagatgctgtttgtggagcccatcctgg |
| | aggtgagcagcctgcccaccaccaacagcaccaccaactctgccactaagatcactgccaacaccactgatgagcccaccacccagcccactactgag |
| | cccaccacccagcctactatccagcccacccagcccactacccagctgcccactgacagccctacccagcccaccactggcagcttctgccctgggcct |
| | gtgaccctgtgctctgacctggagagccattctactgaggctgtgctgggggatgccctggtggacttctctctgaagctgtaccatgctttctctgccatgaa |
| | gaaggtggagactaacatggcttttagccccttcagcattgctagcctgctgacccaggtgctgctgggggctggggagaacactaagaccaacctggaa |
| | tctatcctgagctaccccaaggacttcacctgtgtgcaccaggccctgaagggcttcaccaccaagggggtgacctctgtgagccagatcttccacagccc |
| | tgacctggccatcagggatacttttgtgaatgccagcagaaccctgtactcttctagccccagggtgctgagcaataattctgatgctaacctggagctgatc |
| | aatacctgggtggctaagaataccaacaacaagattagcaggctgctggactctctgccttctgataccaggctggtgctgctgaatgccatttacctgtctg |
| | ccaagtggaagaccacttttgaccctaagaagactaggatggagcctttccacttcaagaactctgtcattaaggtgcccatgatgaacagcaagaagtatc |
| | ctgtggcccactttattgaccagactctgaaggccaaggtgggccagctgcagctgagccataacctgagcctggtgatcctggtgccccagaacctgaa |
| | gcataggctggaggacatggagcaggctctgagcccctctgtgttcaaggctattatggagaagctggagatgagcaagttccagcccactctgctgacc |
| | ctgcccagaatcaaggtgaccacctctcaggacatgctgagcatcatggaaaagctggagttctttgacttttcttatgacctgaatctgtgtggcctgactga |
| | ggatcctgatctgcaggtgtctgccatgcagcaccagactgtgctggagctgactgagactggggggaggctgctgctgcttctgccatctctgtggcca |
| | ggaccctgctggtgtttgaggtgcagcagcccttcctgtttgtgctgtgggaccagcagcacaagtttcctgtgttcatgggcagggtgtatgaccccagag |
| | cctgacatctagagctgaattcctgcagccagggggatcagcctctactgtgccttctagttgccagccatctgttgtttgcccctcccccttgccttccttgac |
| | cctggaaggtgccactcccactgtcctttcctaataaaatgaggaaattgcatcacattgtctgagtaggtgtcattctattctggggggtggggggggcag |
| | gacagcaagggggaggattgggaagacaatagcaggcatgctggggatgcagtgggctctatggcttctgaggcagaaagaaccagctggggctcga |
| | gatccactagggccgcaggaacccctagtgatggagttggccactccctctctgcgcgctcgctcgctcactgaggccgggcgaccaaaggtcgcccga |
| | cgcccgggctttgcccgggcggcctcagtgagcgagcgagcgcgcag |
| 61 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgaggaataacggaggtgagcaatttccaaaaagttcat |
| | tcctttaaaaactgcggagcacattttgtgtacatgctgtttttattgttactcatgaagacgcgggggaagctttggttgtgtaaagctgagaactgcacccaa |
| | gcttccccgttcaccccacctaccaggggatttgggctaaattcctggcctggcccacaaagaagaccaagcggtcagtcccattccgtcccatcctgattt |
| | acaggaactcacaccagcgatcaatcttccttaatttgtaactgggcagtgtcccgggccagccaatagctaagactgccccccccgcaccccaccctccc |
| | tgaccctgggggactctctactctgagaacttcagggtgagtctatgggacccttgatgttttctttccccttcttttctatggttaagttcatgtcataggaaggg |
| | gagaagtaacagggtacacatattgaccaaatcagggtaattttgcatttgtaattttaaaaaatgctttcttcttttaatatacttttttgtttatcttatttctaatacttt |
| | ccctaatctctttctttcagggcaataatgatacaatgtatcatgcctctttgcaccattctaaagaataacagtgataatttctgggttaaggcaatagcaatattt |
| | ctgcatataaatatttctgcatataaattgtaactgatgtaagaggtttcatattgctaatagcagctacaatccagctaccattctgcttttattttctggttgggata |
| | aggctggattattctgagtccaagctaggcccttttgctaatcttgttcatacctcttatcttcctcccacagctcctgggcaacctgctggtctctctgctggccc |
| | atcactttggcaaagcactagtgccaccatggcctctaggctgactctgctgactctgctgctcctgctgctggctggggacagggccagctctaaccccaa |
| | tgctacttctagctcctctcaggaccctgagagcctgcaggacaggggggagggcaaggtggccaccactgtgatctctaagatgctgtttgtggagccca |
| | tcctggaggtgagcagcctgcccaccaccaacagcaccaccaactctgccactaagatcactgccaacaccactgatgagcccaccacccagcccacta |
| | ctgagcccaccacccagcctactatccagcccacccagcccactacccagctgcccactgacagccctacccagcccaccactggcagcttctgccctg |
| | ggcctgtgaccctgtgctctgacctggagagccattctactgaggctgtgctgggggatgccctggtggacttctctctgaagctgtaccatgctttctctgcc |
| | atgaagaaggtggagactaacatggcttttagccccttcagcattgctagcctgctgacccaggtgctgctgggggctggggagaacactaagaccaacc |
| | tggaatctatcctgagctaccccaaggacttcacctgtgtgcaccaggccctgaagggcttcaccaccaagggggtgacctctgtgagccagatcttccac |
| | agccctgacctggccatcagggatacttttgtgaatgccagcagaaccctgtactcttctagccccagggtgctgagcaataattctgatgctaacctggagc |
| | tgatcaatacctgggtggctaagaataccaacaacaagattagcaggctgctggactctctgccttctgataccaggctggtgctgctgaatgccatttacct |
| | gtctgccaagtggaagaccacttttgaccctaagaagactaggatggagcctttccacttcaagaactctgtcattaaggtgcccatgatgaacagcaagaa |
| | gtatcctgtggcccactttattgaccagactctgaaggccaaggtgggccagctgcagctgagccataacctgagcctggtgatcctggtgccccagaacc |
| | tgaagcataggctggaggacatggagcaggctctgagcccctctgtgttcaaggctattatggagaagctggagatgagcaagttccagcccactctgctg |
| | accctgcccagaatcaaggtgaccacctctcaggacatgctgagcatcatggaaaagctggagttctttgacttttcttatgacctgaatctgtgtggcctgac |
| | tgaggatcctgatctgcaggtgtctgccatgcagcaccagactgtgctggagctgactgagactggggggaggctgctgctgcttctgccatctctgtggc |
| | caggaccctgctggtgtttgaggtgcagcagcccttcctgtttgtgctgtgggaccagcagcacaagtttcctgtgttcatgggcagggtgtatgaccccag |
| | agcctgacatctagagctgaattcctgcagccagggggatcagcctctactgtgccttctagttgccagccatctgttgtttgcccctcccccttgccttccttg |
| | accctggaaggtgccactcccactgtcctttcctaataaaatgaggaaattgcatcacattgtctgagtaggtgtcattctattctggggggtggggggggc |
| | aggacagcaagggggaggattgggaagacaatagcaggcatgctggggatgcagtgggctctatggcttctgaggcagaaagaaccagctggggctc |
| | gagatccactagggccgcaggaacccctagtgatggagttggccactccctctctgcgcgctcgctcgctcactgaggccgggcgaccaaaggtcgccc |
| | gacgcccgggctttgcccgggcggcctcagtgagcgagcgagcgcgcag |
| 62 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtagggacccgcagctcagctacagcacagatcagccacc |
| | atggcctctaggctgactctgctgactctgctgctcctgctgctggctggggacagggccagctctaaccccaatgctacttctagctcctctcaggaccctg |
| | agagcctgcaggacaggggggagggcaaggtggccaccactgtgatctctaagatgctgtttgtggagcccatcctggaggtgagcagcctgcccacc |
| | accaacagcaccaccaactctgccactaagatcactgccaacaccactgatgagcccaccacccagcccactactgagcccaccacccagcctactatc |
| | cagcccacccagcccactacccagctgcccactgacagccctacccagcccaccactggcagcttctgccctgggcctgtgaccctgtgctctgacctgg |
| | agagccattctactgaggctgtgctgggggatgccctggtggacttctctctgaagctgtaccatgctttctctgccatgaagaaggtggagactaacatggc |
| | ttttagccccttcagcattgctagcctgctgacccaggtgctgctgggggctggggagaacactaagaccaacctggaatctatcctgagctaccccaagg |
| | acttcacctgtgtgcaccaggccctgaagggcttcaccaccaagggggtgacctctgtgagccagatcttccacagccctgacctggccatcagggatact |
| | tttgtgaatgccagcagaaccctgtactcttctagccccagggtgctgagcaataattctgatgctaacctggagctgatcaatacctgggtggctaagaata |
| | ccaacaacaagattagcaggctgctggactctctgccttctgataccaggctggtgctgctgaatgccatttacctgtctgccaagtggaagaccacttttga |
| | ccctaagaagactaggatggagcctttccacttcaagaactctgtcattaaggtgcccatgatgaacagcaagaagtatcctgtggcccactttattgaccag |
| | actctgaaggccaaggtgggccagctgcagctgagccataacctgagcctggtgatcctggtgccccagaacctgaagcataggctggaggacatgga |
| | gcaggctctgagcccctctgtgttcaaggctattatggagaagctggagatgagcaagttccagcccactctgctgaccctgcccagaatcaaggtgacca |
| | cctctcaggacatgctgagcatcatggaaaagctggagttctttgacttttcttatgacctgaatctgtgtggcctgactgaggatcctgatctgcaggtgtctg |
| | ccatgcagcaccagactgtgctggagctgactgagactggggggaggctgctgctgcttctgccatctctgtggccaggaccctgctggtgtttgaggtg |
| | cagcagcccttcctgtttgtgctgtgggaccagcagcacaagtttcctgtgttcatgggcagggtgtatgaccccagagcctgacatctagagctgaattcct |
| | gcagccagggggatcagcctctactgtgccttctagttgccagccatctgttgtttgcccctcccccttgccttccttgaccctggaaggtgccactcccactg |
| | tcctttcctaataaaatgaggaaattgcatcacattgtctgagtaggtgtcattctattctggggggtggggggggcaggacagcaagggggaggattggg |
| | aagacaatagcaggcatgctggggatgcagtgggctctatggcttctgaggcagaaagaaccagctggggctcgagatccactagggccgcaggaacc |
| | cctagtgatggagttggccactccctctctgcgcgctcgctcgctcactgaggccgggcgaccaaaggtcgcccgacgcccgggctttgcccgggcggc |
| | ctcagtgagcgagcgagcgcgcag |
| 63 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtgccaccatggcctctaggctgactctgctgactctgctgct |
| | cctgctgctggctggggacagggccagctctaaccccaatgctacttctagctcctctcaggaccctgagagcctgcaggacaggggggagggcaaggt |
| | ggccaccactgtgatctctaagatgctgtttgtggagcccatcctggaggtgagcagcctgcccaccaccaacagcaccaccaactctgccactaagatca |
| | ctgccaacaccactgatgagcccaccacccagcccactactgagcccaccacccagcctactatccagcccacccagcccactacccagctgcccactg |
| | acagccctacccagcccaccactggcagcttctgccctgggcctgtgaccctgtgctctgacctggagagccattctactgaggctgtgctgggggatgcc |
| | ctggtggacttctctctgaagctgtaccatgctttctctgccatgaagaaggtggagactaacatggcttttagccccttcagcattgctagcctgctgaccca |
| | ggtgctgctgggggctggggagaacactaagaccaacctggaatctatcctgagctaccccaaggacttcacctgtgtgcaccaggccctgaagggcttc |
| | accaccaagggggtgacctctgtgagccagatcttccacagccctgacctggccatcagggatacttttgtgaatgccagcagaaccctgtactcttctagc |
| | cccagggtgctgagcaataattctgatgctaacctggagctgatcaatacctgggtggctaagaataccaacaacaagattagcaggctgctggactctctg |
| | ccttctgataccaggctggtgctgctgaatgccatttacctgtctgccaagtggaagaccacttttgaccctaagaagactaggatggagcctttccacttcaa |
| | gaactctgtcattaaggtgcccatgatgaacagcaagaagtatcctgtggcccactttattgaccagactctgaaggccaaggtgggccagctgcagctga |
| | gccataacctgagcctggtgatcctggtgccccagaacctgaagcataggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggtggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgacatctagagcttcctcttcactctgctctcaggagacctggctatgaggccctcg |
| | gggcagggatacaaagttagtgaggtctatgtccagagaagctgagatatggcatataataggcatctaataaatgcttaagaggtggaatctagagctgaa |
| | ttcctgcagccagggggatcagcctctactgtgccttctagttgccagccatctgttgtttgcccctcccccttgccttccttgaccctggaaggtgccactccc |
| | actgtcctttcctaataaaatgaggaaattgcatcacattgtctgagtaggtgtcattctattctggggggtggggggggcaggacagcaagggggaggat |
| | tgggaagacaatagcaggcatgctggggatgcagtgggctctatggcttctgaggcagaaagaaccagctggggctcgagatccactagggccgcagg |
| | aacccctagtgatggagttggccactccctctctgcgcgctcgctcgctcactgaggccgggcgaccaaaggtcgcccgacgcccgggctttgcccggg |
| | cggcctcagtgagcgagcgagcgcgcag |
| 64 | cctgcaggcagctgcgcgctcgctcgctcactgaggccgcccgggcaaagcccgggggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtagggacccgcagctcagctacagcacagatcagccacc |
| | atggcctctaggctgactctgctgactctgctgctcctgctgctggctggggacagggccagctctaaccccaatgctacttctagctcctctcaggaccctg |
| | agagcctgcaggacaggggggagggcaaggtggccaccactgtgatctctaagatgctgtttgtggagcccatcctggaggtgagcagcctgcccacc |
| | accaacagcaccaccaactctgccactaagatcactgccaacaccactgatgagcccaccacccagcccactactgagcccaccacccagcctactatc |
| | cagcccacccagcccactacccagctgcccactgacagccctacccagcccaccactggcagcttctgccctgggcctgtgaccctgtgctctgacctgg |
| | agagccattctactgaggctgtgctgggggatgccctggtggacttctctctgaagctgtaccatgctttctctgccatgaagaaggtggagactaacatggc |
| | ttttagccccttcagcattgctagcctgctgacccaggtgctgctgggggctggggagaacactaagaccaacctggaatctatcctgagctaccccaagg |
| | acttcacctgtgtgcaccaggccctgaagggcttcaccaccaagggggtgacctctgtgagccagatcttccacagccctgacctggccatcagggatact |
| | tttgtgaatgccagcagaaccctgtactcttctagccccagggtgctgagcaataattctgatgctaacctggagctgatcaatacctgggtggctaagaata |
| | ccaacaacaagattagcaggctgctggactctctgccttctgataccaggctggtgctgctgaatgccatttacctgtctgccaagtggaagaccacttttga |
| | ccctaagaagactaggatggagcctttccacttcaagaactctgtcattaaggtgcccatgatgaacagcaagaagtatcctgtggcccactttattgaccag |
| | actctgaaggccaaggtgggccagctgcagctgagccataacctgagcctggtgatcctggtgccccagaacctgaagcataggctggaggacatgga |
| | gcaggctctgagcccctctgtgttcaaggctattatggagaagctggagatgagcaagttccagcccactctgctgaccctgcccagaatcaaggtgacca |
| | cctctcaggacatgctgagcatcatggaaaagctggagttctttgacttttcttatgacctgaatctgtgtggcctgactgaggatcctgatctgcaggtgtctg |
| | ccatgcagcaccagactgtgctggagctgactgagactggggtggaggctgctgctgcttctgccatctctgtggccaggaccctgctggtgtttgaggtg |
| | cagcagcccttcctgtttgtgctgtgggaccagcagcacaagtttcctgtgttcatgggcagggtgtatgaccccagagcctgacatctagagcttcctcttc |
| | actctgctctcaggagacctggctatgaggccctcggggcagggatacaaagttagtgaggtctatgtccagagaagctgagatatggcatataataggca |
| | tctaataaatgcttaagaggtggaatctagagctgaattcctgcagccagggggatcagcctctactgtgccttctagttgccagccatctgttgtttgcccctc |
| | ccccttgccttccttgaccctggaaggtgccactcccactgtcctttcctaataaaatgaggaaattgcatcacattgtctgagtaggtgtcattctattctgggg |
| | ggtggggggggcaggacagcaagggggaggattgggaagacaatagcaggcatgctggggatgcagtgggctctatggcttctgaggcagaaaga |
| | accagctggggctcgagatccactagggccgcaggaacccctagtgatggagttggccactccctctctgcgcgctcgctcgctcactgaggccgggcg |
| | accaaaggtcgcccgacgcccgggctttgcccgggcggcctcagtgagcgagcgagcgcgcag |
| 65 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgggcacgtccccagctgggctgcctatttaacgcacca |
| | cactctcattctcccaaggtggggctccaggactaggctggggcagcagaaagtccccctctctacattgtccttggctcaggagccaacttagaaaaagc |
| | atttccaaattggctaagccagcggagcagagattttctgtgctgagaaatatcaggacatccagaggggtggaaggaggcttccagggcacacatgaga |
| | tgtggcaggggtaggctgtccgttttaaagcttaaagctttagacatgaactcacagggatttcagtcagggtcatctgccatgtggcccagcagggcccat |
| | cctgaggaaatgaccggtatagtcaggagctggctgaagagctgccctcactccacaccttccagcagcccaggtgccgccatcacggggctcccactg |
| | gcatctctgcagctgcacttcccccaatgctgaggagcagagctgatctagcaccctgtccattgccaaggcacagcaaacctctcttgttcccataggttac |
| | acaactgggataaatgacccgggatgaagaaaccaccggcatccaggaacttgtcttagaccagtttgtaggggaaatgacctgcagggactttccccag |
| | ggaccacatccagcttttcttccctcccaagagaccagcaaggctctgagaacttcagggtgagtctatgggacccttgatgttttctttccccttcttttctatg |
| | gttaagttcatgtcataggaaggggagaagtaacagggtacacatattgaccaaatcagggtaattttgcatttgtaattttaaaaaatgctttcttcttttaatata |
| | cttttttgtttatcttatttctaatactttccctaatctctttctttcagggcaataatgatacaatgtatcatgcctctttgcaccattctaaagaataacagtgataattt |
| | ctgggttaaggcaatagcaatatttctgcatataaatatttctgcatataaattgtaactgatgtaagaggtttcatattgctaatagcagctacaatccagctacc |
| | attctgcttttattttctggttgggataaggctggattattctgagtccaagctaggcccttttgctaatcttgttcatacctcttatcttcctcccacagctcctgggc |
| | aacctgctggtctctctgctggcccatcactttggcaaagcactagtgagggacccgcagctcagctacagcacagatcagccaccatggcctctaggctg |
| | actctgctgactctgctgctcctgctgctggctggggacagggccagctctaaccccaatgctacttctagctcctctcaggaccctgagagcctgcaggac |
| | aggggggagggcaaggtggccaccactgtgatctctaagatgctgtttgtggagcccatcctggaggtgagcagcctgcccaccaccaacagcaccac |
| | caactctgccactaagatcactgccaacaccactgatgagcccaccacccagcccactactgagcccaccacccagcctactatccagcccacccagcc |
| | cactacccagctgcccactgacagccctacccagcccaccactggcagcttctgccctgggcctgtgaccctgtgctctgacctggagagccattctactg |
| | aggctgtgctgggggatgccctggtggacttctctctgaagctgtaccatgctttctctgccatgaagaaggtggagactaacatggcttttagccccttcag |
| | cattgctagcctgctgacccaggtgctgctgggggctggggagaacactaagaccaacctggaatctatcctgagctaccccaaggacttcacctgtgtgc |
| | accaggccctgaagggcttcaccaccaagggggtgacctctgtgagccagatcttccacagccctgacctggccatcagggatacttttgtgaatgccag |
| | cagaaccctgtactcttctagccccagggtgctgagcaataattctgatgctaacctggagctgatcaatacctgggggctaagaataccaacaacaagatt |
| | agcaggctgctggactctctgccttctgataccaggctggtgctgctgaatgccatttacctgtctgccaagtggaagaccacttttgaccctaagaagacta |
| | ggatggagcctttccacttcaagaactctgtcattaaggtgcccatgatgaacagcaagaagtatcctgtggcccactttattgaccagactctgaaggccaa |
| | ggtgggccagctgcagctgagccataacctgagcctggtgatcctggtgccccagaacctgaagcataggctggaggacatggagcaggctctgagcc |
| | cctctgtgttcaaggctattatggagaagctggagatgagcaagttccagcccactctgctgaccctgcccagaatcaaggtgaccacctctcaggacatg |
| | ctgagcatcatggaaaagctggagttctttgacttttcttatgacctgaatctgtgtggcctgactgaggatcctgatctgcaggtgtctgccatgcagcacca |
| | gactgtgctggagctgactgagactggggtggaggctgctgctgcttctgccatctctgtggccaggaccctgctggtgtttgaggtgcagcagcccttcct |
| | gtttgtgctgtgggaccagcagcacaagtttcctgtgttcatgggcagggtgtatgaccccagagcctgacatctagagctgaattcctgcagccaggggg |
| | atcagcctctactgtgccttctagttgccagccatctgttgtttgcccctcccccttgccttccttgaccctggaaggtgccactcccactgtcctttcctaataaa |
| | atgaggaaattgcatcacattgtctgagtaggtgtcattctattctggggggtggggggggcaggacagcaagggggaggattgggaagacaatagcag |
| | gcatgctggggatgcagtgggctctatggcttctgaggcagaaagaaccagctggggctcgagatccactagggccgcaggaacccctagtgatggagt |
| | tggccactccctctctgcgcgctcgctcgctcactgaggccgggcgaccaaaggtcgcccgacgcccgggctttgcccgggcggcctcagtgagcgag |
| | cgagcgcgcag |
| 66 | cctgcaggcagctgcgcgctcgctcgctcactgaggccgcccgggcaaagcccgggggcctcagtgagcgagcgagcgcgcagagagggagtgg |
| | ccaactccatcactaggggttcctgcggcctagtactgcgcaaagtcaacacaagcctctccaccgtgtgtccatgtttatgtgtatgcgctgtgccccgtcat |
| | gccacctggacgcagggactccagtgacctctccttgcacaagcctctgctggtttgggaaagattggcatgacatcagccaagctctggccttgcctttttt |
| | ccctgcgcaaagtcaacacaagcctctccaccgtgtgtccatgtttatgtgtatgcgctgtgccccgtcatgccacctggacgcagggactccagtgacctc |
| | tccttgcacaagcctctgctggtttgggaaagattggcatgacatcagccaagctctggccttgccttttttccctagtactaggctcagaggcacacaggagt |
| | ttctgggctcaccctgcccccttccaacccctcagttcccatcctccagcagctgtttgtgtgctgcctctgaagtccacactgaacaaacttcagcctactcat |
| | gtccctaaaatgggcaaacattgcaagcagcaaacagcaaacacacagccctccctgcctgctgaccttggagctggggcagaggtcagagacctctct |
| | gggcccatgccacctccaacatccactcgaccccttggaatttcggtggagaggagcagaggttgtcctggcgtggtttaggtagtgtgagaggggtacc |
| | cggggatcttgctaccagtggaacagccactaaggattctgcagtgagagcagagggccagctaagtggtactctcccagagactgtctgactcacgcca |
| | ccccctccaccttggacacaggacgctgtggtttctgagccaggtacaatgactcctttcggtaagtgcagtggaagctgtacactgcccaggcaaagcgt |
| | ccgggcagcgtaggcgggcgactcagatcccagccagtggacttagcccctgtttgctcctccgataactggggtgaccttggttaatattcaccagcagc |
| | ctcccccgttgcccctctggatccactgcttaaatacggacgaggacagggccctgtctcctcagcttcaggcaccaccactgacctgggacagtgaatag |
| | atcttgagaacttcagggtgagtctatgggacccttgatgttttctttccccttcttttctatggttaagttcatgtcataggaaggggagaagtaacagggtaca |
| | catattgaccaaatcagggtaattttgcatttgtaattttaaaaaatgctttcttcttttaatatacttttttgtttatcttatttctaatactttccctaatctctttctttcagg |
| | gcaataatgatacaatgtatcatgcctctttgcaccattctaaagaataacagtgataatttctgggttaaggcaatagcaatatttctgcatataaatatttctgca |
| | tataaattgtaactgatgtaagaggtttcatattgctaatagcagctacaatccagctaccattctgcttttattttctggttgggataaggctggattattctgagtc |
| | caagctaggcccttttgctaatcttgttcatacctcttatcttcctcccacagctcctgggcaacctgctggtctctctgctggcccatcactttggcaaagcact |
| | agtgccaccatggcctctaggctgactctgctgactctgctgctcctgctgctggctggggacagggccagctctaaccccaatgctacttctagctcctctc |
| | aggaccctgagagcctgcaggacaggggggagggcaaggtggccaccactgtgatctctaagatgctgtttgtggagcccatcctggaggtgagcagc |
| | ctgcccaccaccaacagcaccaccaactctgccactaagatcactgccaacaccactgatgagcccaccacccagcccactactgagcccaccacccag |
| | cctactatccagcccacccagcccactacccagctgcccactgacagccctacccagcccaccactggcagcttctgccctgggcctgtgaccctgtgct |
| | ctgacctggagagccattctactgaggctgtgctgggggatgccctggtggacttctctctgaagctgtaccatgctttctctgccatgaagaaggtggaga |
| | ctaacatggcttttagccccttcagcattgctagcctgctgacccaggtgctgctgggggctggggagaacactaagaccaacctggaatctatcctgagct |
| | accccaaggacttcacctgtgtgcaccaggccctgaagggcttcaccaccaagggggtgacctctgtgagccagatcttccacagccctgacctggccat |
| | cagggatacttttgtgaatgccagcagaaccctgtactcttctagccccagggtgctgagcaataattctgatgctaacctggagctgatcaatacctgggtg |
| | gctaagaataccaacaacaagattagcaggctgctggactctctgccttctgataccaggctggtgctgctgaatgccatttacctgtctgccaagtggaaga |
| | ccacttttgaccctaagaagactaggatggagcctttccacttcaagaactctgtcattaaggtgcccatgatgaacagcaagaagtatcctgtggcccacttt |
| | attgaccagactctgaaggccaaggtgggccagctgcagctgagccataacctgagcctggtgatcctggtgccccagaacctgaagcataggctggag |
| | gacatggagcaggctctgagcccctctgtgttcaaggctattatggagaagctggagatgagcaagttccagcccactctgctgaccctgcccagaatcaa |
| | ggtgaccacctctcaggacatgctgagcatcatggaaaagctggagttctttgacttttcttatgacctgaatctgtgtggcctgactgaggatcctgatctgca |
| | ggtgtctgccatgcagcaccagactgtgctggagctgactgagactggggggaggctgctgctgcttctgccatctctgtggccaggaccctgctggtgtt |
| | tgaggtgcagcagcccttcctgtttgtgctgtgggaccagcagcacaagtttcctgtgttcatgggcagggtgtatgaccccagagcctgacatctagagct |
| | gaattcctgcagccagggggatcagcctctactgtgccttctagttgccagccatctgttgtttgcccctcccccttgccttccttgaccctggaaggtgccact |
| | cccactgtcctttcctaataaaatgaggaaattgcatcacattgtctgagtaggtgtcattctattctggggggtggggggggcaggacagcaagggggag |
| | gattgggaagacaatagcaggcatgctggggatgcagtgggctctatggcttctgaggcagaaagaaccagctggggctcgagatccactagggccgc |
| | aggaacccctagtgatggagttggccactccctctctgcgcgctcgctcgctcactgaggccgggcgaccaaaggtcgcccgacgcccgggctttgccc |
| | gggcggcctcagtgagcgagcgagcgcgcag |
| 67 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtgccaccatggcctctaggctgactctgctgactctgctgct |
| | cctgctgctggctggggacagggccagctctaaccccaatgctacttctagctcctctcaggaccctgagagcctgcaggacaggggggagggcaaggt |
| | ggccaccactgtgatctctaagatgctgtttgtggagcccatcctggaggtgagcagcctgcccaccaccaacagcaccaccaactctgccactaagatca |
| | ctgccaacaccactgatgagcccaccacccagcccactactgagcccaccacccagcctactatccagcccacccagcccactacccagctgcccactg |
| | acagccctacccagcccaccactggcagcttctgccctgggcctgtgaccctgtgctctgacctggagagccattctactgaggctgtgctgggggatgcc |
| | ctggtggacttctctctgaagctgtaccatgctttctctgccatgaagaaggtggagactaacatggcttttagccccttcagcattgctagcctgctgaccca |
| | ggtgctgctgggggctggggagaacactaagaccaacctggaatctatcctgagctaccccaaggacttcacctgtgtgcaccaggccctgaagggcttc |
| | accaccaagggggtgacctctgtgagccagatcttccacagccctgacctggccatcagggatacttttgtgaatgccagcagaaccctgtactcttctagc |
| | cccagggtgctgagcaataattctgatgctaacctggagctgatcaatacctgggtggctaagaataccaacaacaagattagcaggctgctggactctctg |
| | ccttctgataccaggctggtgctgctgaatgccatttacctgtctgccaagtggaagaccacttttgaccctaagaagactaggatggagcctttccacttcaa |
| | gaactctgtcattaaggtgcccatgatgaacagcaagaagtatcctgtggcccactttattgaccagactctgaaggccaaggtgggccagctgcagctga |
| | gccataacctgagcctggtgatcctggtgccccagaacctgaagcataggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggtggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgacatctagagcacctctgcagcaggtctagaattcctgcagccagggggatcag |
| | cctctactgtgccttctagttgccagccatctgttgtttgcccctcccccttgccttccttgaccctggaaggtgccactcccactgtcctttcctaataaaatgag |
| | gaaattgcatcacattgtctgagtaggtgtcattctattctggggggggggtggggcaggacagcaagggggaggattgggaagacaatagcaggcatg |
| | ctggggatgcagtgggctctatggcttctgaggcagaaagaaccagctggggctcgagatccactagggccgcaggaacccctagtgatggagttggcc |
| | actccctctctgcgcgctcgctcgctcactgaggccgggcgaccaaaggtcgcccgacgcccgggctttgcccgggcggcctcagtgagcgagcgag |
| | cgcgcag |
| 68 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtgccaccatggcctctaggctgactctgctgactctgctgct |
| | cctgctgctggctggggacagggccagctctaaccccaatgctacttctagctcctctcaggaccctgagagcctgcaggacaggggggagggcaaggt |
| | ggccaccactgtgatctctaagatgctgtttgtggagcccatcctggaggtgagcagcctgcccaccaccaacagcaccaccaactctgccactaagatca |
| | ctgccaacaccactgatgagcccaccacccagcccactactgagcccaccacccagcctactatccagcccacccagcccactacccagctgcccactg |
| | acagccctacccagcccaccactggcagcttctgccctgggcctgtgaccctgtgctctgacctggagagccattctactgaggctgtgctgggggatgcc |
| | ctggtggacttctctctgaagctgtaccatgctttctctgccatgaagaaggtggagactaacatggcttttagccccttcagcattgctagcctgctgaccca |
| | ggtgctgctgggggctggggagaacactaagaccaacctggaatctatcctgagctaccccaaggacttcacctgtgtgcaccaggccctgaagggcttc |
| | accaccaagggggtgacctctgtgagccagatcttccacagccctgacctggccatcagggatacttttgtgaatgccagcagaaccctgtactcttctagc |
| | cccagggtgctgagcaataattctgatgctaacctggagctgatcaatacctgggtggctaagaataccaacaacaagattagcaggctgctggactctctg |
| | ccttctgataccaggctggtgctgctgaatgccatttacctgtctgccaagtggaagaccacttttgaccctaagaagactaggatggagcctttccacttcaa |
| | gaactctgtcattaaggtgcccatgatgaacagcaagaagtatcctgtggcccactttattgaccagactctgaaggccaaggtgggccagctgcagctga |
| | gccataacctgagcctggtgatcctggtgccccagaacctgaagcataggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggtggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgacatctagagcacctctgcagcaggaagtctctgcagaagataacacctgcaga |
| | gtggtctagaattcctgcagccagggggatcagcctctactgtgccttctagttgccagccatctgttgtttgcccctcccccttgccttccttgaccctggaag |
| | gtgccactcccactgtcctttcctaataaaatgaggaaattgcatcacattgtctgagtaggtgtcattctattctggggggtggggggggcaggacagcaa |
| | gggggaggattgggaagacaatagcaggcatgctggggatgcagtgggctctatggcttctgaggcagaaagaaccagctggggctcgagatccacta |
| | gggccgcaggaacccctagtgatggagttggccactccctctctgcgcgctcgctcgctcactgaggccgggcgaccaaaggtcgcccgacgcccgg |
| | gctttgcccgggggcctcagtgagcgagcgagcgcgcag |
| 69 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcttgagaacttcagggtgagtctatgggacccttgatgttttc |
| | tttccccttcttttctatggttaagttcatgtcataggaaggggagaagtaacagggtacacatattgaccaaatcagggtaattttgcatttgtaattttaaaaaat |
| | gctttcttcttttaatatacttttttgtttatcttatttctaatactttccctaatctctttctttcagggcaataatgatacaatgtatcatgcctctttgcaccattctaaag |
| | aataacagtgataatttctgggttaaggcaatagcaatatttctgcatataaatatttctgcatataaattgtaactgatgtaagaggtttcatattgctaatagcag |
| | ctacaatccagctaccattctgcttttattttctggttgggataaggctggattattctgagtccaagctaggcccttttgctaatcttgttcatacctcttatcttcctc |
| | ccacagctcctgggcaacctgctggtctctctgctggcccatcactttggcaaagcactagtgccaccatggcctctaggctgactctgctgactctgctgct |
| | cctgctgctggctggggacagggccagctctaaccccaatgctacttctagctcctctcaggaccctgagagcctgcaggacaggggggagggcaaggt |
| | ggccaccactgtgatctctaagatgctgtttgtggagcccatcctggaggtgagcagcctgcccaccaccaacagcaccaccaactctgccactaagatca |
| | ctgccaacaccactgatgagcccaccacccagcccactactgagcccaccacccagcctactatccagcccacccagcccactacccagctgcccactg |
| | acagccctacccagcccaccactggcagcttctgccctgggcctgtgaccctgtgctctgacctggagagccattctactgaggctgtgctgggggatgcc |
| | ctggtggacttctctctgaagctgtaccatgctttctctgccatgaagaaggtggagactaacatggcttttagccccttcagcattgctagcctgctgaccca |
| | ggtgctgctgggggctggggagaacactaagaccaacctggaatctatcctgagctaccccaaggacttcacctgtgtgcaccaggccctgaagggcttc |
| | accaccaagggggtgacctctgtgagccagatcttccacagccctgacctggccatcagggatacttttgtgaatgccagcagaaccctgtactcttctagc |
| | cccagggtgctgagcaataattctgatgctaacctggagctgatcaatacctgggtggctaagaataccaacaacaagattagcaggctgctggactctctg |
| | ccttctgataccaggctggtgctgctgaatgccatttacctgtctgccaagtggaagaccacttttgaccctaagaagactaggatggagcctttccacttcaa |
| | gaactctgtcattaaggtgcccatgatgaacagcaagaagtatcctgtggcccactttattgaccagactctgaaggccaaggtgggccagctgcagctga |
| | gccataacctgagcctggtgatcctggtgccccagaacctgaagcataggctggaggacatggagcaggctctgagcccctctgtgttcaaggctattatg |
| | gagaagctggagatgagcaagttccagcccactctgctgaccctgcccagaatcaaggtgaccacctctcaggacatgctgagcatcatggaaaagctg |
| | gagttctttgacttttcttatgacctgaatctgtgtggcctgactgaggatcctgatctgcaggtgtctgccatgcagcaccagactgtgctggagctgactgag |
| | actggggtggaggctgctgctgcttctgccatctctgtggccaggaccctgctggtgtttgaggtgcagcagcccttcctgtttgtgctgtgggaccagcag |
| | cacaagtttcctgtgttcatgggcagggtgtatgaccccagagcctgacatctagagcacctctgcagcaggtctagaattcctgcagccagggggatcag |
| | cctctactgtgccttctagttgccagccatctgttgtttgcccctcccccttgccttccttgaccctggaaggtgccactcccactgtcctttcctaataaaatgag |
| | gaaattgcatcacattgtctgagtaggtgtcattctattctggggggtggggggggcaggacagcaagggggaggattgggaagacaatagcaggcatg |
| | ctggggatgcagtgggctctatggcttctgaggcagaaagaaccagctggggctcgagatccactagggccgcaggaacccctagtgatggagttggcc |
| | actccctctctgcgcgctcgctcgctcactgaggccgggcgaccaaaggtcgcccgacgcccgggctttgcccgggcggcctcagtgagcgagcgag |
| | cgcgcag |
| 110 | atggctagcagactgactctgctgaccctgctgctgctgctgctggctggggacagggccagcagcaaccctaatgctacctctagctctagccaggacc |
| | ctgagagcctgcaggataggggggagggcaaggtggccaccactgtgatcagcaagatgctgtttgtggagcctatcctggaggtgagcagcctgccc |
| | accaccaactctactaccaactctgctaccaagatcactgccaataccactgatgaacccaccactcagcccactactgagcccaccacccagcccaccat |
| | tcagcccactcagcccactacccagctgcctactgactctcccacccagcccaccactggcagcttctgccctgggcctgtgaccctgtgctctgacctgg |
| | agagccattctactgaggctgtgctgggggatgccctggtggatttcagcctgaagctgtaccatgccttctctgccatgaagaaggtggagaccaacatg |
| | gccttctctcccttcagcattgccagcctgctgacccaggtgctgctgggggctggggagaacaccaagaccaacctggagagcatcctgtcttaccccaa |
| | ggacttcacctgtgtgcatcaggccctgaagggcttcactactaagggggtgacctctgtgtctcagattttccacagccctgacctggctattagggacact |
| | tttgtgaatgcttctaggaccctgtacagcagctctcccagggtgctgagcaacaactctgatgccaacctggagctgattaacacttgggtggccaagaac |
| | actaacaataagatcagcaggctgctggacagcctgccctctgacaccaggctggtgctgctgaatgccatctacctgtctgccaagtggaagactactttt |
| | gatcctaagaagaccaggatggagcctttccacttcaagaactctgtgatcaaggtgcccatgatgaattctaagaagtaccctgtggctcacttcattgacc |
| | agaccctgaaggccaaggtgggccagctgcagctgagccacaacctgagcctggtgattctggtgccccagaacctgaagcacaggctggaggacatg |
| | gagcaggccctgagcccctctgtgttcaaggccatcatggagaagctggagatgagcaagttccagcctactctgctgactctgcccaggatcaaggtga |
| | ccactagccaggacatgctgagcattatggagaagctggagttctttgacttttcttatgatctgaacctgtgtggcctgactgaggaccctgacctgcaggtg |
| | tctgccatgcagcaccagactgtgctggagctgactgagactggggggaggctgctgctgcctctgccatctctgtggctaggaccctgctggtgtttgag |
| | gtgcagcagcccttcctgtttgtgctgtgggaccagcagcacaagttccctgtcttcatgggcagggtgtatgatcccagggcttga |
| 119 | atggcctctaggctgactctgctgactctgctgctcctgctgctggctggggacagggccagctctaaccccaatgctacttctagctcctctcaggaccctg |
| | agagcctgcaggacaggggggagggcaaggtggccaccactgtgatctctaagatgctgtttgtggagcccatcctggaggtgagcagcctgcccacc |
| | accaacagcaccaccaactctgccactaagatcactgccaacaccactgatgagcccaccacccagcccactactgagcccaccacccagcctactatc |
| | cagcccacccagcccactacccagctgcccactgacagccctacccagcccaccactggcagcttctgccctgggcctgtgaccctgtgctctgacctgg |
| | agagccattctactgaggctgtgctgggggatgccctggtggacttctctctgaagctgtaccatgctttctctgccatgaagaaggtggagactaacatggc |
| | ttttagccccttcagcattgctagcctgctgacccaggtgctgctgggggctggggagaacactaagaccaacctggaatctatcctgagctaccccaagg |
| | acttcacctgtgtgcaccaggccctgaagggcttcaccaccaagggggtgacctctgtgagccagatcttccacagccctgacctggccatcagggatact |
| | tttgtgaatgccagcagaaccctgtactcttctagccccagggtgctgagcaataattctgatgctaacctggagctgatcaatacctgggtggctaagaata |
| | ccaacaacaagattagcaggctgctggactctctgccttctgataccaggctggtgctgctgaatgccatttacctgtctgccaagtggaagaccacttttga |
| | ccctaagaagactaggatggagcctttccacttcaagaactctgtcattaaggtgcccatgatgaacagcaagaagtatcctgtggcccactttattgaccag |
| | actctgaaggccaaggtgggccagctgcagctgagccataacctgagcctggtgatcctggtgccccagaacctgaagcataggctggaggacatgga |
| | gcaggctctgagcccctctgtgttcaaggctattatggagaagctggagatgagcaagttccagcccactctgctgaccctgcccagaatcaaggtgacca |
| | cctctcaggacatgctgagcatcatggaaaagctggagttctttgacttttcttatgacctgaatctgtgtggcctgactgaggatcctgatctgcaggtgtctg |
| | ccatgcagcaccagactgtgctggagctgactgagactggggggaggctgctgctgcttctgccatctctgtggccaggaccctgctggtgtttgaggtg |
| | cagcagcccttcctgtttgtgctgtgggaccagcagcacaagtttcctgtgttcatgggcagggtgtatgaccccagagcctga |
| 142 | atggctagcagactgactctgctgaccctgctgctgctgctgctggctggggacagggccagcagcaaccctaatgctacctctagctctagccaggacc |
| | ctgagagcctgcaggataggggggagggcaaggtggccaccactgtgatcagcaagatgctgtttgtggagcctatcctggaggtgagcagcctgccc |
| | accaccaactctactaccaactctgctaccaagatcactgccaataccactgatgaacccaccactcagcccactactgagcccaccacccagcccaccat |
| | tcagcccactcagcccactacccagctgcctactgactctcccacccagcccaccactggcagcttctgccctgggcctgtgaccctgtgctctgacctgg |
| | agagccattctactgaggctgtgctgggggatgccctggtggatttcagcctgaagctgtaccatgccttctctgccatgaagaaggtggagaccaacatg |
| | gccttctctcccttcagcattgccagcctgctgacccaggtgctgctgggggctggggagaacaccaagaccaacctggagagcatcctgtcttaccccaa |
| | ggacttcacctgtgtgcatcaggccctgaagggcttcactactaagggggtgacctctgtgtctcagattttccacagccctgacctggctattagggacact |
| | tttgtgaatgcttctaggaccctgtacagcagctctcccagggtgctgagcaacaactctgatgccaacctggagctgattaacacttgggtggccaagaac |
| | actaacaataagatcagcaggctgctggacagcctgccctctgacaccaggctggtgctgctgaatgccatctacctgtctgccaagtggaagactactttt |
| | gatcctaagaagaccaggatggagcctttccacttcaagaactctgtgatcaaggtgcccatgatgaattctaagaagtaccctgtggctcacttcattgacc |
| | agaccctgaaggccaaggtgggccagctgcagctgagccacaacctgagcctggtgattctggtgccccagaacctgaagcacaggctggaggacatg |
| | gagcaggccctgagcccctctgtgttcaaggccatcatggagaagctggagatgagcaagttccagcctactctgctgactctgcccaggatcaaggtga |
| | ccactagccaggacatgctgagcattatggagaagctggagttctttgacttttcttatgatctgaacctgtgtggcctgactgaggaccctgacctgcaggtg |
| | tctgccatgcagcaccagactgtgctggagctgactgagactggggggaggctgctgctgcctctgccatctctgtggctaggaccctgctggtgtttgag |
| | gtgcagcagcccttcctgtttgtgctgtgggaccagcagcacaagttccctgtcttcatgggcagggtgtatgatcccagggcttga |
| 181 | NPNATSSSSQDPESLQDRGEGKVATTVISKMLFVEPILEVSSLPTTNSTTNSATKITANTTDEPTTQP |
| | TTEPTTQPTIQPTQPTTQLPTDSPTQPTTGSFCPGPVTLCSDLESHSTEAVLGDALVDFSLKLYHAFS |
| | AMKKVETNMAFSPFSIASLLTQVLLGAGENTKTNLESILSYPKDFTCVHQALKGFTTKGVTSVSQI |
| | FHSPDLAIRDTFVNASRTLYSSSPRVLSNNSDANLELINTWVAKNTNNKISRLLDSLPSDTRLVLLN |
| | AIYLSAKWKTTFDPKKTRMEPFHFKNSVIKVPMMNSKKYPVAHFIDQTLKAKVGQLQLSHNLSL |
| | VILVPQNLKHRLEDMEQALSPSVFKAIMEKLEMSKFQPTLLTLPRIKVTTSQDMLSIMEKLEFFDFS |
| | YDLNLCGLTEDPDLQVSAMQHQTVLELTETGVEAAAASAISVARTLLVFEVQQPFLFVLWDQQH |
| | KFPVFMGRVYDPRA |
| 192 | MASRLTLLTLLLLLLAGDRASSNPNATSSSSQDPESLQDRGEGKVATTVISKMLFVEPILEVSSLPT |
| | TNSTTNSATKITANTTDEPTTQPTTEPTTQPTIQPTQPTTQLPTDSPTQPTTGSFCPGPVTLCSDLESH |
| | STEAVLGDALVDFSLKLYHAFSAMKKVETNMAFSPFSIASLLTQVLLGAGENTKTNLESILSYPKD |
| | FTCVHQALKGFTTKGVTSVSQIFHSPDLAIRDTFVNASRTLYSSSPRVLSNNSDANLELINTWVAK |
| | NTNNKISRLLDSLPSDTRLVLLNAIYLSAKWKTTFDPKKTRMEPFHFKNSVIKVPMMNSKKYPVA |
| | HFIDQTLKAKVGQLQLSHNLSLVILVPQNLKHRLEDMEQALSPSVFKAIMEKLEMSKFQPTLLTLP |
| | RIKVTTSQDMLSIMEKLEFFDFSYDLNLCGLTEDPDLQVSAMQHQTVLELTETGVEAAAASAISV |
| | ARTLLVFEVQQPFLFVLWDQQHKFPVFMGRVYDPRA |
| 226 | MAADGYLPDWLEDNLSEGIREWWDLKPGAPKPKANQQKQDNGRGLVLPGYKYLGPFNGLDKG |
| | EPVNAADAAALEHDKAYDQQLQAGDNPYLRYNHADAEFQERLQEDTSFGGNLGRAVFQAKKR |
| | VLEPLGLVESPVKTAPGKKRPVEPSPQRSPDSSTGIGKKGQQPAKKRLNFGQTGDSESVPDPQPIGE |
| | PPAAPSGVGPNTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKLF |
| | NIQVKEVTQNEGTKTIANNLTSTIQVFTDSEYQLPYVLGSAHQGCLPPFPADVFMIPQYGYLTLNN |
| | GSQAVGRSSFYCLEYFPSQMLRTGNNFEFSYNFEDVPFHSSYAHSQSLDRLMNPLIDQYLYYLSRT |
| | QSTGGTAGTQQLLFSQAGPNNMSAQAKNWLPGPCYRQQRVSTTLSQNNNSNFAWTGATKYHLN |
| | GRDSLVNPGVAMATHKDDEERFFPSSGVLMFGKQGAGKDNVDYSSVMLTSEEEIKTTNPVATEQ |
| | YGVVADNLQQQNAAPIVGAVNSQGALPGMVWQNRDVYLQGPIWAKIPHTDGNFHPSPLMGGFG |
| | LKHPPPQILIKNTPVPADPPTTFNQAKLASFITQYSTGQVSVEIEWELQKENSKRWNPEIQYTSNYY |
| | KSTNVDFAVNTEGTYSEPRPIGTRYLTRNL |
| 229 | cctgcaggcagctgcgcgctcgctcgctcactgaggccgcccgggcaaagcccgggcggcctcagtgagcgagcgagcgcgcagagagggagtgg |
| | ccaactccatcactaggggttcctgcggcctagtactaggctcagaggcacacaggagtttctgggctcaccctgcccccttccaacccctcagttcccatc |
| | ctccagcagctgtttgtgtgctgcctctgaagtccacactgaacaaacttcagcctactcatgtccctaaaatgggcaaacattgcaagcagcaaacagcaa |
| | acacacagccctccctgcctgctgaccttggagctggggcagaggtcagagacctctctgggcccatgccacctccaacatccactcgaccccttggaatt |
| | tcggtggagaggagcagaggttgtcctggcgtggtttaggtagtgtgagaggggtacccggggatcttgctaccagtggaacagccactaaggattctgc |
| | agtgagagcagagggccagctaagtggtactctcccagagactgtctgactcacgccaccccctccaccttggacacaggacgctgtggtttctgagcca |
| | ggtacaatgactcctttcggtaagtgcagtggaagctgtacactgcccaggcaaagcgtccgggcagcgtaggcgggcgactcagatcccagccagtgg |
| | acttagcccctgtttgctcctccgataactggggtgaccttggttaatattcaccagcagcctcccccgttgcccctctggatccactgcttaaatacggacga |
| | ggacagggccctgtctcctcagcttcaggcaccaccactgacctgggacagtgaatagatcctgagaacttcagggtgagtctttgggacccttgttgttttc |
| | tttccccttcttttctttggttaagttcttgtcataggaaggggagaagtaacagggtacacatattgaccaaatcagggtaattttgcatttgtaattttaaaaattg |
| | ctttcttcttttaatatacttttttgtttatcttatttctaatactttccctaatctctttctttcagggcaatattgatacattgtatcttgcctctttgcaccattctaaagaat |
| | aacagtgataatttctgggttaaggcaatagcaatatttctgcatataaatatttctgcatataaattgtaactgttgtaagaggtttcatattgctaatagcagctac |
| | aatccagctaccattctgcttttattttctggttgggataaggctggattattctgagtccaagctaggcccttttgctaatcttgttcatacctcttatcttcctccca |
| | cagctcctgggcaacctgctggtctctctgctggcccatcactttggcaaagcactagtgccaccatggcctctaggctgactctgctgactctgctgctcct |
| | gctgctggctggggacagggccagctctaaccccaatgctacttctagctcctctcaggaccctgagagccttcaggacaggggggagggcaaggtgg |
| | ccaccactgtgatctctaagatgctgtttgtggagcccatcctggaggtgagcagcctgcccaccaccaacagcaccaccaactctgccactaagatcact |
| | gccaacaccactgatgagcccaccacccagcccactactgagcccaccacccagcctactatccagcccacccagcccactacccagctgcccactga |
| | cagccctacccagcccaccactggcagcttctgccctgggcctgtgaccctgtgctctgacctggagagccattctactgaggctgtgctgggggatgccc |
| | tggtggacttctctctgaagctgtaccatgctttctctgccatgaagaaggtggagactaacatggcttttagccccttcagcattgctagcctgctgacccag |
| | gtgctgctgggggctggggagaacactaagaccaacctggaatctatcctgagctaccccaaggacttcacctgtgtgcaccaggccctgaagggcttca |
| | ccaccaagggggtgacctctgtgagccagatcttccacagccctgacctggccatcagggatacttttgtgaatgccagcagaaccctgtactcttctagcc |
| | ccagggtgctgagcaataattctgatgctaacctggagctgatcaatacctgggtggctaagaataccaacaacaagattagcaggctgctggactctctgc |
| | cttctgataccaggctggtgctgctgaatgccatttacctgtctgccaagtggaagaccacttttgaccctaagaagactaggatggagcctttccacttcaag |
| | aactctgtcattaaggtgcccatgatgaacagcaagaagtatcctgtggcccactttattgaccagactctgaaggccaaggtgggccagcttcagctgagc |
| | cataacctgagcctggtgatcctggtgccccagaacctgaagcataggctggaggacatggagcaggctctgagcccctctgtgttcaaggctattatgga |
| | gaagctggagatgagcaagttccagcccactctgctgaccctgcccagaatcaaggtgaccacctctcaggacatgctgagcatcatggaaaagctgga |
| | gttctttgacttttcttatgacctgaatctgtgtggcctgactgaggatcctgatcttcaggtgtctgccatgcagcaccagactgtgctggagctgactgagact |
| | ggggtggaggctgctgctgcttctgccatctctgtggccaggaccctgctggtgtttgaggtgcagcagcccttcctgtttgtgctgtgggaccagcagcac |
| | aagtttcctgtgttcatgggcagggtgtatgaccccagagcctgaagatctagagctgaattccttcagccagggggatcagcctctactgtgccttctagttg |
| | ccagccatctgttgtttgcccctcccccttgccttccttgaccctggaaggtgccactcccactgtcctttcctaataaaatgaggaaattgcatcacattgtctg |
| | agtaggtgtcattctattctggggggtggggggggcaggacagcaagggggaggattgggaagacaatagcaggcatgctggggatgcagtgggctc |
| | tatggcttctgaggcagaaagaaccagctggggctcgagatccactagggccgcaggaacccctagtgatggagttggccactccctctctgcgcgctcg |
| | ctcgctcactgaggccgggcgaccaaaggtcgcccgacgcccgggctttgcccgggcggcctcagtgagcgagcgagcgcgcagctgcctgcaggg |
| | gcccatgggcagatgcaccacctgtctcagtgcaaagccctgcctaagtaggctggtcataagaccatgtgtctggctgtaactccaattgattgtcagcatc |
| | aataaaacttggccaacactgttatatactggtattgatagttacaactgaacatatttgtttaagcaattggaattaagaattcacatgcaatgatatcagggtcc |
| | ttctcctctggttagtgtattggggggaaattggacatctctcagctcagtaggctagttaggccaggatggatgacatccacagcccctgggcagagagatt |
| | atgatgtagctagtctgactcctgacaaagacttgcttcctggagcttctactactttctggtggatggctaagaaatatggttgtgttcttttaagtctgaagagc |
| | attatttttgccaacccctgaccaaacatccttgccaaggaaaaggcctaaaatatatttgcatttaaagatattacaaactacttggttttggaatgtttggccttt |
| | caggatcatagctatcaaaatattagctatttggggtatgagatgtctgcttggtcaaggacaagttcttaaagacatcatgttggggaataatggggaaaatg |
| | ggaaggcttatgctctgagtaagacatctgagttatcatctgtcaaacatttttgttagtcatagtctaatgggagcctgttttccctctttaatatacattcacatct |
| | gaatttatgctcttcattgacaatgccagcccagaacaacagctcttaccctttggttttcttcctaacctttaactccaatgtaaccattacctgccatttcagtaa |
| | aaccattattctccctacttacccacccaagttgtacaataaagagtgtttgctctcactcatatacaaagcaaattcatttgtttgtgatgtacagcttgctatgcc |
| | cacagatgtggtttgcctagtcctttgctctaggtcatttgactgggaacagatgggatgctcactttggtttttaatggttaatctagtcattgaaatgcatttcatc |
| | aaataatcttagaggataattgtttaaatgtctgtccagactagctttgtagagccaggtgccattacacatgtcaccttcttatttctcttaattgaatttttatcatct |
| | gagataggaataatagagggctttttcaagtgaagatattactatagtctaaagaccttagtgtaacatcctggcccctaaggaaaaacaagttctggttcata |
| | catataataactttgcatgttatctgccactgagatgtgtcctaatccaacagaaaggattgaatctctgtagctaggtgtacagggcaagagctgtacaggga |
| | acctttaaagatagcttcaggccaaagctgaggaaagtggatggagactggggaaaatgctaagacattttaaagattttctttaggtcaaaaatagaataag |
| | aaatagaccatttccctggacattttctgtaggttaatactgttgaactattggtaaatgcttatgctacaacttaatatgtctgctttgtgagtttagcattgtctcctt |
| | gtcattccagaaatgaaatggcaaatacatttaaatcagaactaaaaaggggaacagggtataaaggctcaatttagtcacatcatttccctttctcacccacc |
| | ccctttaaaccagatgtttgccaatgcattaacaatgcagatgtttcctgaaagaaagtttagtaactcaagcagacaccttattttcttttcaagcagaaaagac |
| | tatgagatggtggttgtggttgttctgggagggagaagatataaatgatacacattatttcaaatcatttcatgacctcactgcacacttatagttattgtacctgtt |
| | gtctttttgctgtcaagcctagctaagatcatttggaatgttcaagatcactcatacatgcatgtgcacacatacacatgcacatatgttcactccctatttcatcca |
| | catgaactaagattactgatgtgtacagattcaaagcacttttattcttttccaaaggcaagaagctgagctactttccagaatagttgtgaaagaccctgtcata |
| | cttctgcattgtttcctccacaccacctccatccagttccttatgaatggttactggttttcaaaaatatgagataaattgagtgtataaaagtcatttttagacaaaa |
| | tgaaacaggaaatgaaagaaaccagaatctctcctcatttgtggatgggccagctccaccatgtcatggttaatctgcagggaggaaatactagatttgattg |
| | cagatcagactgcagcaaacctgctgtgactaaggcatcaagagaaagcaagcaacagctggggcttcagtggtgaaaacattatatatctagctttgaaat |
| | atgaaatactgtttagcagtgtcacctagaaaagagtgtttcaaaatgctgatgcttcataagaacctttctcttcagagttgtttcttttatctttcaaattagccag |
| | ggtgggaaataaagtgatcacttggtgaagaaatctcacaaagaagaacatagagagttcactttcatctggagtaatgaacagattgaacaaactagaaat |
| | ggttagtctgttaaagaaaaggtgtaggtgagctgtttgcaagagccacaagggaaaggggaagacaacttctttgtggacttaagggtgaaagttgcaag |
| | caggcaagaccattctgacctccattaagaaagccctttccaaccaacaaccactgggttggttactcaggttgggcagcattgggagcaaatgttgattga |
| | acaaatgtttgtcagaattgttgacttaaagagctgttctgtcactggggacagcagcagctagatagccccattcagggagagggcatttgttcacctggcc |
| | agagatcagagcaggctaagggactgctgggatcctgtccagctttgagaccctacagagccatgttcacctagcaggtatcccttctgaggtcactctcat |
| | ttcttaccttattccagggctttcacctcagcttgccaggctggagccaagggccaaggcagcctcaccttgttggctatggtagcttcccaggagcccccta |
| | tggttcaggaacagctctgcctgccccatcctgtttgctacctcctaaagccaaaggcactggtgggccaggccagcttctaaagtcacacaaggttagaa |
| | ggttcctgacaggaagggcttgaggccaatggaaggaggtacttcagtttccctccagatgcccagtgatgggctcagagctccttgagaacttgggaaa |
| | ggaagcagggtctctgaagaaatacttcaggagtagaaagaggaagctagagggttaaatgcactacacaggaacagaaatgagtttttcttagagttagt |
| | atatgtcttagaggtgtagtaaactaaaacaagtcttgaattgcatacagccacttagggaagaaatgaaaacctttgaatattagtgaaaaaagggaaactg |
| | caacccctgtattactagatagctttcatcaacagctcaaaacagacagatttttataggtttactgtgtgcactttaatacaagggcagtggttcagaacttagt |
| | caggtcctgaaaaggatttaccaaatgttgagtgtgccctctagtgttcacacttcccagctttcttcctataaaggtggatcaaggcacttgcttacaactgga |
| | actgaaatcctccaagtggaactagacattgagatggagaaaatattcattgtccactgtaattatgcaaggaatatccagttgagataatggacttgcctctta |
| | tctaataatacccaggctcaatgggtcactgctttgtccactttgcccaaaattcaagcacagctaagttgatattttaggacaaaggcagcttactatccagcc |
| | agaggggagtagaatatggttaagagagagtggaaagaatgaatgagccctgctattcctcactgcctggatggctataagcacagcccttatggaggcct |
| | taggtcttgcttcataatattccagtttgaaaagggtttgaaaagacctcctagaaaaatcagtagtttttctcttttgagtaacatgtagcaaaaaaaatttcatca |
| | tgtaggtacagggaacaccctaataactattaatctcaaggagtcaagccagtgtgtttcctaatgtatctgctgtatccccatgaagcaaattttgccatcaga |
| | gaaactgactcatggggaaaaaatccaaggacctcaaatcaccaaaagaagccattcctcagatttgcctaagcttaagcttccctgtctctcattgtgtgttg |
| | ctttcaatgcagttacataaatggcttttttgtttatgcaccaaaaacactaattcatctgcaaagctataggtcaaagcaaccatagtatgcaccctgctagctg |
| | gcgcattaagcgcggcgggtgtggtggttacgcgcagcgtgaccgctacacttgccagcgccttagcgcccgctcctttcgctttcttcccttcctttctcgc |
| | cacgttcgccggctttccccgtcaagctctaaatcgggggctccctttagggttccgatttagtgctttacggcacctcgaccccaaaaaacttgatttgggtg |
| | atggttcacgtagtgggccatcgccctgatagacggtttttcgccctttgacgttggagtccacgttctttaatagtggactcttgttccaaactggaacaacact |
| | caactctatctcgggctattcttttgatttagacctgcaggcatgcaagcttggcactggccgtcgttttacaacgtcgtgactgggaaaaccctggcgttacc |
| | caacttaatcgccttgcagcacatccccctttcgccagctggcgtaatagcgaagaggcccgcaccgatcgcccttcccaacagttgcgcagcctgaatgg |
| | cgaatgcgatttattcaacaaagccgccgtcccgtcaagtcagcgtaatgctctgccagtgttacaaccaattaaccaattctgattagaaaaactcatcgag |
| | catcaaatgaaactgcaatttattcatatcaggattatcaataccatatttttgaaaaagccgtttctgtaatgaaggagaaaactcaccgaggcagttccatag |
| | gatggcaagatcctggtatcggtctgcgattccgactcgtccaacatcaatacaacctattaatttcccctcgtcaaaaataaggttatcaagtgagaaatcac |
| | catgagtgacgactgaatccggtgagaatggcaaaagcttatgcatttctttccagacttgttcaacaggccagccattacgctcgtcatcaaaatcactcgc |
| | atcaaccaaaccgttattcattcgtgattgcgcctgagcgagacgaaatacgcgatcgctgttaaaaggacaattacaaacaggaatcgaatgcaaccggc |
| | gcaggaacactgccagcgcatcaacaatattttcacctgaatcaggatattcttctaatacctggaatgctgttttcccggggatcgcagtggtgagtaaccat |
| | gcatcatcaggagtacggataaaatgcttgatggtcggaagaggcataaattccgtcagccagtttagtctgaccatctcatctgtaacatcattggcaacgc |
| | tacctttgccatgtttcagaaacaactctggcgcatcgggcttcccatacaatcgatagattgtcgcacctgattgcccgacattatcgcgagcccatttatacc |
| | catataaatcagcatccatgttggaatttaatcgcggcttcgagcaagacgtttcccgttgaatatggctcataacaccccttgtattactgtttatgtaagcaga |
| | cagttttattgttcatgatgatatatttttatcttgtgcaatgtaacatcagagattttgagacacaacgtggctttgttgaataaatcgaacttttgctgagttgaagg |
| | atcagatcacgcatcttcccgacaacgcagaccgttccgtggcaaagcaaaagttcaaaatcaccaactggtccacctacaacaaagctctcatcaaccgt |
| | ggctccctcactttctggctggatgatggggcgattcaggcctggtatgagtcagcaacaccttcttcacgaggcagacctctcgacggagttccactgagc |
| | gtcagaccccgtagaaaagatcaaaggatcttcttgagatcctttttttctgcgcgtaatctgctgcttgcaaacaaaaaaaccaccgctaccagcggtggttt |
| | gtttgccggatcaagagctaccaactctttttccgaaggtaactggcttcagcagagcgcagataccaaatactgttcttctagtgtagccgtagttaggccac |
| | cacttcaagaactctgtagcaccgcctacatacctcgctctgctaatcctgttaccagtggctgctgccagtggcgataagtcgtgtcttaccgggttggactc |
| | aagacgatagttaccggataaggcgcagcggtcgggctgaacggggggttcgtgcacacagcccagcttggagcgaacgacctacaccgaactgaga |
| | tacctacagcgtgagctatgagaaagcgccacgcttcccgaagggagaaaggcggacaggtatccggtaagcggcagggtcggaacaggagagcgc |
| | acgagggagcttccagggggaaacgcctggtatctttatagtcctgtcgggtttcgccacctctgacttgagcgtcgatttttgtgatgctcgtcaggggggc |
| | ggagcctatggaaaaacgccagcaacgcggcctttttacggttcctggccttttgctggccttttgctcacatgt |
| 232 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggcctga |
| 233 | aaccctaatgctacctctagctctagccaggaccctgagagcctgcaggataggggggagggcaaggtggccaccactgtgatcagcaagatgctgtttg |
| | tggagcctatcctggaggtgagcagcctgcccaccaccaactctactaccaactctgctaccaagatcactgccaataccactgatgaacccaccactcag |
| | cccactactgagcccaccacccagcccaccattcagcccactcagcccactacccagctgcctactgactctcccacccagcccaccactggcagcttttg |
| | tcctggccctgtgaccctgtgctctgacctggagagccattctactgaggctgtgctgggggatgccctggtggacttcagcctgaagctgtaccatgccttc |
| | tctgccatgaagaaggtggagaccaacatggccttcagccccttctctattgcctctctgctgacccaggtgctgctgggggctggggagaatactaagac |
| | caacctggagagcattctgagctatcccaaggacttcacctgtgtgcaccaggccctgaaggggttcactactaaaggggtgacctctgtgtctcagatctt |
| | ccacagccctgacctggccatcagggacacttttgtgaatgccagcaggaccctgtacagcagcagccccagagtgctgagcaataattctgatgctaatc |
| | tggagctgattaacacttgggtggccaagaacaccaacaacaagatctctaggctgctggattctctgccctctgacaccaggctggtgctgctgaatgcca |
| | tctatctgtctgccaagtggaagaccacctttgatcccaagaagaccaggatggagcccttccatttcaagaactctgtgattaaagtgcccatgatgaactct |
| | aagaagtatcctgtggcccacttcattgatcagactctgaaggccaaggtggggcagctgcagctgagccacaacctgagcctggtgatcctggtgcccc |
| | agaatctgaagcacaggctggaggacatggagcaggccctgagcccctctgtgttcaaggccatcatggagaagctggagatgagcaagttccagccc |
| | accctgctgactctgcccagaattaaggtgaccaccagccaggacatgctgagcatcatggagaagctggagttctttgacttctcttatgacctgaacctgt |
| | gtggcctgactgaggatcctgacctgcaggtgtctgccatgcagcaccagactgtgctggagctgactgagactggggggaggctgctgctgcctctgc |
| | catttctgtggccaggaccctgctggtgtttgaggtgcagcagcccttcctgtttgtgctgtgggaccagcagcacaagtttcctgtgtttatgggcagggtgt |
| | atgaccctagggcctga |
| 234 | aaccctaatgccacctccagcagcagccaggaccctgagagcctgcaggataggggggagggcaaggtggctactactgtgatcagcaagatgctgttt |
| | gtggagcctatcctggaggtgagcagcctgcccaccactaactctactactaattctgccactaagatcactgccaataccactgatgagccaaccactcag |
| | cccaccactgagcccaccacccagcccaccattcagcccactcagcctaccacccagctgcctactgacagccccactcagcccactactggctctttctg |
| | tcctgggcctgtgactctgtgctctgacctggaatcccacagcactgaggctgtgctgggggatgccctggtggatttcagcctgaagctgtaccatgctttc |
| | tctgccatgaagaaggtggagaccaacatggccttcagccctttctctattgccagcctgctgactcaggtgctgctgggggctggggagaataccaagac |
| | caacctggagtctatcctgagctatcccaaggacttcacttgtgtgcaccaggccctgaaggggttcaccaccaagggggtgacctctgtgtctcagatcttt |
| | cacagccctgacctggccatcagggatacctttgtgaatgccagcaggaccctgtacagcagcagccccagggtgctgagcaacaactctgatgccaac |
| | ctggagctgatcaacacctgggtggccaagaacaccaacaacaagatcagcaggctgctggacagcctgccctctgataccaggctggtgctgctgaat |
| | gccatctacctgtctgctaagtggaagaccacttttgatcctaagaagaccaggatggagccctttcacttcaagaactctgtgatcaaggtgcccatgatga |
| | actctaagaaataccctgtggcccacttcattgatcagaccctgaaggccaaggtgggccagctgcagctgagccacaacctgagcttggtgatcctggtg |
| | cctcagaacctgaagcacaggctggaggacatggagcaggccctgagcccctctgtgttcaaggccattatggagaagctggagatgagcaagttccag |
| | cctaccctgctgaccctgcccaggatcaaggtgactactagccaggatatgctgagcatcatggagaagctggagttttttgatttcagctatgacctgaacc |
| | tgtgtggcctgactgaggaccctgacctgcaggtgtctgccatgcagcatcagactgtgctggagctgactgagactggggggaggctgctgctgcctct |
| | gccatctctgtggccaggaccctgctggtgtttgaggtgcagcagcctttcctgtttgtgctgtgggatcagcagcacaagtttcctgtgttcatgggcagggt |
| | gtatgaccccagggcttga |
| 235 | aaccctaatgctacctctagctctagccaggaccctgagagcctgcaggataggggggagggcaaggtggccaccactgtgatcagcaagatgctgtttg |
| | tggagcctatcctggaggtgagcagcctgcccaccaccaactctactaccaactctgctaccaagatcactgccaataccactgatgaacccaccactcag |
| | cccactactgagcccaccacccagcccaccattcagcccactcagcccactacccagctgcctactgactctcccacccagcccaccactgggagcttct |
| | gtccaggccctgtgactctgtgttctgacctggagagccacagcactgaagctgtgctgggggatgctctggtggacttcagcctgaagctgtaccatgcct |
| | tctctgccatgaagaaggtggaaaccaacatggcctttagccccttcagcattgctagcctgctgactcaggtgctgctgggggctggggagaacactaag |
| | actaacctggagtctatcctgtcttatcccaaggacttcacctgtgtgcatcaggccctgaaggggttcaccaccaagggggtcacctctgtgagccagatct |
| | ttcacagccctgatctggccatcagggacacctttgtgaatgcctctaggactctgtacagcagcagccccagggtgctgagcaacaactctgatgccaac |
| | ctggagctgatcaacacctgggtggccaagaacactaacaacaagatcagcaggctgctggacagcctgccctctgacactaggctggtgctgctgaatg |
| | ccatctatctgtctgccaagtggaagaccacctttgaccccaagaagactaggatggagccctttcattttaagaactctgtgatcaaggtgcccatgatgaa |
| | cagcaagaaataccctgtggcccatttcattgaccagactctgaaggctaaggtgggccagctgcagctgagccacaacctgagcctggtgatcctggtg |
| | ccacagaatctgaagcacaggctggaggacatggagcaggctctgtctccctctgtgttcaaggccatcatggagaagctggagatgtctaagttccagcc |
| | caccctgctgaccctgcccaggatcaaggtgaccacttctcaggacatgctgtctatcatggagaagctggagttttttgacttttcttatgacctgaacctgtg |
| | tggcctgactgaggaccctgatctgcaggtgtctgccatgcagcaccagactgtgctggagctgactgagactggggggaggctgctgctgcttctgcca |
| | tttctgtggctaggactctgctggtgtttgaggtgcagcagcccttcctgtttgtgctgtgggaccagcagcacaagttccctgtgttcatgggcagggtgtat |
| | gatcccagggcttga |
| 236 | aaccctaatgctacctctagctctagccaggaccctgagagcctgcaggataggggggagggcaaggtggccaccactgtgatcagcaagatgctgtttg |
| | tggagcctatcctggaggtgagcagcctgcccaccaccaactctactaccaactctgctaccaagatcactgccaataccactgatgaacccaccactcag |
| | cccactactgagcccaccacccagcccaccattcagcccactcagcccactacccagctgcctactgactctcccacccagcccaccactggcagcttct |
| | gccctgggcctgtgaccctgtgctctgacctggagagccattctactgaggctgtgctgggggatgccctggtggatttcagcctgaagctgtaccatgcct |
| | tctctgccatgaagaaggtggagaccaacatggccttctctcccttcagcattgccagcctgctgacccaggtgctgctgggggctggggagaacaccaa |
| | gaccaacctggagagcatcctgtcttaccccaaggacttcacctgtgtgcatcaggccctgaagggcttcactactaagggggtgacctctgtgtctcagat |
| | tttccacagccctgacctggctattagggacacttttgtgaatgcttctaggaccctgtacagcagctctcccagggtgctgagcaacaactctgatgccaac |
| | ctggagctgattaacacttgggggccaagaacactaacaataagatcagcaggctgctggacagcctgccctctgacaccaggctggtgctgctgaatg |
| | ccatctacctgtctgccaagtggaagactacttttgatcctaagaagaccaggatggagcctttccacttcaagaactctgtgatcaaggtgcccatgatgaat |
| | tctaagaagtaccctgtggctcacttcattgaccagaccctgaaggccaaggtgggccagctgcagctgagccacaacctgagcctggtgattctggtgcc |
| | ccagaacctgaagcacaggctggaggacatggagcaggccctgagcccctctgtgttcaaggccatcatggagaagctggagatgagcaagttccagc |
| | ctactctgctgactctgcccaggatcaaggtgaccactagccaggacatgctgagcattatggagaagctggagttctttgacttttcttatgatctgaacctgt |
| | gtggcctgactgaggaccctgacctgcaggtgtctgccatgcagcaccagactgtgctggagctgactgagactggggtggaggctgctgctgcctctgc |
| | catctctgtggctaggaccctgctggtgtttgaggtgcagcagcccttcctgtttgtgctgtgggaccagcagcacaagttccctgtcttcatgggcagggtg |
| | tatgatcccagggcttga |
| 237 | aaccccaatgctactagcagcagcagccaggatcctgagagcctgcaggacagaggggaagggaaggtggccaccactgtgatcagcaagatgctgtt |
| | tgtggagcctatcctggaggtgagctctctgcccaccactaacagcaccaccaattctgctactaagatcactgccaacaccactgatgagcccaccaccc |
| | agcctaccactgagccaactacccagcccaccatccagcccacccagcccaccactcagctgcctactgattctcctactcagcctaccactgggagcttc |
| | tgccctgggcctgtgaccctgtgctctgacctggagtctcattctactgaggctgtgctgggggatgccctggtggacttcagcctgaagctgtaccatgcttt |
| | ctctgccatgaagaaggtggagactaacatggccttctctcctttctctattgccagcctgctgactcaggtgctgctgggggctggggagaataccaagac |
| | taacctggagagcatcctgtcttatcctaaggacttcacttgtgtgcatcaggccctgaagggcttcaccaccaagggggtgacttctgtgagccagattttcc |
| | acagccctgacctggccatcagagacacctttgtgaatgccagcaggaccctgtattctagctctcccagggtgctgagcaacaactctgatgccaatctgg |
| | aactgatcaacacttgggtggccaagaacaccaacaacaagatcagcaggctgctggatagcctgccctctgacaccaggctggtgctgctgaatgccat |
| | ctacctgtctgccaagtggaagaccacctttgaccccaagaagactaggatggagcccttccacttcaagaactctgtgatcaaggtgcccatgatgaatag |
| | caagaagtaccctgtggcccacttcattgaccagaccctgaaggccaaggtggggcagctgcagctgagccacaacctgagcctggtgatcctggtgcc |
| | ccagaacctgaagcacagactggaggacatggagcaggccctgagcccatctgtgttcaaggctattatggagaagctggagatgtctaagtttcagccta |
| | ccctgctgaccctgcccaggatcaaggtgactaccagccaggacatgctgagcatcatggagaagctggaattctttgacttcagctatgatctgaatctgt |
| | gtggcctgactgaagaccctgatttgcaggtgtctgctatgcagcaccagactgtgctggaactgactgagactggggtggaggctgctgctgcctctgcc |
| | atctctgtggccaggaccctgctggtgtttgaggtgcagcagcctttcctgtttgtgctgtgggatcagcagcacaagttccctgtgttcatgggcagggtgta |
| | tgaccctagggcttga |
| 238 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagctgcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggtggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctga |
| 239 | aatcccaatgccacctctagctctagccaggatcctgagagcctgcaggacaggggggagggcaaggtggccaccactgtgatcagcaagatgctgttt |
| | gtggagcccattctggaggtgtcttctctgcccaccaccaatagcaccactaattctgccaccaagatcactgctaacaccactgatgagcccaccactcag |
| | cctaccactgagcccaccacccagcccactatccagcccacccagcccaccacccagctgcctactgacagccctacccagcccactactgggagcttc |
| | tgccctggccctgtgaccctgtgctctgacctggagagccacagcactgaggctgtgctgggggatgccctggtggacttcagcctgaagctgtaccatg |
| | ccttttctgccatgaagaaggtggagaccaacatggcctttagccctttcagcattgccagcctgctgactcaggtgctgctgggggctggggagaacacc |
| | aagaccaacctggagagcatcctgagctaccccaaggacttcacttgtgtgcaccaggccctgaagggcttcactaccaagggggtgacttctgtgagcc |
| | agatcttccacagccctgacctggccatcagggatacttttgtgaatgccagcaggaccctgtacagcagcagccccagggtgctgagcaacaactctgat |
| | gctaacctggagctgatcaatacttgggtggccaagaacaccaacaacaagatttctaggctgctggactctctgccctctgacactaggctggtgctgctg |
| | aatgccatctacctgtctgccaagtggaagaccacctttgatcccaagaagactaggatggagcccttccacttcaagaactctgtgatcaaggtgcccatg |
| | atgaacagcaagaagtaccctgtggcccatttcattgaccagaccctgaaggccaaggtggggcagctgcagctgagccacaatctgagcctggtgattc |
| | tggtgccccagaatctgaagcacagactggaggacatggagcaggctctgtctccctctgtgttcaaggccatcatggagaagctggagatgagcaagtt |
| | ccagcccactctgctgactctgcctaggatcaaggtgaccaccagccaggacatgctgagcattatggagaagctggagttctttgacttcagctatgacct |
| | gaacctgtgtggcctgactgaggaccctgatctgcaggtgtctgccatgcagcaccagactgtgctggagctgactgagactggggtggaggctgctgct |
| | gcctctgctatttctgtggccaggaccctgctggtgtttgaggtgcagcagcccttcctgtttgtgctgtgggaccagcagcacaagtttcctgtgttcatggg |
| | gagggtgtatgaccccagggcctga |
| 240 | aatccaaatgctaccagctcctcctcccaggatcctgaatcccttcaagacaggggggagggcaaggtggcaactactgtgatttccaagatgctgtttgtg |
| | gagcccatcctggaggttagcagccttcctactaccaactcaacaaccaattcagccaccaaaattacagctaataccacagatgagccaacaactcaacc |
| | caccactgagcccaccactcagccaaccatccaacccacccaaccaactacccagctcccaacagattctcctacccagcctaccactggctccttctgcc |
| | caggacctgttactctctgctctgacttggaaagccattctactgaggctgtcctgggggatgctttggtagacttctccctgaagctctaccatgcattttcagc |
| | aatgaagaaggtggagaccaacatggccttttccccattcagtatagcaagtctcctgactcaggtcctgctgggggcaggggagaatacaaagaccaac |
| | ctggagagcatcctcagttatcccaaggacttcacctgtgtccaccaggccctgaagggcttcacaacaaagggggtgacctcagtctctcagattttccac |
| | agcccagacctggccataagggacacctttgtgaatgcctctaggaccctgtactcaagctcccccagagtcctaagcaacaacagtgatgcaaatttgga |
| | gctcatcaacacctgggtggccaagaacaccaacaacaagatcagcaggctgctagacagtctgccaagtgatacaaggcttgtcctcctcaatgctatct |
| | acctgagtgccaagtggaagacaacatttgatcccaagaaaaccagaatggaaccctttcacttcaaaaactcagttataaaagtgcccatgatgaatagca |
| | agaagtaccctgtggcccatttcattgaccaaactttgaaagccaaggtggggcagctgcagctctcccacaatctgagtctggtaatcctggtaccccaga |
| | acctgaaacataggcttgaagacatggaacaggctctcagcccttctgttttcaaggccataatggagaagctggagatgtccaagttccagcccactctcc |
| | taacactacccaggatcaaagtgacaaccagccaggatatgctctcaatcatggagaaattggaattttttgatttttcttatgaccttaacctgtgtgggctgac |
| | agaggaccctgatcttcaggtttctgcaatgcagcaccagacagtgctggaactgacagagactggggtggaagctgctgcagcaagtgcaatatctgtg |
| | gccagaaccctgctggtctttgaagtgcagcagcccttcctgtttgtgctgtgggaccagcagcacaagttccctgtcttcatggggagggtatatgacccc |
| | agggcctga |
| 241 | aaccccaatgcaaccagcagttcctctcaggatccagagtccctgcaggacaggggggagggcaaggtggccaccactgtgatctccaagatgctgttt |
| | gtggagcccatcctggaggtatccagcctccctaccaccaacagcaccaccaactcagccaccaaaatcactgctaacaccactgatgagcccacaacc |
| | cagccaactactgagccaaccactcagcctaccatccaacccacccagcctactacccagctccccactgactctcctacccagcccaccactggcagctt |
| | ctgcccaggacctgtgaccctctgctctgacttggagtcccattccacagaggcagtgctgggggatgccctggtagacttctccctgaagctctaccatgc |
| | attctctgccatgaagaaggtggagaccaacatggccttctcccccttcagcattgccagcctcctgacccaggtcctgctgggggcaggggagaacaca |
| | aagacaaacctggagtccatcctcagctaccccaaggacttcacctgtgtccaccaggccctgaagggcttcacaaccaagggggtgaccagtgtctcac |
| | agattttccacagcccagacctggccatcagggacacctttgtgaatgccagtaggaccctgtacagctccagtcccagagtcctcagcaacaactctgat |
| | gccaacctggagctgatcaacacctgggtggccaagaacaccaacaacaagatcagcaggctgctagactcactgccctctgacacaaggctggtcctc |
| | ctcaatgcaatctacctgagtgccaagtggaagaccacctttgaccccaagaagaccaggatggagcccttccacttcaagaacagtgtcattaaggtgcc |
| | catgatgaacagcaagaagtaccctgtggcccacttcattgaccagaccttgaaggccaaggtggggcagctgcagctctcccacaacctgtcactggtc |
| | atcctggttccccagaacctgaagcacaggcttgaggacatggagcaggccctcagcccttctgtattcaaggccataatggagaagctggagatgagca |
| | agttccagcccactctgctgaccctacccaggatcaaggtgaccaccagccaggacatgctctcaatcatggagaagttggagttctttgacttcagctatg |
| | acctgaacctgtgtgggctgacagaggaccctgacctgcaggtgtcagccatgcagcaccagacagtgctggaactgacagagactggggtggaagct |
| | gctgcagcaagtgctatttcagtggccaggaccctgctggtctttgaggtccagcagcccttcctgtttgtgctctgggaccagcagcacaagttccctgtctt |
| | catggggagggtctatgaccccagggcctga |
| 242 | aaccccaatgccaccagctccagctctcaggacccagagagcctgcaggacaggggggagggcaaggtggccaccacagtgatcagcaagatgctgt |
| | ttgtggagcccatcctggaggtgagcagcctccccaccaccaacagcaccaccaattctgccaccaagatcacagccaacaccacagatgagcccacaa |
| | cccagcctaccacagagccaaccacacagcccaccatccaacccacccaacctactacccagctgcccacagacagccctacccagcctaccacaggc |
| | agcttctgccctggccctgtgaccctgtgctctgacttggagagccactccacagaggctgtgctgggggatgccctggtggatttcagcctgaagctgtac |
| | catgccttctctgccatgaagaaggtggagaccaacatggccttcagccccttcagcattgccagcctcctgacccaggtcctgctgggggctggggaga |
| | acacaaagaccaacctggagagcatcctgagctaccccaaggacttcacctgtgtgcaccaggccctgaagggcttcaccaccaagggggtgacctctg |
| | tgagccagattttccacagccctgacctggccatcagggacacctttgtgaatgcctccaggaccctgtacagctctagccccagggtgctgagcaacaac |
| | tctgatgccaacttggagctgatcaacacctgggtggccaagaacaccaacaacaagatcagcaggctgctggacagcctgccctctgacaccaggctg |
| | gtgctcctgaatgccatctacctgtctgccaagtggaagaccacctttgaccccaagaagaccagaatggagcccttccacttcaagaactctgtgataaag |
| | gtgcccatgatgaacagcaagaagtaccctgtggcccacttcattgaccagaccctgaaagccaaggtgggccagctgcagctcagccacaacctgagc |
| | ctggtcatcctggtgccccagaacctgaagcacaggctggaggacatggaacaggccctcagcccctctgtgttcaaggccattatggagaagctggag |
| | atgagcaagttccagcccactctgctgacactgcccaggatcaaggtgaccaccagccaggacatgctgagcatcatggagaagttggagttctttgactt |
| | cagctatgacctgaacctgtgtggcctgacagaggaccctgacctccaggtgtctgccatgcagcaccagacagtgctggaactgacagagacaggggt |
| | ggaagctgctgctgcctctgccatctctgtggccagaaccctgctggtgtttgaggtgcagcagcccttcctgtttgtgctgtgggaccagcagcacaagttc |
| | cctgtgttcatgggcagggtgtatgaccccagggcctga |
| 243 | aaccccaatgctacttctagctcctctcaggaccctgagagccttcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagcttcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatcttcaggtgtctgccatgcagcaccagactgtgctggagctgactgagactggggtggaggctgctgctgcttctgccatct |
| | ctgtggccaggaccctgctggtgtttgaggtgcagcagcccttcctgtttgtgctgtgggaccagcagcacaagtttcctgtgttcatgggcagggtgtatga |
| | ccccagagcctga |
| 244 | aatcccaccatccaacccacccaaccaactacccagctcccaacagattctcctacccagcccactactgggtccttctgcccaggacctgttactctctgct |
| | ctgacttggagagtcattcaacagaggccgtgttgggggatgctttggtagatttctccctgaagctctaccacgccttctcagcaatgaagaaggtggaga |
| | ccaacatggccttttccccattcagcatcgccagcctccttacccaggtcctgctcggggctggggagaacaccaaaacaaacctggagagcatcctctctt |
| | accccaaggacttcacctgtgtccaccaggccctgaagggcttcacgaccaaaggtgtcacctcagtctctcagattttccacagcccagacctggccata |
| | agggacacctttgtgaatgcctctcggaccctgtacagcagcagccccagagtcctaagcaacaacagtgacgccaacttggagctcatcaacacctggg |
| | tggccaagaacaccaacaacaagatcagccggctgctagacagtctgccctccgatacccgccttgtcctcctcaatgctatctacctgagtgccaagtgg |
| | aagacaacatttgatcccaagaaaaccagaatggaaccctttcacttcaaaaactcagttataaaagtgcccatgatgaatagcaagaagtaccctgtggcc |
| | catttcattgaccaaactttgaaagccaaggtggggcagctgcagctctcccacaatctgagtttggtgatcctggtaccccagaacctgaaacatcgtcttg |
| | aagacatggaacaggctctcagcccttctgttttcaaggccatcatggagaaactggagatgtccaagttccagcccactctcctaacactaccccgcatca |
| | aagtgacgaccagccaggatatgctctcaatcatggagaaattggaattcttcgatttttcttatgaccttaacctgtgtgggctgacagaggaccctgatcttc |
| | aggtttctgcgatgcagcaccagacagtgctggaactgacagagactggggtggaggcggctgcagcctccgccatctctgtggcccgcaccctgctgg |
| | tctttgaagtgcagcagcccttcctcttcgtgctctgggaccagcagcacaagttccctgtcttcatggggcgagtatatgaccccagggcctga |
| 245 | aatgggtccttctgcccaggacctgttactctctgctctgacttggagagtcattcaacagaggccgtgttgggggatgctttggtagatttctccctgaagctc |
| | taccacgccttctcagcaatgaagaaggtggagaccaacatggccttttccccattcagcatcgccagcctccttacccaggtcctgctcggggctgggga |
| | gaacaccaaaacaaacctggagagcatcctctcttaccccaaggacttcacctgtgtccaccaggccctgaagggcttcacgaccaaaggtgtcacctca |
| | gtctctcagattttccacagcccagacctggccataagggacacctttgtgaatgcctctcggaccctgtacagcagcagccccagagtcctaagcaacaa |
| | cagtgacgccaacttggagctcatcaacacctgggtggccaagaacaccaacaacaagatcagccggctgctagacagtctgccctccgatacccgcctt |
| | gtcctcctcaatgctatctacctgagtgccaagtggaagacaacatttgatcccaagaaaaccagaatggaaccctttcacttcaaaaactcagttataaaagt |
| | gcccatgatgaatagcaagaagtaccctgtggcccatttcattgaccaaactttgaaagccaaggtggggcagctgcagctctcccacaatctgagtttggt |
| | gatcctggtaccccagaacctgaaacatcgtcttgaagacatggaacaggctctcagcccttctgttttcaaggccatcatggagaaactggagatgtccaa |
| | gttccagcccactctcctaacactaccccgcatcaaagtgacgaccagccaggatatgctctcaatcatggagaaattggaattcttcgatttttcttatgacct |
| | taacctgtgtgggctgacagaggaccctgatcttcaggtttctgcgatgcagcaccagacagtgctggaactgacagagactggggtggaggcggctgc |
| | agcctccgccatctctgtggcccgcaccctgctggtctttgaagtgcagcagcccttcctcttcgtgctctgggaccagcagcacaagttccctgtcttcatg |
| | gggcgagtatatgaccccagggcctga |
| 246 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagctgcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggtggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctga |
| 247 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagctgcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctga |
| 248 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggtaagaccctgcttgaattct |
| | ctccaggtcatttgttggacactcccataagagtcaccaatccagacacttacaaagccatgcctctgggaagaagctgtaaaaatgggctattatatattgg |
| | gggtggggtagagggatgtatcttttcattcttgaacattccatcatttcacagtgatgtaataggcacgattgcttgtaaaactctgtgactatacaagaacata |
| | taaaataaggtcgcagccactaaccatgtttcatggcaaggagaggtgataagaaagatgaaattaggcgcagtggctcacgcctgtaatcccagcacttt |
| | gggaggccaaggcgggtggatcacctgaggtcaggagttcaagaccagcctggccaacatggtgaaaccctgtctctactaaaaatacaaaaattatcag |
| | ggagtggtggtgcatgcctgtaatcccagctacttgggcagctgaggcaggagaatcgcttgaacccaggaggtggaggttgcagtgagccgagaccg |
| | caccattgcactacagtctgggtgacagagcgagactctgtctcaaaaaaaaaaaaaaattatcagagatagacctagagtagatgtggttagtactgccttc |
| | tagctctgtgaccttgggcagatcactttaacctctctgagccttgagtcctcttgtgtaaaatagtgatgatgctatctacctcaaaagattaagaagcagaaa |
| | gccaggccggatgcggtggttcacacctgtaatcccagcattttaggaggccgaggcgggcagatcacgaggtcaggagttcgagaccagcctgactaa |
| | catggtgaaaccccgtctctactaaaaataaaaagaaattagctaggcatggtggtgcacacctgtaaccccagctactcaggaggctgaggcaggagaa |
| | tcacttgaacacgggaggcagaggttgcagtgagccgaaatcatgccactgcactccagcctgggaagactgagcaagactctgtctcaaaaaaaaaaa |
| | agaagcagcctagtgtctgacttagtgggaggtcaaaaaaatgtaaatcctctgccatcttgagggattactgtcaagtcccatttggtaattaccctaggaat |
| | ggcacaaacaaattactacaagcagtggggacagagctattactccccagagagaattctaaaaaggctacagaatctttcttggctgggcacggtgactc |
| | acacctataatcctggcactttgggaggccaaggcaggagttcaagaccagcctggccaacatgttgaaaccccatctctactaaaaatgcaaaaattagc |
| | caggcatagtgatgcatgcttattgtcccagctacttgggaggcagaggtgggaggattgcttgaacctggagattcaagtgagctgagattgcaccactgc |
| | attccagcctgggcaacaaagcaagactctgtctcaaaaaaaaaaaaaaaaaaaaaaaaaagagagattgagagaacacttccagctcagatgatctgtg |
| | atcccctccaaagcagggaataccctccattccagcctggtccccaaccctcattcccaaggaaggcccccgactcatcctgcaagtatctttcatctctgcc |
| | ctttgttgcaggggctggggagaacactaagaccaacctggaatctatcctgagctaccccaaggacttcacctgtgtgcaccaggccctgaagggcttca |
| | ccaccaagggggtgacctctgtgagccagatcttccacagccctgacctggccatcagggatacttttgtgaatgccagcagaaccctgtactcttctagcc |
| | ccagggtgctgagcaataattctgatgctaacctggagctgatcaatacctgggtggctaagaataccaacaacaagattagcaggctgctggactctctgc |
| | cttctgataccaggctggtgctgctgaatgccatttacctgtctgccaagtggaagaccacttttgaccctaagaagactaggatggagcctttccacttcaag |
| | aactctgtcattaaggtgcccatgatgaacagcaagaagtatcctgtggcccactttattgaccagactctgaaggccaaggtgggccagctgcagctgag |
| | ccataacctgagcctggtgatcctggtgccccagaacctgaagcataggctggaggacatggagcaggctctgagcccctctgtgttcaaggctattatgg |
| | agaagctggagatgagcaagttccagcccactctgctgaccctgcccagaatcaaggtgaccacctctcaggacatgctgagcatcatggaaaagctgg |
| | agttctttgacttttcttatgacctgaatctgtgtggcctgactgaggatcctgatctgcaggtgtctgccatgcagcaccagactgtgctggagctgactgaga |
| | ctggggtggaggctgctgctgcttctgccatctctgtggccaggaccctgctggtgtttgaggtgcagcagcccttcctgtttgtgctgtgggaccagcagc |
| | acaagtttcctgtgttcatgggcagggtgtatgaccccagagcctga |
| 249 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggtaagaccctgcttgaattct |
| | ctccaggtcatttgttggacactcccataagagtcaccaatccagacacttacaaagccatgcctctgggaagaagctgtaaaaatgggctattatatattgg |
| | gggtggggtagagggatgtatcttttcattcttgaacattccatcatttcacagtgatgtaataggcaccattgcttgtaaaactctgtgactatacaagaacatat |
| | aaaataaggtcccagccactaaccatgtttcatggcaaggagaggtgataagaaagatgaaattattatcagaagaagcagaaagccagcagcctagtgt |
| | ctgacttagtgggaggtcaaaaaaatgtaaatcctctgccatcttgagggattactgtcaagtcccatttggtaattaccctaggaatggcacaaacaaattact |
| | acaagcagtggggacagagctattactccccagagagaattctaaaaaggctacagaatctttcttgagagattgagagaacacttccagctcagatgatct |
| | gtgatcccctccaaagcagggaataccctccattccagcctggtccccaaccctcattcccaaggaaggccccccactcatcctgcaagtatctttcatctct |
| | gccctttgttgcaggggctggggagaacactaagaccaacctggaatctatcctgagctaccccaaggacttcacctgtgtgcaccaggccctgaagggc |
| | ttcaccaccaagggggtgacctctgtgagccagatcttccacagccctgacctggccatcagggatacttttgtgaatgccagcagaaccctgtactcttcta |
| | gccccagggtgctgagcaataattctgatgctaacctggagctgatcaatacctgggtggctaagaataccaacaacaagattagcaggctgctggactct |
| | ctgccttctgataccaggctggtgctgctgaatgccatttacctgtctgccaagtggaagaccacttttgaccctaagaagactaggatggagcctttccactt |
| | caagaactctgtcattaaggtgcccatgatgaacagcaagaagtatcctgtggcccactttattgaccagactctgaaggccaaggtgggccagctgcagc |
| | tgagccataacctgagcctggtgatcctggtgccccagaacctgaagcataggctggaggacatggagcaggctctgagcccctctgtgttcaaggctatt |
| | atggagaagctggagatgagcaagttccagcccactctgctgaccctgcccagaatcaaggtgaccacctctcaggacatgctgagcatcatggaaaag |
| | ctggagttctttgacttttcttatgacctgaatctgtgtggcctgactgaggatcctgatctgcaggtgtctgccatgcagcaccagactgtgctggagctgact |
| | gagactggggtggaggctgctgctgcttctgccatctctgtggccaggaccctgctggtgtttgaggtgcagcagcccttcctgtttgtgctgtgggaccag |
| | cagcacaagtttcctgtgttcatgggcagggtgtatgaccccagagcctga |
| 250 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctcatgatctcccggacccctgaggtcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccgtcctgcaccaggactggctgaat |
| | ggcaaggagtacaagtgcaaggtctccaacaaagccctcccagcccccatcgagaaaaccatctccaaagccaaagggcagccccgagaaccacagg |
| | tgtacaccctgcccccatcccgggatgagctgaccaagaaccaggtcagcctgacctgcctggtcaaaggcttctatcccagcgacatcgccgtggagtg |
| | ggagagcaatgggcagccggagaacaactacaagaccacgcctcccgtgctggactccgacggctccttcttcctctacagcaagctcaccgtggacaa |
| | gagcaggtggcagcaggggaacgtcttctcatgctccgtgatgcatgaggctctgcacaaccactacacgcagaagagcctctccctgtctccgggt |
| 251 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctcatgatctcccggacccctgaggtcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccgtcctgcaccaggactggctgaat |
| | ggcaaggagtacaagtgcaaggtctccaacaaagccctcccagcccccatcgagaaaaccatctccaaagccaaagggcagccccgagaaccacagg |
| | tgtacaccctgcccccatcccgggatgagctgaccaagaaccaggtcaatctgacctgcctggtcaaaggcttctatcccagcgacatcgccgtggagtg |
| | ggagagcaatgggcagccggagaacaactacaagaccacgcctcccgtgctggactccgacggctccttcttcctcaatagcaagctcaccgtggacaa |
| | gagcaggtggcagcaggggaacgtcttctcatgctccgtgatgcatgaggctctgcacaaccactacacgcagaagagcctctccctgtctccgggt |
| 252 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctcatgatctcccggacccctgaggtcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccgtcctgcaccaggactggctgaat |
| | ggcaaggagtacaagtgcaaggtctccaacaaagccctcccagcccccatcgagaaaaccatctccaaagccaaagggcagccccgagaaccacagg |
| | tgtacaccaagcccccatcccgggatgagctgaccaagaaccaggtcagcctgagctgcctggtcaaaggcttctatcccagcgacatcgccgtggagt |
| | gggagagcaatgggcagccggagaacaactacaagaccacggtgcccgtgctggactccgacggctccttcagactcgccagctacctcaccgtggac |
| | aagagcaggtggcagcaggggaacgtcttctcatgctccgtgatgcatgaggctctgcacaaccactacacgcagaagagcctctccctgtctccgggt |
| 253 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctctacatcacccgggagcctgaggtcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccgtcctgcaccaggactggctgaat |
| | ggcaaggagtacaagtgcaaggtctccaacaaagccctcccagcccccatcgagaaaaccatctccaaagccaaagggcagccccgagaaccacagg |
| | tgtacaccctgcccccatcccgggatgagctgaccaagaaccaggtcagcctgacctgcctggtcaaaggcttctatcccagcgacatcgccgtggagtg |
| | ggagagcaatgggcagccggagaacaactacaagaccacgcctcccgtgctggactccgacggctccttcttcctctacagcaagctcaccgtggacaa |
| | gagcaggtggcagcaggggaacgtcttctcatgctccgtgatgcatgaggctctgcacaaccactacacgcagaagagcctctccctgtctccgggt |
| 254 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctcatgatctcccggacccctgaggtcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccgtcctgcaccaggactggctgaat |
| | ggcaaggagtacaagtgcaaggtctccaacaaagccctcccagcccccatcgagaaaaccatctccaaagccaaagggcagccccgagaaccacagg |
| | tgtacaccctgcccccatcccgggatgagctgaccaagaaccaggtcagcctgacctgcctggtcaaaggcttctatcccagcgacatcgccgtggagtg |
| | ggagagcaatgggcagccggagaacaactacaagaccacgcctcccgtgctggactccgacggctccttcttcctctacagcaagctcaccgtggacaa |
| | gagcaggtggcagcaggggaacgtcttctcatgctccgtgctgcatgaggctctgcacagccactacacgcagaagagcctctccctgtctccgggt |
| 255 | aatccaaatgctaccagctccagctcccaggatccagagagtttgcaagacagaggcgaagggaaggtcgcaacaacagttatctccaagatgctattcg |
| | ttgaacccatcctggaggtttccagcttgccgacaaccaactcaacaaccaattcagccaccaaaataacagctaataccactgatgaacccaccacacaa |
| | cccaccacagagcccaccacccaacccaccatccaacccacccaaccaactacccagctcccaacagattctcctacccagcccactactgggtccttct |
| | gcccaggacctgttactctctgctctgacttggagagtcattcaacagaggccgtgttgggggatgctttggtagatttctccctgaagctctaccacgccttct |
| | cagcaatgaagaaggtggagaccaacatggccttttccccattcagcatcgccagcctccttacccaggtcctgctcggggctggggagaacaccaaaac |
| | aaacctggagagcatcctctcttaccccaaggacttcacctgtgtccaccaggccctgaagggcttcacgaccaaaggtgtcacctcagtctctcagattttc |
| | cacagcccagacctggccataagggacacctttgtgaatgcctctcggaccctgtacagcagcagccccagagtcctaagcaacaacagtgacgccaac |
| | ttggagctcatcaacacctgggtggccaagaacaccaacaacaagatcagccggctgctagacagtctgccctccgatacccgccttgtcctcctcaatgc |
| | tatctacctgagtgccaagtggaagacaacatttgatcccaagaaaaccagaatggaaccctttcacttcaaaaactcagttataaaagtgcccatgatgaat |
| | agcaagaagtaccctgtggcccatttcattgaccaaactttgaaagccaaggtggggcagctgcagctctcccacaatctgagtttggtgatcctggtaccc |
| | cagaacctgaaacatcgtcttgaagacatggaacaggctctcagcccttctgttttcaaggccatcatggagaaactggagatgtccaagttccagcccact |
| | ctcctaacactaccccgcatcaaagtgacgaccagccaggatatgctctcaatcatggagaaattggaattcttcgatttttcttatgaccttaacctgtgtggg |
| | ctgacagaggaccctgatcttcaggtttctgcgatgcagcaccagacagtgctggaactgacagagactggggtggaggcggctgcagcctccgccatc |
| | tctgtggcccgcaccctgctggtctttgaagtgcagcagcccttcctcttcgtgctctgggaccagcagcacaagttccctgtcttcatggggcgagtatatg |
| | accccagggccgacaaaactcacacatgcccaccgtgcccagcacctgaactcctggggggaccgtcagtcttcctcttccccccaaaacccaaggaca |
| | ccctcatgatctcccggacccctgagatcacatgcgtggtggtggacgtgagccacgaagaccctgaggtcaagttcaactggtacgtggacggcgtgg |
| | aggtgcataatgccaagacaaagccgcgggaggagcagtacaacagcacgtaccgtgtggtcagcgtcctcaccttcctgcaccaggactggctgaatg |
| | gcaaggagtacaagtgcaaggtctccaacaaagccctcccagcccccatcgagaaaaccatctccaaagccaaagggcagccccgagaaccacaggt |
| | gtacaccctgcccccatcccgggatgagctgaccaagaaccaggtcagcctgacctgcctggtcaaaggcttctatcccagcgacatcgccgtggagtg |
| | ggagagcaatgggcagccggagaacaactacaagaccacgcctcccgtgctggactccgacggctccttcttcctctacagcaagctcaccgtggacaa |
| | gagcaggtggcagcaggggaacgtcttctcatgctccgtgctgcatgaggctctgcacaaccactacacgcagaagagcctctccctgtctccgggt |
| 256 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctcagacctggagagccactctactgaggctgtgctgggggatgccctggtggacttctctctgaagctgtatcatg |
| | ctttctctgccatgaagaaggtggagactaacatggcttttagccccttcagcattgctagcctgctgacccaggtgctgctgggggctggggagaacacta |
| | agaccaacctggaatctatcctgagctaccccaaggacttcacctgtgtgcaccaggccctgaagggcttcaccaccaagggggtgacctctgtgagcca |
| | gatcttccacagccctgacctggccatcagggatacttttgtgaatgccagcagaaccctgtactcttctagccccagggtgctgagcaataattctgatgcta |
| | acctggagctgatcaatacctgggtggctaagaataccaacaacaagattagcaggctgctggactctctgccttctgataccaggctggtgctgctgaatg |
| | ccatttacctgtctgccaagtggaagaccacttttgaccctaagaagactaggatggagcctttccacttcaagaactctgtcattaaggtgcccatgatgaac |
| | agcaagaagtatcctgtggcccactttattgaccagactctgaaggccaaggtgggccagctgcagctgagccacaacctgagcctggtgatcctggtgc |
| | cccagaacctgaagcacaggctggaggacatggagcaggctctgagcccctctgtgttcaaggctattatggagaagctggagatgagcaagttccagc |
| | ccactctgctgaccctgcccagaatcaaggtgaccacctctcaggacatgctgagcatcatggaaaagctggagttctttgacttttcttatgacctgaatctg |
| | tgtggcctgactgaggatcctgatctgcaggtgtctgccatgcagcaccagactgtgctggagctgactgagactggggggaggctgctgctgcttctgc |
| | catctctgtggccaggaccctgctggtgtttgaggtgcagcagcccttcctgtttgtgctgtgggaccagcagcacaagtttcctgtgttcatgggcagggtg |
| | tatgaccccagagcctga |
| 257 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagctgcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctga |
| 258 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggtaagaccctgcttgaattct |
| | ctccaggtcatttgttggacactcccataagagtcaccaatccagacacttacaaagccatgcctctgggaagaagctgtaaaaatgggctattatatattgg |
| | gggtggggtagagggatgtatcttttcattcttgaacattccatcatttcacagtgatgtaataggcacgattgcttgtaaaactctgtgactatacaagaacata |
| | taaaataaggtcgcagccactaaccatgtttcatggcaaggagaggtgataagaaagatgaaattattatcagaagaagcagaaagccagcagcctagtgt |
| | ctgacttagtgggaggtcaaaaaaatgtaaatcctctgccatcttgagggattactgtcaagtcccatttggtaattaccctaggaatggcacaaacaaattact |
| | acaagcagtggggacagagctattactccccagagagaattctaaaaaggctacagaatctttcttgagagattgagagaacacttccagctcagatgatct |
| | gtgatcccctccaaagcagggaataccctccattccagcctggtccccaaccctcattcccaaggaaggcccccgactcatcctgcaagtatctttcatctct |
| | gccctttgttgcaggggctggggagaacactaagaccaacctggaatctatcctgagctaccccaaggacttcacctgtgtgcaccaggccctgaagggc |
| | ttcaccaccaagggggtgacctctgtgagccagatcttccacagccctgacctggccatcagggatacttttgtgaatgccagcagaaccctgtactcttcta |
| | gccccagggtgctgagcaataattctgatgctaacctggagctgatcaatacctgggtggctaagaataccaacaacaagattagcaggctgctggactct |
| | ctgccttctgataccaggctggtgctgctgaatgccatttacctgtctgccaagtggaagaccacttttgaccctaagaagactaggatggagcctttccactt |
| | caagaactctgtcattaaggtgcccatgatgaacagcaagaagtatcctgtggcccactttattgaccagactctgaaggccaaggtgggccagctgcagc |
| | tgagccataacctgagcctggtgatcctggtgccccagaacctgaagcataggctggaggacatggagcaggctctgagcccctctgtgttcaaggctatt |
| | atggagaagctggagatgagcaagttccagcccactctgctgaccctgcccagaatcaaggtgaccacctctcaggacatgctgagcatcatggaaaag |
| | ctggagttctttgacttttcttatgacctgaatctgtgtggcctgactgaggatcctgatctgcaggtgtctgccatgcagcaccagactgtgctggagctgact |
| | gagactggggtggaggctgctgctgcttctgccatctctgtggccaggaccctgctggtgtttgaggtgcagcagcccttcctgtttgtgctgtgggaccag |
| | cagcacaagtttcctgtgttcatgggcagggtgtatgaccccagagcctga |
| 259 | atggctagcagactgactctgctgaccctgctgctgctgctgctggctggggacagggccagcagc |
| 260 | atggccagcaggctgaccctgctgactctgctgctgctgctgctggctggggacagggccagcagc |
| 261 | atggctagcagactgactctgctgaccctgctgctgctgctgctggctggggacagggccagcagc |
| 262 | atggctagcagactgactctgctgaccctgctgctgctgctgctggctggggacagggccagcagc |
| 263 | atggccagcaggctgaccctgctgaccctgctgctgctcctgctggctggggatagggccagcagc |
| 264 | atggcctctaggctgactctgctgactctgctgctcctgctgctggctggggacagggccagctct |
| 265 | atggctagcaggctgaccctgctgaccctgctgctcctgctgctggctggggacagggcctctagc |
| 266 | atggcctccaggctgaccctgctgaccctcctgctgctcctgctggctggggatagagcctctagc |
| 267 | atggcctccaggctgaccctgctcaccctcctgctgctcctgctggctggggacagggcctccagc |
| 268 | atggcctccaggctgaccctgctgaccctcctgctgctcctgctggctggggacagagccagcagc |
| 269 | aaccccaatgctacttctagctcctctcaggaccctgagagcctgcaggacaggggggagggcaaggtggccaccactgtgatctctaagatgctgtttgt |
| | ggagcccatcctggaggtgagcagcctgcccaccaccaacagcaccaccaactctgccactaagatcactgccaacaccactgatgagcccaccaccc |
| | agcccactactgagcccaccacccagcctactatccagcccacccagcccactacccagctgcccactgacagccctacccagcccaccactggcagct |
| | tctgccctgggcctgtgaccctgtgctctgacctggagagccattctactgaggctgtgctgggggatgccctggtggacttctctctgaagctgtaccatgc |
| | tttctctgccatgaagaaggtggagactaacatggcttttagccccttcagcattgctagcctgctgacccaggtgctgctgggggctggggagaacactaa |
| | gaccaacctggaatctatcctgagctaccccaaggacttcacctgtgtgcaccaggccctgaagggcttcaccaccaagggggtgacctctgtgagccag |
| | atcttccacagccctgacctggccatcagggatacttttgtgaatgccagcagaaccctgtactcttctagccccagggtgctgagcaataattctgatgctaa |
| | cctggagctgatcaatacctgggtggctaagaataccaacaacaagattagcaggctgctggactctctgccttctgataccaggctggtgctgctgaatgc |
| | catttacctgtctgccaagtggaagaccacttttgaccctaagaagactaggatggagcctttccacttcaagaactctgtcattaaggtgcccatgatgaaca |
| | gcaagaagtatcctgtggcccactttattgaccagactctgaaggccaaggtgggccagctgcagctgagccataacctgagcctggtgatcctggtgccc |
| | cagaacctgaagcataggctggaggacatggagcaggctctgagcccctctgtgttcaaggctattatggagaagctggagatgagcaagttccagccca |
| | ctctgctgaccctgcccagaatcaaggtgaccacctctcaggacatgctgagcatcatggaaaagctggagttctttgacttttcttatgacctgaatctgtgtg |
| | gcctgactgaggatcctgatctgcaggtgtctgccatgcagcaccagactgtgctggagctgactgagactggggtggaggctgctgctgcttctgccatc |
| | tctgtggccaggaccctgctggtgtttgaggtgcagcagcccttcctgtttgtgctgtgggaccagcagcacaagtttcctgtgttcatgggcagggtgtatg |
| | accccagagcctga |

Additional embodiments are directed to the C1-INH encoding sequence provided in any one of SEQ ID NOs: 232-258 and 269. In certain embodiments, the polynucleotide comprises a sequence at least 85%, at least 90%, at least 93%, at least 95%, at least 96%, at least 97%, at least 98%, at least 99% or 100% identical to a C1-INH encoding sequence provided in any one SEQ ID NOs: 232-258 and 269; and independently C1-INH has a sequence identify to SEQ ID NO: 181 of at least 93%, at least 95%, at least 96%, at least 97%, at least 98%, at least 99% or 100%. Reference to independently indicates any of the provided sequence identities to the CT-INH encoding sequence may be combined with any of the provided sequence identities of SEQ ID NO: 181.

A set of illustrative examples of independently include, a nucleic acid having a sequence identity to SEQ ID NO: 236 or bases 1 to 1500 of SEQ ID NO: 236 of at least 90%, and encoding C1-INH having a sequence identity of at least 93%, at least 95%, at least 96%, at least 97%, at least 98%, at least 99% or 100% to SEQ ID NO: 181; a nucleic acid having a sequence identity to SEQ ID NO: 236 or bases 1 to 1500 of SEQ ID NO: 236 of at least 95%, and encoding C1-INH having a sequence identity of at least 93%, at least 95%, at least 96%, at least 97%, at least 98%, at least 99% or 100% to SEQ ID NO: 181; and a nucleic acid having a sequence identity to SEQ ID NO: 236 of at least 99% or bases 1 to 1500 of SEQ ID NO: 236 and encoding C1-INH having a sequence identity of at least 99% to the sequence of SEQ ID NO: 181.

Another set of illustrative examples of independently include, a nucleic acid having a sequence identity to SEQ ID NO: 238 or bases 1 to 1500 of SEQ ID NO: 238 of at least 90%, and encoding C1-INH having a sequence identity of at least 93%, at least 95%, at least 96%, at least 97%, at least 98%, at least 99% or 100% to SEQ ID NO: 181; a nucleic acid having a sequence identity to SEQ ID NO: 238 or bases 1 to 1500 of SEQ ID NO: 238 of at least 95%, and encoding C1-INH having a sequence identity of at least 93%, at least 95%, at least 96%, at least 97%, at least 98%, at least 99% or 100% to SEQ ID NO: 181; and a nucleic acid having a sequence identity to SEQ ID NO: 238 or bases 1 to 1500 of SEQ ID NO: 238 of at least 99% and encoding C1-INH having a sequence identity of at least 99% to the sequence of SEQ ID NO: 181.

Another set of illustrative examples of independently include, a nucleic acid having a sequence identity to SEQ ID NO: 243 or bases 1 to 1540 of SEQ ID NO: 243 of at least 90%, and encoding C1-INH having a sequence identity of at least 93%, at least 95%, at least 96%, at least 97%, at least 98%, at least 99% or 100% to SEQ ID NO: 181; a nucleic acid having a sequence identity to SEQ ID NO: 243 or bases 1 to 1540 of SEQ ID NO: 243 of at least 93%, at least 95%, and encoding C1-INH having a sequence identity of at least 93%, at least 95%, at least 96%, at least 97%, at least 98%, at least 99% or 100% to SEQ ID NO: 181; and a nucleic acid having a sequence identity to SEQ ID NO: 243 or bases 1 to 1540 of SEQ ID NO: 243 of at least 99% and encoding C1-INH having a sequence identity of at least 99% to SEQ ID NO: 181.

In certain embodiments the encoding C1-INH further comprises a signal peptide encoding sequence at least 95%, at least 97% or 100% identical to the sequence of any one of SEQ ID NOs: 84-103 and 259-268; and independently and encode C1-INH having a sequence identity of at least 93%, at least 95%, at least 96%, at least 97%, at least 98%, at least 99% or 100% to SEQ ID NO: 192.

The human C1-INH protein is well characterized and sequences from different species including primates are well known in the art. The different sequences illustrate sequence diversity of C1-INH. Examples of different C1-INH proteins are provided in the Tables below. The percent identity in the tables below were determined using BLAST with the default selling and numbers listed are the 00 pairwise identities. In different embodiments, the percent identity provided throughout the application is determined using BLAST with the default selling to obtain % pairwise identities.

| | | % Identity to |
|---|---|---|
| | | Full length human |
| Species | Reference/SEQ ID NO | C1-INH |
| Human | SEQ ID NO: 192 | 100 |
| Rhesus | Protein ID: 703928 | 93 |
| Cynomolgus monkey | Protein ID: XP_005577905.2 | 93 |
| African Green | Protein ID: XP_007996463.1 | 94 |
| Chimpanzee | Protein ID: XP_003318026.1 | 98 |

| | | % Identity to |
|---|---|---|
| | | Mature Human |
| Species | Reference/SEQ ID NO | C1-INH |
| Human | SEQ ID NO: 181 | 100 |
| Rhesus | Protein ID: 703928 | 92 |
| | Mature sequence | |
| Cynomolgus monkey | Protein ID: XP_005577905.2 | 92 |
| | Mature sequence | |
| African Green | Protein ID: XP_007996463.1 | 94 |
| | Mature sequence | |
| Chimpanzee | Protein ID: XP_003318026.1 | 98 |
| | Mature sequence | |

### EXAMPLES

### Example 1—Expression Cassette Overview

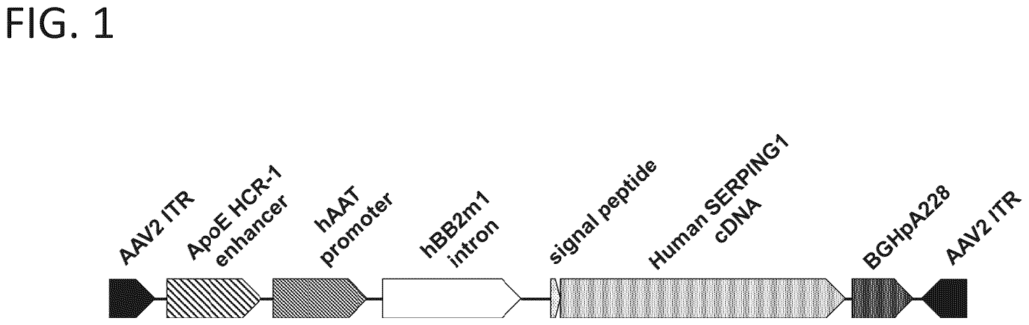

C1 inhibitor expression cassettes were designed as shown in Table 1. See also FIG. 1 for a schematic of a sample expression cassette. The expression cassettes contained 5′ and 3′ flanking AAV inverted terminal repeats (ITRs), a liver-specific ApoE/hAAT enhancer/promoter sequence, a signal peptide, a human C1 inhibitor coding sequence, with or without a P2A linker or an Fc domain, and a bovine growth hormone (bGH) polyadenylation (poly A) sequence. Some of the expression cassettes further contained a human hemoglobin subunit beta (HBB2) intron, an additional enhancer sequence, an miRNA binding site, and/or a RIDD sequence.

TABLE 1
SERPING1 expression cassette components

| | | | | | | Additional | |
|---|---|---|---|---|---|---|---|
| | SEQ | | SEQ | | SEQ | Downstream | SEQ |
| | ID | signal | ID | | ID | Regulatory | ID |
| Expression Cassette | NO | peptide | NO | CDNA | NO | Element | NO |
| pAAV_ApoE_hAAT_HBB2ml_ | 1 | SERPING1 | 84 | SERPING1 | 232 | none | |
| SERPING1_BGH228 | | | | | | | |
| pAAV_ApoE_hAAT_HBB2ml_ | 2 | SP7 | 85 | SERPING1 | 232 | none | |
| SP7.SERPING1_BGH228 | | | | | | | |
| pAAV_ApoE_hAAT_HBB2ml_ | 3 | ALB | 90 | SERPING1 | 232 | none | |
| ALB.SERPING1_BGH228 | | | | | | | |
| pAAV_ApoE_hAAT_HBB2ml_ | 4 | ORMI | 91 | SERPING1 | 232 | none | |
| ORM1.SERPING1_BGH228 | | | | | | | |
| pAAV_ApoE_hAAT_HBB2ml_ | 5 | TF | 92 | SERPING1 | 232 | none | |
| TF.SERPING1_BGH228 | | | | | | | |
| pAAV_ApoE_hAAT_HBB2ml_ | 6 | AMBP | 93 | SERPING1 | 232 | none | |
| AMBP.SERPING1_BGH228 | | | | | | | |
| pAAV_ApoE_hAAT_HBB2ml_ | 7 | LAMPI | 94 | SERPING1 | 232 | none | |
| LAMP1.SERPING1_BGH228 | | | | | | | |
| pAAV_ApoE_hAAT_HBB2ml_ | 8 | BTN2A2 | 95 | SERPING1 | 232 | none | |
| BTN2A2.SERPING1_BGH228 | | | | | | | |
| pAAV_ApoE_hAAT_HBB2ml_ | 9 | CD300 | 96 | SERPING1 | 232 | none | |
| CD300.SERPING1_BGH228 | | | | | | | |
| pAAV_ApoE_hAAT_HBB2ml_ | 10 | NOTCH2 | 97 | SERPING1 | 232 | none | |
| NOTCH2.SERPING1_BGH228 | | | | | | | |
| pAAV_ApoE_hAAT_HBB2ml_ | 11 | STRC | 98 | SERPING1 | 232 | none | |
| STRC.SERPING1_BGH228 | | | | | | | |
| pAAV_ApoE_hAAT_HBB2ml_ | 12 | AHSG | 99 | SERPING1 | 232 | none | |
| AHSG.SERPING1_BGH228 | | | | | | | |
| pAAV_ApoE_hAAT_HBB2ml_ | 13 | SYN1 | 100 | SERPING1 | 232 | none | |
| SYN1.SERPING1_BGH228 | | | | | | | |
| pAAV_ApoE_hAAT_HBB2ml_ | 14 | SYN2 | 101 | SERPING1 | 232 | none | |
| SYN2.SERPING1_BGH228 | | | | | | | |
| pAAV_ApoE_hAAT_HBB2ml_ | 15 | SYN3 | 102 | SERPING1 | 232 | none | |
| SYN3.SERPING1_BGH228 | | | | | | | |
| pAAV_ApoE_hAAT_HBB2ml_ | 16 | SYN4 | 103 | SERPING1 | 232 | none | |
| SYN4.SERPING1_BGH228 | | | | | | | |
| pAAV_ApoE_hAAT_HBB2ml_ | 17 | SERPING1 | 259 | SERPING1c | 233 | none | |
| SERPING1coI2_BGH228 | | | | o12 | | | |
| pAAV_ApoE_hAAT_HBB2ml_ | 18 | SERPING1 | 260 | SERPING1c | 234 | none | |
| SERPING1coI3_BGH228 | | | | o13 | | | |
| pAAV_ApoE_hAAT_HBB2ml_ | 19 | SERPING1 | 261 | SERPING1c | 235 | none | |
| SERPING1coI4_BGH228 | | | | oI4 | | | |
| pAAV_ApoE_hAAT_HBB2ml_ | 20 | SERPING1 | 262 | SERPING1c | 236 | none | |
| SERPING1coI7_BGH228 | | | | o17 | | | |
| pAAV_ApoE_hAAT_HBB2ml_ | 21 | SERPING1 | 263 | SERPING1c | 237 | none | |
| SERPING1coI20_BGH228 | | | | oI20 | | | |
| pAAV_ApoE_hAAT_HBB2ml_ | 22 | SERPING1 | 264 | SERPING1c | 238 | none | |
| SERPING1co21_BGH228 | | | | o21 | | | |
| pAAV_ApoE_hAAT_HBB2ml_ | 23 | SERPING1 | 265 | SERPING1c | 239 | none | |
| SERPING1coI24_BGH228 | | | | oI24 | | | |
| pAAV_ApoE_hAAT_HBB2ml_ | 24 | SERPING1 | 266 | SERPING1c | 240 | none | |
| SERPING1coH9_BGH228 | | | | oH9 | | | |
| pAAV_ApoE_hAAT_HBB2ml_ | 25 | SERPING1 | 267 | SERPING1c | 241 | none | |
| SERPING1coH13_BGH228 | | | | oH13 | | | |
| pAAV_ApoE_hAAT_HBB2ml_ | 26 | SERPING1 | 268 | SERPING1c | 242 | none | |
| SERPING1coBC2_BGH228 | | | | oBC2 | | | |
| pAAV_ApoE_hAAT_HBB2ml_ | 27 | SERPING1 | 269 | SERPING1c | 243 | none | |
| SERPING1coI21(-RIDD)_ | | | | oI21 | | | |
| BGH228 | | | | (−RIDD) | | | |
| pSwap_ApoE_hAAT_HBB2ml_ | 28 | SERPING1 | 264 | SERPING1c | 238 | none | |
| SERPING1coI21_BGH228 | | | | o21 | | | |
| pSwap_ApoE_hAAT_HBB2ml_ | 29 | SERPING1 | 265 | SERPING1c | 239 | none | |
| SERPING1coI24_BGH228 | | | | oI24 | | | |
| pAAV_ApoE_hAAT_HBB2ml_ | 30 | SERPING1 | 84 | A76.SERPI | 244 | none | |
| A76.SERPING1_BGH228 | | | | NG1 | | | |
| AAV_ApoE_hAAT_HBB2ml_ | 31 | SERPING1 | 84 | A98.SERPI | 245 | none | |
| A98.SERPING1_BGH228 | | | | NG1 | | | |
| pSwap_ApoE_hAATv18_HBB2ml_ | 32 | SERPING1 | 264 | SERPING1c | 238 | none | |
| SERPING1coI21_BGH228 | | | | o21 | | | |
| pSwap_ApoE_hAATv18_HBB2ml_ | 33 | SERPING1 | 265 | SERPING1c | 239 | none | |
| SERPING1coI24_BGH228 | | | | oI24 | | | |
| pSwap_2xApoE_hAAT_HBB2ml_ | 34 | SERPING1 | 264 | SERPING1c | 238 | none | |
| SERPING1co21_BGH228 | | | | o21 | | | |
| pSwap_4xApoE_hAAT_HBB2ml_ | 35 | SERPING1 | 264 | SERPING1c | 238 | none | |
| SERPING1co21 BGH228 | | | | o21 | | | |
| pSwap_hAAT_HBB2ml_ | 36 | SERPING1 | 264 | SERPING1c | 238 | 2xApoE | 75 |
| SERPING1co21_2xApoE_BGH228 | | | | o21 | | | |
| pSwap_hAAT_HBB2ml_ | 37 | SERPING1 | 264 | SERPING1c | 238 | 4xApoE | 76 |
| SERPING1co21_4xApoE_BGH228 | | | | o21 | | | |
| pSwap_ApoE_hAAT_HBB2ml_ | 38 | SERPING1 | 264 | SERPING1c | 238 | hAATenh | 173 |
| SERPING1co21_hAATenh_ | | | | o21 | | | |
| BGH228 | | | | | | | |
| pSwap_ApoE_hAAT_HBB2ml | 39 | SERPING1 | 264 | SERPING1c | 238 | hAATenh | 174 |
| SERPING1co21_hAATenh(col)_ | | | | o21 | | (col) | |
| BGH228 | | | | | | | |
| pSwap_ApoE_hAAT_HBB2ml | 40 | SERPING1 | 264 | SERPING1c | 238 | WPRE3 | 175 |
| SERPING1co21_WPRE3_ | | | | o21 | | | |
| BGH228 | | | | | | | |
| pSwap_ApoE_hAAT_HBB2ml_ | 41 | SERPING1 | 264 | SERPING1c | 238 | WPRE3col | 176 |
| SERPING1co21_WPRE3col_ | | | | o21 | | | |
| BGH228 | | | | | | | |
| pSwap_ApoE_hAAT HBB2ml_ | 42 | SERPING1 | 264 | SERPING1c | 238 | WPRE | 177 |
| SERPING1co21 WPRE_BGH228 | | | | o21 | | | |
| pSwap_ApoE_hAAT HBB2ml_ | 43 | SERPING1 | 264 | SERPING1c | 238 | WPREcol | 178 |
| SERPING1co21_WPREcol_ | | | | o21 | | | |
| BGH228 | | | | | | | |
| pSwap_ApoE_hAAT_HBB2ml | 44 | SERPING1 | 264 | SERPING1c | 238 | none | |
| (−ATG)_SERPING1co21_ | | | | o21 | | | |
| BGH228 | | | | | | | |
| pSwap_ApoE_hAAT_SERPING | 45 | SERPING1 | 264 | SERPING1c | 246 | none | |
| 1co21(Intron 1-2, 2-3)_BGH228 | | | | o21(Intron | | | |
| | | | | 1-2, 2-3) | | | |
| pSwap_ApoE_hAAT_HBB2ml_ | 46 | SERPING1 | 264 | SERPING1c | 247 | none | |
| SERPING1co21(Intron 2- | | | | o21(Intron | | | |
| 3)_BGH228 | | | | 2-3) | | | |
| pSwap_ApoE_hAAT_HBB2ml_ | 47 | SERPING1 | 264 | SERPING1c | 248 | none | |
| SERPING1co21(Intron 3- | | | | o21(Intron | | | |
| 4)_BGH228 | | | | 3-4) | | | |
| pSwap_ApoE_hAAT_HBB2ml_ | 48 | SERPING1co21.P2A.SERPING1co24 | | | | none | |
| SERPING1co21.P2A.SERPING | | (SEQ ID NO: 158) | | | | | |
| lco24_BGH228 | | | | | | | |
| pAAV_ApoE_hAAT_HBB2ml_ | 49 | SERPING1 | 84 | SERPING1. | 250 | none | |
| SERPING1.Fc_BGH228 | | | | Fc | | | |
| pAAV_ApoE_hAAT_HBB2ml_ | 50 | SERPING1 | 84 | SERPING1. | 251 | none | |
| SERPING1.Fc(Ishino)_BGH228 | | | | Fc(Ishino) | | | |
| pAAV_ApoE_hAAT_HBB2ml_ | 51 | SERPING1 | 84 | SERPING1. | 252 | none | |
| SERPING1.Fc(Ying)_BGH228 | | | | Fc(Ying) | | | |
| pAAV_ApoE_hAAT_HBB2ml_ | 52 | SERPING1 | 84 | SERPING1. | 253 | none | |
| SERPING1.Fc(YTE)_BGH228 | | | | Fc(YTE) | | | |
| pAAV_ApoE_hAAT_HBB2ml_ | 53 | SERPING1 | 84 | SERPING1. | 254 | none | |
| SERPING1.Fc(LS)_BGH228 | | | | Fc(LS) | | | |
| pAAV_ApoE_hAAT_HBB2ml_ | 54 | SERPING1 | 84 | SERPING1. | 255 | none | |
| SERPING1.Fc(IFL)_BGH228 | | | | Fc(IFL) | | | |
| pSwap_ApoE_hAAT_HBB2ml_ | 55 | SERPING1 | 264 | SERPING1c | 238 | 4x mir-142-3p | 179 |
| SERPING1co21_(4x mir-142- | | | | o21 | | | |
| 3p)_BGH228 | | | | | | | |
| pAAV_ApoE_hAAT_HBB2ml_ | 56 | SERPING1 | 264 | SERPING1c | 256 | none | |
| SERPING1co21_BGH228 | | | | o21(−ORFs) | | | |
| (-ORFs) | | | | | | | |
| pSwap_ApoE_hAAT_ | 57 | SERPING1 | 264 | SERPING1c | 238 | (SERPING1 | 180 |
| SERPING1co21(SERPING1 | | | | o21 | | 5′UTR) | |
| 5′UTR)_BGH228 | | | | | | | |
| pSwap_ApoE_hAAT_HBB2ml_ | 58 | SERPING1 | 264 | SERPING1c | 257 | none | |
| SERPING1co21(Intron 2- | | | | o21(Intron | | | |
| 3)_BGH228 - SINES_LINES | | | | 2-3) | | | |
| | | | | (SINES_ | | | |
| | | | | LINES) | | | |
| pSwap_ApoE_hAAT_HBB2ml_ | 59 | SERPING1 | 264 | SERPING1c | 172 | none | |
| SERPING1co21(Intron 3-4)_ | | | | o21(Intron | | | |
| BGH228 - SINES_LINES | | | | 3-4) | | | |
| | | | | (SINES_ | | | |
| | | | | LINES) | | | |
| pSwap_ApoE_hAAT_ | 60 | SERPING1 | 264 | SERPING1c | 238 | SERPING1 | 181 |
| SERPING1co21(SERPINGIAPRE/ | | | | o21 | | APRE/5′UTR | |
| 5′UTR)_BGH228 | | | | | | | |
| pSwap_ApoE_hAAT_ | 61 | SERPING1 | 264 | SERPING1c | 238 | SERPING1 | 182 |
| (SERPING1 APRE) | | | | o21 | | APRE | |
| HBB2ml_SERPINGIco21_BG | | | | | | | |
| H228 | | | | | | | |
| pSwap_ApoE_hAAT_HBB2ml_ | 62 | SERPING1 | 264 | SERPING1c | 238 | SAA2 5′UTR | 183 |
| SERPING1co21_BGH228 | | | | o21 | | | |
| (SAA2 5′UTR) | | | | | | | |
| pSwap_ApoE_hAAT_HBB2ml_ | 63 | SERPING1 | 264 | SERPING1c | 238 | SAA2 3′ UTR | 184 |
| SERPING1co21_BGH228 | | | | o21 | | | |
| (SAA2 3′ UTR) | | | | | | | |
| pSwap_ApoE_hAAT_HBB2ml_ | 64 | SERPING1 | 264 | SERPING1c | 238 | SAA2 5′&3′ | 183, |
| SERPING1co21_BGH228 | | | | o21 | | UTR | 184 |
| (SAA2 5′&3′ UTR) | | | | | | | |
| pSwap_ApoE_hAAT(APRE- | 65 | SERPING1 | 264 | SERPING1c | 238 | SAA2 5′ | 183 |
| SAA2)_HBB2ml_(SAA2 | | | | o21 | | | |
| 5′)SERPING1co21_BGH228 | | | | | | | |
| pSwap_(2xADRES) | 66 | SERPING1 | 264 | SERPING1c | 238 | none | |
| ApoE_hAAT_HBB2ml_ | | | | o21 | | | |
| SERPING1co21_BGH228 | | | | | | | |
| pSwap_ApoE_hAAT_HBB2ml_ | 67 | SERPING1 | 264 | SERPING1c | 238 | RIDD1x | 185 |
| SERPING1co21(+RIDD1x)_BG | | | | o21 | | | |
| H228 | | | | | | | |
| pSwap_ApoE_hAAT_HBB2ml_ | 68 | SERPING1 | 264 | SERPING1c | 238 | RIDD3x | 186 |
| SERPING1co21(+RIDD3x)_BG | | | | o21 | | | |
| H228 | | | | | | | |
| pSwap_ApoE_hAAT_HBB2ml_ | 69 | SERPING1 | 264 | SERPING1c | 238 | RIDD1xBlos | 187 |
| SERPING1co21(+RIDD1xBlos) | | | | o21 | | | |
| BGH228 | | | | | | | |

pAAV = 5′ ITR SEQ ID NO: 70, 3′ ITR SEQ ID NO: 71; pSWAP = 5′ ITR SEQ ID NO: 72, 3′ ITR SEQ ID NO: 73; ApoE = SEQ ID NOs: 225, 74-76; SSA2 APRE = SEQ ID NO: 77; 2x ADRES = SEQ ID NO: 78; hAAT = SEQ ID NO: 79; hAATv18 = SEQ ID NO: 80; HBB2ml = SEQ ID NO: 81; HBB2ml(-ATG) = SEQ ID NO: 82; BGH228 = SEQ ID NO: 83.

Expression cassettes are packaged in an AAV viral particle by being encapsidated in an AAV capsid, e.g., AAV-4-1 capsid variant, described in International Patent Application publication WO 2016/210170, the contents of which are incorporated by reference herein in their entirety, or LK03 capsid variant, described in U.S. Pat. No. 9,169,299, the contents of which are incorporated by reference herein in their entirety. Viral particles are generally produced using the triple transfection protocol well-known in the art.

### Example 2—Assessment of Vector Potency of SERPING1-Expressing Vectors Encapsidated in AAV in C57BL/6J Mice at 2 Doses

### Objective

In an effort to develop a SERPING1 transgene with improved secretion from the liver, the endogenous SERPING1 signal peptide was replaced with a panel of naturally occurring and synthetic peptides that were selected from initial in vitro screening studies. The naturally occurring signal peptides corresponded to those of alpha-2-HS-glycoprotein (AHSG) and chymotrypsinogen B2 (sp7); the synthetic peptides, referred to as Synthetic 1 (Syn1) and Synthetic 4 (Syn4), were derived through rational design. A non-GLP study was performed in C57BL/6J mice to evaluate the potency of 5 SERPING1-expressing vectors with different signal peptide sequences at two doses via intravenous injection (Vector SEQ ID NOs: 1, 13, 16, 12, and 2).

The study included 100 male C57BL/6J wild-type mice aged 11-12 weeks. Four candidate cassettes containing heterologous signal peptides were benchmarked against the native human SERPING1 signal peptide. All expression cassettes contained the native SERPING1 cDNA sequence. Other regulatory elements of the cassette that were common for the 5 vectors included an apolipoprotein E hepatic control region 1 (ApoE HCR-1) enhancer, a human alpha-1 antitrypsin (hAAT) promoter, a modified human hemoglobin R (HBB)-derived synthetic intron (HBB2), and a CpG-reduced bovine growth hormone (bGH) polyadenylation (polyA) signal sequence. All cassettes were packaged in a bioengineered AAV capsid. The vectors utilized in this study are detailed below in Table 2. Mice (n=10-11/group) were injected with a low (1.0×10¹² vg/kg) or high (4.0×10¹² vg/kg) dose of the specified vector (Table 3). Animals were monitored over the course of either 18 weeks for Groups 7-11 (low dose cohort) or 28 weeks for Groups 1-5 (high dose cohort). Human C1-INH antigen levels, C1-INH activity, and liver transduction were assessed as detailed in Table 4.

TABLE 2
Characteristics of 5 vectors evaluated in this study

| | Vector SEQ | signal | sp length |
|---|---|---|---|
| SERPING1 sequence | ID NO | peptide (sp) | (bp) |
| Native SERPING1 | 1 | SERPING1 | 66 |
| cDNA SEQ ID NO: 104; | 13 | Syn1 | 63 |
| without signal peptide | 16 | Syn4 | 66 |
| coding sequence (SEQ ID | 12 | AHSG | 54 |
| NO: 232) | 2 | sp7 | 54 |

TABLE 3
Group designation and dose level

| Dose (vg/kg)ª | Vector SEQ ID NO | Group | No. of Males |
|---|---|---|---|
| 4.0 × 10¹² | 1 | 1 | 10 |
| | 13 | 2 | 10 |
| | 16 | 3 | 10 |
| | 12 | 4 | 10 |
| | 2 | 5 | 10 |
| 1.0 × 10¹² | 1 | 7 | 10 |
| | 13 | 8 | 10 |
| | 16 | 9 | 11 |
| | 12 | 10 | 10 |
| | 2 | 11 | 11 |

^{a}The vector dose was estimated based on the assumed mouse body weight of 25 g.

TABLE 4
Analyses performed

| Analysis | Parameter | Assay | Timepoints |
|---|---|---|---|
| Circulating | Human C1-INH | ELISA | Kit-based: Weeks 3, 6, |
| Human | Antigen in Plasma | (Kit-based | 12, 14, 16, 18, 20, 22, 25, |
| C1-INH | | and custom) | and 27 |
| | | | Custom: Weeks 8, 28 |
| Functional | Human C1-INH | Enzymatic | Weeks 22, 25, 27 |
| Human | Activity in Plasma | | (high dose cohort only) |
| C1-INH | | | |
| Liver | Vector Genome | qPCR | Terminusª |
| Transduction | Concentration | | |
| | in Liver | | |

^{a}Terminus was defined as Week 18 for the low dose cohort (1.0 × 10¹² vg/kg, Groups 7-11), and Week 28 for the high dose cohort (4.0 × 10¹² vg/kg, Groups 1-5).
ELISA, enzyme-linked immunosorbent assay; qPCR, real-time quantitative polymerase chain reaction.

Results

Human C1-INH Antigen Levels in Mouse Plasma

To assess the production and secretion of protein encoded by the SERPING1 expression cassette, C1-INH antigen levels were assessed in plasma of animals administered one of the 5 vectors at a low (1.0×10¹² vg/kg) or high (4.0×10¹² vg/kg) dose starting at Week 3 until terminus of study, either at Week 18 (low dose cohort, Groups 7-11) or Week 28 (high dose cohort, Groups 1-5).

C1-INH antigen levels were quantified using a kit-based ELISA Kit (Molecular Innovations, #HC1INHKIT-TOT) and also using a custom sandwich-style ELISA capture assay using plates coated with anti-human C1-INH IgG (Affinity Biologicals, #GACINH-AP) and anti-human C1-INH HRP-conjugated IgG (Affinity Biologicals, #GACINH-HRP) for detection.

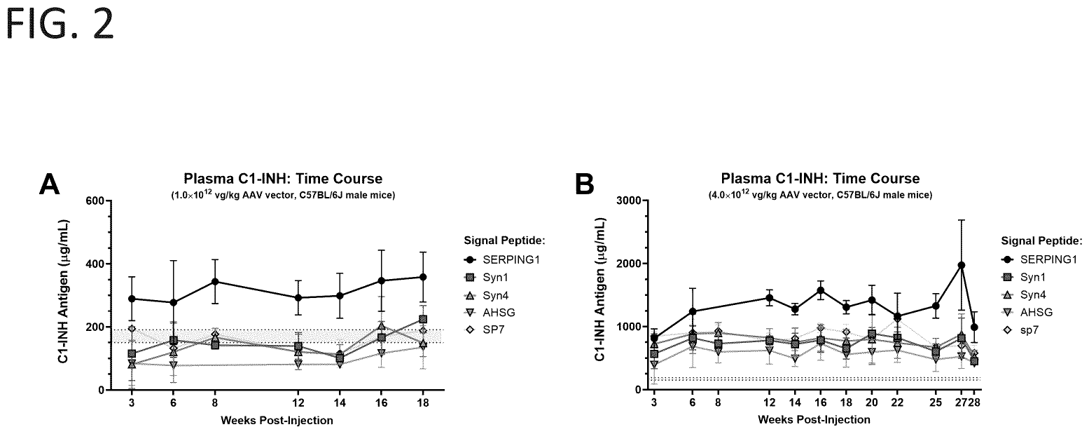

Regardless of dose, mice that received AAV-encapsidated SEQ ID NO: 1 (Group 7, low dose cohort or Group 1, high dose cohort) consistently expressed the highest mean levels of detectable human C1-INH antigen compared with all other groups (FIG. 2). In the low dose cohort, only mice treated with AAV-encapsidated SEQ ID NO:1 had levels of C1-INH consistently greater than pooled normal human plasma. Conversely, administration of all signal peptide variants at the high vector dose resulted in sustained, supraphysiologic levels of plasma C1-INH for at least 28 weeks following vector administration. Over the 6 month experiment, expression levels were 9-fold greater than normal and no morbidity or mortality were observed.

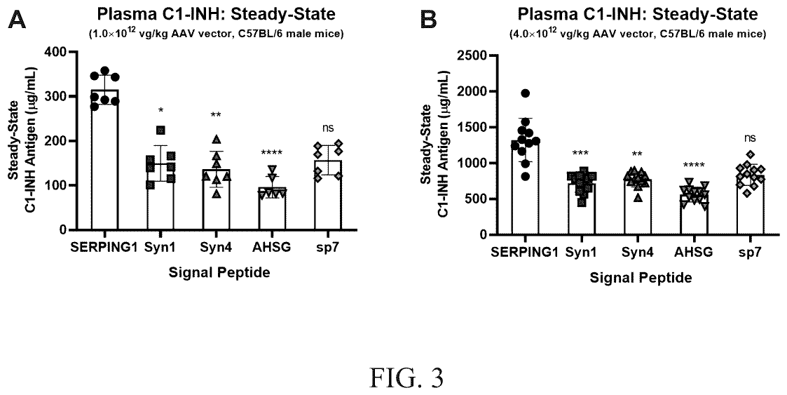

Steady-state C1-INH antigen levels, defined as no significant difference in mean antigen levels across assessed timepoints (linear regression analysis, not shown), were significantly greater in animals that received AAV-encapsidated SEQ ID NO: 1 compared with animals that received AAV-encapsidated SEQ ID NOs: 13, 16, and 12 at both vector doses (FIG. 3). In animals treated with AAV-encapsidated SEQ ID NO: 1 at the low and high dose, mean±SD human C1-INH levels in plasma remained stable at 315.01±32.98 μg/mL and 1321.79±302.97 μg/mL, respectively; these steady-state levels corresponded to a 1.58- to 3.27-fold higher level of plasma C1-INH antigen relative to all other constructs across both dose cohorts.

In summary, the vector cassette containing the native SERPING1 signal peptide (AAV-encapsidated SEQ ID NO: 1) outperformed the other 4 vectors in vivo in terms of C1-INH antigen production and secretion, regardless of dose.

### Activity of Human C1-INH in Mouse Plasma

To assess human C1-INH function in plasma, samples from the high dose cohort (Groups 1-5) were tested for activity via a chromogenic assay at Weeks 22, 25, and 27 using a modified version of the Technochrom C1-INH kit (Diapharma, 5345003). In the assay, plasma C1-INH was titrated against an excess of C1-esterase, and the residual C1-esterase activity was measured. Activity levels were reported as percentage of activity relative to a coagulation reference standard.

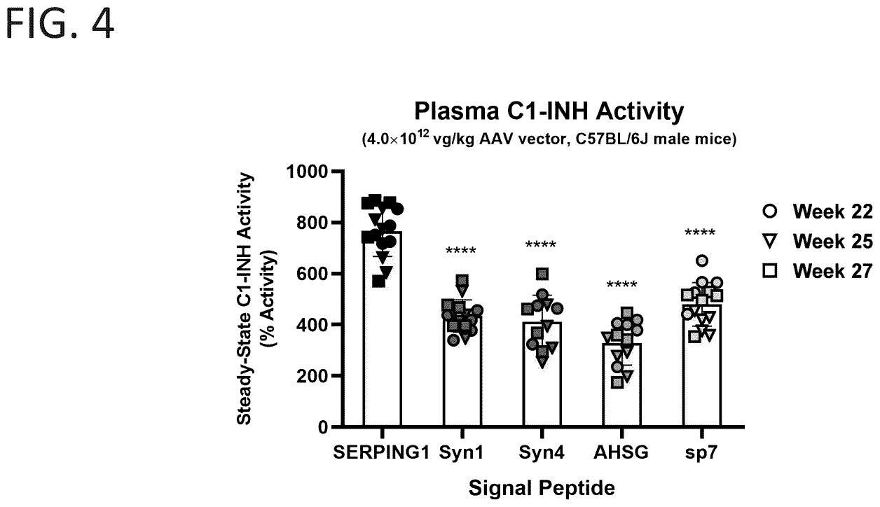

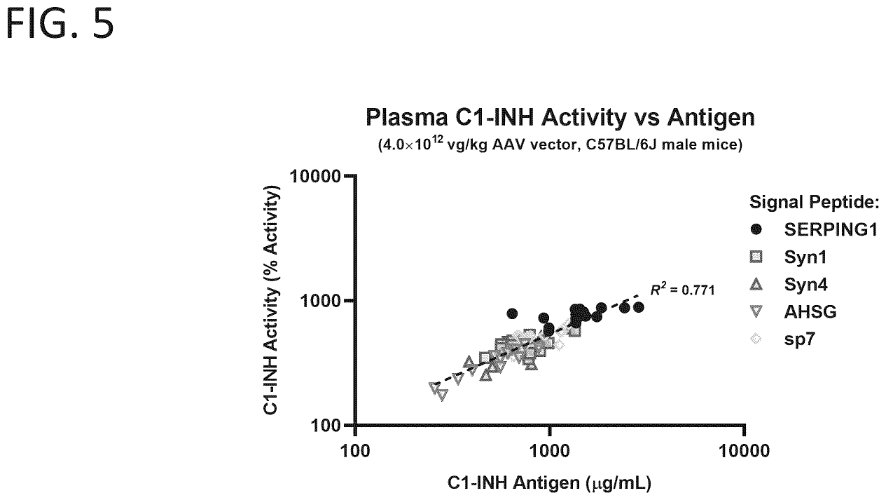

Steady-state human C1-INH activity levels were significantly elevated in animals dosed with AAV-encapsidated SEQ ID NO: 1 compared with the other signal peptide variants (p<0.0001) (FIG. 4). Activity levels in animals dosed with AAV-encapsidated SEQ ID NO: 1 at 4.0×10¹² vg/kg reached a steady-state value of 766.30% activity. Additionally, human C1-INH activity had a positive correlation with antigen levels, suggesting a direct linear relationship and that functional C1-INH was produced (Pearson r=0.8778, p<0.0001, R²=0.771) (FIG. 5).

Normalized Vector Genome Concentration in Liver Tissue-qPCR

To assess target tissue transduction following intravenous AAV vector administration, terminal liver samples were analyzed for vector genome concentration using a real-time quantitative PCR (qPCR) assay. DNA was extracted from terminal liver samples using a modified DNA extraction method with the QIAamp Fast DNA Tissue Kit (Qiagen, Cat #51404).

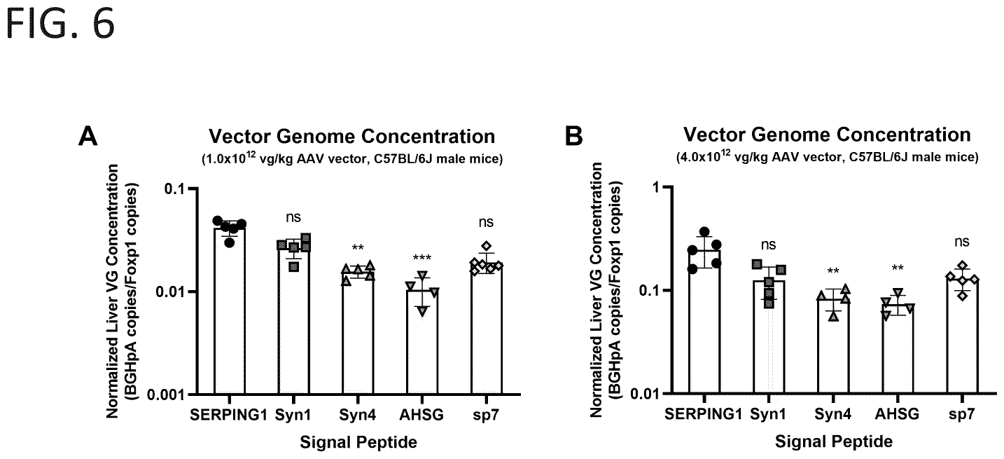

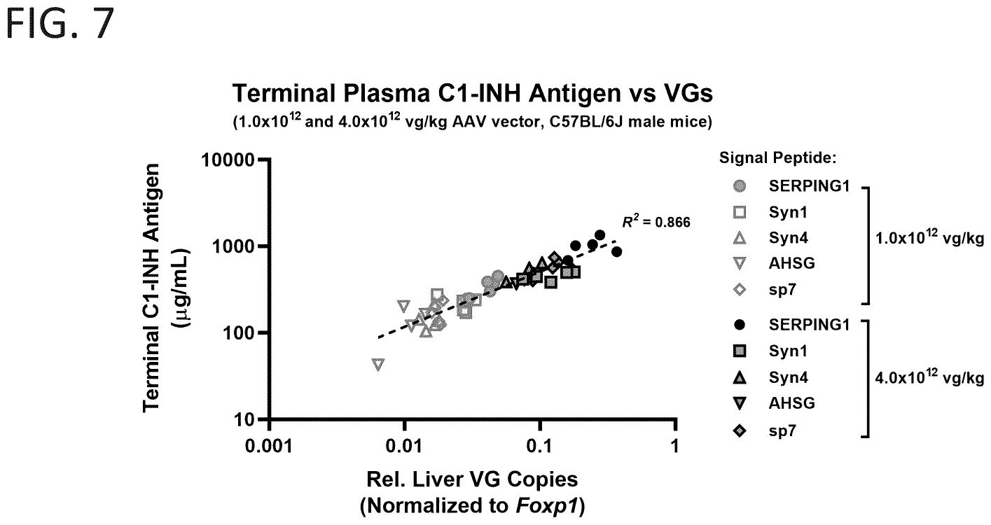

The bGH polyA signal was selected as the AAV target, as it was a component of all candidate expression cassettes. Mouse forkhead box P1 (Foxp1) was targeted to measure genomic DNA concentration between samples and to remove non-biological variation. Final copy numbers for bGH polyA were normalized to Foxp1 and were reported as bGH polyA copies per Foxp1 copies (FIG. 6). As expected, vector genome copies in terminal liver were greater in animals that received the higher vector dose compared with the lower vector dose (FIG. 5). In both dose cohorts, animals administered AAV-encapsidated SEQ ID NO: 1 consistently displayed the highest mean vector genome concentration in the target tissue, with significantly greater levels compared with animals administered AAV-encapsidated SEQ ID NOs: 16 and 12. Additionally, C1-INH antigen levels had a positive correlation with relative vector genome copies across both dose cohorts, demonstrating the expected direct relationship between these two parameters (Pearson r=0.9306, p<0.0001, R²=0.866) (FIG. 7).

### Vector Potency of Signal Peptide Variants

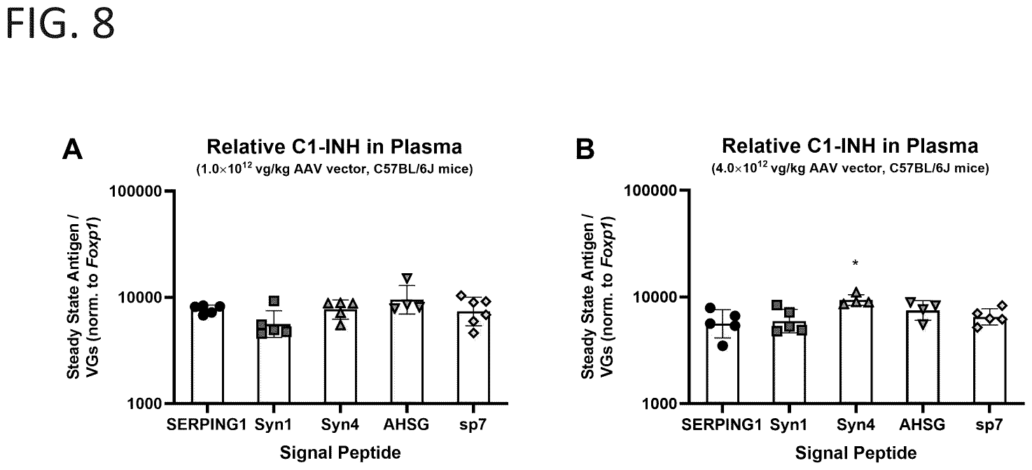

To compare the potency of the signal peptide variants, steady-state C1-INH antigen levels were normalized to terminal liver vector genome copies in both low and high vector dose cohorts (FIG. 8). While no difference in vector potency was observed across animals in the low vector dose groups, animals that received the high vector dose of AAV-encapsidated SEQ ID NO: 16 displayed an increase in normalized steady-state C1-INH antigen levels compared with AAV-encapsidated SEQ ID NO: 1; variability in vector genome copies for AAV-encapsidated SEQ ID NO: 1 may have contributed to the observed difference in potency.

Conclusions

This study evaluated C1-INH antigen levels, C1-INH activity, and vector genome copies derived from 5 candidate expression cassettes containing unique signal peptide encoding sequences. Out of the 5 vectors tested, the vector containing the native human SERPING1 signal peptide-encoding sequence in its transgene cassette (AAV-encapsidated SEQ ID NO: 1; Groups 7 and 1) exhibited the highest steady-state C1-INH antigen, C1-INH activity levels, and terminal vector genome concentration, irrespective of dose. As differences in potency were minimal, the results indicate that AAV-encapsidated SEQ ID NO: 1 had the strongest response compared with vectors containing other signal peptides in the context of the AAV capsid in wild-type mice.

### Example 3—Assessment of Vector Potency of Optimized SERPING1-Expressing Vectors Encapsidated in AAV in C57BL/6J Mice at a Single Dose Objective

Two non-GLP studies (A and B) were performed in C57BL/6J mice to evaluate the vector potency of 13 unique human SERPING1-expressing vectors via intravenous injection. The SERPING1 transgene variants were generated using codon optimization (Integrated DNA Technologies (IDT) Codon Optimization Tool, Codon Harmonizer, and Best Codon scripts) as well as truncation strategies (Table 5). The selected variants previously demonstrated high C1-INH secretion in an in vitro screening study.

The two studies included a total of 80 male C57BL/6J wild-type mice aged 9-10 weeks. Twelve candidate cassettes were evaluated against the native human SERPING1 cDNA sequence. Other regulatory elements of the cassette that were maintained in all vectors included an apolipoprotein E hepatic control region 1 (ApoE HCR-1) enhancer, a human alpha-1 antitrypsin (hAAT) promoter, a modified human hemoglobin R (HBB)-derived synthetic intron (HBB2), and a CpG-reduced bovine growth hormone (bGH) polyadenylation (polyA) signal sequence. All cassettes were packaged in a bioengineered AAV capsid. Five animals per group were injected with a 1.0×10¹² vg/kg dose of the specified vector (Tables 6 and 7) and animals were monitored over the course of either 7 weeks in Study A or 6 weeks in Study B. Human C1-INH antigen, bradykinin antigen, and vector genome concentration were assessed as detailed in Table 8.

TABLE 5
Characteristics of 13 vectors evaluated in studies A and B

| Vector | | | Codon |
|---|---|---|---|
| SEQ | | Transgene | Optimization |
| ID NO | CDNA | Modification | Script |
| 1 | SERPING1 | — | — |
| 18 | I3 | Codon Optimized | IDT |
| 19 | I4 | Codon Optimized | IDT |
| 20 | I7 | Codon Optimized | IDT |
| 21 | I20 | Codon Optimized | IDT |
| 22 | I21 | Codon Optimized | IDT |
| 23 | I24 | Codon Optimized | IDT |
| 17 | I2 | Codon Optimized | IDT |
| 24 | H9 | Codon Optimized | Harmonized |
| 25 | H13 | Codon Optimized | Harmonized |
| 26 | BC2 | Codon Optimized | Best Codon |
| 30 | Δ76 | 76 amino acid truncation | — |
| | | from 5' end | |
| 31 | Δ98 | 98 amino acid truncation | — |
| | | from 5' end | |

TABLE 6
Group designation and dose level for Study A

| | Vector SEQ | No. of Animals | Dose Level | Dose Concentration |
|---|---|---|---|---|
| Group | ID NO | (Male) | (vg/kg) | (vg/mouse) ª |
| 1 | 1 | 5 | 1.0 × 10¹² | 2.5 × 10¹⁰ |
| 2 | 18 | 5 | 1.0 × 10¹² | 2.5 × 10¹⁰ |
| 3 | 19 | 5 | 1.0 × 10¹² | 2.5 × 10¹⁰ |
| 4 | 20 | 5 | 1.0 × 10¹² | 2.5 × 10¹⁰ |
| 5 | 21 | 5 | 1.0 × 10¹² | 2.5 × 10¹⁰ |
| 6 | 22 | 5 | 1.0 × 10¹² | 2.5 × 10¹⁰ |
| 7 | 23 | 5 | 1.0 × 10¹² | 2.5 × 10¹⁰ |
| 8 | Excipient | 5 | n/a | n/a |

^{a} The vg/kg vector dose was estimated based on the assumed mouse body weight of 25 g.

TABLE 7
Group designation and dose level for Study B

| | Vector SEQ | No. of Animals | Dose Level | Dose Concentration |
|---|---|---|---|---|
| Group | ID NO | (Male) | (vg/kg) | (vg/mouse) ^{a} |
| 1 | 1 | 5 | 1.0 × 10¹² | 2.5 × 10¹⁰ |
| 2 | 17 | 5 | 1.0 × 10¹² | 2.5 × 10¹⁰ |
| 3 | 24 | 5 | 1.0 × 10¹² | 2.5 × 10¹⁰ |
| 4 | 25 | 5 | 1.0 × 10¹² | 2.5 × 10¹⁰ |
| 5 | 26 | 5 | 1.0 × 10¹² | 2.5 × 10¹⁰ |
| 6 | 30 | 5 | 1.0 × 10¹² | 2.5 × 10¹⁰ |
| 7 | 31 | 5 | 1.0 × 10¹² | 2.5 × 10¹⁰ |
| 8 | Excipient | 5 | n/a | n/a |

^{a} The vg/kg vector dose was estimated based on the assumed mouse body weight of 25 g.

TABLE 8
Analyses performed

| Analysis | Parameter | Assay | Timepoints |
|---|---|---|---|
| Circulating Human | Human C1-INH | ELISA | Weeks 1, 2, 3, |
| C1-INH in Plasma | Antigen | | 4, 5, 6, 7ª |
| Circulating | Bradykinin Antigen | ELISA | Week 7ª |
| Bradykinin in Plasma | | | |
| Liver Transduction | Vector Genome | qPCR | Terminus^{b} |
| | Concentration in Liver | | |

^{a}Time point was evaluated in Study A only. For circulating bradykinin in plasma, only Groups 4, 6, and 8 from Study A were included in the analysis.
^{b}Terminus was defined as Week 7 for Study A and Week 6 for Study B. ELISA, enzyme-linked immunosorbent assay; qPCR, quantitative polymerase chain reaction.

### Results

### C1-INHAntigen in Plasma

To assess the in vivo production of C1-INH antigen from the modified (codon optimized or truncated) SERPING1 expression cassettes, circulating C1-INH antigen levels were assessed by ELISA (Molecular Innovations, #HC1INHKIT-TOT). C1-INH antigen assessments began 1 week following vector administration and continued weekly until study terminus.

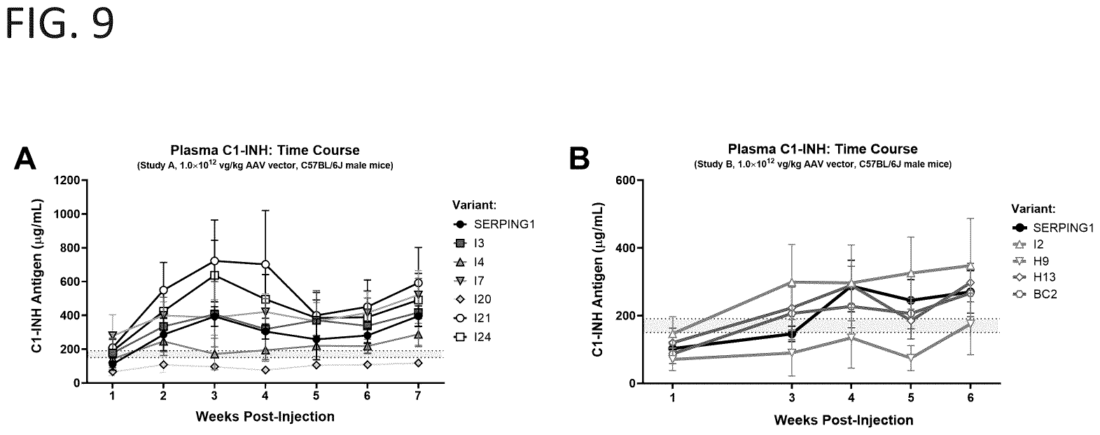

The candidate vectors in Study A generally yielded higher levels of plasma C1-INH antigen compared with Study B and are the focus of Example 3. Following vector administration, levels of mean plasma C1-INH peaked around Week 3 (±1 week) in most groups, followed by a decline (FIG. 9). The highest peak antigen level at Week 3 (±1 week) reached a mean±SD of 722.31±242.03 μg/mL in mice that received AAV-encapsidated SEQ ID NO: 22. Compared with the parental cassette (AAV-encapsidated SEQ ID NO: 1), mice administered AAV-encapsidated SEQ ID NOs: 22, 23, 20, and 18 displayed similar or higher levels of plasma C1-INH throughout the course of the study; the vectors also exceeded mean±SD levels of pooled normal human plasma (179.2±20.88 μg/mL) by Week 2 that persisted for the duration of the study.

In Study A, the relative steady-state (i.e., no significant difference in mean antigen levels across all timepoints tested, one-way ANOVA analysis not shown) C1-INH antigen levels in animals that received either AAV-encapsidated SEQ ID NO: 22 or AAV-encapsidated SEQ ID NO: 23 was significantly greater than in animals that received AAV-encapsidated SEQ ID NO: 1 (1.75-fold and 1.3-fold greater, respectively) (FIG. 9).

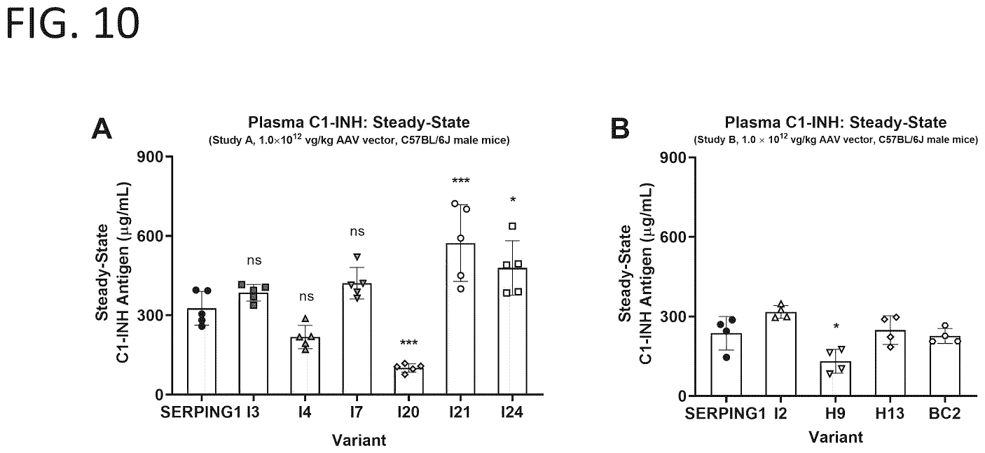

In Study B, steady-state C1-INH antigen levels were similar in animals that received AAV vectors containing codon-optimized SERPING1 variants relative to AAV-encapsidated SEQ ID NO: 1 (FIG. 9). However, animals that received AAV-encapsidated SEQ ID NO: 24 exhibited a 50% reduction in steady-state C1-INH antigen levels compared with AAV-encapsidated SEQ ID NO: 1 (119.89±69.29 μg/mL versus 237.10±55.88 μg/mL, respectively). An initial peak in C1-INH levels was observed around Weeks 3 and 4 in most groups, followed by a decline in antigen levels (FIG. 10). The highest peak antigen levels at Weeks 3 and 4 reached a mean±SD of 299.19±110.92 μg/mL in mice dosed with AAV-encapsidated SEQ ID NO: 17, followed by 289.45±56.12 μg/mL in the AAV-encapsidated SEQ ID NO: 25 group. Both variants expressed higher mean±SD peak levels of C1-INH antigen relative to the parental cassette (287.32±76.21 μg/mL). Similar to Study A, an increase in circulating C1-INH levels was observed in several groups following the initial decline in antigen levels. At study terminus, the highest mean±SD levels of C1-INH antigen were 347.69±139.64 μg/mL in the AAV-encapsidated SEQ ID NO: 17 and 297.57±56.74 μg/mL in the AAV-encapsidated SEQ ID NO: 25 groups. Although peak C1-INH levels in these groups exceeded both the parental cassette and PNP values, the highest antigen levels observed in Study B were less than those displayed by the 3 highest C1-INH-expressing variants in Study A. In animals that received AAV vectors containing truncated SERPING1 variants (Δ76 and Δ98), the levels of C1-INH antigen declined and remained below the limit of quantitation from Week 4 through study end.

Taken together, administration of several codon-optimized, SERPING1-expressing variants resulted in steady-state C1-INH antigen levels 1.13-fold to 1.75-fold greater than the parental cassette across both studies as well as the pooled normal human plasma range. Across both studies, AAV-encapsidated SEQ ID NO: 22, AAV-encapsidated SEQ ID NO: 23, and AAV-encapsidated SEQ ID NO: 20 from Study A displayed the highest levels of plasma C1-INH antigen.

### Bradykinin Levels in Plasma

To evaluate the downstream effect of C1-INH, plasma bradykinin was assessed by ELISA at study terminus (Enzo Life Sciences, product #ADI-900-206, at room temperature). Bradykinin was measured in 2 variants with higher levels of C1-INH antigen, AAV-encapsidated SEQ ID NO: 22 and 20 (Study A), at Week 7.

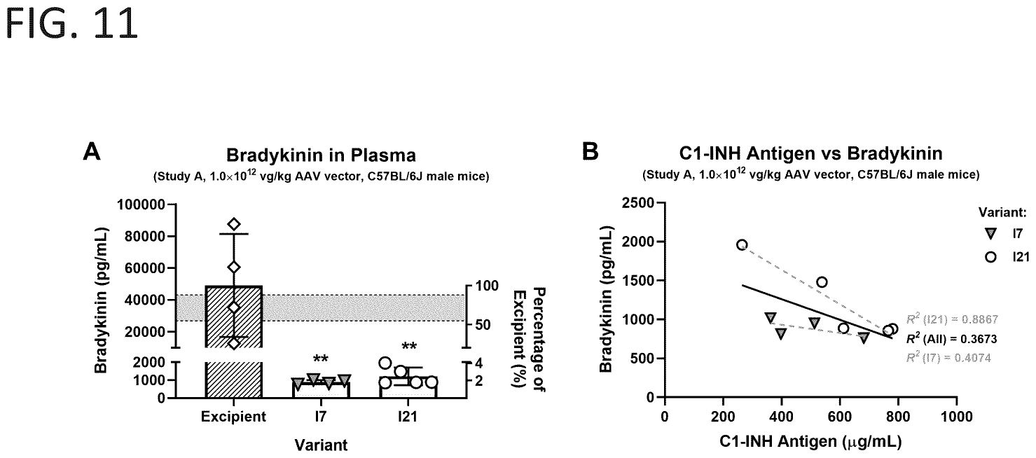

At study terminus, circulating bradykinin levels were significantly reduced in mice administered either AAV-encapsidated SEQ ID NO: 22 or 20, reaching levels less than 3% of plasma bradykinin in excipient-treated mice (FIG. 11). As expected, levels of plasma bradykinin were inversely correlated with plasma C1-INH, with a stronger inverse correlation observed in the AAV-encapsidated SEQ ID NO: 22 group (Pearson r=−0.9417, p<0.0168, R²=0.8867) compared with the AAV-encapsidated SEQ ID NO: 20 group (Pearson r=−0.6383, p=ns, R²=0.4074). Taken together, sustained supraphysiologic levels of human C1-INH were associated with significant reductions in plasma bradykinin in wild-type mice.

### Normalized Vector Genome Concentration in Liver Tissue

To assess target tissue transduction following intravenous AAV vector administration, vector genome concentration in terminal liver samples was evaluated using a real-time quantitative PCR (qPCR) assay. Copy numbers of the shared polyadenylation signal (bGH poly A) were normalized to copies of the mouse Foxp1 gene.

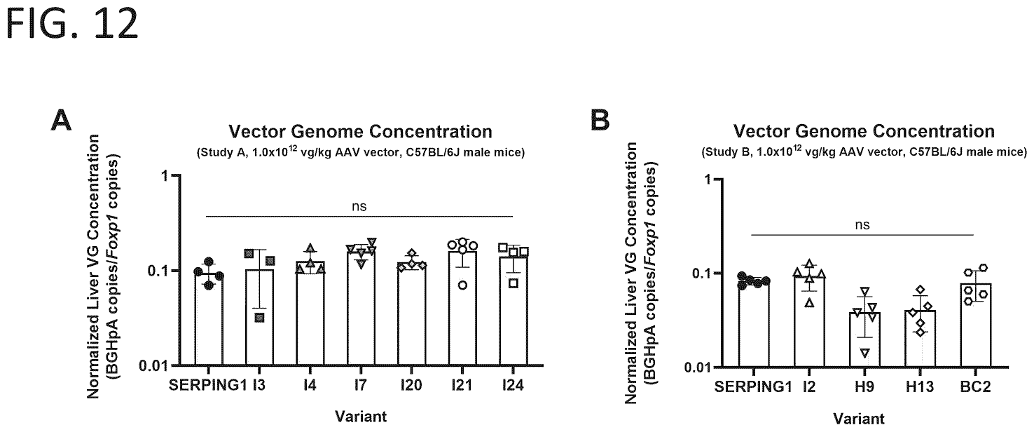

There was no statistical difference in normalized vector genome concentration at terminus among AAV-encapsidated SEQ ID NO: 1 and the six codon-optimized variants in Study A (FIG. 12A).

In Study B, mean terminal liver vector genome copies among AAV-encapsidated SEQ ID NO: 1 and the six codon-optimized or truncated SERPING1 variants ranged from 0.0388 to 0.0936 BGHpA copies per Foxp1 copies in the AAV-encapsidated SEQ ID NO: 24 and 17 groups, respectively. Subsequent to the AAV-encapsidated SEQ ID NO: 17 group, vector genome concentration was highest in animals administered AAV-encapsidated SEQ ID NO: 1 and 26 (0.0829 and 0.0785 BGHpA copies per Foxp1 copies, respectively). There was no statistical difference in normalized vector genome concentration at terminus between all SERPING1-expressing vectors (FIG. 12B). Terminal vector genome concentration in animals that received AAV-encapsidated SEQ ID NO: 30 and 31 (0.0417 and 0.0606, respectively) indicated that liver transduction occurred and was comparable to remaining vectors in the study. AAV encapsidated SEQ ID NO: 30 and 31 provided lower levels of circulating C1EI (data not shown).

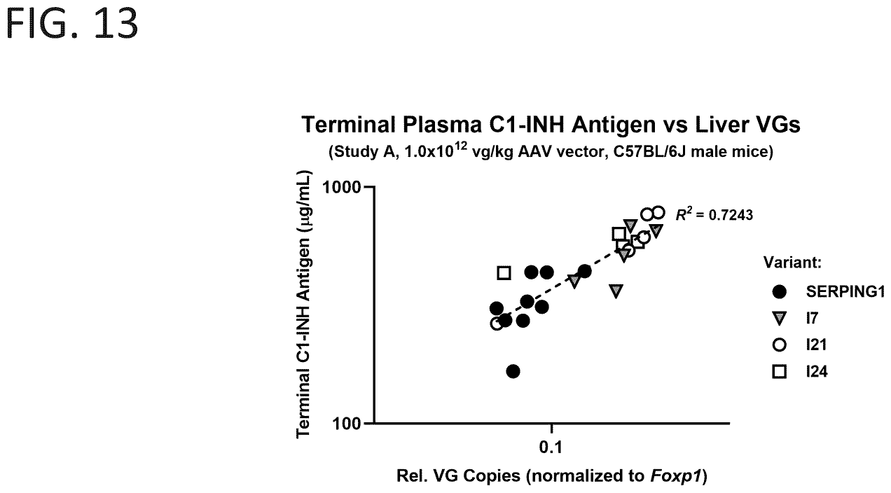

A positive correlation between C1-INH antigen and relative vector genome copies was observed for AAV-encapsidated SEQ ID NO: 1 and the codon-optimized variants with the highest C1-INH antigen expression (SEQ ID NOs: 20, 22, and 23) (Pearson r=0.8510, p<0.0001, R²=0.7243), demonstrating a direct relationship between these two parameters (FIG. 13).

### Vector Potency of Codon Optimized Variants

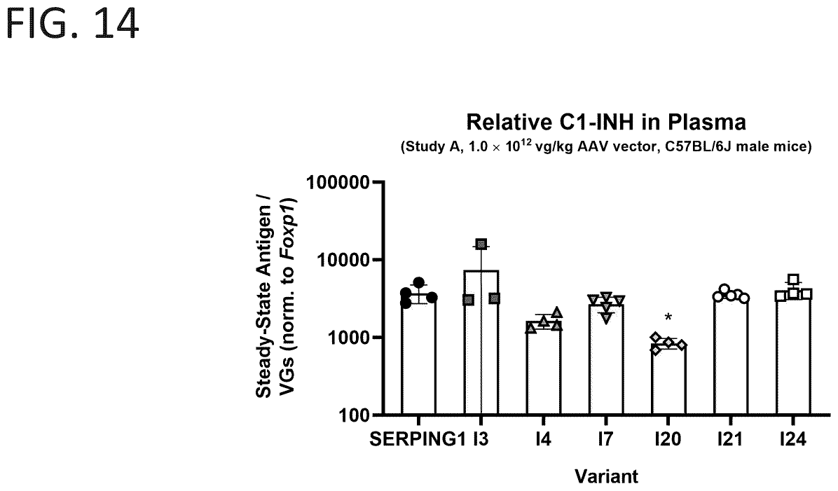

To evaluate the effect of codon-optimization on vector potency, steady-state C1-INH antigen levels were normalized to relative vector genome copies in terminal liver samples (FIG. 14). With the exception of mice that received AAV-encapsidated SEQ ID NO: 21, no differences in vector potency were observed between the parental cassette (AAV-encapsidated SEQ ID NO: 1) and the codon optimized variants in Study A; mice that received AAV-encapsidated SEQ ID NO: 21 showed a reduction in vector potency.

### Conclusions

Taken together, AAV-mediated delivery of codon optimized SERPING1 transgenes resulted in elevated and sustained levels of functional C1-INH in wild-type mice.

### Example 4—Dose Range Assessment of Codon-Optimized SERPING1 Vectors Encapsidated in AAV in Serping1 Knockout Mice

### Objective

A non-GLP study was performed in 129/S5×C57BL/6J-Tyrc-Brd (Serping1^{-/-} or C1-INH knockout or C1-INH null) mice to evaluate the vector potency of a codon-optimized human SERPING1-expressing vector (AAV-encapsidated SEQ ID NO: 20) at three doses via intravenous injection in a disease model.

The current study included 20 male and 19 female C1-INH knockout mice aged 13-18 weeks. Up to five animals per group were injected with one of three doses (1.0×10¹², 4.0×10¹², or 1.0×10¹³ vg/kg) of AAV-encapsidated SEQ ID NO: 20 (Table 9). In addition to the codon-optimized transgene, regulatory elements of the cassette included an apolipoprotein E hepatic control region 1 (ApoE HCR-1) enhancer, a human alpha-1 antitrypsin (hAAT) promoter, a modified human hemoglobin R (HBB)-derived synthetic intron (HBB2), and a CpG-reduced bovine growth hormone (bGH) polyadenylation (polyA) signal sequence. The cassette was packaged in the AAV-4-1 capsid. Animals were monitored over the course of 8 weeks. Plasma C1-INH antigen levels, C1-INH activity, liver vector genome concentration, and SERPING1 mRNA expression were assessed as detailed in Table 10.

TABLE 9
Group designation and dose level

| | | | | Approxi- | | Dose |
|---|---|---|---|---|---|---|
| | | | | mate | | Concen- |
| | | | | Age at | Dose | tration |
| | | Vector | No. of | Dosing | Level | (vg/ |
| Sex | Group | sequence | Animals | (Weeks) | (vg/kg) | mouse) ^{a} |
| Male | 1 | SEQ ID | 3 | 18 | 1.0 × 10¹² | 2.5 × 10¹⁰ |
| | | NO: 20 | | | | |
| | 2 | SEQ ID | 5 | 16 | 4.0 × 10¹² | 1.0 × 10¹¹ |
| | | NO: 20 | | | | |
| | 3 | SEQ ID | 5 | 16 | 1.0 × 10¹³ | 4.0 × 10¹¹ |
| | | NO: 20 | | | | |
| | 4 | Excipient | 1 | 14 | n/a | n/a |
| | 5 | SEQ ID | 2 | 13 | 1.0 × 10¹² | 2.5 × 10¹⁰ |
| | | NO: 20 | | | | |
| | 6 | Excipient | 4 | 13 | n/a | n/a |
| Female | 7 | SEQ ID | 5 | 14-16 | 1.0 × 10¹² | 2.5 × 10¹⁰ |
| | | NO: 20 | | | | |
| | 8 | SEQ ID | 5 | 13-14 | 4.0 × 10¹² | 1.0 × 10¹¹ |
| | | NO: 20 | | | | |
| | 9 | SEQ ID | 5 | 13 | 1.0 × 10¹³ | 4.0 × 10¹¹ |
| | | NO: 20 | | | | |
| | 10 | Excipient | 4 | 13 | n/a | n/a |

^{a} The vg/kg vector dose was estimated based on the assumed mouse body weight of 25 g.
n/a, not applicable.

TABLE 10
Analyses performed

| Analysis | Parameter | Assay | Timepoints |
|---|---|---|---|
| Circulating Human | Human C1-INH | ELISA (Kit- | Kit-based: Weeks |
| C1-INH in Plasma | Antigen | based and | 1, 4, and 5 |
| | | custom) | Custom: Weeks |
| | | | 6, 7, and 8 |
| Functional Human | Human C1-INH | Enzymatic | Weeks 5, 8 |
| C1-INH in Plasma | Activity | | |
| Liver Transduction | Vector Genome | qPCR | Terminusª |
| | Concentration | | |
| AAV Transcription | mRNA Expression | RT-qPCR | Terminusª |
| in Liver | Levels | | |

^{a}Terminus was defined as Week 8.
AAV, adeno-associated virus;
C1-INH, C1 inhibitor;
ELISA, enzyme-linked immunosorbent assay;
qPCR, quantitative polymerase chain reaction;
RT-qPCR, quantitative reverse transcription polymerase chain reaction.

### Results

C1-INHAntigen in Plasma

C1-INH antigen levels were quantified using a kit-based ELISA and custom sandwich-style ELISA capture assay, as described in Example 2.

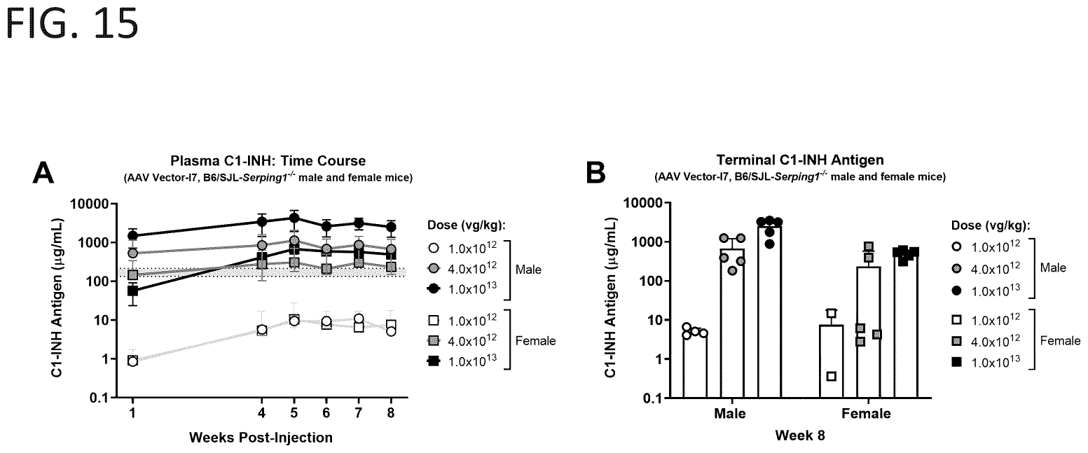

Circulating C1-INH antigen was assessed by ELISA beginning 1-week post-injection and was monitored weekly from Week 4 to Week 8 (study terminus). A dose-dependent increase in mean plasma C1-INH antigen levels was observed in male and female mice from Week 4 to Week 8 (FIG. 15). At the intermediate (4.0×10¹² vg/kg) and high (1.0×10¹³ vg/kg) vector doses, all animals displayed detectable levels of C1-INH by Week 4. Regardless of sex, mice that received the intermediate or high dose vector displayed mean plasma C1-INH levels at or above pooled normal human plasma (PNP; mean±SD=175.4±41.3 μg/mL) by Week 4 that persisted to study terminus. Male mice displayed higher mean antigen levels compared with female mice at the intermediate and high vector doses, which may be attributed to higher hepatic transduction following AAV administration in male mice compared with female mice (Davidoff et al., Blood. 2003; 102(2):480-488).

Taken together, administration of AAV-encapsidated SEQ ID NO: 20 yielded a dose-dependent increase in CT-INH antigen levels, with higher absolute antigen levels observed in males than females.

An orthogonal approach was used to compare methods of human CT-INH antigen detection. At Week 5, C1-INH antigen was assessed by ELISA as well as an additional capillary electrophoretic immunoassay (Wes) in male mice administered the low or intermediate vector dose.

At the low vector dose (1.0×10¹² vg/kg), mean C1-INH values and variation (standard deviation) were comparable across ELISA and Wes approaches, although the relative levels of C1-INH antigen between animals was not consistent between quantification methods. At the intermediate vector dose (4.0×10¹² vg/kg), an increase in C1-INH values was observed using both techniques compared with the lower vector dose. However, mean C1-INH values detected by ELISA were nearly twice the levels quantified by Wes (1063.99 μg/mL vs 547.25 μg/mL); the sample variability using Wes was also greater than ELISA at the intermediate vector dose.

Correlation analysis of C1-INH antigen and activity using two different methods of antigen detection revealed a slightly stronger correlation when C1-INH antigen was assessed by ELISA (R²=0.995) compared with Wes (R²=0.993) (data not shown). As the sample size for this comparison was relatively small (n=9 total samples) and limited by vector dose and animal sex, a larger and more comprehensive sample population may provide further insight on the utility of each antigen detection method.

C1-INHAntigen vs. Activity in Mouse Plasma Correlation

To determine the biological activity of the transgene product encoded by SEQ ID NO: 20, C1-INH activity was assessed by a chromogenic assay in which C1-INH is titrated against an excess of C1-esterase and residual C1-esterase activity is measured. C1-INH activity was assessed in male and female mice of all dose cohorts on Weeks 5 and 8 post-vector administration. C1-INH activity, reported as a percentage of activity relative to a coagulation reference standard, was compared with C1-INH antigen levels.

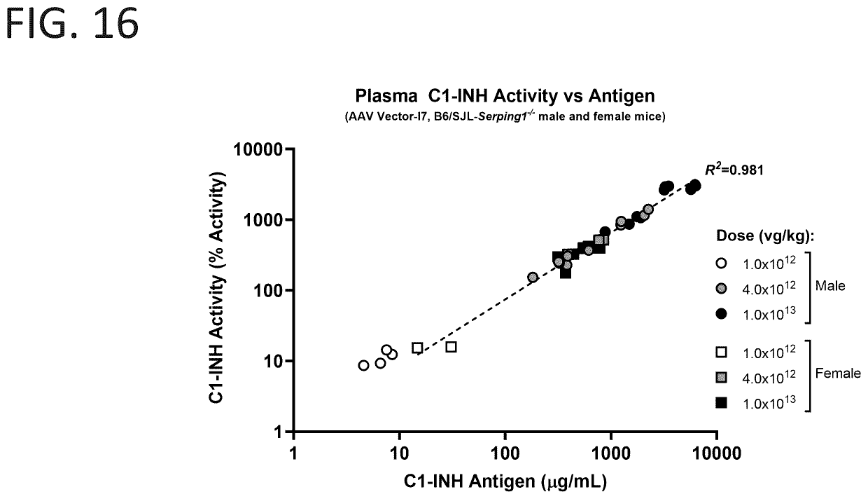

A positive correlation was observed between C1-INH activity and C1-INH antigen across all SEQ ID NO: 20 treatment groups (R²=0.981; FIG. 16). Consistent with antigen levels, male mice that received the high vector dose had the greatest levels of C1-INH activity. In addition, male mice in the high and intermediate vector dose cohorts often displayed higher levels of C1-INH activity compared with dose-matched females.

### Normalized Vector Genome Concentration in Liver Tissue-qPCR

To assess target tissue transduction following vector administration, terminal (Week 8) liver samples were analyzed for vector genome concentration as previously described.

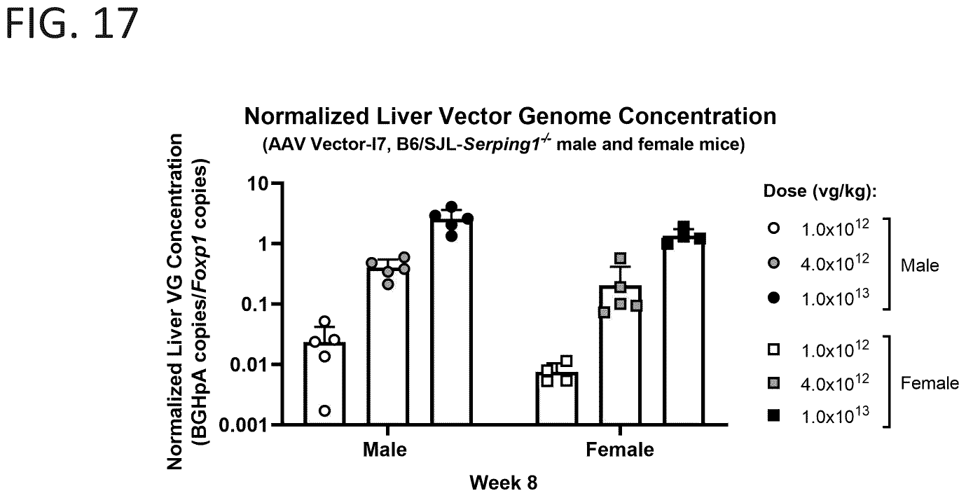

A dose-dependent increase in vector genome concentration was observed in the liver of male and female mice, confirming functional transduction of the target organ (FIG. 17). Higher levels of hepatic transduction were observed in male mice administered AAV-encapsidated SEQ ID NO: 20 compared with dose-matched female mice, consistent with previous reports of sex influencing AAV transduction of the murine liver (Davidoff et al., Blood. 2003; 102(2):480-488). The mean liver vector genome concentrations in male mice administered the high and intermediate dose of AAV-encapsidated SEQ ID NO: 20 were approximately twice the concentration observed in dose-matched females.

Normalized mRNA Expression Levels in Liver Tissue

SERPING1 mRNA expression was quantified using qRT-PCR in terminal (Week 8) liver samples. SERPING1 mRNA was normalized to mouse peptidyl-prolyl cis-trans isomerase E (Ppie) and was reported as SERPING1 copies per Ppie copies.

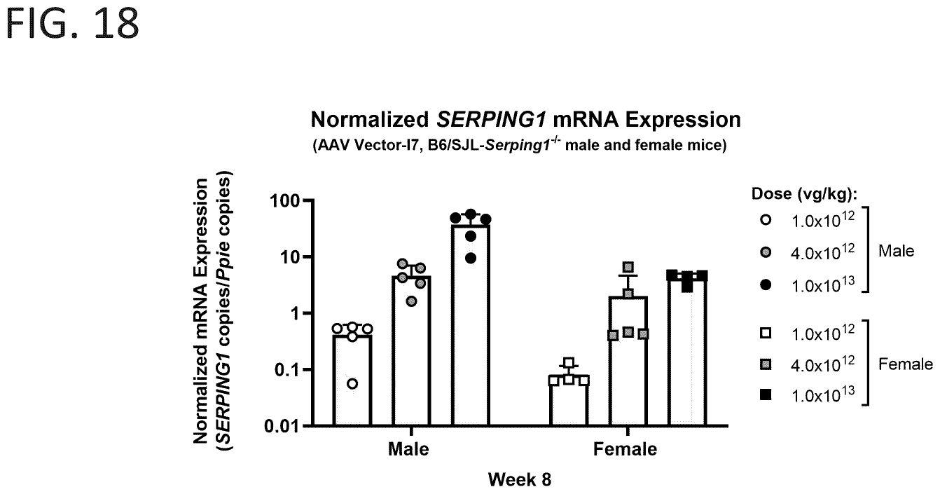

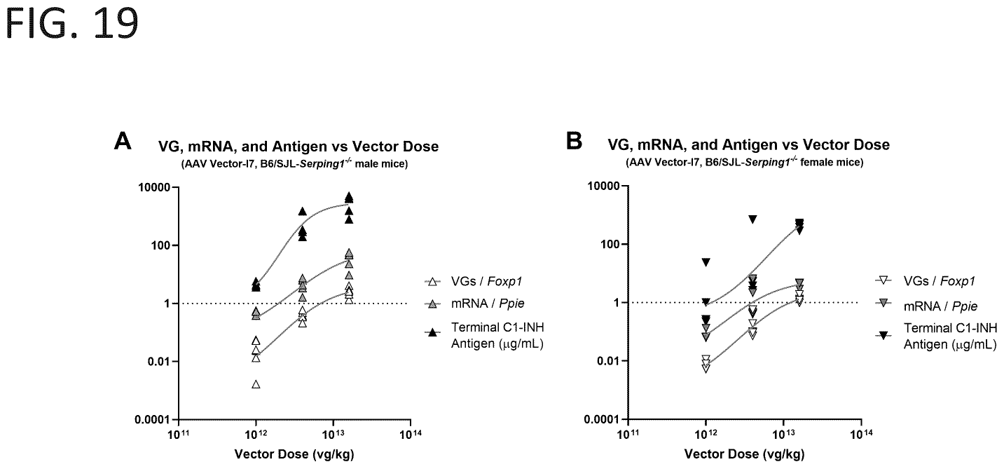

Consistent with vector genome concentration, a dose-dependent increase in transgene expression was observed in male and female mice (FIG. 18). Furthermore, average transgene expression in males was greater than females administered the same vector dose.

A comparison of vector dose with vector are stabilized in the null mice (C1-INH knockout) study.

### Conclusions

Administration of AAV-encapsidated SEQ ID NO: 20 at doses of 4.0×10¹² vg/kg (intermediate dose) and 1.0×10¹³ vg/kg (high dose) were capable of restoring circulating levels of C1-INH antigen to physiologic or supraphysiologic levels in C1-INH knockout male and female mice. Consistent with previous reports in mice, a sex-specific response was observed following AAV-mediated, liver-directed gene delivery.

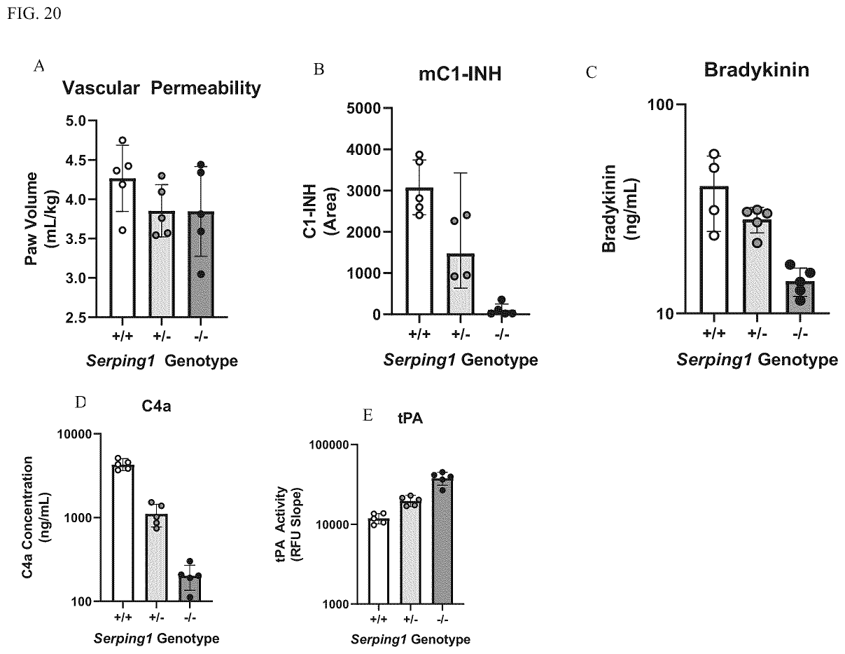

### Example 5—Characterization of Serping1 Deficiency on a B6.SJL Mouse Background (FIG. 20)

Serping1^{-/-} mice on the C57BL/6×SJL background (Molecular Innovations) were used as a model of HAE. In experiments conducted to characterize the commercially available mouse model, no vascular permeability differences were observed between the strain-matched wild-type control and the Serping1^{-/-} mice, as assessed by Evan's blue dye extravasation (data not shown and FIG. 20A) paw volume plethysmography. Clear differences were observed in FIG. 20B) plasma C1-INH, FIG. 20C) bradykinin, FIG. 20D) C4a, and FIG. 20E) tissue plasminogen activator (tPA). While C1-INH, C4a, and tPA followed an expected relationship with C1-INH, bradykinin levels appeared to be increased, rather than decreased, in the presence of endogenous mCI-INH.

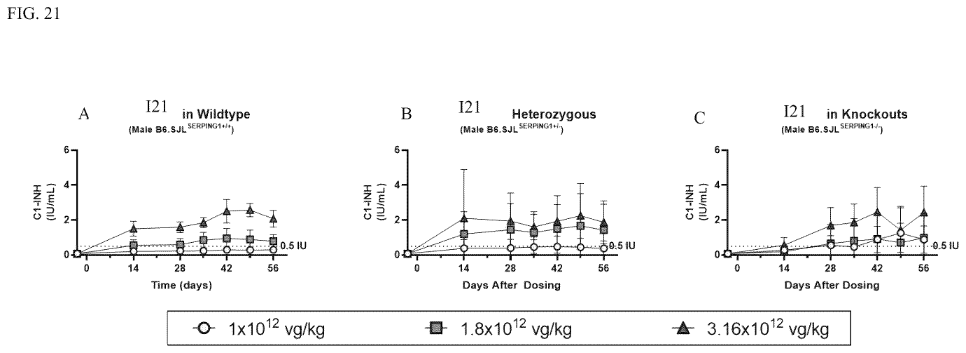

### Example 6—I21 (SEQ ID NO: 22) Drives Dose-Dependent, Durable hC1-INH Antigen Levels in Plasma of HAE Model Mice (FIG. 21)

Plasma hC1-INH levels in FIG. 21A) B6/SJL^{serpng+/+}, FIG. 21B) B6/SJL^{serping1+/-} and FIG. 21C) B6/SJL^{serping-1-/-} male mice were injected with one of three doses of AAV-encapsidated I21 (SEQ ID 22), ranging from 1.0×10¹² to 3.16×10¹² vg/kg in quarter-log increments. These doses were intended to span a potential threshold of functional C1-INH in the presence of varied levels of endogenous mC1-INH, under which it is theoretically possible that constitutive low-level complement activation would result in accelerated C1-INH catabolism. While no overt differences were observed in peak circulating C1-INH, Serping1^{-/-} animals exhibited delayed time-to-threshold kinetics. Differences in peak expression were found to be derived from variation in dosing formulation, rather than driven by genotype (data not shown).

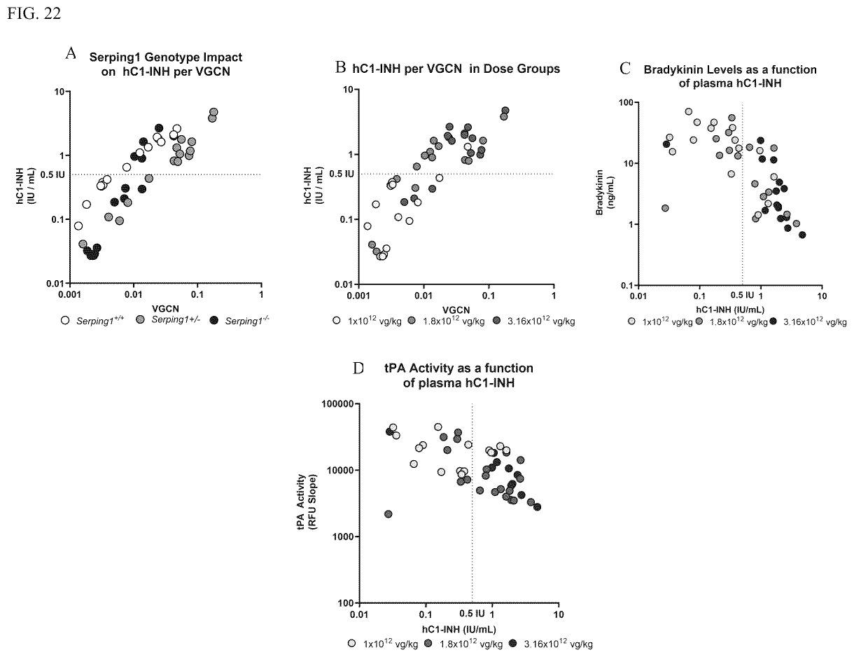

### Example 7—C1-INH Expression and Pharmacodynamics (FIG. 22)

Data comparing hC1-INH expression in different Serping1 genotypes indicated a potential effect of endogenous mC1-INH on hC1-INH dose response (FIG. 22A & FIG. 22B), however a clear relationship was not established. Circulating plasma hC1-INH had a clear and consistent negative correlation with plasma bradykinin (FIG. 22C) and tPA activity (FIG. 22D), indicating functional regulation of elements of the contact system and hemostasis.

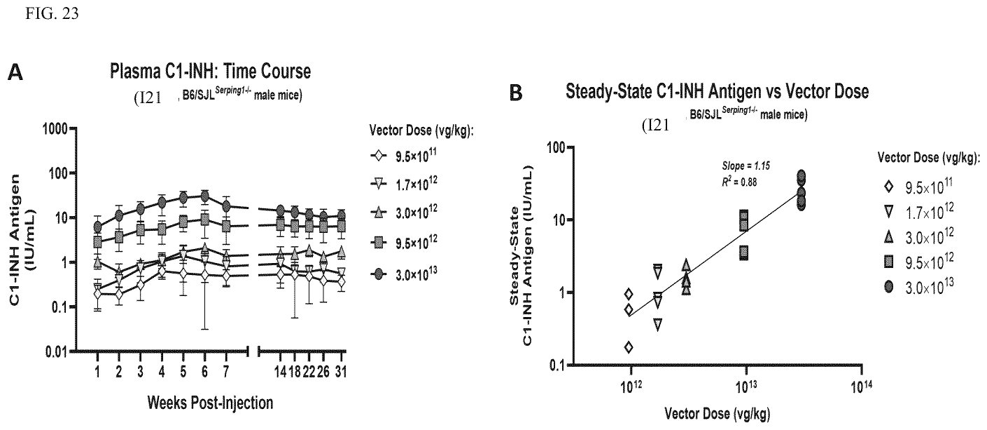

### Example 8—I21 (SEQ ID NO: 22) Drives Dose-Dependent, Durable hC1-INH Antigen Levels in Plasma of HAE Model Mice (FIG. 23)

B6/SJL^{serping1-/-} male mice were injected with one of five doses of I21 (SEQ ID NO 22), ranging from 9.5×10¹¹ to 3.0×10¹³ vg/kg. (FIG. 23A) Plasma C1-INH levels as measured by ELISA as a function of time. Values are in units of IU/mL and presented as mean±SD. (FIG. 23B) Steady-state C1-INH antigen levels in B6/SJL^{serping-/-} male mice as a function of vector dose. All values were log₁₀ transformed and a linear fit was generated using regression analysis (R²=0.88). C1-INH activity as measured by C1-esterase inhibition exhibited a linear dose-response and correlated with antigen levels (data not shown).

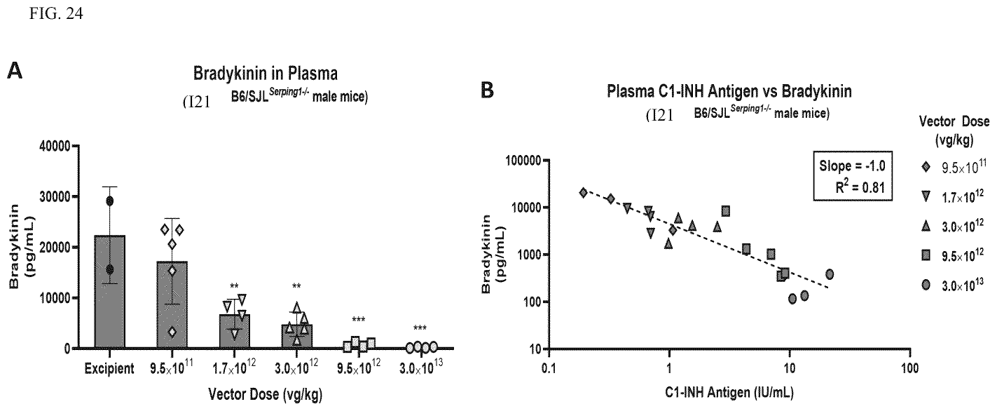

### Example 9-I21 (SEQ ID NO: 22) Mediated Reduction in Bradykinin, the Primary Driver of Angioedemic Attack, in HAE Disease Model Mice (FIG. 24)

FIG. 24A) Individual and mean±SD plasma bradykinin levels in each dose cohort. With the exception of the lowest vector dose cohort, animals that received I21 vector displayed significant reductions in mean plasma bradykinin relative to excipient-treated mice. Statistical comparison relative to excipient was performed by one-way ANOVA with post-hoc Dunnett's multiple comparisons test (**, p<0.01; ***, p<0.001). FIG. 24B). Plasma C1-INH levels were inversely correlated with plasma bradykinin levels and exhibited a linear relationship with respect to C1-INH antigen (R²=0.81).

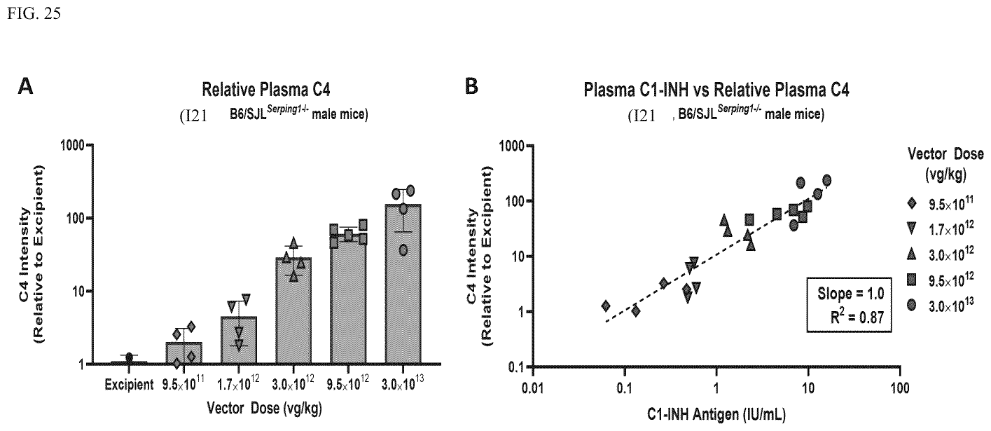

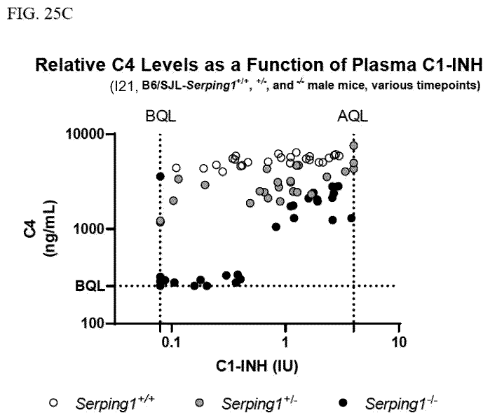

### Example 10—I21 (SEQ ID NO: 22) Mediated C1-INH Rescues C4 Levels in the Mouse Model of HAE (FIG. 25)

Plasma C4 was analyzed at study terminus by an automated capillary-based immunoassay system (Wes™, ProteinSimple). FIG. 25A) Individual and mean±SD relative C4 expression in each dose cohort. FIG. 25B) Individual relative plasma C4 values plotted relative to each animal's respective C1-INH antigen level at Week 31. All values were log₁₀ transformed and a linear fit was generated using regression analysis (R²=0.87). The level of C4 was also examined as a function of plasma C1-INH (FIG. 25C).

### Example 11—Toxicology Study

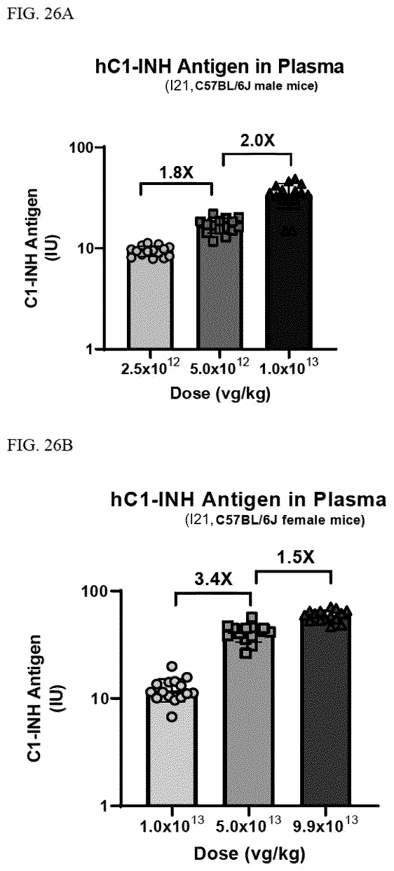

AAV-encapsidated I21 (SEQ ID NO: 22) toxicology was assessed in C57BL/6J male and female mice. Male mice were dosed with AAV-encapsidated I21 at 2.5×10¹² vg/kg, 5.0×10¹² vg/kg, or 1.0×10¹³. Female mice were dosed with AAV-encapsidated I21 at 1.0×10¹³ vg/kg, 5.0×10¹³ vg/kg, or 9.9×10¹³ vg/kg. All dosed mice survived to term (30 days) and no microscopic findings in a histopathological evaluation were considered to be directly related to administration of AAV-encapsidated 121. C1-INH levels in the dosed mice exceeded 100% of normal by >30 to 60 fold (FIG. 26A and FIG. 26B).

### Example 12—Non-Human Primate (NHP) Dose Study

The objectives of this NHP dose estimation study was to assess AAV-encapsidated I21 vector safety and to elucidate a range of doses to achieve therapeutic levels of human C1-INH to inform the starting clinical dose. Three ascending vector doses of 1.0×10¹³ vg/kg, 3.2×10¹³ vg/kg, and 1.0×10¹⁴ vg/kg were aimed to target 20%, 70%, and 200% of normal circulating C1-INH in both male and female cynomolgus macaques.

TABLE 11

| Group No. | | | | |
|---|---|---|---|---|
| 1 | Vehicle | 0 | 6 | 6 |
| 2 | AAV- | 1.0 × 10¹³ | 4 | 4 |
| | encapsidated I21 | (Low) | | |
| 3 | AAV- | 3.2 × 10¹³ (Mid) | 4 | 4 |
| | encapsidated I21 | | | |
| 4 | AAV- | 1.0 × 10¹⁴ | 3 | 3 |
| | encapsidated I21 | (High) | | |

Human C1-INH Antigen Levels in NHP Plasma: Human C1-INH was quantified using a LC-MS/MS method. Plasma-derived human C1-INH (Molecular Innovations, #HC1INH-1.0MG) was used to prepare standards that ranged from 10 μg/mL to 0.1 μg/mL and QC samples targeting 30 and 300 ug/mL following a 50-fold dilution. Samples were diluted up to 5-fold prior to quantification.

Activity of Human C1-INH in NHP Plasma: C1-INH activity was quantified using a modified version of the Technochrom C1-INH kit (Diapharma, 5345003), a chromogenic C1-esterase inhibitor assay in which C1-INH is titrated against an excess of C1-esterase and the residual C1-esterase activity is measured. Provided vials of C1-esterase and substrate were reconstituted with the indicated, on each vial, volume of nuclease-free water, and National Institute for Biological Standards and Control (NIBSC) concentrate (08/256) was reconstituted in cell culture grade water to obtain a stock concentration of 19.2 IU/mL (1920% Normal Activity). The provided Buffer B was placed in a water bath at 37° C. to allow equilibration to the appropriate temperature prior to use. In a 96-well tissue culture plate, samples were diluted 1:10 in provided Buffer A. A standard curve was prepared using NIBSC concentrate (08/256) in Buffer A, ranging from 200% Normal Activity to 17.3% Normal Activity for a total of 8 reference points, with the final point consisting of buffer alone (0% of normal C1-INH activity). A 1:6 dilution of substrate was prepared in Buffer B and QC samples were prepared consecutively in Cyno Normal Pooled Plasma and Buffer A to achieve 75% and 3-0% Normal Activity. 20 μL of Human C1-esterase was added to a fresh 96-well tissue culture plate. 20 μL of standard, QCs and samples were added to all wells containing human C1-esterase in duplicate and the plate was incubated at 37° C. for 5 minutes and 30 seconds, after which, 120 μL of diluted prewarmed substrate was added to each well and the plate was incubated for an additional 20 minutes at 37° C. Following the 20 minute incubation, the plate was immediately read every 54 seconds for 5 minutes on a SpectraMax i3x plate reader (Molecular Devices) set to 37° C. for optical density at 405 nm.

The SoftMax Pro software was used to plot standards using a Log-Log transformed fit and calculations were performed automatically. Reported results are the back-calculated dilutions obtained from interpolation using this standard curve and reported as percentage of normal C1-INH activity following normalization for % normal C1-INH activity obtained in pre-dose Day 1 plasma samples.

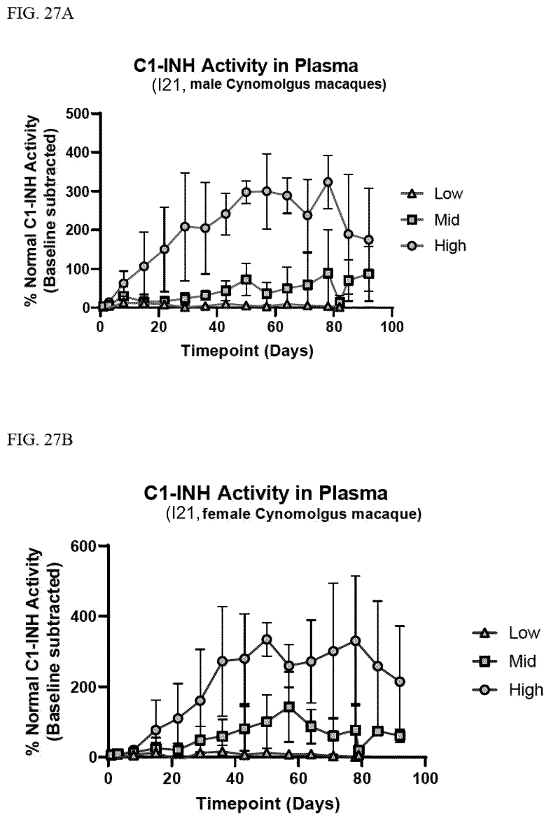

FIG. 27A and FIG. 27B illustrate the results of the dosing study looking at different timepoints. AAV-encapsidated I21 (SEQ ID NO: 22) generated a sustained increase in both hC1-INH antigen and activity. In addition, C1-INH expression spanned the therapeutic range.

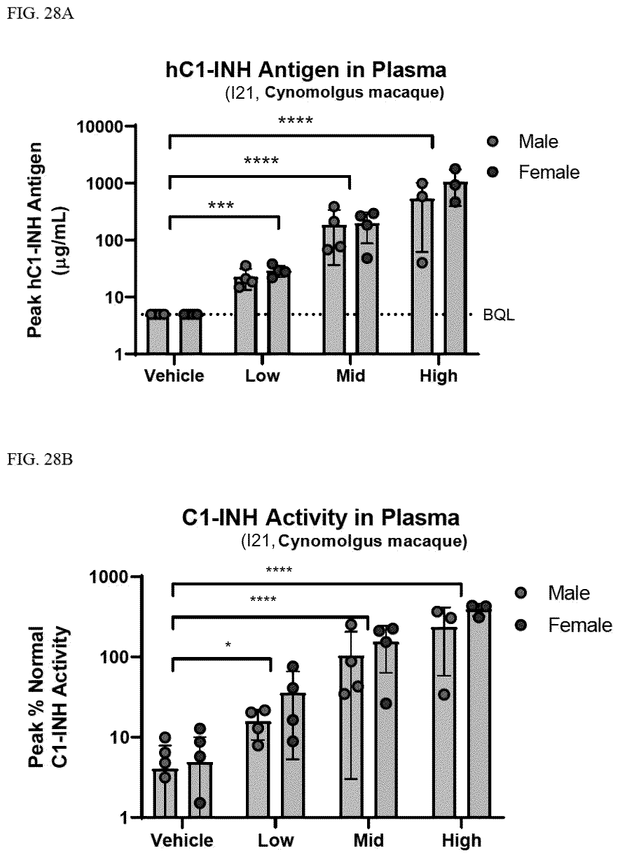

FIG. 28A and FIG. 28B illustrates hC1-INH antigen level and peak percent normal C1-INH activity produced with different doses of AAV-encapsidated 121. Two-way ANOVA of log₁₀-trasnsformed data was performed for both analysis. Antigen and activity correlated well over all doses tested, regardless of sex. No statistical difference was observed in the expression between males and females.

### Example 13—In Vitro hC-INH Expression from Plasmids

Huh7 cells were transfected in vitro with different SERPINGA expression plasmids and hC1-NH antigen levels in supeatant were measured. The different expression plasmids contained the AAV expression cassettes as indicated in Table 12.

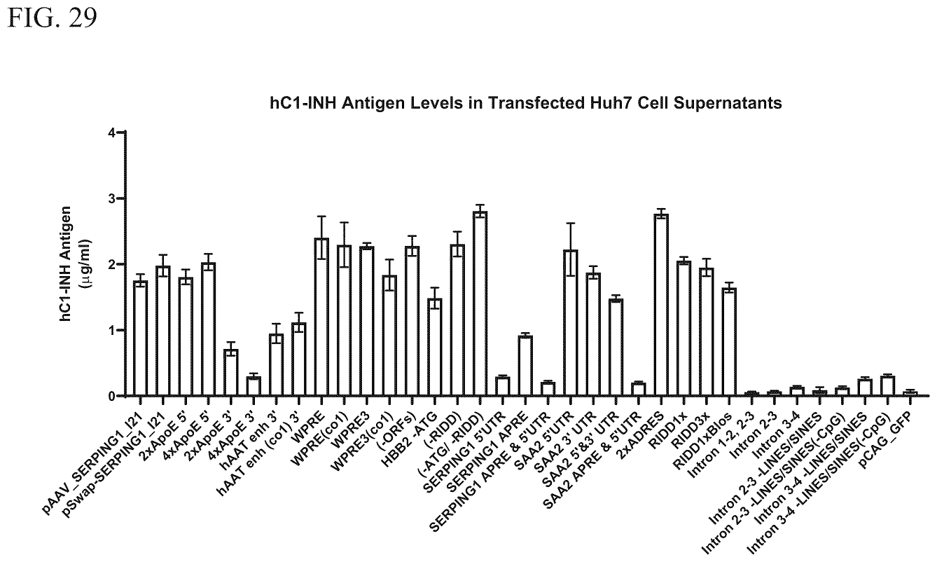

TABLE 12
List of plasmids along with their abbreviations:

| | | FIG. 29 |
|---|---|---|
| SEQ | | Abbreviated |
| ID | | Plasmid |
| NO | | Name |
| 22 | pAAV_ApoE_hAAT_HBB2m1_ | pAAV_ |
| | SERPING1co21_BGH228 | SERPING1_I21 |
| 28 | pSwap ApoE hAAT_HBB2m1_ | pSwap- |
| | SERPING1coI21 BGH228 | SERPING1_I21 |
| 34 | pSwap_2xApoE_hAAT_HBB2m1_ | 2xApoE 5' |
| | SERPING1co21_BGH228 | |
| 35 | pSwap_4xApoE_hAAT_HBB2m1_ | 4xApoE 5′ |
| | SERPING1co21_BGH228 | |
| 36 | pSwap_hAAT_HBB2m1_ | 2xApoE 3′ |
| | SERPING1co21_2xApoE_BGH228 | |
| 37 | pSwap_hAAT_HBB2m1_ | 4xApoE 3′ |
| | SERPING1co21_4xApoE_BGH228 | |
| 38 | pSwap_ApoE_hAAT_HBB2m1_ | hAAT enh 3′ |
| | SERPING1co21_hAATenh_BGH228 | |
| 39 | pSwap_ApoE_hAAT_HBB2m1_ | hAAT enh |
| | SERPING1co21_hAATenh(co1)_BGH228 | (co1) 3′ |
| 42 | pSwap_ApoE_hAAT_HBB2m1_ | WPRE |
| | SERPING1co21_WPRE_BGH228 | |
| 43 | pSwap_ApoE_hAAT_HBB2m1_ | WPRE(co1) |
| | SERPING1co21_WPREco1_BGH228 | |
| 40 | pSwap_ApoE_hAAT_HBB2m1_ | WPRE3 |
| | SERPING1co21_WPRE3_BGH228 | |
| 41 | pSwap_ApoE_hAAT_HBB2m1_ | WPRE3(co1) |
| | SERPING1co21_WPRE3co1_BGH228 | |
| 56 | pAAV_ApoE_hAAT_HBB2m1_ | (-ORFs) |
| | SERPING1co21_BGH228 (-ORFs) | |
| 44 | pSwap_ApoE_hAAT_HBB2m1(-ATG)_ | HBB2-ATG |
| | SERPING1co21_BGH228 | |
| 27 | pAAV_ApoE_hAAT_HBB2m1_ | (-RIDD) |
| | SERPING1coI21(-RIDD)_BGH228 | |
| 229 | pAAV_ApoE_hAAT_HBB2m1(-ATG)_ | (HBB2- |
| | SERPING1coI21(-RIDD)_BGH228 | ATG/-RIDD) |
| 57 | pSwap_ApoE_hAAT_ | SERPING1 5′UTR |
| | SERPING1co21(SERPING1 5′UTR)_BGH228 | |
| 61 | pSwap_ApoE_hAAT_(SERPING1 APRE) | SERPING1 APRE |
| | HBB2m1_SERPING1co21_BGH228 | |
| 60 | pSwap_ApoE_hAAT_SERPING1co21 | SERPING1 |
| | (SERPING1APRE/5′UTR) BGH228 | APRE & 5′UTR |
| 62 | pSwap_ApoE_hAAT_HBB2m1_ | SAA2 5′UTR |
| | SERPING1co21_BGH228 (SAA2 5′UTR) | |
| 63 | pSwap_ApoE_hAAT_HBB2m1_ | SAA2 3′ UTR |
| | SERPING1co21_BGH228 (SAA2 3′UTR) | |
| 64 | pSwap_ApoE_hAAT_HBB2m1_ | SAA2 5′&3′ UTR |
| | SERPING1co21_BGH228 (SAA2 5′&3′UTR) | |
| 65 | pSwap_ApoE_hAAT(APRE-SAA2)_ | SAA2 APRE |
| | HBB2m1_(SAA25′)SERPING1co21_BGH228 | & 5′UTR |
| 66 | pSwap_(2x ADRES)ApoE_hAAT_ | 2xADRES |
| | HBB2m1_SERPING1co21_BGH228 | |
| 67 | pSwap_ApoE_hAAT_HBB2m1_ | RIDD1x |
| | SERPING1co21(+RIDD1x)_BGH228 | |
| 68 | pSwap_ApoE_hAAT_HBB2m1_ | RIDD3x |
| | SERPING1co21(+RIDD3x)_BGH228 | |
| 69 | pSwap_ApoE_hAAT_HBB2m1_ | RIDD1xBlos |
| | SERPING1co21(+RIDD1xBlos)_BGH228 | |
| 45 | pSwap_ApoE_hAAT_SERPING1co21 | Intron 1-2, 2-3 |
| | (Intron 1-2, 2-3)_BGH228 | |
| 46 | pSwap_ApoE_hAAT_HBB2m1_ | Intron 2-3 |
| | SERPING1co21(Intron 2-3)_BGH228 | |
| 47 | pSwap_ApoE_hAAT_HBB2m1_ | Intron 3-4 |
| | SERPING1co21(Intron 3-4)_BGH228 | |
| 58 | pSwap_ApoE_hAAT_HBB2m1_ | Intron 2-3- |
| | SERPING1co21(Intron 2-3)_BGH228- | LINES/SINES |
| | SINES_LINES | |
| 227 | pSwap_ApoE_hAAT_HBB2m1_ | Intron |
| | SERPING1co21(Intron 2-3)_BGH228- | 2-3-LINES/ |
| | SINES_LINES-CpG | SINES(-CpG) |
| 59 | pSwap_ApoE_hAAT_HBB2m1_ | Intron 3-4- |
| | SERPING1co21(Intron 3-4)_BGH228- | LINES/SINES |
| | SINES_LINES | |
| 228 | pSwap_ApoE_hAAT_HBB2m1_ | Intron 3-4-LINES/ |
| | SERPING1co21(Intron 3-4)_BGH228- | SINES(-CpG) |
| | SINES_LINES-CpG | |

The results are shown in FIG. 29. Results are the average of biological replicates n=3. Error bars represent standard deviation. Nearly all plasmids demonstrated increased expression levels of secreted human C1-INH compared to a control plasmid having green fluorescent protein (GFP) driven by CAG (pCAG_GFP), indicating that these plasmids resulted in expression of the SERPING1 transgene product.

Huh7 cells were transfected in vitro with different SERPING1 expression plasmids containing SEQ ID NOs: 49, 50, 51, 52, 53, or 54; and C1-INH antigen levels in the supernatant were undetectable (data not shown).

### Example 14—SERPING1 Expression Plasmid with miR-142-3p Target Site

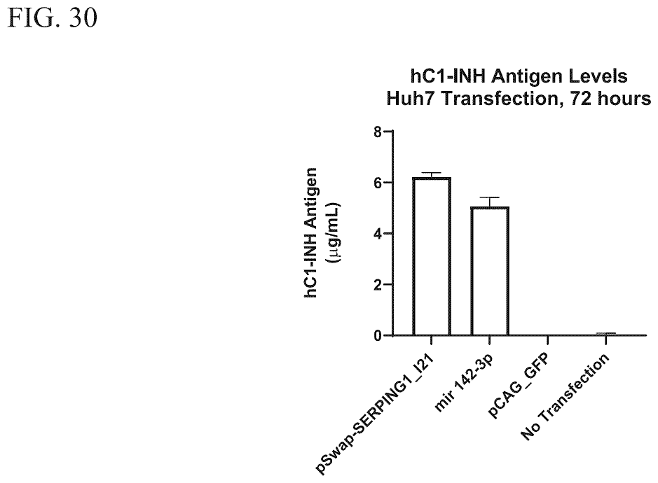

Huh7 cells were transfected in vitro with plasmid containing a nucleic acid of SEQ ID NO: 28 (pSwap-SERPING1_I21), SEQ ID NO: 55 (mir 142-3p), or control plasmid pCAG_GFP; and hC1-INH antigen secreted into the supernatant was assayed. The results are shown in FIG. 30. Results are the average of biological replicates n=3. Error bars represent standard deviation.

The miR-142-3p target site plasmid demonstrated increased expression levels of secreted human C1-INH compared to a green fluorescent protein (pCAG_GFP) control plasmid, indicating that it resulted in expression of the SERPING1 transgene product. In addition, the miR-142-3p plasmid yielded levels of human C1-INH relatively similar to those of pSwap-SERPING1_I21.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

I/We claim:

1. A polynucleotide comprising a nucleic acid encoding a C1 inhibitor, wherein the nucleic acid is CpG-reduced and/or codon optimized compared to the wild-type coding sequence of the C1 inhibitor.

2. The polynucleotide of claim 1, wherein said polynucleotide comprises a nucleic acid sequence at least 85% identical to SEQ ID NO: 238, 236 or 243 and said C1 inhibitor comprises a sequence at least 93% identical to SEQ ID NO: 181.

3. The polynucleotide of claim 2, wherein said polynucleotide comprises a nucleic acid sequence at least 85% identical to SEQ ID NO: 238 said C1 inhibitor comprises a sequence at least 95% identical to SEQ ID NO: 181.

4. The polynucleotide of claim 3, wherein said polynucleotide comprises a nucleic acid sequence at least 95% identical to SEQ ID NO: 238 and said C1 inhibitor comprises a sequence at least 95% identical to SEQ ID NO: 181.

5. The polynucleotide of claim 4, wherein said polynucleotide comprises a nucleic acid sequence at least 97% identical to SEQ ID NO: 238 said C1 inhibitor comprises SEQ ID NO: 181.

6. The polynucleotide of any one of claims 2-5, wherein said polynucleotide further comprises a signal peptide sequence at the 5′ end of said nucleic acid sequence and said signal peptide sequence is at least 95% identical to the nucleic acid of SEQ ID NO: 264.

7. The polynucleotide of claim 1, wherein the nucleic acid is selected from the group consisting of: (1) a polynucleotide having at least 83% sequence identity to the sequence of SEQ ID NO: 105; (2) a polynucleotide having at least 83% sequence identity to the sequence of SEQ ID NO: 106; (3) a polynucleotide having at least 80% sequence identity to the sequence of SEQ ID NO: 107; (4) a polynucleotide having at least 80% sequence identity to the sequence of SEQ ID NO: 108; (5) a polynucleotide having at least 83% sequence identity to the sequence of SEQ ID NO: 109; (6) a polynucleotide having at least 84% sequence identity to the sequence of SEQ ID NO: 110; (7) a polynucleotide having at least 80% sequence identity to the sequence of SEQ ID NO: 111; (8) a polynucleotide having at least 83% sequence identity to the sequence of SEQ ID NO: 112; (9) a polynucleotide having at least 82% sequence identity to the sequence of SEQ ID NO: 113; (10) a polynucleotide having at least 82% sequence identity to the sequence of SEQ ID NO: 114; (11) a polynucleotide having at least 82% sequence identity to the sequence of SEQ ID NO: 115; (12) a polynucleotide having at least 80% sequence identity to the sequence of SEQ ID NO: 116; (13) a polynucleotide having at least 80% sequence identity to the sequence of SEQ ID NO: 117; (14) a polynucleotide having at least 83% sequence identity to the sequence of SEQ ID NO: 118; (15) a polynucleotide having at least 80% sequence identity to the sequence of SEQ ID NO: 119; (16) a polynucleotide having at least 80% sequence identity to the sequence of SEQ ID NO: 120; (17) a polynucleotide having at least 80% sequence identity to the sequence of SEQ ID NO: 121; (18) a polynucleotide having at least 83% sequence identity to the sequence of SEQ ID NO: 122; (19) a polynucleotide having at least 93% sequence identity to the sequence of SEQ ID NO: 123; (20) a polynucleotide having at least 92% sequence identity to the sequence of SEQ ID NO: 124; (21) a polynucleotide having at least 89% sequence identity to the sequence of SEQ ID NO: 125; (22) a polynucleotide having at least 86% sequence identity to the sequence of SEQ ID NO: 126; (23) a polynucleotide having at least 92% sequence identity to the sequence of SEQ ID NO: 127; (24) a polynucleotide having at least 89% sequence identity to the sequence of SEQ ID NO: 128; (25) a polynucleotide having at least 89% sequence identity to the sequence of SEQ ID NO: 129; (26) a polynucleotide having at least 91% sequence identity to the sequence of SEQ ID NO: 130; (27) a polynucleotide having at least 92% sequence identity to the sequence of SEQ ID NO: 131; (28) a polynucleotide having at least 93% sequence identity to the sequence of SEQ ID NO: 132; (29) a polynucleotide having at least 93% sequence identity to the sequence of SEQ ID NO: 133; (30) a polynucleotide having at least 87% sequence identity to the sequence of SEQ ID NO: 134; (31) a polynucleotide having at least 89% sequence identity to the sequence of SEQ ID NO: 135; (32) a polynucleotide having at least 93% sequence identity to the sequence of SEQ ID NO: 136; (33) a polynucleotide having at least 93% sequence identity to the sequence of SEQ ID NO: 137; (34) a polynucleotide having at least 87% sequence identity to the sequence of SEQ ID NO: 138; (35) a polynucleotide having at least 86% sequence identity to the sequence of SEQ ID NO: 139; (36) a polynucleotide having at least 86% sequence identity to the sequence of SEQ ID NO: 140; (37) a polynucleotide having at least 86% sequence identity to the sequence of SEQ ID NO: 141, optionally, the C1 inhibitor comprises the amino acid sequence of SEQ ID NO: 181 or 192.

8. The polynucleotide of claims 1-7, wherein the nucleic acid contains fewer than 24 CpG dinucleotides, optionally 0 CpG dinucleotides.

9. The polynucleotide of claim 1, wherein the nucleic acid has a polynucleotide sequence of any one of SEQ ID NOs: 105-142, 145-147, 156, 171 and 172.

10. A polynucleotide comprising a nucleic acid encoding a variant C1 inhibitor, wherein the nucleic acid is selected from the group consisting of SEQ ID NOs: 143-144, 158, and 165-170, optionally the variant C1 inhibitor comprises an amino acid sequence of any one of SEQ ID NOs: 193-201.

11. A polynucleotide comprising a nucleic acid encoding a C1 inhibitor, wherein the nucleic acid comprises a sequence of SEQ ID NO: 119, and wherein the C1 inhibitor comprises the amino acid sequence of 192.

12. The polynucleotide of any one of claims 1-5 and 7-11, comprising a nucleic acid encoding a signal peptide sequence operably linked to the 5′ end of the polynucleotide sequence encoding the C1 inhibitor or the variant C1 inhibitor.

13. The polynucleotide of claim 12, wherein the signal peptide is selected from the group consisting of C1 inhibitor signal peptide, human chymotrypsinogen B2 signal peptide, ALB signal peptide, ORM1 signal peptide, TF signal peptide, AMBP signal peptide, LAMP1 signal peptide, BTN2A2 signal peptide, CD300 signal peptide, NOTCH2 signal peptide, STRC signal peptide, AHSG signal peptide, SYN1 signal peptide, SYN2 signal peptide, SYN3 signal peptide, and SYN4 signal peptide.

14. The polynucleotide acid of claim 13, wherein the signal peptide has the polynucleotide sequence of one of SEQ ID NOs: 84-103.

15. An expression cassette comprising the polynucleotide of any one of claims 1-14, operably linked to an expression control element.

16. The expression cassette of claim 15, further comprising a polyadenylation sequence operably linked to the 3′ end of the nucleic acid encoding the C1 inhibitor.

17. The expression cassette of claim 15 or 16, wherein the expression control element or polyadenylation sequence is CpG-reduced compared to the wild-type expression control element or polyadenylation sequence.

18. The expression cassette of any one of claims 15-18, wherein the expression control element comprises an ApoE/hAAT enhancer/promoter sequence.

19. The expression cassette of any one of claims 15-18, wherein the polyadenylation sequence comprises a bovine growth hormone (bGH) polyadenylation sequence.

20. The expression cassette of claim 18 or 19, wherein the ApoE/hAAT enhancer/promoter sequence or the bGH polyadenylation sequence is CpG-reduced compared to wild-type ApoE/hAAT enhancer/promoter sequence or bGH polyadenylation sequence.

21. The expression cassette of any one of claims 18-20, wherein the ApoE enhancer sequence comprises the polynucleotide sequence of one of SEQ ID NOs: 225 and 74-76.

22. The expression cassette of any one of claims 18-21, wherein the hAAT promoter sequence comprises the polynucleotide sequence of SEQ ID NOs: 79 or 80.

23. The expression cassette of any one of claims 18-22, wherein the bGH polyadenylation sequence comprises the polynucleotide sequence of SEQ ID NO: 83.

24. The expression cassette of any one of claims 15-23, further comprising an intron.

25. The expression cassette of claim 24, wherein the intron comprises a human hemoglobin R (HBB)-derived intron.

26. The expression cassette of claim 25, wherein the human hemoglobin R (HBB)-derived intron sequence comprises the polynucleotide sequence of SEQ ID NOs: 81 or 82.

27. The expression cassette of any one of claims 15-26, further comprising one or more enhancers.

28. The expression cassette of claim 27, wherein the enhancer is selected from the group consisting of ApoE, 2×ApoE, 4×ApoE, hAAT, WPRE3, and WPRE.

29. The expression cassette of claim 28, wherein the enhancer sequence is codon optimized.

30. The expression cassette of claim 28 or 29, wherein the enhancer comprises the polynucleotide sequence of one of SEQ ID NOs: 225 and 74-78 and 173-178.

31. The expression cassette of any one of claims 15-28, further comprising one or more response elements selected from the group consisting of an miRNA binding site, a regulated Irel-dependent decay (RIDD) sequence, an acute phase response element (APRE), a 5′ UTR, and a 3′ UTR.

32. The expression cassette of claim 31, wherein the expression cassette further comprises an miRNA binding site, optionally the miRNA binding site comprises the polynucleotide sequence of SEQ ID NO: 179.

33. The expression cassette of claim 31, wherein the expression cassette further comprises a RIDD sequence, optionally the RIDD sequence comprises the polynucleotide sequence of one of SEQ ID NOs: 185-187.

34. The expression cassette of claim 31, wherein the expression cassette further comprises an APRE sequence, optionally the APRE sequence comprises the polynucleotide sequence of one of SEQ ID NOs: 77, 78, 180, 182 and 183.

35. The expression cassette of claim 31, wherein the expression cassette further comprises a 3′ UTR, optionally the 3′ UTR comprises the polynucleotide sequence of SEQ ID NO: 184.

36. An adeno-associated virus (AAV) vector comprising the polynucleotide or expression cassette of any one of claims 1-35.

37. The AAV vector of claim 36, wherein the AAV vector comprises: (a) one or more of an AAV capsid, and (b) one or more AAV inverted terminal repeats (ITRs), wherein the AAV ITR(s) flanks the 5′ or 3′ terminus of the polynucleotide or the expression cassette.

38. The AAV vector of claim 37, wherein at least one or more of the ITRs is modified to have reduced CpGs.

39. The AAV vector of any one of claims 36-38, wherein the AAV vector has a capsid serotype comprising a modified or variant AAV VP1, VP2 and/or VP3 capsid having 90% or more sequence identity to AAV1, AAV2, AAV3, AAV4, AAV5, AAV6, AAV7, AAV8, AAV9, AAV10, AAV11, AAV12, Rh10, Rh74 (SEQ ID NO: 188), AAV3B, AAV-2i8, SEQ ID NO: 226, SEQ ID NO: 189, SEQ ID NO: 190, and/or SEQ ID NO: 191; or a capsid having 95% or more sequence identity to AAV1, AAV2, AAV3, AAV4, AAV5, AAV6, AAV7, AAV8, AAV9, AAV10, AAV11, AAV12, Rh10, Rh74 (SEQ ID NO: 188), AAV3B, AAV-2i8, SEQ ID NO: 226, SEQ ID NO: 189, SEQ ID NO: 190, and/or SEQ ID NO: 191; or a capsid having 100% sequence identity to AAV1, AAV2, AAV3, AAV4, AAV5, AAV6, AAV7, AAV8, AAV9, AAV10, AAV11, AAV12, Rh10, Rh74 (SEQ ID NO: 188), AAV3B, AAV-2i8, SEQ ID NO: 226, SEQ ID NO: 189, SEQ ID NO: 190, and/or SEQ ID NO: 191.

40. The AAV vector of any one of claims 36-39, wherein the ITRs comprise one or more ITRs of any of AAV1, AAV2, AAV3, AAV4, AAV5, AAV6, AAV7, AAV8, AAV9, AAV10, AAV11, AAV12, Rh10, Rh74, AAV3B, AAV serotypes, or a combination thereof.

41. An AAV vector comprising,
(1) a 5′ AAV ITR, optionally a 5′ ITR of AAV2, optionally a 5′ ITR comprising the polynucleotide sequence of SEQ ID NO: 70 or 72;
(2) one or more tissue specificity elements, wherein the tissue specificity element is a promoter, optionally the promoter comprises the polynucleotide sequence of SEQ ID NO: 79 or 80;
(3) one or more potency elements, wherein the one or more potency elements are enhancers or polyA sequences, optionally wherein the one or more potency elements have a polynucleotide sequence selected from the group consisting of SEQ ID NOs: 225, 74-76, 83, and 173-178;
(4) one or more response elements, wherein the one or more response elements are miRNA binding sites, regulated Irel-dependent decay (RIDD) sequences, acute phase response elements (APREs), introns, or 5′ or 3′ UTR sequences, optionally wherein the one or more response elements have a polynucleotide sequence selected from the group consisting of SEQ ID NOs: 77-78, 81-82, and 179-187;
(5) a nucleic acid encoding a signal peptide, optionally the signal peptide comprising an amino acid sequence selected from the group consisting of SEQ ID NOs: 203-218, optionally a polynucleotide sequence selected from the group consisting of SEQ ID NOs: 84-103;
(6) a nucleic acid encoding at least one of a C1 inhibitor, a variant C1 inhibitor, and a fusion or combination thereof,
(a) optionally, a C1 inhibitor having the amino acid sequence of SEQ ID NO: 181 or 192, optionally a polynucleotide sequence selected from the group consisting of SEQ ID NOs: 104-142, 145-147, 156, and 171-172;
(b) optionally, a variant C1 inhibitor having an amino acid sequence selected from the group consisting of SEQ ID NOs: 193-201, optionally a polynucleotide selected from the group consisting of SEQ ID NOs: 143, 144, 158, and 165-170; and
(7) a 3′ AAV ITR, optionally a 3′ ITR of AAV2, optionally a 3′ ITR comprising the polynucleotide sequence of SEQ ID NO: 71 or 73.

42. The AAV vector of claim 36 comprising a polynucleotide sequence having at least 99% identity to a sequence selected from the group consisting of SEQ ID NOs: 1-69 and 227-229.

43. The AAV vector of any one of claims 36-42, wherein said polynucleotide is encapsidated by a capsid comprising VP1 of SEQ ID NO: 226.

44. The AAV vector of claim 43, wherein said capsid comprises VP1 of SEQ ID NO: 226, VP2 of SEQ ID NO: 189 and VP3 of SEQ ID NO: 190.

45. The AAV vector of claim 43 or 44, wherein said polynucleotide comprises a nucleic acid sequence at least 99% identical to SEQ ID NO: 22, provided that said nucleic acid sequence encodes the sequence of SEQ ID NO: 192.

46. The AAV vector of claim 43 or 44, where said polynucleotide comprises the nucleic acid sequence of SEQ ID NO: 22.

47. An AAV vector comprising the polynucleotide sequence of SEQ ID NO: 22.

48. A pharmaceutical composition comprising one or a plurality of AAV vectors of any of claims 36-47 in a biologically compatible carrier or excipient.

49. The pharmaceutical composition of claim 48, further comprising empty AAV capsids.

50. The pharmaceutical composition of claim 49, wherein the ratio of said empty AAV capsids to the AAV vector is from about 100:1 to about 50:1, from about 50:1 to about 25:1, from about 25:1 to about 10:1, from about 10:1 to about 1:1, from about 1:1 to about 1:10, from about 1:10 to about 1:25, from about 1:25 to about 1:50, or from about 1:50 to about 1:100.

51. The pharmaceutical composition of any one of claims 48-50, further comprising a surfactant.

52. A method of treating a subject in need of C1 inhibitor, comprising administering to the subject a therapeutically effective amount of the polynucleotide or expression cassette of any one of claims 1-35, or the AAV vector of any one of claims 36-47, or the pharmaceutical composition of any one of claims 48-51, wherein the C1 inhibitor is expressed in the subject.

53. The method of claim 52, wherein the subject has hereditary angioedema (HAE).

54. The method of claim 42 or 53, wherein the polynucleotide, expression cassette, AAV vector, or pharmaceutical composition is administered to the subject intravenously, intraarterially, intra-cavity, intramucosally, or via catheter.

55. The method of any one of claims 52-54, wherein the AAV vector is administered to the subject in a range from about 1×10⁸ to about 1×10¹⁴ vector genomes per kilogram (vg/kg) of the weight of the subject.

56. The method of any one of claims 52-55, wherein said subject is a human and the method reduces, decreases or inhibits one or more symptoms of the need for C1 inhibitor or of HAE; or prevents or reduces progression or worsening of one or more symptoms of the need for C1 inhibitor or of HAE; or stabilizes one or more symptoms of the need for C1 inhibitor or of HAE; or improves one or more symptoms of the need for C1 inhibitor or of HAE.

57. A cell comprising the polynucleotide or expression cassette of any one of claims 1-35.

58. A cell that produces the AAV vector of any one of claims 36-47.

59. A method of producing the AAV vector of any one of claims 36-47, comprising (a) introducing the AAV vector into a packaging helper cell; and (b) culturing the helper cell under conditions to produce the AAV vector.

60. A polypeptide comprising a sequence at least 95%, or 100% to identical to any one of SEQ ID NOs: 215, 216, 217, and 218.

61. A nucleic acid comprising a sequence encoding the polypeptide of claim 60.
